(19) United States
(12) Patent Application Publication
(10) Pub. No.: US 2019/0264184 A1
(43) Pub. Date: Aug. 29, 2019

(54) COMPOSITIONS AND METHODS FOR THE PRODUCTION OF COMPOUNDS

(71) Applicant: Ginkgo Bioworks, Inc., Boston, MA (US)

(72) Inventors: Daniel C. GRAY, Medford, MA (US); Enhu LI, Newton, MA (US); Brian R. BOWMAN, New Rochelle, NY (US); Gregory L. VERDINE, Boston, MA (US); Keith ROBISON, Andover, MA (US); Marc CHEVRETTE, Madison, WI (US); Dan UDWARY, Davis, CA (US); Pam Shouping WANG, Cambridge, MA (US); Anna LI, Cambridge, MA (US); Jay P. MORGENSTERN, Boston, MA (US)

(21) Appl. No.: 16/345,595

(22) PCT Filed: Oct. 27, 2017
(86) PCT No.: PCT/US17/58800

§ 371 Date: Apr. 26, 2019

Related U.S. Application Data
(60) Provisional application No. 62/414,410, filed on Oct. 28, 2016.

(51) Int. Cl. C12N 9/10; C12N 15/76; C12P 17/18; A61P 17/06; A61P 25/00; C12N 15/52; C12N 1/20; C12R 1/485; C12R 1/55

(52) U.S. Cl. CPC C12N 9/1029; C12N 15/76; C12P 17/188; A61P 17/06; C12Y 203/01; C12N 15/52; C12N 1/20; C12R 1/485; C12R 1/55; A61P 25/00

(57) ABSTRACT

The present disclosure provides proteins, nucleic acids, vectors, and host molecules useful for the production of compounds of interest, and methods for their use.

### BACKGROUND

Polyketide natural products are produced biosynthetically by polyketide synthases (PKSs), e.g., type I polyketide synthases, in conjunction with other tailoring enzymes. Polyketide synthases (PKSs) are a family of large, multi-domain proteins whose catalytic functions are organized into modules to produce polyketides. The basic functional unit of polyketide synthase clusters is the module, which encodes a 2-carbon extender unit, e.g., derived from malonyl-CoA. The modules generally present in a polyketide synthase include i) a loading module; ii) extending modules; and iii) releasing modules. Within the module, the minimal domain architecture required for polyketide chain extension and elongation includes the ketosynthase (KS), acyl-transferase (AT) and the ACP (acyl-carrier protein) domains, and the specific chemistry of each module is encoded by the AT domain and by the presence of the β-ketone processing domains: ketoreductase (KR), dehydratase (DH), and enoylreductase (ER) domains. Polyketide synthase biosynthesis proceeds by two key mechanisms: polyketide chain elongation with a polyketide synthase extending module and translocation of the polyketide intermediate between modules. Productive chain elongation depends on the concerted function of the numerous catalytic domains both within and between modules.

Combinatorial biosynthesis is a general strategy that has been employed to engineer polyketide synthase (PKS) gene clusters to produce novel drug candidates (Weissman and Leadlay, Nature Reviews Microbiology, 2005). To date, these strategies have relied on engineering PKS domain deletions and/or domain swaps within a module or by swapping an entire module from another cluster to produce a chimeric cluster. The problem with this approach is that protein engineering of the polyketide megasynthases via wholesale domain and/or module replacement, insertion, or deletion can perturb the “assembly line” architecture of the PKS, thus drastically reducing the amount of polyketide synthesized.

### SUMMARY OF THE INVENTION

The present disclosure provides compositions and methods for use in combinatorial biosynthesis of polyketides without a significant loss of compound production by module swapping between polyketide synthase genes. Bioinformatics approaches may be used to predict module interface compatibility and therefore, the likelihood that a heterologous module may be swapped into a PKS gene. The resulting compatibility information may be used to engineer a polyketide synthase with an increased likelihood of functioning in assembly-line polyketide biosynthesis.

Accordingly, in one aspect, the disclosure provides an engineered polyketide synthase that includes one or more heterologous modules with altered enzymatic activity relative to a reference polyketide, wherein the engineered polyketide synthase is capable of producing a polyketide when expressed under conditions suitable to allow expression of a compound by the engineered polyketide synthase and wherein the one or more heterologous modules do not substantially inhibit polyketide translocation during polyketide biosynthesis.

In another aspect, the disclosure provides an engineered polyketide synthase including one or more heterologous modules with altered enzymatic activity relative to a reference polyketide, wherein the engineered polyketide synthase is capable of producing a polyketide when expressed under conditions suitable to allow expression of a compound by the engineered polyketide synthase and wherein the one or more heterologous modules include linking sequences which are compatible to the linking sequences of the modules adjacent thereto.

In another aspect, the disclosure provides an engineered polyketide synthase including one or more heterologous modules with altered enzymatic activity relative to a reference polyketide, wherein the engineered polyketide synthase is capable of producing a polyketide when expressed under conditions suitable to allow expression of a compound by the engineered polyketide synthase and wherein the polyketide expression level of the engineered polyketide synthase is at least 1% (e.g., at least 1%, at least 2%, at least 3%, at least 4%, at least 5%, at least 6%, at least 7%, at least 8%, at least 9%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150%) of the polyketide expression level of the reference polyketide synthase.

In some embodiments, the polyketide expression level of the engineered polyketide synthase is at least 1-10% (e.g. at least 1-10%, at least 11-20%, at least 21-30%, at least 31-40%, at least 41-50%, at least 51-60%, at least 61-70%, at least 71-80%, at least 81-90%, at least 91-100%, at least 101-110%, at least 1111-120%, at least 121-130%, at least 131-140%, at least 141-150%). In some embodiments, the engineered polyketide synthase includes one or more heterologous modules with native linking sequences.

In some embodiments, the engineered polyketide synthase may include one, two, three, or more heterologous modules. In some embodiments in which the engineered polyketide synthase contains multiple heterologous modules, the heterologous modules may be adjacent in the engineered polyketide synthase. In some embodiments in which the polyketide synthase contains multiple heterologous modules, any of the modules may be separated by one or more native modules in the engineered polyketide synthase.

In some embodiments of any of the above described aspects, at least one of the one or more heterologous modules is an elongation module which modifies a β-carbonyl unit in the variable region of the polyketide.

In some embodiments of any of the above described aspects, at least one of the one or more heterologous modules includes a portion having at least 90% identity to any one of SEQ ID NO: 1-174.

In some embodiments of any of the above described aspects, at least one of the one or more heterologous modules includes a portion having the sequence of any one of SEQ ID NO: 1-174.

SEQ ID NO: 1
QPLAIVGMACRLPGGVSSPEDLWRLVESGTDAISGFPVDRGWDVDGLYDPDPDVPGKSYTVEGGFLDAVTGFDAPFF
GISPREALAMDPQQRLVLEASWEAFERAGIEPGSVRGSDTGVFMGAFPGGYGTGADLGGFGMTGGAASVLSGRVSYF
FGLEGPAMTVDTVCSSSLVALHQAGYALRHGECSLALVGGVTVMSTPQTFVEFSRQRGLAADGRCKAFSDDADGTGW
SEGVGVLLVERLSDAQARGHNILAVVRGSAVNQDGASNGLTAPNGPSQQRVIRQALANAGLTGADVDVVEAHGTGTT
LGDPIEAQAVIATYGQDRDQPVLLGSLKSNLGHTQAAAGVSGVIKMVMALQNGVVPRTLHVEEPSRHVDWTAGAVEL
VTENQPWPELGRARRAAVSSFGLSGTNAHVILESAPDQPPAPSTDSPVSAVTAGVVPLPISAKTLPALADLEDRLRT
YLTTTPDTDLPAVASTLATTRSLFEHRAVLLGEDTVTGTAIPDPRVVFVFPGQGWQWQGMGSALLTSSTVFAERMAE
CAAALSEFVDWDLLTVLDDPSVVDRVDVVQPACWAVMISLAAVWQAAGIHPDIVLGHSQGEIAAACLAGAISLPDAA
RIVAQRSQLIAHQLGHGAMASISLPADDIPTTDQVWIAAHNGTSTVIAGDPQAVEAVLATCETRGARVRKINVDYAS
HTPHVEQIRTELLDITTGIEAHTPAVPWLSTTDNTWIDQPLDPTYWYRNLREPVRFGPAIDLLQTQDNNLFIEISAS
PVLLQTMDNAATVATLRRDEDTTHRLLTAFAEAHVHGATINWPTVLDTTTTPVDLPTYPFQRQRYWATSNGHPADLT
PEALLKVVRDSAAMVLGHASADTVPTATAFQELGLDSLTAVELRNSLTKATGLRLPATMAFDYPTPDALAARL
SEQ ID NO: 2
EPLAIVGMACRLPGGVSSPEDLWRLVESGTDAITDFPTDRGWDTDTLFDPDPDTPGKTYTVHGGFLDDVAGFDAPFF
GISPREAVAMDPQQRLVLESSWEAFERAGIQPDSIRGSDTGVFMGAYPDGYGIGADLAGFGVTAGAGSVLSGRVSYF
FGLEGPAMTVDTACSSSLVALHQAAYALRQGECSLALVGGVTVMPSPRTFIEFSRQRGLAADGRSKAFADAADGTGF
SEGVGVLLVERLSDAQAKGHNILALVRSSAVNQDGASNGLTAPNGPSQQRVIQSALAGAGLTSADVDVVEAHGTGTT
LGDPIEAQAVLATYGQDRDRPVLLGSLKSNLGHTQAAAGVSGVIKMVMALQHNTVPATLHVDAPSRHVDWTAGAVRL
ATENQPWPETNRPRRAGVSSFGVSGTNAHVILEQAPAASPVEPVDTTDVVVPLVVSARSSGSLSDQADRLAALVGSP
DAPALTSLADALLTRRTVFSQRAVVVAGSHEQAAAGLRALAAGDSHPALVTGAAGPARVVLVFPGQGSQWAGMGAEL
LDASPVFAARIAECAEALRPWVDWSLDEVLRGDASADVLGRVDVVQPASFAVMVGLAAVWESAGVRPDAVLGHSQGE
IAAAYVAGALSLTDAAKIVAVRSRLIAARLGRGGMASVALAPEEAAKLGRTELAAVNSPASVVIAGDAEALDETLAM
LEGEGVRVRRVAVDYASHTPHVEELEQSMAEALADVRSRQPRVRFLSTVTGDWVTEAGALDGGYWYRNLRQPVRFGP
AVASLAEAGYTVFVEASAHPVLVQPVAETLDRTDAVVTGTLRRQDGGLPRLLTSMAELFVGGVPVNWPVLLPAGAVR
GWVDLPTYAFDHQRYWLENRELTPEALLKLVCGRAAAVLGHVDADAVPVAAAFRDLGVDSLTAVELRNSLAKATGLR
LPATLVFDYPTPTVLAGRL
SEQ ID NO: 3
EPLAIVGMACRLPGGVLSPEDLWRLVESGGDAISGFPVDRGWDVENLFDPDPDAAGRTYAVRGGFLDGAAGFDASFF
GISPREAQAMDPQQRLVLEVSWEAFERAGIEPGSVRGSDTGVFMGAYPGGYGMGTDLGGFGMTSVAVSVLAGRVSYF
FGLEGPAMTVDTACSSSLVALHQAGSALRQGECSLALVGGVTVMPTPQTFVEFSRQRGLAADGRCKAFADAADGTGF
SEGVGVLLVERLSDAQARGHNILAVVRGSAVNQDGASNGLTAPNGPSQQRVIQSALAGAGLTSADVDVVEAHGTGTT
LGDPIEAQAVIATYGQDRDQPVLLGSLKSNLGHTQAAAGVSGVIKMVMALQNGVVPRTLHIDEPSRHIDWTAGAVEL
VTENQSWPETGRDRRAAVSSFGISGTNAHVILESAPAQPVPPVDTPVSDVTAGVVPLPISARTVPALADLEDRLRAY
LTTTPETDLPAVASTLAMTRSVFEHRAVLLGEETVTGIAVSDPRVVFVFSGQGSQRVGMGEELAAAFPLFARLHRQV
WDLLDVPDLEVDDTGYVQPALFALQVALFGLLESWGVRPQAVLGHSVGEVAAGYVAGVWSLEDACTLVSARARLMQA
LPAGGAMVAVPVSEEQARAVLVDGVEIAAVNGPASVVLSGDESAVLRVAEGLGRWTRLSASHAFHSVRMEPMLEEFR
QVASELTYREPRIVMAAGEQVTTPEYWVRQVRDTVRFGDQVAAFGDAVFLEIGPDRTLSRLIDGIAMLDGDDEVRAA
VAALAVMHVQGVGVDWPAILGTTTGRVLDLPTYAFQHERYWMVIQELSPEALLKIVRDSAAMVLGHANADTVPTATA
FQELGLDSLTAVELRNSLTKATGLRLPATMAFDYPTPAALAGRL
SEQ ID NO: 4
EPLAIVGMACRLPGGVSTPEDLWRLVESGTDAITDFPTDRGWDTDDLFDPDPDTPGKTYTVHGGFLDDVAGFDASFF
GISPREALAMDSQQRLVLEASWEAFERAGIEPGSVRGSDTGVFMGAYPDGYGIGVDLGGFGATAGAGSVLSGRLSYF
FGLEGPAMTVDTACSSSLVALHQAGSALRQGECSLALVGGVTVIANPQIFVEFSRQRGLAADGRCKAFADNADGTGF
SEGVGVLLVERLSDAQARGHNILALVRSSAVNQDGASNGLTAPNGPSQQRVIRQALANAGLTGAEVDVVEAHGTGTT
LGDPIEAQAVLATYGQDRDQPVLLGSLKSNLGHTQAAAGVSGVIKMVMALRHDTVPATLHIDEPSRHIDWTAGAVEL
VTENQPWPVLDRPRRAAVSAFGVSGTNAHVILESAPDQPPASATDTPAPAVTAGVVPLPISAKTVPALADLEDRLRA
YLTTTPETDLPAVASTLATTRSLFEHRAVLLGEDTVTGTTIPDPRIVFVFPGQGWQWQGMGSALLTSSTVFAERMAE
CAAALSEFVDWDLLTVLDDPSIVDRVDVVQPACWAVMISLAAVWQAAGIHPDIVLGHSQGEIAAACLAGAISLPDAA
RIVAQRSQLIAHQLGHGAMASISLPADDIPTTDKVWIAAHNGTSTVIAGDPQAVEAILATCETRGARVRKINVDYAS
HTPHVEQIRTELLDITTGIEAHTPTVPWLSTTDNTWIDQPLDPTYWYRNLREPVRFGPAIDLLQTQDNNLFIEISAS
PVLLQTMDNATTVATLRRDEDTTQRLLTAFAEAHVHGATINWPTVLNTTTTPVDLPTYPFQRQRYWATSNDRLNGRT
SVEQHRIMVELVLAHATSVLGHESPDAIAPDRAFKDLGMDSLTAIELRNHLVAETGVRLPATTAFDHPTADDLAKRL
SEQ ID NO: 5
EPIAIVSMSCRAPGGVDSPESLWRLVESGTDAITDFPGDRGWDVAGLYSPDPTGYKTYCVQGGFLDAAADFDAAFFG
ISPREALGMDPQQRLLLETSWEAIERARIDPRSLPGRNVGVYVGGAAQGYGVGAIDQQRDNVITGSSISLLSGRLSY
ALGLEGPGVTVDTACSSSLVALHLACQALRQRECSMALVSGVSVIPTPDVFVEFSRQRGLAADGRCKSFSASADGTI
WAEGVGVLVLERLSEATRLGHRVLAVVRGSAVNSDGASNGLTAPNGVSQQRVIRQALTGAGLTAADVDVVEAHGTGT
KLGDPIEAEAILATYGQDRSTPVCLGSLKSNIGHAMAASGVLAVIKMVEAMRHGLIPRTLHVEEPSPHVDWASGDVA
LLTENQPWPDDAKLRRAGVSSFGLSGTNAHVVLEQYRAPAAPDITTTEHQPLAWTLSARDPKALREQAGRLHAALTE
SPRWRPLDIGYSLATTRSNFAHRAVAVGSDRELLRALSKLADGSAWPALVTATAKDRRVAYLFDGQGSQRPDMGSGL
YERFPAFARAWDRISAEFGKHLDHSLTDVYLGRGDAATADLVDDTLYAQAGLFTMEIALFELLAEWGVRPDFVSGHS
IGETAAAYAAGVLSLEDVTKLIVARGRALRQVPPGAMVALRAGEDEAREFLGRTGAALDLAAVNSPTSVVVSGASEA
VAGFRARWTESGREARTLNVRHAFHSRHVEAVLGEFREVLESLTFRTPALPVVSTVTGRLIEPTELSTSEYWLRQVR
QTVRFHDAVRELSGQGVGTFVEIGPSGALASAGLECLGDEASFHAVQRPGSPGDVCLMTAVAELHAGGTTVDWATVL
AGGRATDLPVYPFQHGSYWLAPARPSAPEEPRTMLELVRLEAAIALSITDPGLIADDSSFLDLGFDSISALRLSNRL
AAVTGLDLPPSLLFDHPTPAELAARLD
SEQ ID NO: 6
EPLAIVGMACRLPGGVSSPDDLWRLVASGTDAISEFPADRGWDVDNLYDPDPDAPGKTYTVLGGFLDGVAGFDASFF
GISPREALAMDPQQRLMLEVSWEAFEHAGIPPRSVRGSDTGVFMGAFPSGYNAGLEEFGMTGDAVSVLSGRVSYFFG
LEGPAITVDTACSSSLVALHQASSALRQGECSLALVGGVTVLATPQTFVEFSRQRGLALDGRSKAFADAADGAGWAE
GVGVLVVERLSDARAKGHQIWGVIRGSAVNQDGASNGLSAPNGPSQQRVIRQALANAGLAPHEVDVVEAHGTGTMLG
DPIEAQAVIATYGQDREQPLLLGSLKSNVGHTQAAAGVSGVIKMVMALQHDTVPATLHVDAPSRHVDWTAGAVELVT
ENRPWPETGRVRRAGVSSFGISGTNAHVILESAPEQPASPPEAVAPVVASDRVPLVISAKTPAALAEMENRLRAYLA
AAPGADPRAVASTLATARSVFEHRAVLLGENTITGTVAGADPRVVFVFPGQGWQQLGMGRALRESSPVFAARMAECA
AALSEFVDWDLFTMLDDPAVIDRIDVLQPACWAVMMSLAAVWQAAGVRPDAVIGHSQGEIAAACVAGALSLRDAARI
VALRSQLLAREMGHGVMAAVALPADDIPLVDGVWIGARNGPSSTVISGTPEAVEVVVAACEERGARVRRITAAVASH
SPLGEKIRTELLGISASIPSRTPVVPWLSTADGIWIEAPLDPAYWWRNLREPVGFGPAVDLLQARGENVFLEMSASP
VLLPAMNDAVTVATLRRDDDTPDRMLTALAEAHAHGVIVDWPRVFGSTTRVLDLPTYAFEHQRYWAVNGRPADLTPE
ALLKLVCGRAAAVLGHVDADAVPVAVAFRDLGVDSLTAVELRNSLAKATGLRLPATLVFDYPTPTVLAGRL
SEQ ID NO: 7
EPLAIVGMACRLPGGVLSPEDLWRLVESGGDAISGFPVDRGWDVENLFDPDPDAAGRTYAVRGGFLDGAAGFDASFF
GISPREAQAMDPQQRLVLEVSWEAXERAGIEPGSVRGSDTGVFMGAYPGGXGXGTDLGGFGMTSVAVSVLAGRVSYF
FGLEGPAMTVDTACSSSLVALHQAGSALRQGECSLALVGGVTVMPTPQTFVEFSRQRGLAADGRCKAFADAADGTGF
SEGVGVLLVERLSDAQARGHNILAVVRGSAVNQDGASNGLTAPNGPSQQRVIRQALANAGLAGAEVDVVEAHGTGTT
LGDPIEAQAVIATYGQDRDQPVLLGSLKSNLGHTQAAAGVSGVIKMVMALRHDTVPATLHIDEPSRHIDWTAGAVEL
VTENQSWPETGRDRRAAVSSFGISGTNAHVILESAPAQPVPPMDTPVSAVTAGVVPLPISARTVPALADLEDRLRAY
LTATPETDLPAVASTLAVTRSVFEHRAVLLGEETVTGIAVSDPRVVFVFSGQGSQRVGMGEELAAAFPLFARLHRQV
WDLLDVPDLEVDDTGYVQPALFALQVALFGLLESWGVRPQAVIGHSVGEVAAGYVAGVWSLEDACTLVSARARLMQA
LPAGGAMVAVPVSEERARAVLVDGVEIAAVNGPASVVLSGDESAVLRVAEGLGRWTRLSASHAFHSVRMEPMLEEFR
QVASELTYREPRIVMAAGEQVTTPEYWVRQVRDTVRFGDQVAAFGDAVFLEIGPDRTLSRLIDGIPTLHGDDEQHAV
VAALAELHVQGVPIDWSSILGVNPARVDLPTYAFQHERYWMVIQELSPEALLKIVRDSAAMMLGHPNTDAIAATTAF
RDLGVDSLIAVELRNSLAKATGLRLPATLVFDYPTPTVLAGRL
SEQ ID NO: 8
EPLAIVGMACRLPGGVSSPEDLWRLVESGTDAITDFPTDRGWDTDDLFDPDPDTPGKTYTVHGGFLDDVAGFDASFF
GISPREAQAMDPQQRLVLEAAWEAFERAGIEPGSVRGSDTGVFMGAYPGGYGIGVDLGGFGATAGAGSVLSGRLSYF
FGLEGPAMTVDTACSSSLVALHQAGSALRQGECSLALVGGVTVIANPQIFVEFSRQRGLAADGRCKAFADSADGTGW
SEGVGVLLVERLSDAQARGHNILAVVRGSAVNQDGASNGLTAPNGPSQQRVIRQALANAGLAGAEVDVVEAHGTGTT
LGDPIEAQAVIATYGQDRDQPVLLGSLKSNLGHTQAAAGVSGVIKMVMALQNGVVPRTLHADQPSRHIDWTAGAVEL
VTENQPWPELGRPRRAAVSAFGVSGTNAHVILESAPAQPVPPVDTPVSAVTAGVVPLPISARTVPALADLEDRLRAY
LTATPETDLPAVASTLATTRSVFEHRAVLLGEDTVTGTAIPDPRIVFVFSGQGSQRVGMGEELAAAFPLFARLHRQV
WDLLDVPDLDVDDTGYVQPALFALQVALFGLLESWGVRPQAVIGHSVGEVAAGYVAGVWSLEDACTLVSARARLMQA
LPAGGAMVAVPVSEEQARAVLVDGVEIAAVNGPASVVLSGDEAAVLRVAEGLGRWTRLSASHAFHSVRMEPMLEEFR
QVVSRLTYREPRIVMAAGEQVTTPEYWVRQVRETVRFGDQVAAFGDAVFLEIGPDRTLSRLIDGIAMLDGDDEVRAA
VAALAVMHVQGVGVDWPAILGTTTGRVLDLPTYAFQHERYWMANNGRPADLTPEALLKVVRDSAAMVLGHANADTVP
AATAFQELGLDSLIAVELRNSLAKATGLRLPATMVFDYPTPAALAGRL
SEQ ID NO: 9
EPLAIVGMACRLPGGVSSPEDLWRLVESGFDAITGFPTDRGWDVDNLYDPDPDAPGKSTTLHGGFLDDVAGFDASFF
GISPREAVAMDPQQRLAMEVSWEAFERAGIEPGSVRGSDTGVFMGAYPGGYGIGAELGGFMLTGRAGSVLAGRVSYF
FGLEGPAMTVDTACSSSLVALHQAAYALRQGECSLALVGGVTVMPTPVMFVEFSQQQNLADDGRCKAFADSADGTGW
SEGVGVLLVERLSDAQARGHNILAVVRGSAVNQDGASNGLTAPNGPSQQRVIRSALTSAGLTTADVDVVEAHGTGTT
LGDPIEAQAVLATYGQDRDQPVLLGSLKSNLGHTQAAAGVSGVIKMVMALQNGVVPRTLHVEEPSRHVDWTAGAVEL
VTENQSWPETGRARRAAVSSFGFSGTNAHVILESAPAQPVPPMDTPAPTVTTGVVPLPISAKSLPALADLEDQLRAY
LTATPETDLPAVASTLAMTRSVFEHRAVLLGEETVTGTAIPDPRIVFVFSGQGSQRVGMGEELAAAFPLFARLHRQV
WDLLDVPDLDVDDTGYVQPALFALQVALFGLLESWGVRPQAVIGHSVGEVAAGYVAGVWSLEDACTLVSARARLMQA
LPAGGAMVAVPVSEEQARAALVDGVEIAAVNGPASVVLSGDEAAVLRVAEGLGRWTRLSASHAFHSVRMEPMLEEFG
QVASELTYQEPRIVMAAGEQVTTPEYWVRQVRDTVRFGDQVAAFGDAVFLEIGPDRTLSRLIDGIAMLDGDDEVRAA
VAALAELHVQGVPIDWPAILGTTTGRVLDLPTYAFQHQRYWAASTWLAGLAPEEREGALMKVVRDTAAVVLGHADAG
TIPVTAAFKDLGLDSLTAVELRNSLAKSTGLRLPATMVFDYPTPASLAARLD
SEQ ID NO: 10
EPLAIVGMACRLPGGVESPEDLWRLVESGTDAISGFPADRGWADLSLRGGFLGDAAHFDAAFFGISPREALAMDPQQ
RLILEASWEAFERAGIEPGSVRGSDTGVFMGAFSGGYGAGADLAGFGVTAGAVSVLSGRVSYLFGLEGPAVTVDTAC
SSSLVALHQAGHALRQGECSLALVGGVTVMPTPDIFVEFSRQGGLASDGRCKAFADAADGTSWSEGAGVLVVERLSD
AERRGHTVLALVRGSAVNQDGASNGLTAPNGPSQQRVIQAALANAGLTPHEVDVVEAHGTGTRLGDPIEAQAVIATY
GRDREHPLLLGSLKSNVGHTQAASGVSGLIKMVMALRRGTVPRTLHVDEPSRHVDWTAGAVQLAIENQPWPETGRPR
RAAVSSFGVSGTNAHVILEGVPEEPADSEEPAGLTPLLISAKTPAALAEFEDRLRARLTTEPNLSAVASTLVRTRSL
FDHRAVLLDGETVSGMAEPDPRVVFVFSGQGSQRAGMGDDLAAAFPVFAKIRQQVWDLLDIPDLPVDETGHAQPALF
ALQVALFGLLDSWGVRPDALVGHSIGELAAGYVAGIWSLEDACALVSARARLMETLPPGGVMVAVPVSEEQARAVLT
DGVEIAAVNGPASVVLSGEETAVLQAAAALGGRSKRLATSHAFHSARMEPMLDEFRAVAEQLTYGSPRIPMAVGDGP
DYWVRQVRDTVRFGEQVAAHDGAIFVELGPDGSLARLVDGIAVLDREDEPRAALTALARLHVRGVKVDWPIAAGRRE
LDLPTYPFQRQRYWAETPTARRAPTDLLTLVRDTTATVLGYPDNTAVTPTTAFTDLGIDSLTAIELRNNMATTTGLR
LPATLVFDYPTPATLAARLD
SEQ ID NO: 11
EPLAIIGMACRLPGGVTTPEDLWQLVETGTDAISGFPTDRGWDVESLYDPDPDAAGKSYCVEGGFLDAVADFDASFF
GISPREALAMDPQQRLILETSWEAFERAGIDPADARGSDTGVFMGAFTSGYGADLEGFGGTAGALSVLSGRVSYFFG
LEGPAATVDTACSSSLVALHQAGYSLRHGECSLALVGGVTVMATPRTFVEFSRQRGLASDGRCKAFGDTADGTGWSE
GVGVLLVERLSDAERNGHRVLAVVRSSAVNQDGASNGLTAPNGPSQQRVIQAALDNAGLAPQDVDVVEAHGTGTTLG
DPIEAQAVIATYGQNREQPLLLGSLKSNVGHTQAAAGVSGVIKMIMALRHGVVPRTLHVDEPSRHVDWTAGAVHLVR
ENQPWPDVDRPRRAGVSSFGVSGTNAHIILESPPSQPAPEPAPALSPLVISAKTPQALAAYEDRLRTYLTAAPSTDA
RALAVTRSLFEHRAVLLGEDTVTGTALTEPRVVFVFPGQGWQWLGMGAALMESVVFAERMAECAAALSEFVDWNLIT
VLNDPAVIDQVDVVQPACWAVMVSLAAVWQAAGVRPDAVIGHSQGEIAAACVAGAISLRDAARIVALRSRLISERLG
KGAMASITLPADQITLAEGAWIAAYNGPTSTVVAGTPQAIEQMHGERVRRIAVDYASHTPHVEQIRAELLDLTTDVS
SQTPTLPWYSTVDGTWIDSPLDGDYWYRNLRQPVGFHPAVQTLQALGETVFVEVSASPVLLPAMDDAVTIATLRRDE
GTLTRMHTALAEAHVLGVTIDWPTVLGVTTRHVDLPTYAFQRQRYWVAELASLGPAERERALRKLVSDTAAGILGHA
DSGTVPVTAAFRELGVDSLTAVELRNGLAKATGLRLPATMVFDYPTPQALADRL
SEQ ID NO: 12
EPLAIVGMACRLPGGVSSPEDLWRLVESGTDAIADFPADRGWDVESLYDPDPDAAGKSYCVRGGFLAAAAEFDAAFF
GISPREALAMDPQQRLVLETSWEAFERAGIEPGSVRGSDTGVFMGAFAGGYGAAVEGFGATAGATSVLSGRVSYFFG
LQGPAITVDTACSSSLVALHQAGYSLRQGECSMALVGGVTVMATPQSFVEFSRQRGLAPDGRCKAFADTADGTGWSE
GVGVLLVERLSDAERNGHRVLAVVRSSAVNQDGASNGLSAPNGPAQQRVIRQALANAGLAAADVDVVEAHGTGTTLG
DPIEAQAVLATYGQDRERPLLLGSLKSNVGHTQAAAGVSGVIKMVMALRHGVVPRTLHVDEPSRHVDWTAGAVHLVT
ENQPWPDTDRPRRAGVSSFGVSGTNAHVIIEGSPTSSPVAEPSGDVLPLVVSAKTPQALTAYEDRLRAFLAAAPVTD
TRAVASTLAVTRSLFEHRAVLVGDNTVTGTALAEPRVVFVFPGQGWQWLGMGAALMESVVFAERMAECAAALGEFVD
WDLLAVLDDSAVVDRVDVVQPACWAVMVSLAAVWQDAGVRPDAVIGHSQGEIAAACVAGAISLRDAARIVALRSRLI
SERLGKGAMASITLPADQITLAEGAWIAAYNGPASTVVAGTPDAIEQMQGDRVRRIAVDYASHTPHVEQIRAELLDL
TAEVGSRTPTVPWYSTVDGTWIDSPLDGEYWYRNLRQPVGFHPAVQTLQALGETVFVEVSASPVLLPAMDDAVTVAT
LRRDEGTLTRMHTALAESHVLGVSIDWPHVLGDTGERMLDLPTYAFERHRYWSTARRNPSIAPDDLLTVVRDSAAVV
LGYADGGAVPVTGAFKDLGIDSLTAVELRNGLAKATGLRLPATVAFDYPTPQALAARL
SEQ ID NO: 13
EPLAIVGMACRLPGGVSSPEDLWRLVESGTDAITGFPADRGWAEYSFQGGFLDDAADFDAAFFGISPREALAMDPQQ
RLVLETAWEAFERAGIEPGSLRGSDTGVFMGAYPGGYGIGADRAGFGATAGAGSVLSGRVSYFFGLEGPAVTVDTAC
SSSLVALHQAGHALRLGECSLALVGGVTVMATPDTFVEFSRQGGLAADGRSKAFADSADGAGFAEGAGVLLVERLSD
AQRHGHQVLALVRGSAVNQDGASNGLTAPNGPSQQRVIQAALDNAGLTAAEVDVVEAHGTGTTLGDPIEAQAVIAAY
GQGRGEPLLLGSIKSNVGHTQAAAGVSGVIKVVMALRHGVVPRTLHVDEPSRHVDWTAGAVRLATENQSWPETGRPR
RAGVSSFGISGTNAHVILEGVPEEPAGHEEPAGLTPLLISAKTPAALAEFEDRLRAYLTTEPSLPAVASTLARTRSL
FDHRAVVLDGDVVRGVAEPDRRVVFVFSGQGSQRAGMGDDLAAAFPVFAKIRQQVWDQLDIPDLPVDQTGYAQPALF
ALQVALFGLLDSWGVRPDALVGHSIGELAAGYVAGIWSLEDACALVSARARLMQALPPGGVMVAVPVSEQQARGALT
DGVEIAAVNGPASVVLSGDEAAVLRAAAALGGRSKRLATSHAFHSARMEPMLDEFRMVAERLSYGSPRISMAVGDGP
DYWVRQVREAVRFGEQVAAHDGAVFVELGPDGSLARLIDGIAMLDRDDEPRAALTALARLHVQGVKVDWPIGAGRRV
DLPTYPFQRQRYWIDRPTARRAPTDLLTLVRDTAATVLGYPDSSAVPATTAFKDLGVDSLTAIELRNGMATTTGLRL
PATLVFDYPTPAALAARL
SEQ ID NO: 14
EPLAIVGMACRLPGGVSSPEDLWRLVESGTDAISGFPTDRGWAEHSFQGGFLDGAGDFDAPFFGISPREARVMDPQQ
RLVLEASWEAFERAGIEPGTVRGSDTGVFMGAYSGGYAAGADLAGFAATAGAGSVLSGRVSYFFGLEGPAVTVDTAC
SSSLVALHQAGHALRQGECSLALVGGVTVMATPDLFVEFARQQGLAADGRCKAFADNADGTGWSEGVGVLLVERLSD
AERNGHRVLAVVRSSAVNQDGASNGLTAPNGPSQQRVIQAALDNAGLTPADIDVVEAHGTGTTLGDPIEAQAVIATY
GQTREQPLLLGSLKSNVGHTQAAAGVSGVIKMVMALRHGVVPRTLHVDEPSRHVDWTAGAVQLAVENQPWPNTGRPR
RAGVSAFGVSGTNAHVIIEGSPTPSPVAEPSGDVLPLVISAKTPQALTAYEDRLRTYLNATPEIDTRAVASTLAVTR
SLFEHRAVLLGDNTVSGTALTEPRVVFVFPGQGWQWLGMGAALMESVVFAERMAECAAALSEFVDWNLITVLNDPAV
VDQVDVVQPACWAVMVSLAAVWQDAGVRPAAVIGHSQGEIAAACVAGAISLRDAARIVALRSRLIGERLGRGAMASV
ALPADEIALVDEVWVAAYNGPASTVIAGAPDAIEQMLGDRVRRIAVDYASHTPQVEQIRAELLDLTAEVSSQAPTVP
WYSTVDGTWIDGPLDSDYWYRNLRQPVGFHPAVEALGGLGETVFVEVSASPVLLPAMDDAVTVATLRRDEGTLTRMH
TALAEAHVLGVTIDWPAVVGDTGERMLDLPTYAFQHHRYWTTATARLEGRTGAEKHRLLLDIVLANAATVLGHDTAD
TIASDKPFKDLGIDSLTAVELRNSLARATELRLPATTAFDYPTPEALATRL
SEQ ID NO: 15
EPLAIVGMACRLPGGVSSPEDLWRLVESGTDAITGFPADRGWPDDSRQGGFLDDAADFDAAFFGISPREALAMDPQQ
RLVLEAAWEAFERAGIEPGSLRGSDTGVFMGAYPGGYGIGADQAGFGTTAGAGSVLSGRVSYLFGLEGPAVTVDTAC
SSSLVALHQAGHALRLGECSLALVGGVTVMGTPDIFAEFSRQGGLASDGRCKPFADAADGTGWAEGVGVLLVERLSD
AERHGHQVLAVVRGSAVNQDGASNGLTAPNGPSQQRVIQSALIQAGLAPHEVDVVEAHGTGTTLGDPIEAQAVIAAY
GQDRAQPLLLGSIKSNVGHTQAAAGVSGVIKMVMALRHGVVPRTLHVDEPSRHVDWSAGAVRLATESQPWPDTGHPR
RAGVSSFGISGTNAHVILEGVPEEPADTGEPSGLVPLLLSAKTPAALTHLEDRLRAYLTTEPNLPAVASTLAQTRSL
FDHRAVLLDGDVVRGVAEPDRRVVFVFSGQGSQRAGMGDDLAAAFPVFAKIRQQVWDLLDIPDLPVDETGHAQPALF
ALQVALFGLLDSWGVRPDALVGHSIGELAAGYVAGIWSLEDACALVSARARLMQALPPGGVMVAVSVSEEQARAVLT
DGVEIAAVNGPASVVLSGEETAVLQAAAALGGRSKRLATSHAFHSARMEPMLDEFRMVAERLSYGSPQIPMAVGDGP
DYWVRQVRETVRFGEQVAAHDGGIFVELGPDGSLARLVDGIAVLDRDDEPRAALTALARLHVQGVKVDWPIAAGRRV
LDLPTYPFQHQRYWATRPAARRAPTDLLTLVRDTAATVLGYPDSSAVPATTAFKDLGVDSLTAVELRNNLATSTGLR
LPATLVFDYPTPATLAARLD
SEQ ID NO: 16
EPLAIVGMACRLPGGVSTPEDLWQLVESGTDAISGFPADRGWDDYPYQGGFLTTAADFDAAFFGISPREALAMDPQQ
RLILEASWEAFERAGINPADARGSDTGVFMGAFSAGYGDDRDDSPATAGAVSVLSGRVSYFFGLEGPAMTVDTACSS
SLVALHQAGYSLRHGECSMALVGGVTVMATPRTFVEFARQGGLAEDGRCKAFADTADGTGWAEGVGVLLVERLSDAE
RNGHRVLAVVRSSAVNQDGASNGLTAPNGPSQQRVIQAALDNAGLAPQDVDVVEAHGTGTTLGDPIEAQAVIATYGQ
NRQQPLLLGSIKSNVGHTQAAAGVSGIIKMIMALRHGVVPRTLHVDEPSRHVDWTAGAVRLVTENQPWPDADRPRRA
GVSSFGISGTNAHIILEGVPEEPAQPDESPELTPLVISAKTAPALTQFEARLRSYLTTEPALSAVASTLAQTRSLFD
HRAVLLGGDTITGVAEPSPRVVFVFSGQGSQRAGMGDELAAAFPVFAKIRQQVWDLLDIPDLPVDETGHAQPALFAL
QVALFGLLDSWGVRPDALIGHSIGELAAGYVSGIWSLEDACALVSARARLMQASPPGGAMVAVPVSEQQARAVLTDG
VELAAVNGPSSVVLSGDETAVLQAAAALGGRSKRLATSHAFHSARMEPMLDEFRAVAEQLSYRSPQIPMAVGDGPEY
WVRQVRDTVRFGEQVAAHDGAIFVELGPDGSLVRLIDGIPMLDRDDEPRAALTALARLHVRGVNVAWPIAADRRELD
LPTYPFQRERYWSTASLSALAPAEREQALRKVVSDSSAMVLGYAEGRAVAPTAAFKDLGVDSLTAVELRNSLTKATG
LRLPATIVFDYPTPGALAVRL
SEQ ID NO: 17
EPLAIVGMACRLPGGVSSPEDLWRLVESGTDAISRFPADRGWDVDGLYDPDPDAPGKSYSVEGGFLDAVADFDAAFF
GISPREALAMDPQQRLILEASWEAFERAGIEPGSLRGSDTGVFMGAYSSGYGIGADIPGLGVTAGAVSVVSGRVSYF
FGLEGPAVTVDTACSSSLVALHQAGHALRRRECSLALVGGVTVMATPFGFVEFSRQRGLASDGRCKAFADTADGTSW
SEGAGVLVVERLSDAERHGHQVLAVVRGSAVNQDGASNGLTAPNGPSQQRVIQAALANAGLTPHEVDVVEAHGTGTR
LGDPIEAQAVIATYGQARGEPLLLGSIKSNVGHTQAAAGVSGVIKMVMALRHGVVPRTLHVDEPTRHVDWTTGAVRL
ATENQPWPETERPRRAGVSSFGVSGTNAHIILEGVAAEPAQPGESPELTPLLLSAKTPAALTHLEDRLRAYLTTEPN
LPAVASTLAQTRSLFDHRAVLLGGETVTGVAEPDPRVVFVFSGQGSQRAGMGDDLAAAFPAFAKIRQQVWDQLDIPN
LPVDETGHAQPALFALQVALFGLLDSWGVRPDALVGHSIGELAAGYVAGIWSLEDACALVSARARLMQALPPGGVMV
AVSVSEEQARAVLTDGVEIAAVNGPASVVLSGEETAVLQAAAALGGRSKRLATSHAFHSARMEPMLDEFRAVAEQLS
YGSPRIPMAVGDGPDYWVRQVRDTVRFGEQVAAHDGAIFVELGPDGSLARLIDGIAVLDRDDEPRAALTALARLHVR
GVKVDWPIAAGRRELDLPTYPFQHQRYWIDSRPTARRAPTDLLTLVRDTTATVLGYPDNTAVTPTTAFTDLGIDSLT
AIELRNNMATTTGLRLPATLVFDYPTPATLAARLD
SEQ ID NO: 18
EPLAIIGMACRLPGGVTTPEDLWQLVETGTDAISALPTDRGWADHPYQGGFLTTAADFDAAFFGISPREALAMDPQQ
RLILETSWEAFERAGINPADAHGSDTGVFMGAYSGGYGIGADLAGFGATAGATSVLSGRVSYFFGLEGPAITVDTAC
SSSLVALHQAGHALRHGECSLALVGGVTVMATPDIFVEFARQRGLAADGRCKAFADTADGTGWAEGVGVLLVERLSD
AERNGHRVLAVVRSSAVNQDGASNGLTAPNGPSQQRVIQAALDNAGLTPADIDVVEAHGTGTTLGDPIEAQALIATY
GQNREQPLLLGSLKSNIGHTQAAAGVSGVIKMVMALQHGVVPRTLHVDKPSRHVDWTAGAVRLLTESQPWPDTDRPR
RAGVSSFGVSGTNAHVIIEGSPTPSPVADPSGDVLPLVISAKTPAALAAYEDRLRTYLNATPEIDTRAVASTLAVTR
SLFEHRAVLLGEDTVSGTALTEPRVVFVFPGQGWQWLGMGAALMESVVFAERMTECATALSEFVDWNLITVLNDPAV
IDQVDVVQPACWAVMVSLAAVWQAAGVRPDAVIGHSQGEIAAACVAGAISLRDAARIVALRSRLISERLGKGAMASI
TLPADQITLAEGAWIAAYNGPTSTVVAGTPQAIEQMHGERVRRIAVDYASHTPHVEQIRAELLDLTTDVSSQTPTLP
WYSTVDGTWIDSPLDGDYWYRNLRQPVGFHPAVQTLQALGETVFVEVSASPVLLPAMDDAVTIATLRRDEGTLTRMH
TALAEAHVLGVTIDWPHVLGDTGERMLDLPTYAFQHHRYWTTAARLTGRTTAAQHRLMLDFVLGNVAAVLGHGSAGD
VAPDKPFKELGMDSLTSVELRNSLAKATGQRLPATIVFDHPTADALATYL
SEQ ID NO: 19
EPIAIVSMACRVPGGVTSPEGLWRLVESGTDAISEFPGDRGWDVANLYSPDPDAPGKSYSLQGGFLDGAAAFDASFF
GISPREALGMDPQQRLLLETSWEAVERARIDPKSLRGRDVGVYVGGAAQGYGLGAAEAQRDNLITGGSISLLSGRLS
YALGLEGPGLTVDTACSSSLVALHLAAQALRQGECSLALVSGVSVMPTPDVFVEFSRQRGLAADGRCKSFAAAADGT
SWSEGVGVLLLERLSDARRLGHEILAVVRGTAVNSDGASNGLTAPNGASQQRVIRQALASAGLGPADVDAVEAHGTG
TKLGDPIEAEAILATYGKDRPTPVWLGSLKSNIGHTMAASGVLGVIKMVESMRHGVLPRTLHVDEPSPHVDWAAGDV
ALLTSNQPWPAGRKPRRAGVSSFGLSGTNAHVVLEQYRMPAAPVTTKEAGPLPWVLSAQTPEALRERAGQLATALAG
DPAWHPLDVGYSLAATRSTFAHRAVVVGGDREFVRTLGKLADGAGWPGLTTGVAKSRRIAFMFDGQGTQRLAMGQGL
YARFPAFTRTWDTVSAEFAKHLDHTLTDVYLGGGGTAAAELVDDPLYAQAGIFAVEVALVELLAEWGVRPDVVTGHS
IGEAAAAYTAGMFSLADVTALITARGAALRSAPPGAMLALRAGEPEVRDFLDRTGAALDVAAVNGPAAVVVSGAPDA
VAGFASAWTASGRECRQLKVRRAFHSRHVEGVLGDFRTVLKSLTFRTPALPIVSTVTGRLIDPAEMGTPEYWLSQVR
QPVRFQDAVGELAGQGVSAFLEVGPSGTLASAGMECLDASFHALLRPRPAEDIGVLTALAELYAGGTAVDWATVLAG
GRPVDLPVYPFQHQSYWLRSAPDEPRTVLEMVHLEVASILGITDPDAVQDDSSFLELGFDSLSGVRLRNRLTQVTGL
TLPATLLFDHDTPSALATELD
SEQ ID NO: 20
EPLAVVGMACRLPGGITSPEELWELVEDGGDAVGDFPTDRGWDVAALHAAAESATSRAGALMGAADFDAAFFGISPR
EATALDPQQRILLEIAWEAIERAGIKADVLRGTDTGVFVGGFYYGYGAGADLGGFGAYSTQPAVLAGRLSYFFGLEG
PAVTVDTACSSSLVALHQAGQALRAGECSLALVGGVTVMASPQSFVEFSRQGGVAPDGRCKAFADAADGTGFAEGAG
VLVVERLSDAERNGHTVLAVVRGSAVNQDGASNGISAPNGPAQQRVIRQALGSAGLAPADVDVVEAHGTGTVLGDPI
EAQAVLATYGQGREVPLLLGSLKSNIGHAQAAAGVAGVIKMVMAMRRGVVPRTLHVDEPSSHVDWTTGAVELLTEAR
PWPESDRPRRAGVSAFGVSGTNAHVILEEVAESSVRSGGSSGLVPLPVSARTESSLAVQVERLGAYVRSGADLSAVA
DGLVRERVVFGHRAVLLGESTVAGVAEGELRTVFVFPGQGSQWVGMGRELMGASEVFAARMRECAAALEPHTGWDLL
DVLGEAVVADRVEVLQPASWAVAVSLAALWQAHGGTPDAVIGHSQGEIAAASVAGALSLEDAARIVALRSQTIAARL
GRGAMASIAIPSAEVEVMEGVWVAARNGPSSTVIAGDPAAVEQVLARYEAEGVRVRRIAVDYASHTPHVEAIQDELA
EVLEGVTAQVPTIPWWSTVDSDWVTEPVDDDYWYRNLRQPVAMDTAIGELDGSLFIECSAHPVLLPALDQERTVASL
RTDDGGWERFLTALAEAWTQGADVDWTILVEPAPHRLDLPTYPFDHKRYWLLERLGAMTGADRDAALLTLVRDCAAA
VLGHVDAAGVPADAAFKDLGVDSLTAVELRNRLAAATGVRLPATLAFDHPTPRAIASRLD
SEQ ID NO: 21
EPLAIVGMACRLPGGVASPGDLWQMLDSGGDAVTGFPVDRGWDPSGLTGGPDADRGGFLSDAADFDAAFFGISPREA
LAMDPQQRILLETTWEAFENAGIVPGTLRGSDTGVFMGAFSYGYGVGADLGGFGSIGVQPSVLTGRISYFYGLQGPA
FTVDTACSSSLVALHQAGHALRHGECSLALVGGVTVMANPDGFVEFEQQGGLSPDGRCRAFADAANGTGWAEGAGVL
VVERLSDAERNGHTVLAVVRGSAVNQDGASNGLSAPNGPSQQRVIRQALANAGLGAADVDVVEAHGTGTVLGDPIEA
QAVLATYGQGREVPLLLGSLKSNIGHAQAAAGVAGVIKMVMAMRRGVVPRTLHVDEPSSHVDWTAGAVEVVTEARPW
PESGRVPRAGVSSFGVSGTNAHVVLEGAPEPSSGAEASSGGGLVPLPVSARTESSLAVQVERLGAYVRGGADLGAVA
DGLVRGRAVFDRRAVLLGESTVAGVAVEGARTVFVFPGQGSQWVGMGRELMGVSEVFAARMRECAAALEPYTGWDVL
DVLGEAVVADRVEVLQPASWAVAVSLAALWQAHGVVPDAVVGHSQGEIAAACVAGALSLEDAARVVALRSQTIAARL
GHGAMASIALPASAVEVMEGVWIAARNGPESTVVAGDPAAVERVLARYEAAGVRVRRIAVDYASHTPHVEAIQDELA
DVLGGITSSAPDISWWSTVDSGWVTEAVGDDYWYRNLRQPVAMDTAVSELDGSLFIECSAHPVLLPALDQERTVASL
RTDDGGWDRFLTALAQAWTQGADVDWTTLIEPAQHRLDLPTYPFDHKRYWLQPAGARNEVARHTDLLTLVRQKAAAL
LGHAGPEDVPEDAAFRQLGVDSLIAVQLRNGLNEATGLRLSATLVFDYPTPRALAGRI
SEQ ID NO: 22
EPVAIVGMACRLPGGVTSPEDLWRLVASGTDAITEFPADRGWDVDALFDPDPDAVGRSTTRHGGFLTEATGFDAAFF
GISPNEALAMDPQQRLVLETSWEAFEHAGIVPDTLRESDTGVFMGAFHQGYGAGRDLGGLGVTATQTSVLSGRLSYF
YGLQGPAVTVDTACSSSLVALHQAAQALRSGECSLALAGGVTVMATPGSFVEFSRQRGLSPDGRCKAFADSADGTGF
AEGVGVLVVERLSDAERNGHTVLAVVRGSAVNQDGASNGLSAPNGVAQQRVIRQALANAGLNGTDVDAVEAHGTGTV
LGDPIEAQAVLATYGQEREVPLLLGSVKSNVGHTQAAAGVAGVIKMVMAMRRGVVPRTLHVDESSSHVDWSAGAVEV
VTEARPWPESGGARRAGVSSFGVSGTNAHVILEGVAESSVRSGGSSAGLVPLPVSARTESSLALQVERLGEYVRGGA
DLGAVADGLVRGRAVFGRRAVLLGESTVAGVAVEGARTVFVFPGQGSQWVGMGRELMGVSEVFAARMRECAAALEPH
TGWDVLDVLGEAVVADRVEVLQPASWAVAVSLAALWQAHGVVPDAVVGHSQGEIAAACVAGALSLEDAARVVALRSQ
TIAARLGHGAMASIALPASAVEVMEGVWIAARNGPESTVVAGDPAAVERVLARYEAAGVRVRRIAVDYASHTPHVEA
IQDELADVLGGITSSAPSVPWWSTVDSGWVTEPVDDDYWYRNLRQPVAMDTATGELDGSLFIECSAHPVLLPALDQE
RTVASLRTDDGGWERFLTALAEAWTQGADVDWTTLIEPAQHRVDLPTYPFDHKRYWLQPARRTVRTGEDSGRDLLAV
VCGATAAVLGHADASEIGPATAFKDLGIDSLSGIRLRNSLAETTGVRLSATAVFDHPTPDALAARL
SEQ ID NO: 23
EPLAIVAMACRMPGGVDTPEDLWRLVESGGDAITEFPTDRGWDLAALYDPDPDAIGKVSVRHGGFLAGAADFDAEFF
GISPREALAMDPQQRLILEVSWEAFERAGILPASVRGSDAGVFMGAFTQGYGAGVDLGGFGATGTPTSVLSGRLSYY
FGLEGPSVTVDTACSSSLVALHQAARSLRSGECSLALVGGVTVMATTTGFVEFSRQRGLAPDGRAKAFADTADGTSF
AEGAGVLIVERLSDATRLGHPVLAVVRGSAVNSDGASNGLSAPNGPAQRRVIERALDDAGLVPGDIDAVEAHGTGTR
LGDPIEAQALEAAYGLDRVHPLLIGSLKSNLGHTQAAAGVAGVIKMVLAMRHGVLPRTLHVDEPSRHVDWGGGVRLL
RRNEPWPVTGRVRRAGVSSFGISGANAHVVIEAGPPAAPATLPATEPVPEGVVWPVSARTPDGVRDVAGRLVALTAP
AAAIGHSLATTRTAMRHRAVVPARDAEAFARGEEVPGVVRGTADVTDARAVFVFPGQGSQWDGMGAELLATEPVFAR
RLGECAEALAPYTGWDLLDVIARRPGAPALDRVDVVQPVSFAMMVALAELWRSRGVAPAAVVGHSQGEVAAACVAGV
LTLDDAAKVVALRSRLVATELGHGGMVSVPPADFDAAAWAGRLEVAAVNGPASIVVAGAADAVEELLAATPHARRIA
VDYASHTAHVETIRDALLDALADLTPGAPEVPFFSTVDEAWLDRPADAAYWYDNVRRPVRFGAATARLAELGYRVFV
EASPHPVLTTALADTLAGHPNTAVTGTLRRGDGGARRFTSSLAELWVRGVPVSWPSGESRRVPLPTYPFRRDRYWID
AEAAPTAARDMLELVRTSAALVLGHRDAHAIEPTRAFKEVGFDSLTGVELRNRLADATGLTLPATLVFDHPTAQALA
AHLD
SEQ ID NO: 24
EPLAIVGMACRLPGGVASPEDLWRLLESGGDGITTFPGDRGWDVEALYDPDPEHPGTSTVRHGGFLSGAGDFDAGFF
GISPREAVAMDPQQRVVMETSWEALEYAGIDPHTLRGSDTGVFMGGYFYGYGSGADRGGFGATSTQTSVLSGRLSYF
YGLEGPAVTVDTACSSSLVALHQAGQSLRTGECSLALVGGVTVMASPSGFVDFSQQRGLAPDGRCKAFAEAADGTGF
AEGSGVLVVERLSDAERHGHRVLAVVRGSAVNQDGASNGLSAPNGPSQERVIRQALANAGLQPSDVDAVEAHGTGTR
LGDPIEATALLATYGQDRATPLLLGSLKSNIGHTQAAAGVAGIIKMVLAMHHDTLPSTLHVDTPSSHVDWTAGTVEL
LTDARPWPETSRPHRAAVSSFGVSGTNAHVILESHPRPTPAPDTGSSTHPVPLLISARTPRALSEHTTRVSAFLDAG
GGDERAVASALLTRTAFTHRAALIGTDLITGTAVPDRRLVWLFSGQGSQRPGMGDELAAAYDVFARTRRDVLDALQV
PAGLDIHDTGYAQPAVFALQVALSAQLDAWGVRPDALVGHSIGELAAAYVAGVWSLDDACALVSARARLMQALPPGG
AMAAVIASERDALPLLREGVEIAAVNGPASIVLSGDEDAVLDVAARLGRFTRLRTSHAFHSARMEPMLDEFRDVAQR
LTYHEPKLPMAAGADCATPEYWVRQVRDTVRFGEQVAAYDGAALLEIGPDRNLARLVDGIPVLHGDDEARSAMTALA
RLHTGGVAVDWPEVIGAAPTHLNLPTYPFERTRYWLGSRDRIAGLTAADAEKAALAVVRECAAAVLGHEGPARIEAT
ATFKELGVDSLTAVRLRNAFTEATGVRLPATAVFDFPTPQAVAAKL
SEQ ID NO: 25
EPLAIVGMACRLPGGVASPEDLWRLLESGGDGITAFPADRGWDVEALYDPDPEHPGTSTVRHGGFLSGAGDFDAGFF
GISPREAIAMDPQQRVVLETSWEALEQAGIVPGTLRGSDTGVFMGAFSDGYGLGTDLGGFGATGTQTSVLSGRLSYF
YGLEGPAVTVDTACSSSLVALHQAGQSLRTGECSLALVGGVTVMASPGGFVEFSQQRGLAPDGRCKAFAEAADGTAF
AEGSGVLVVERLSDAERRGHRILAVVRGSAVNQDGASNGLSAPNGPSQERVIRQALANAGLRPSDVDAVEAHGTGTR
LGDPIEATALLATYGQDRATPLLLGSLKSNIGHTQAAAGVAGIIKMVLAMRHGSLPRTLYVDTPSSHVDWTAGGVEL
LTDARPWPATTGPRRAAVSSFGVSGTNAHVILEAHAAPEPPALDSPVVEPSASLFATELTPLPVSARTSEAVDGQVQ
RLREHLATHPGDDPRAVAAALLATRTDFPHRAVLLGDGVVTGTALTAPRTVFVFPGQGSQWLGMGRKLMAESPVFAA
RMRQCADALAEHTGRDLIAMLDDPAVKSRVDVVHPVCWAVMVSLAAVWEAAGVRPDAVIGHSQGEIAAACVAGAISL
EDGARLVALRSALLVELAGRGAMGSIAFAAADVEAAAARIDGVWVAGRNGTATTIVSGRPDAVETLIADYETRGVWV
TRLVVDCPTHTPFVDPLYDELQRIVAATTSRAPEIPWFSTADERWIDAPLDDEYWFRNMRNPVGFAAAVAAAREPGD
TVFIEVSAHPVLLPAINGTTVGTLRRGGGADRLLDSLAKAHTVGVAVDWAAHDAATGTADLPTYAFHHERYWIEPAE
RLPDLSRKEQEQVLLDVVRDTAATLLGHADARAVTATAAFKDLGVDSLTALGLRDRLAEALGIPLPATLVFDHPAAG
TLSRHL
SEQ ID NO: 26
EPLAIVGMACRLPGGVASPDDLWRLLESGGDGIGAFPGDRGWETGADGRGGFLSGAAGFDAAFFGVSPREALAMDPQ
QRVVLETSWEALEHAGIDPHTLNGSDTGVFLGAFFQGYGIGADFDGYGTTSIHTSVLSGRLSYFYGLEGPAVTVDTA
CSSSLVALHQAGQSLRTGECSLALVGGVTVMASPAGFADFSEQGGLAPDGRCKAFAEAADGTAFSEGSGVLVVERLS
DAERHGHRILAVVRGSAVNQDGASNGLSAPNGPSQERVIRQALANAGLQPSDVDAVEAHGTGTRLGDPIEATALLAT
YGQHRTTPLLLGSLKSNIGHTQAAAGVAGIIKMVLAMHHDTLPPTLHVDTPSSHVDWTTGGVELLTDARPWPTTTGP
RRAGISSFGVSGTNAHVILESPTPVPSPGAEPGARPVPLPISARTPEALDEHTIRIRAFLDDNPGADHVAVAQTLAR
RTPFEHRAVLLGDTLITADPNAGSGPVVFVYSGQSTLHPHTGRQLAATYPVFADAWGEVLGHLDADQGPATHFAHQI
ALTALLRSWGIAPHAVIGHSLGEISAACAAGVLSLGDASALLAARSRLMDELPAGGAMVTVLTSEENALRALRPGVE
IAAVNGPHSVVLSGDEGPVLAVAQQLGIHHRLPTRHAGHSARMDPLVAPLLEAASGLTYHQPRIAIPGDPTTAAYWA
RQVRDQVRFQAHAERYPGATFLEIGPNQDLSPVVDGIPTQTGTPDEVQALHTALARLHTRGGVVDWPTVLGGDRAPV
ALPTYPFQHKDYWLRATELAVLPDDERADALLAFVRNSTATVLGHLGAEDIPATATFKELGIDSLTAVQLRNALTTA
TGVRLNATAVFDFPTPRALAARL
SEQ ID NO: 27
EPLAIVGMACRLPGGVASPEGLWRLVASGTDAITEFPADRGWDVDALYDPDPAIGKTFVRHGGFLDGATGFDAGFFG
ISPREALAMDPQQRVLLETSWEAFESAGITPDSARGSDTGVFIGAFSYGYGTGADTNGFGATGSQTSVLSGRLSYFY
GLEGPSVTVDTACSSSLVALHQAGQSLRSGECSLALVGGVTVMASPGGFVEFSRQRGLAPDGRAKAFGAGADGTSFA
EGAGALVVERLSDAERHGHTVLAVVRGSAVNSDGASNGLSAPNGPSQERVIRQALANAKLTPADVDAVEAHGTGTRL
GDPIEAQALLATYGQDRATPLLLGSLKSNIGHAQAASGVAGIIKMVQAIRHGELPPTLHADEPSPHVDWTAGAVELL
TSARPWPGTGRPRRAAVSSFGVSGTNAHIILEAGPVKAGPVEAGPVPAAPPSAPGEDLPLLVSARSPEALDEQIGRL
RTYLDTRPGVDRAAVAQTLARRTHFAHRAVLLGDTVITTSPSHQADELVFVYSGQGTQHPAMGEQLAAAFPVFAETW
HDALRRLDDPDPHDPTRSQHTLFAHQAALTALLRSWDITPHAVIGHSLGEITAAYAAGILSLDDACTLITTRARLMH
TLPPPGAMVTVLTGEEEARQALRPGVEIAAVNGAHSVVLSGDEDAVLDVAQRLGIHHRLPAPHAGHSAHMEPVAAEL
LATTRRLRYDRPHTAIPNDPTTAEYWAEQVRNPVLFHAHTQQYPDAVFVEIGPGQDLSPLVDGIALQNGPANEAHAL
RTALARLFSRGATLDWPLVLGGASRHDPDVPSYAFQQRPYWIESARLAELPDADRDTALSTLVMDATAAVLGHADAS
EIGPTTTFKDLGIDSLTAIELRNRLAEATGLRLSATMVFDHPTPRVLAAKL
SEQ ID NO: 28
EPLAIVGMACRLPGGVTSPEDLWRLVASGTDAITEFPTDRGWDIDRMFDPDPDAPGKTYVRHGGFLSEAAGFDAAFF
GISPREAWAMDPQQRVILETVWEAFENAGIVPDTLRGSDTGVFMGAFSHGYGAGVDLGGFGATATQNSVLSGRLSYF
FGMEGPAVTIDTACSSSMVALHQAAQSLRDGECSLALAGGVTVMPTPLGYVEFCRQRGLAPNGRAKAFAEGADGTSF
SEGAGVLVVERLSDAERNGHTVLALVRSSAVNQDGASNGISAPNGPSQQRVIRQALDKAGLTPADVDVVEAHGTGTP
LGDPIEAQAIIATYGQDRDTPLYLGSVKSNIGHTQTTAGLAGVIKMVMAMRHGLLPKTLHVDEPSSHVDWSAGAVEL
LTEARPWPDSDRPRRAGVSSLGISGTNAHVILEGVAESSVRSGGSSGLVPLPVSARTESSLALQVERVGEYVRGGAD
LGAVADGLVRGRAVFDRRAVLLGESTVAGVAVEGARTVFVFPGQGSQWVGMGRELMGASEVFAARMRECAAALEPHT
GWDVLDVLGEAVVADRVEVLQPASWAVAVSLAALWQAHGVVPDAVIGHSQGEIAAACVAGALSLEDAARVVALRSQT
IAARLGHGAMASIALPASAVEVAEGVWIAARNGPESTVVAGDPGAVERVLARYEAAGVRVRRIAVDYASHTPHVEAI
EEQLADVLGGITSSAPDISWWSTVDSGWVTEPVGDDYWYRNLRQPVAMDTAISELDGSLFIECSAHPVLLPALDQEH
TVASLRTDDGDWDRFLTALAQAWTQGAPVDWTTLIEPAPHRLDLPTYPFDHKRYWIEAAARLAGHTAAEQRRVMQEV
VLRQAAAVLAYGLGEQVAADRPFRDLGFDSLTAVDLRNRLAAETGLRLPTTVVFSHPTAEALATHL
SEQ ID NO: 29
EPIAIVAMACRLPGGVTSPEELWRLVESGTDAITMAPGDRGWDLDALYDPDPDAVGKAYNLRGGFLEGAAEFDAAFF
DISPRESLGMDPQQRLLLETAWEAIERGRINPASLHGREIGVYVGAAAQGYGLGAEDTEGNAITGGSTSLLSGRLAY
VLGLEGPSVTVDTACSSSLVALHLACQGLRLGECELALAGGVSVLSSPAAFVEFSRQRGLAADGRCKSFGSGADGTT
WAEGVGVLVLERLSDAERLGHTVLAVVRGSAVTSDGASNGLTAPNGLAQQRVIRKALAAAGLTAADVDLVEGHGTGT
RLGDPVEADALLATYGQNRQEPVWLGSLKSNIGHATAAAGVAGVIKTVQAIGAGTMPRTLHADEPSPAVDWTAGRVS
LLTGNRPWPDDERARRAAVSAFGLSGTNAHVILEQHRPEPVAPRPPREEPRPLPWVLSARTPAALRAQAARLRDHLA
AVPDADPLDIGYALATSRARFTHRAAVVATSSDEFRAGLDSVADGVEAPGVVGGTARERRVAFLFDGQGAQRVGMGR
ELHGRFPVFAAAWDEVSDAFGKHLEHSPTDVFHGEHGDLAHDTLYAQVGLFTLEVALLRLLEHWGVRPDVLVGHSVG
EVTAAYAAGVLTLADATALIVARGRALRALPPGAMTAVDGSPAEVGAFTGLDIAAVNGPSAVVLTGSPDDVTAFERE
WAAAGRRAKRLDVGHAFHSRHVDGALDDFRTVLESLSFGAARLPVVSTTTGRDAAGDLATPEHWLRHARRPVLFADA
VRELADLGVNMFVAVGPSGALASAASENTGGSAGTYHAVLRARTGEENAALTAVAELHAHGAPVDLAAVLAGGRPVD
LPVYPFQHRSYWLAPDDLTVAEIVRRRAAALLGIADPGDVDADTTFFALGFDSLAVQRLRNQLTAATGLDLPTAVLF
DHDTPSALTAYL
SEQ ID NO: 30
EPLAIVGMACRLPGGVSSPEDLWRLVESGTDAISGFPTDRGWDVENLYDPDPDASGKSYCVQGGFLDAAAGFDAGFF
GISPREALAMDPQQRLVLEVSWEAFERAGIEPGSVRGTDTGVFIGAYPGGYGAGAGTELEGYGTTSGPSVLSGRVSY
FFGLEGPAITVDTACSSSLVALHQAGYALRQGECSLALVGGVTVMATPDVFTEFARQRGLAADGRSKAFSDSADGAG
FSEGIGVLLVERLSDAQAKGHQVLAVVRSSAVNQDGASNGLTAPNGPSQQRVIQAALGNAGLTTAEVDVVEGHGTGT
TLGDPIEAQALLATYGQDRERPLLLGSLKSNIGHTQAAAGVSGVIKMVMALRHGLVPRTLHVDEPSSHVDWTAGAVE
LVTANQPWPDADRPRRAGVSSFGVSGTNAHVILESAPSTQAVDDVRPVETPVVGSELVPLVLSAKTLPALSGYEDRL
RAYLAGSPGVDLRAVASTLAVTRSVFEHRAVLLGDDTVTGTAVTDPRVVFIFPGQGSQRAGMGEELAAAFPVFARIH
QQVWDLLDVPDLEVNETGYAQPALFALQVALFGLLESWGVGPDAVVGHSVGELAAAYVSGVWSLEDACTLVSARARL
MQALPPGGVMVAVPVPEDEARAVLGEGVEIAAVNGPSSVVLSGDEAAVLRAAATLGKWMRLATSHAFHSARMEPMLD
EFRAVAERLTYQTPHLTMAAGEQVTTPDYWVRQVRDVVRFGEQVASFEDAVFVELGADRSLARLVDGVAMLHGAHEA
QAAISALAHLYVNGVTVDWPAVLGDVPGRVLDLPTYAFQHQRYWLEGWLAALTPAEREKALLKLVSDGAATVLGHAD
TSTIPVTGAFKDLGINSLTAVELRNSLAKATELRLPATLVFDYPTPATLAARLD
SEQ ID NO: 31
EPLAIVGMACRLPGGVSSPEDLWRLVESGTDAISGFPTDRGWDVENLDSAGKSYRAEGGFLDAAAGFDASFFGISPR
EALAMDPQQRLVLEVSWEAFERAGIEPGSLRGSDTGVFMGAYPGGYGIGADLGGFGATAGATSVLSGRVSYFFGLEG
PAFTVDTACSSSLVALHQAGYALRQGECSLALVGGVTVMATPQTFVEFSRQGGLASDGRCKAFADAADGTGWAEGVG
VLLVERLSDAQAKGHQVLAVVRSSAVNQDGASNGLTAPNGPSQQRVIQAALSNAGLTTAEVDVVEAHGTGTTLGDPI
EAQALLATYGQDRERPLLLGSVKSNLGHTQAAAGVSGVIKMVMALRHGLVPRTLHVDEPSRHVDWSTGAVELVTENQ
PWPETGRPRRAGVSSFGISGTNAHVILESAPSAQVVENTVVESAPEWVPLVVSARTQSALADYEDRLRAYLAGSPGV
DLRAVASTLAVTRSVFEHRAVLVGDDTVTGSAVSDPRVVFVFPGQGSQRAGMGEELAAAFPLFAQIHQQVWDLLDVP
DLEVNETGYAQPALFALQVALFGLLESWGVRPDAVIGHSVGELAAGYVCGVWSLEDACTMVSARARLMQALPAGGVM
VAVPVSEDEARAVLGEGVEIAAVNGPLSVILSGDEAAVLRAAATLGKWTRLATSHAFHSARMEPMLEKFRAVAEGLT
YRTPRLTMAAGDQVATAEYWVRQVRDVVRFGEQVASFEDAVFLELGADRSLARLVDGIAMLHGDHEAQAAISALAHL
YVNGMAVDWPAVLGDVRGRVLDLPTYAFEHQRYWLEGWLAVLAPAEREKALLKLVRDSAALVLGHADASTIPVAAAF
KDLGIDSLTAVELRNSLAKATGLRLPNTTVFDYPTPAILAARL
SEQ ID NO: 32
EPLAIVGMACRLPGGVSSPEDLWRLVESGTDAISGFPTDRGWDAESLYDADPDAPGKSYCVEGGFLDNASSFDAGFF
GISPREALAMDPQQRLVLEVSWEAFERAGIEPGSIRGTDTGVFMGAYAGGYGAGADLGGFAATASATSVLSGRVSYF
FGLEGPAITVDTACSSSLVALHQAGYALRQGECSLALVGGATVMATPQSFVEFSRQRGLASDGRCKAFADAADGTGW
AEGVGVLLVERLSDARRNGHQVLAVVRSSAVNQDGASNGLTAPNGPSQQRVIQAALSNAGLAAHEVDVVEAHGTGTT
LGDPIEAQAVLATYGQDRERPLLLGSLKSNIGHTQAASGVSGVIKMVMALQHHTVPRTLHVNEPSRHVDWSAGAVEL
VRENQSWPEGDRPRRAGVSSFGVSGTNAHIILESAPAQSAEEVQPVEVPVVASDVLPLVVSAKTHSALTEAEDRLRA
YLTASPEADMPAVASTLAVTRSVFEHRAVLLGDDTVTGTGTAMSDPRVVFVFPGQGWQWLGMGSALRESSVVFAERM
AECAAALSDFVDWDLFTVLDDPAVVDRVDVVQPASWAVMVSLAAVWQAAGVRPDAVIGHSQGEIAAACIAGAVSLRD
AARIVTLRSQAIARGPAGRGAMASIALPAQEIELADGAWIAAHNGPASTVIAGTPEAVDLVLTAHEAQGTRVRRITV
DYASHTPHVELIRDELLHITAGIGSQVPVVPWLSTVDGTWVEGPLDAEYWYRNLREPVGFAPAISQLQAQGETVFIE
VSASPVLLQAMDDDAVTVATLRRDDGDATRILTALAQAYTHGVTVDWPAILGTTTTRALDLPTYAFQHQRYWLNNRL
TGRTSVEQHRVMLELVLGEAASVLGHGSPDAIATDTSFKDLGMDSLTAIELRNRLMAETGLQLPATMVFDYPTANAL
ATHL
SEQ ID NO: 33
EPIAIVAMACRVPGGVSSPEGLWRLVESGTDVISGFPTDRGWDVEGLFDPDPDAPGKSYCVQGGFLDTAADFDAPFF
GISPREALGMDPQQRLLLETTWEAIERARIDPKSLRGRDVGVYVGGAAQGYGVGVDQQRDNGITGSSVSLLSGRVSY
ALGLEGPGVTVDTACSSSLVALHLASQALRQRECSLALVSGVSVMSSPAMFVEFSRQRGLSSDGRCKSFAASADGTI
WSEGVGVLVVERLSDARRLGHRFLAVVRGSAVNSDGASNGLTAPNGASQQRVIRQALAGAGLTASDVDVVEAHGTGT
KLGDPIEAEAILATYGQERSTPAWLGSLKSNIGHTMAASGVLGVIKMVEAMRHGSLPRTLHVDDPSPHVDWTSGSVA
LLTEHQPWPDDAKPRRAGVSSFGLSGTNAHVVLEQYQAPAPSVTPVTPVTPVTPVTPNEPRPLAWVLSAQSPKALRE
QAGRLYASLAEAPEWNSLDIGYSLATTRSDFAHRAVAVGSGREFLRALSKLADGASWPGLTTATAKARRVAFLFDGQ
GAQRLGMGKELYDSSPVFARAWDTVSAGFDKHLDHSLTDVYFGEGGSTTAELVDDTLYAQAGIFAMEVALFGLLEDW
GVRPDFVAGHSIGEATAAYASGMLSLEHVTTLIVARGRALRATPPGAMVALRAGEEEVRAFLDQTGAALDLAAVNSP
EAVVVAGEPDAVAGFEAAWAASGREARKLRVRHAFHSRHVEAVLDEFRTTLESLKFSAPALPVVSTVTGQLIEPDEM
GTPEYWLRQVRQPVRFQDAVRELAEAGVGTFVEIGPSGALASAGMECLGGDASFHAVLRPRSPEDVCLMTAIAELYA
GGTAIDWAKVLSGGRAVDLPVYPFQHQSYWLAPAEPSYADEPRTMLELVHMEVASVLGMTDPGVILDDSSFLELGFD
SLSAVRLRNRLSKATGLDLPSTLLFEHPTSAELASHLD
SEQ ID NO: 34
EPLAIVGMACRLPGGVSSPEDLWRLVESGTDAISGFPTDRGWDVEGLFDPDPDASGKSYCVRGGFLDSVGGFDASFF
GISPREALAMDPQQRLLLEVSWEAFERAGIEPGSVRGSDTGVFMGGFPGGYGAGADLEGFGATAGAASVLSGRVSYF
FGLEGPAITVDTACSSSLVALHQAGYALRQGECSLALVGGVTVMATPQSFVEFSRQRGLASDGRCKAFADAADGTGW
AEGVGVLLVERLSDAQAKGHQVLGVVRGSAVNQDGASNGLSAPNGPSQQRVIRAALSNAGLTTAEVDVVEAHGTGTT
LGDPIEAQAVLATYGQDREQPLLLGSLKSNIGHAQAAAGVSGVIKMVMALRHGLVPRTLHVDEPSRHVDWSAGAVEL
VTENQSWPVTGRPRRAGVSAFGVSGTNAHVILESAPAQASEEAQPVVTPVVTPVVASELVPLVVSAKTESALAEVEG
RLRAYLAVSPGVDLRAVGSTLAVARSVFEHRAVLLGDDTVTGTVTGTAVSDPRVVFVFPGQGWQWLGMGSALRGASV
VFAERMAECAAALGEFVDWDLFAVLDDPAVVDRVDVVQPASWAVMVSLAAVWEAAGVRPDAVVGHSQGEIAAACVAG
AVSLRDAARIVTLRSQVIAGLAGRGAMASVALPAHEIELVEGAWIAACNGPASTVIAGEPDAVDRVLAVHEARGVRV
RRITVDYASHTPHVELIRDELLNITAGIGSQAPVVPWLSTVDGTWVEGPLDAEYWYRNLREPVGFDSAVGELRAQGD
TVFVEVSASPVLLQAMDDDVVSVATLRRDDGGAARMLTALAQAFVEGVTVDWPAVLGNAPGRVLDLPTYAFEHQRYW
LKSRWLARLAPVEREKALLKVVCDGAATVLGHADASTIPAAGAFRDLGVDSLTAVELRNRLAKATGLRLPATLVFDY
PTPTALAARL
SEQ ID NO: 35
EPLAIVGMACRLPGGVSSPEDLWRLVESGTDAVSGFPTDRGWDVEDLFGPAAGDSYRLRGGFLDAAGGFDASFFGIS
PREALAMDPQQRLVLEVSWEAFERAGIEPGSVRGTDTGVFMGAYPGGYGIGADLGGFGTTAGAVSVLSGRVSYFFGL
EGPAFTVDTACSSSLVALHQAGYALRQGECSLALVGGVTVMATPQTFAEFARQGGLAGDGRSKAFADSADGAGFSEG
VGVLLVERLSDARRNGHQVLAVVRGSAVNQDGASNGLTAPNGPSQQRVIQAALNNAGLTTAEVDVVEAHGTGTTLGD
PIEAQALLAAYGQDRERPLLLGSVKSNLGHTQAAAGVSGVIKMVMALRHGLVPRTLHVDEPSRHVDWSEGAVELVTE
NQSWPDTGRPGRAGVSSFGISGTNAHVILESAPSAQTVENTVVESAPEWVPLVMSARTQSALADYEGRLRAYLAGSP
GVDLRAVASTLAVTRSVFEHRAVLMGDDTVTGSAVSDPRVVFVFPGQGSQRAGMGEELAAAFPVFAQIHQQVWDLLD
VPDLDVNETGYAQPALFALQVALFGLLESWGVGPDAVVGHSVGELAAAYASGVWSLEDACTLVSARARLMQALPAGG
VMVAVPVSEDEARAVLGEGVEIAAVNGPSSVVLSGDEAAVLRAAAGLGKWTRLATSHAFHSARMEPMLEEFRAVAER
LTYQTPHLTMAAGEQVTTPDYWVRQVRDVVRFGEQVASFEDAVFLELGADRSLARLVDGIAMLHGDHEAQDAISAMA
HLYVSGVAVDWPAVLGDVRGRVLDLPTYAFQHERYWLEGRWLAALAPAEREKALLKLVSDGAATVLGHADASTVPVS
AVFRDLGVDSLTAVELRNRLAKATGLRLPATLVFDYPTPTALAARL
SEQ ID NO: 36
EPLAIVGMACRLPGGVASPEDLWRLVESGTDAVSGFPTDRGWDVEDFDSAGKSYRAEGGFLDAAAGFDASFFGISPR
EALAMDPQQRLLLEVSWETFERAGIEPGSVRGTDTGVFMGAYPGGYGIGADLGGFGATAGATSVLSGRVSYFFGLEG
PAFTVDTACSSSLVALHQAGYALRQGECSMALVGGATVMATPELFTEFSRQGGLASDGRCKAFADSADGTGWAEGVG
VLLVERLSDAQAKGHQVLAVVRSSAVNQDGASNGLTAPNGPSQQRVIQAALSNAGLAAYEVDVVEAHGTGTTLGDPI
EAQAVLATYGQDRERPLLLGSLKSNIGHTQAASGVSGVIKMVMALQRGLVPRTLHVDEPSRHVDWSAGAVELVRENQ
SWPDTEGPRRAGVSSFGVSGTNAHVILESAPAQPAEEAQPVVTPVVASELVPLVVSAKSQSALTEAEGRLRAYLAAS
PGVDTRAVGATLAVARSVFEHRAVLLGDDTVTGTGTAMSDPRVVFVFPGQGWQWLGMGSALRDSSVVFAERMAECAA
ALSDFVDWDLFTVLDDPAVVDRVDVVQPASWAVMVSLAAVWEAAGVRPDAVIGHSQGEIAAACIAGALSLRDAARIV
SLRSQVIAGLAGRGAMASIALPAQDVELAEGAWIAAHNGPASTVIAGAPEAVDRVLAVHEARGVRVRRITVDYASHT
PHVELIRDELLHITAGIGSQAPVVPWLSTVDGTWVEGPLDAEYWYRNLREPVGFAPAIRQLQDQGETVFIEVSASPV
LLQAMDDDVVSVATLRRDDGGAARMVTALAQAYVQGVTVDWPAVLGNVPGRVLDLPTYAFEHQRYWLKSWLAALAPA
EREKALLKVVCDSAAVVLGHADARSIPAAGAFKDLGVDSLMAVELRNRLVKATGLRLPATLVFDYPTPAALAARL
SEQ ID NO: 37
EPLAIVGMACRLPGGVSSPEDLWRLVESGTDAVSGFPTDRGWDLEDLFDPDPEAAGKSYCVQGGFLDAAAGFDAGFF
GISPREALAMDPQQRLLLEVSWEAFERAGIEPGSVRGSDTGVFIGAFPVGYGVGFDREGYGATSGPSVLSGRVSYFF
GLEGPAITMDTACSSSLVALHLAAQALRNGECSMALAGGVTVMATPEVFTEFARQRGLASDGRCKAFADSADGAGFS
EGAGLLLVERLSDARRNGHQVLAVVRGSAVNQDGASNGLTAPNGPSQQRVIQAALINAGLTTAEVDVVEAHGTGTTL
GDPIEAQAVLATYGQGRERPLLLGSLKSNIGHTQAASGVSGVIKMVMALQRGLVPRTLHVDEPSRHVDWSAGAVELV
RENQSWPDSEGPRRAGVSSFGVSGTNAHVILESAPAQPAEEAQPVVTPVVASELVPLVVSAKTESALTEVEGRLRVY
LAASPGVDTRAVASTLAVTRSVFEHRAVLLGDDTVTGTGTAVSDPRVVFVFPGQGWQWLGMGSALRDSSVVFAERMA
ECAAALSEFVDWDLFAVLDDPAVVDRVDVVQPASWAVMVSLAAVWQAAGVRPDAVIGHSQGEIAAACVAGVLSLRDA
ARIVTLRSQAIAGLAGRGAMASIALPAQDVELVEGAWVAAHNGPASTVIAGAPEAVDRVLAVHEARGVRVRRIAVDY
ASHTPHVELIRDELLDITAGIGSQAPVVPWLSTVDGTWVEGPLDAEYWYRNLREPVGFAPAVSQLQVQGETVFVEVS
ASPVLLQAMDDDVVSVATLRRDDGGAARMLTALAQAYTQGVAVDWPAVLGTTTAQVLDLPTYAFQHRRYWVEWLAAL
APEEREKALLRVVCDGAATVLGHADVGSIPVTAAFKDLGVDSLTAVELRNRLAKATGLRLPATLAFDYPTPTALAAR
L
SEQ ID NO: 38
EPLAIVGMACRLPGGVSSPEDLWRLVESGTDAISGFPTDRGWDVEHLYDPDPDAPGKAYCVQGGFLDSAGGFDASFF
GISPREALAMDPQQRLVLEASWEAFERAGIEPGSLRGTDTGVFMGAYPGGYGIGADLGGFGATAGAVSVLSGRVSYF
FGLEGPAVTVDTACSSSLVALHQAGYALRQGECSLALVGGVTVMATPQSFVEFSRQRGLAGDGRCKAFADAADGTGW
AEGVGVLLVERLSDAQAKGHQVLAVVRGSAVNQDGASNGLSAPNGPSQQRVIRQALANAGLTTAEVDVVEAHGTGTT
LGDPIEAQALLATYGQDRERPLLLGSVKSNLGHTQAAAGVSGVIKMVMALRHGLVPRTLHVDEPSRHVDWSEGAVEL
VTENQPWPDADRPRRAGVSSFGISGTNAHVILESAPSTQAVDDVRPVEAPVVASEWVPLVVSARTLPALVEYEGRLR
AYLAGSPGVDMRAVGSTLAVTRSVFEHRAVLMGDDTVTGSAVSGPRVVFVFPGQGSQRAGMGEELAAAFPVFARIHQ
QVWDLLDVPDLEVNETGYAQPALFALQVALFGLLESWGVGPDAVIGHSVGELAAGYVSGLWSLEDACTLVSARARLM
QALPPGGVMVAVPVSEEEAKAVLCEGVEIAAVNGPSSVVLSGDETAVLRAAAALGKSTRLATSHAFHSARMEPMLDE
FRAVAERLTYQTPRLPMAAGEQVTTPDYWVRQVREPVRFGEQAASCGDAVFVELGADRSLARLVDGVAMLHGDHEAQ
AAISALAHLYVNGVTVDWPAVLGDVPGRVLDLPTYAFQHQRYWLEGWLAALAPEERAKALLKVVCDTAATVLGHADA
RTIPMTGAFRDLGIDSLTAVELRNGLAKATGLRLPATLVFDYPTPTVLAARL
SEQ ID NO: 39
EPLAIVGMACRLPGGVSSPEDLWRLVESGTDAISGFPTDRGWDAESLYDPDPDAPGKSYCVEGGFLDNAASFDAGFF
GISPREALAMDPQQRLVLEVSWEAFERAGIEPGSVRGTDTGVFMGAYAGGYGAGADLGGFAATASATSVLSGRVSYF
FGLEGPAFTVDTACSSSLVALHQAGYALRQGECSLALVGGATVMATPQSFVEFSRQRGLASDGRCKAFADAADGTGW
AEGVGVLLVERLSDARRNGHQVLAVVRSSAVNQDGASNGLSAPNGPSQQGVIRQALANAGLTPAEVDVVEAHGTGTT
LGDPIEAQAVLATYGQDRERPLLLGSLKSNIGHTQAASGVSGVIKMVMALQHHTVPRTLHVNEPSRHVDWSAGAVQL
VRENQSWPEGDRPRRAGVSSFGVSGTNAHIILESAPAQSAEEVQPVEVPVVASDVLPLVVSAKTHSALTEAEDRLRA
YLTASPEADMPAVASTLAVTRSVFEHRAVLLGDDTVTGTGTAVSDPRVVFVFPGQGWQWLGMGSALRDSSVVFAERM
AECAAALSDFVDWDLFAVLDDPAVVDRVDVVQPASWAVMVSLAAVWQAAGVRPDAVIGHSQGEIAAACIAGALSLRD
AARIVTLRSQVIAGLAGRGAMASIALPAQEVELAEGAWIAAHNGPASTVIAGTPEAVDLVLTAHEAQGTRVRRIAVD
YASHTPHVELIRDELLDITAGIGSQAPVVPWLSTVDGTWVEGPLDAEYWYRNLREPVGFAPAVRQLQDQGETVFIEV
SASPVLLQAMGDDAVTVATLRCDDGGAARMLTALAQAYTQGVAVDWPAVLGTTTARVLDLPTYAFQRQRYWVEWLAG
LAPEERAKALLKVVCDTAATVLGHADARTIPLTGAFKDLGVDSLTAVELRNSLTKATGLRLPATLVFDYPTPTALAV
RL
SEQ ID NO: 40
EPLAIVGMACRLPGGVSSPEDLWRLVESGTDAVSGFPTDRGWDLEDLFDPDPEAAGKSYCAEGGFLDAAAGFDAGFF
GISPREALAMDPQQRLLLEVSWEAFERAGIEPGSVRGSDTGVFIGAFPVGYAAGAAREGYGATAAPNVLSGRLSYFF
GLEGPAITMDTACSSSLVALHLAAQAVRNGECSMALAGGVTVMATPEVFTEFARQRGLASDGRCKAFADSADGAGFS
EGAGLLLVERLSDARRNGHQVLAVVRGSAVNQDGASNGFTAPNGPSQQRVIQQALANAGLTTAEVDVVEAHGTGTTL
GDPIEAQAVLATYGQDREQPLLLGTLKSNIGHTQAAAGVSGVIKMVMALQHDTVPRTLHVNEPSRHVDWTAGAVELV
TENQSWPVTDRPRRAGVSAFGVSGTNAHVILESAPAPSVNNAQPVETPVVASELVPLVISAKTLPALTEHEDRLRAY
LAASPEADMPAVASTLAVTRSVFEHRAVLLGDDTVTGTGAAVSDPRVVFVFPGQGWQWLGMGSGLRGSSVVFAERMA
ECAAALREFVDWDLFAVLDDPAVVDRVDVVQPASWAVMVSLAAVWEAAGVRPDAVVGHSQGEIAAACVAGAVSLRDA
ARIVTLRSQVIAGLAGRGGMASVALPAHEIELVEGAWIAARNGPAATVIAGEPDAVDRVLAIHEAQGVRVRRIAVDY
ASHTPHVELIHDELLGVIAGVDSQAPVVPWLSTVDGTWVEGPLDAEYWYRNLREQVGFDPAVSQLRAEGDTVFVEVS
ASPVLLQAMDDDAATVATLRRDDGDAARMLTALAQAFVEGVTVDWPAILGTATPGVLDLPTYAFQHQRFWAERWLAR
LAPVEREKALLKVVCDGAATVLGHADASTIPATAAFKDLGIDSLTAVELRNGLAKATGLRLPATLVFDYPTPTALAA
RL
SEQ ID NO: 41
EPLAIVGMACRLPGGVSSPEDLWRLVESGTDAVSGFPTDRGWDVGDLFGPAAGDSYRLRGGFLDAAGGFDASFFGIS
PREALAMDPQQRLVLEVSWEAFERAGIEPGSVRGTDTGVFMGAYPGGYGIGADLGGFGATASATSVLSGRVSYFFGL
EGPAITVDTACSSSLVALHQAGYALRQGECSLALVGGVTVMATPQTFVEFARQGGLAGDGRSKAFADSADGAGFSEG
VGVLLVERLSDAQAKGHQVLAMLRSSAVNQDGASNGLTAPNGPSQQRVIQAALSNAGLAPHEVDVVEAHGTGTTLGD
PIEAQALLATYGQDRERPLLLGSVKSNLGHTQAAAGVSGVIKMVMALRNGLVPRTLHVDEPSRHVDWSVGAVELVTE
NQSWPDSGRPRRAGVSSFGISGTNAHVILESEPPAQVVENTVVEPAPEWVPLVMSARTQSALADYEDRLRAYLAGSP
GVDLRAVGSTLAVTRSVFEHRAVLLGDDTVTGTAVSDPRVVFVFPGQGSQRAGMGEELAAAFPVFARIHQQVWDLLD
VPDLEVNETGYAQPALFALQVALFGLLESWGVGPDAVIGHSVGELAAGYVSGLWSLEDACTLVSARARLMQALPAGG
VMVAVPVSEEEAEAVLCEGVEIAAVNGPSSVVLSGDEAAVLRAAATLGKWTRLATSHAFHSARMEPMLEEFRAVAEG
LTYRTPRLTMAAGDQIATAEYWVRQVRDVVRFGEQAASCGDAVFVELGADRSLARLVDGVAMLHGDHEAQAAISALA
HLYVSGVAVDWPAVLGDVPGRVLDLPTYAFQHQRYWLEGRWLAALTPEERAKALVKVVCDSAATVLGHADASTIPVT
AAFRDLGVDSLTAVELRNSLTKATGLRLPATLVFDYPTAGALAARL
SEQ ID NO: 42
EPLAIVGMACRLPGGVFSPEDLWRLVESGTDAISGFPTDRGWDAENLFDPDPDAAGKSYCLEGGFLETAANFDASFF
EISPREALAMDPQQRLVLEASWEAFERAGIEPGSVRGSDTGVFMGAFPGGYGIGADLEGYGATSGLNVLSGRLSYFF
GLEGPAVTVDTACSSSLVALHQAGYALRQGECSLALVGGVTVMATPHTFVEFSRQRGLASDGRCKAFADSADGTGWS
EGVGVLLVERLSDAQAKGHQVLAVVRGSAVNQDGASNGLSAPNGPSQQRVIRQALANAGLTIAEVDVVEAHGTGTTL
GDPIEAQALLATYGQDREQPLLLGSVKSNVGHTQAAAGVSGVIKMVMALRNGLVPRTLHVDEPSRHVDWSEGAVELV
TENQPWPETGRPRRAGVSSFGVSGTNAHVILESAPPAQVVDNTVVESAPEWVPLVMSARTQSALADYEDRLRAYLAG
SPGVDLRAVASTLAVTRSVFEHRAVLMGDDTVTGTAVSDPRVVFVFPGQGSQRAGMGEELAAAFPVFARIHQQVWDL
LDVPDLEVNETGYAQPALFALQVALFGLLESWGVRPDAVVGHSAGELAAAYVSGVWSLEDACALVSARARLMQALPA
GGVMVAVPVSEEEAEAVLCEGVEIAAVNGPSSVVLSGDEAAVLRAAAGLGKWTRLATSHAFHSARMEPMLEEFRAVA
EGLTYRTPRLTMAAGDQVATAEYWVRQVRDVVRFGEQVASFEDAVFLELGADRSLARLVDGVAMLHGDHEAQAAISA
LAHLYVNGVTIDWPAVLGGVPGRVLDLPTYAFQHERYWAEAWLAALAPAEREKALLKLVSDGAATVLGHADASTIPV
TAAFKDLGIDSLTAVELRNSLAKATGLRLPATLVFDYPTPTALAARLD
SEQ ID NO: 43
EPLAIVGMACRLPGGVSSPEDLWRLVESGTDAISGFPTDRGWDVENLYDPDPDAPGKSYSVRGGFLDAAANFDASFF
GISPREALAMDPQQRLMLEVSWEAFERAGIEPRSVRGSDTGVFIGAYPGGYGIGVDFEGFGATAGAASVLSGRVSYF
FGLEGPAFTVDTACSSSLVALHQAGYALRQGDCSLALVGGVTVMATPQTFVEFSRQRGLSADGRCKAFADSADGTGW
AEGVGVLLVERLSDAQAKGHQVLGVVRGSAVNQDGASNGLSAPNGPSQQRVIRAALSNAGLAPHEVDVVEAHGTGTT
LGDPIEAQALLATYGQGRGEPLLLGSLKSNIGHTQAAAGVSGVIKMVMALQYGLVPRTLHVDEPSRHVDWTAGAVEL
VGENQPWPETGRPHRAGVSSFGISGTNAHVILESAPAQPAEEAQPVVTPVVASELVPLVVSAKTESALTEVEGRLRA
YLAASPGVDTRAVASTLAVTRSVFEHRAVLLGDDTVTGTGTAMSDPRVVFVFPGQGWQWLGMGSALRDSSVVFAERM
AECAAALSDFVDWDLFTVLDDPAVVDRVDVVQPASWAVMVSLAAVWQAAGVRPDAVIGHSQGEIAAACIAGAVSLRD
AARIVTLRSQAIAGLAGRGAMASIALPAQEIGLADGAWIAAHNGPASTVIAGAPEAVDRVLTAHEAQGARVRRIAVD
YASHTPHVELIRDELLDITAGIGSQAPVVPWLSTVDGTWVEGPLDAEYWYRNLREPVGFAPAVRQLQAQGETVFVEV
SASPVLLQAMDDDAVTVATLRRDDGDATRMLTALAQAYTHGVTVDWPAILGTTTTRALDLPTYAFQHERYWAEAWLV
GLAPEERAKALLKLVSDSAAAVLGHADARGIPATGAFKDLGVDSLTAVELRNTLTKATGLRLPATMVFDYPTPADLA
ARL
SEQ ID NO: 44
EPLAIVGMACRLPGGVSSPEELWQLVESGGDAISPFPTDRGWDLETPYRGGFLTDPAGFDAGFFGISPREAVAMDPQ
QRVLLEASWEAFERAGIKPDSLRGSDTGVFVGGFSQGYGTGADLGGFGATSTQTSVLSGRLSYFYGFEGPAVTVDTA
CSSSLVALHQAASSLHIGECSLAVVGGVTVVATPGGFVEFARQGGLALDGRCKAFADAADGIGLAEGVGVLLVERLS
DAQRNGHTVLAVVRGSAVNQDGASNGLSAPNGPSQQRVIRQALANARLTPADIDVVEAHGTGTRLGDPIEAQAILAT
YGQDRDQPLLLGSLKSNIGHTQAAAGVAGVIKTVMAMRHGTAPRTLHADEPSRHVDWSAGAVELLSENRLWPETDRP
RRAAVSSFGVSGTNAHVILESAPAESAEGPAGMGSESMGSESGPVVVVLSAKSASALAGQEERLRAYLASGADVRAV
AAGLARRSVFEHRSVILGDSTVSGVAAGVPRVVFLFPGQGTQWAGMGADLLESSPVFAARMRQCAAELSKYTDWDLF
TALSDPALLDRVDVVQPVSWALMVSLAALWQHCGVQPDAVIGHSQGEIAAACVAGALTLQDGARLITGRSALIAHLS
GRGTMASIALPADDLTLPDDVCIAAVNGPATTIIAGPTPAIEHLLATYEASNIHTRRIPVDYPSHTPHVEDLHDPLL
AITTHLTPHTPTTPWLSTVDNTWIHTPPHPDYWYRNLRHPVQLAPAITTLTHPHPTHLIEISTHPVLLPAIDTTTTL
TTTATLRRNHGTPHQLLTSLAHAHTHGATINWPALLGNPPTATTADLPTYPFQHKRYWLQDTERVAGLPAAEREQVV
VKAVCETAAVVLGHAHADDILATTLFKDLGVDSLIAVELRNRLAADAGLRLPATLVFDYPTPHALATWL
SEQ ID NO: 45
EPLAIVGMACRFPGGVSSPEDLWRLVESGGDAISDIPADRGWDLETPYRGGFLADAGGFDAGFFGISPREALAMDPQ
QRVLLETSWEALERAEIEPGSLRGSDTGVFIGGFSQSYGIGADLGGFGTTGIQTSVLSGRLSYFFGFEGPAFTVDTA
CSSSLVALHQASSALRQGECSLALVGGVTVLADPSGFVEFARQGGLAADGRCKAFADTADGTSLAEGVGVLLVERLS
DAQRNGHTVLAVVRGSAVNQDGASNGLSAPNGPSQQRVIRQALANARLTPADIDVVEAHGTGTRLGDPIEAQAILAT
YGQDRDQPVLIGSLKSNIGHTQAAAGVAGVIKMVMAMRQGTVPRTLHVDEPSHHVDWTAGSVQPITQNQEWPQAGRV
RRAGVSSFGISGTNAHVIIEGVPVAEPVVVADSGVVPLVLSARTPGALLEQEERLRAYLACGADVRAVAAGLARRSV
FEHRSVLVGDTVVSGTAADARLVLVFSGQGSQRAGMGEELAARFPVFAEIHQRVWDLLDVGPGLDVDDTGYAQPALF
ALQVALFGLLESWGVRPDVLIGHSIGELAAACVSGVWSLQDACALVSARARLMQALPAGGVMAAVPVSEAEAEAVLR
EGVEIAAVNGPASIVLSGDEDAVLQAAASLGRFTRLSTSHAFHCARMDPMLDEFRQVAESIAYQPPRIAMAAGDQVI
TPDYWVRQVREPVRFGDQVAAHADAVFLEIGPDRHLARLIDGIPTLSVDEVQSAMTALGELHVRGIDVDWATLLGTT
PATPTDIPTYPFQHKHYWIDNRRISGLEPAERGQALLEIVREAAAVVLGHTDAREIAPTTAFRDLGIDSLTAIELRN
RVATETGLRLPATLVFDHPTPTTLATWI
SEQ ID NO: 46
EPLAIVGMACRLPGGISSPEDLWQLVQSGGDAITDLPTDRGWDLTHLYDNDAPPVYRGGFLTDAGDFDAAFFGISPR
EALAMDPQQRILLETSWEAFERGGINPEAIRGSNTGVFIGGFSYGYGTGADLGGFGATSTQTSVLSGRLSYFYGFEG
PAVTVDTACSSSLVALHQASSALRQGECSLALAGGVTVMATPAGFEEFARQGGLAADGRCKAFSDTADGTGWAEGVG
VLLVERLSDAQRNGHTVLAVVRGSAVNQDGASNGLSAPNGPSQQRVIRQALANARLTPADIDVVEAHGTGTRLGDPI
EAQAILATYGQDRDQPLLLGSLKSNIGHTQAAAGVAGIIKTVMAMRHGTAPRTLHADEPSRHVDWSAGAVELLSENR
LWPETDRPRRAAVSSFGVSGTNAHVIIEQPPHTPAPEAERTTGLDVVPWLLSARTPAGLRAQAEQVSSLNEDFANIG
FSLATTRTPMEHRAVVVADVSGISEAAVFAGGGSPTDVVSGLANVRGKTVFVFPGQGAQWAGMGAELFATSPVFAER
MTECAAAFAALVDWSLIDVLQQREGAPSPDRVDVVQPLSFAVMVSLAALWKSHGVVPDAVTGHSQGEVAAACVSGAL
SLSDAATVVALRSRVIAQLAGHGGMVALPATEFAAEYWAGRLELAAVNGPASVVVAGEPEALEELLAENPNARRIPV
DYASHTSRVERIREELTGLLSGLAPRQPIVPFYSTVDNQWLDKPLDAEYWYRNLRQTVRFADAVHGLADAGFRAFVE
VSPHPVLTSSMRDILDERETTAVVTGTLRRDAHGVREFVRSLARLWVSGFSVDWSGLFGNGPRRIPLPTYPFQRNRY
WLQAELDLVRTHAAAVLGHAGPEAVAADHPFRDLGVDSLIAVELRNRLAAETGLRLPATLVFDYPTPRALAAWLD
SEQ ID NO: 47
EPLAIVGMACRFPGGVSSPEDLWRLVETSGDAISDIPADRGWDLETPYRGGFLIGAAGFDAGFFGISPREALAMDPQ
QRLLLEISWEALERAGINPESVRGSDTGVFVGGSSYGYGVGADLGGFGATSTHISVLSGRVSYFFGFEGPAFTVDTA
CSSSLVALHQASSALRQGECSLALVGGVTVMATPAGFEEFARQGGLAADGRCKAFSDTADGTSLAEGVGVLLVERLS
DAQRNGHTVLAVVRGSAVNQDGASNGLSAPNGPSQQRVIRQALANARLTPADIDVVEAHGTGTTLGDPIEAQAILAT
YGQDRDQPVLIGSLKSNIGHTQAAAGVAGVIKMVMAMRQGTVPRTLHVDEPSHHVDWTAGSVQPITQNQEWPQAGRV
RRAGVSSFGISGTNAHVIIEGVPVAEPVVVADSGVVPLVLSARTPGALLEQEERLRAYLACGADVRAVAAGLARRSV
FEHRSVLVGDTVVSGTAADARLVLVFSGQGSQRAGMGEELAARFPVFAEIHQRVWDLLDVGPGLDVDDTGYAQPALF
ALQVALFGLLESWGVRPDVLIGHSIGELAAACVSGVWSLQDACALVSARARLMQALPAGGVMAAVPVSEAEAEAVLR
EGVEIAAVNGPASIVLSGDEDAVLQAAASLGRFTRLSTSHAFHCARMDPMLDEFRQVAESIAYQPPRIAMAAGDQVI
TPDYWVRQVREPVRFGDQVAAHADAVFLEIGPDRHLARLIDGIPTLSVDEVQSAMTALGELHVRGIDVDWATLLGTT
PATPTDIPTYPFQHKHYWIDNTRISGLEPAERGQALLEIVREAAAVVLGHTDAREIAPTTAFRDLGIDSLTAIELRN
RVATETGLRLPATLVFDHPTPTTLATWI
SEQ ID NO: 48
EPLAIVGMACRLPGGISSPEDLWQLVQSGGDAITDLPTDRGWDLETPYRGGFLTDPAGFDAGFFGISPREALAMDPQ
QRVLLEASWEAFERAGIKPDSLRGSDTGVFVGGFSQGYGTGADLGGFGATSTQTSVLSGRLSYFYGFEGPAVTVDTA
CSSSLVALHQASSALRQGECSLALAGGVTVMATPAGFEEFARQGGLAADGRCKAFADTADGTGWAEGVGVLLVERLS
DAQRNGHTVLAVVRGSAVNQDGASNGLSAPNGPSQQRVIRQALANARLTPADIDVVEAHGTGTRLGDPIEAQAILAT
YGQDRDQPLLLGSLKSNIGHTQAAAGVAGIIKTVMAMRHGTAPRTLHADEPSRHVDWSAGAVELLSENRLWPETDRP
RRAAVSSFGVSGTNAHVILESAPAESAEGPAGMGSESMGSESGPVVVVLSAKSASALAGQEERLRAYLASGADVRAV
AAGLARRSVFEHRSVILGDSTVSGVAAGVPRVVFLFPGQGTQWAGMGADLLESSPVFAARMRQCAAELSKYTDWDLF
TALSDPALLDRVDVVQPVSWALMVSLAALWQHCGVQPDAVIGHSQGEIAAACVAGALTLQDGARLITGRSALIAHLS
GRGTMASIALPADDLTLPDDVCIAAVNGPATTIIAGPTPAIEHLLATYEASNIHTRRIPVDYPSHTPHVEDLHDPLL
AITTHLTPHTPTTPWLSTVDNTWIHTPPHPDYWYRNLRHPVQLAPAITTLTHPHPTHLIEISTHPVLLPAIDTTTTL
TTTATLRRNHGTPHQLLTSLAHAHTHGATINWPALLGNPPTATTADLPTYPFQHKRYWLQDTERLTTQSSVEQHRLM
LDLVTSHAAAVLGHSSAAAITTDTPFRDLGFDSLTAVELRNRVAADTGLRLPATLVFNHPNADALTQYL
SEQ ID NO: 49
DPIAIVAMACRLPGGVSSPEDLWRLVETGTDAIGPFPTDRGWDTELYPVPDAPGKTYCVEGGFLTGAAEFDAAFFDI
SPREALAMDPQQRLLLETSWEAVERARINPKSLCGKDVGVYVGAAAQGYGLGAGDQTEGTAITGGSTSLSSGRVSYA
LGLEGPAVTVDTACSSSLVAMHLAGQALRQGECSLALVGGVSVMASPALFVEFSRQRGLAADGRCKSFSDAADGTNW
AEGVGVLILERLSDAQRNGHPVLAVIRGSAINSDGASNGLTAPNGLSQQRVIRQALTAAGLRPEDVDAVEAHGTGTR
LGDPVEAEAILATYGQNREQPLLLGSLKSNIGHAAAASGVAGVIKMVQAMRNGVLPRTLHIDEPSSQVDWTSGNVAL
LTESRPWPDEDKPRRAGVSSFGISGTNAHIVLEQYRAAEPEDRPGDGPGERRPVAWVLSGKSPAAVRAQAGRLRAHL
VGTQGWRPVDVGYALATTRADFAHRAVAVGSGPEFLHALEKLAEGASWPRLTTNRASARRVAFLFDGQGTQRLGMGR
ELHQRFPAFAEAWDTVDAEFAPYLDRSLTEVFFSDGGSGLMDDTLYAQAGLFAVETALFRLLAGWGVRPDFVAGHSA
GEITAAHVAGVLSVTDAVRLIVARGQALRLAPPGAMASVRSSAQEVRDFIAQSGLPVDLAAINSPGSVVVAGSPETI
AEFEGAWTASGRQAKRLAVRHAFHSRHVDGVLDEFRAALGGCRFGVAELPLVSTATGELASPDELGTPEHWLRHARQ
TVRFQDAIRALTEQGVDTFVEIGPSGTLASAGMECGGGTAAFHAVMRARQPEEVSLMTAVAELYAGGTPVEWSRVLD
GRSVVDLPVYPFQRQPYWLAPADELSQPEQQKALLELVKAEAAVLLGITDATAIEDDARFLELGFDSLSATRLRNQL
AKATGLALEQTLLFDFPTPAALAAHL
SEQ ID NO: 50
EPLAIVGMACRLPGGVSSPEDLWRLVESGGDVISDFPTDRGWDTTGEDSSFIRGGFLTDAGGFDAGFFGISPREAVA
MDPQQRLVLETSWEVLERAGIEPGSLRGSDTGVFIGGFSQGYGAGADLGGFGATGTQTSVLSGRVSYYLGLEGPAVT
VDTACSSSLVALHQAASALRQGECSLALVGGVTVMATTHSFVEFARQGGLSSDGRCRSFADSADGTGWAEGVGVLLV
ERLSDARRSGHPVLALVRGSAVNQDGASNGLSAPNGLSQQRVIRQALATAGLDAADVDVVEAHGTGTVLGDPIEAQA
ILATYGQGREEPLLLGSLKSNVGHTQAAAGVAGVIKMVMAMRQGTVPRTLHVDEPSHHVDWTAGRVELLTENRPWPQ
AGRVRRAGVSSFGISGTNAHVIIEGVPVAEPVVVADSGVVPLVLSARTPGALLEQEERLRAYLACGADVRAVAAGLA
RRSVFEHRSVLVGDTVVSGTAADARLVLVFSGQGSQRAGMGEELAARFPVFAEIHQRVWDLLDVGPGLDVDDTGYAQ
PALFALQVALFGLLESWGVRPDVLIGHSIGELAAACVSGVWSLQDACALVSARARLMQALPAGGVMAAVPVSEAEAE
AVLREGVEIAAVNGPASIVLSGDEDAVLQAAASLGRFTRLSTSHAFHCARMDPMLDEFRQVAESIAYQPPRIAMAAG
DQVITPDYWVRQVREPVRFGDQVAAHADAVFLEIGPDRHLARLIDGIPTLSVDEVQSAMTALGELHVRGIDVDWATL
LGTTPATPTDIPTYPFQHKHYWIDNTRISGLEPAERGQALLEIVREAAAVVLGHTDAREIAPTTAFRDLGIDSLTAI
ELRNRVATETGLRLPATLVFDHPTPTTLATWI
SEQ ID NO: 51
EPLAIVGMACRLPGGISSPEDLWQLVQSGGDAITDLPTDRGWDLETPYRGGFLTDPAGFDAGFFGISPREALAMDPQ
QRVLLEASWEAFERAGIKPDSLRGSDTGVFVGGFSQGYGTGADLGGFGATSTQTSVLSGRLSYFYGFEGPAVTVDTA
CSSSLVALHQASSALRQGECSLALAGGVTVMATPAGFEEFARQGGLAADGRCKAFADTADGTGWAEGVGVLLVERLS
DAQRNGHTVLAVVRGSAVNQDGASNGLSAPNGPSQQRVIRQALANARLTPADIDVVEAHGTGTRLGDPIEAQAILAT
YGQDRDQPLLLGSLKSNIGHTQAAAGVAGIIKTVMAMRHGTAPRTLHADEPSRHVDWSAGAVELLSENRLWPETDRP
RRAAVSSFGVSGTNAHVILESAPAESAEGPAGMGSESMGSESGPVVVVLSAKSASALAGQEERLRAYLASGADVRAV
AAGLARRSVFEHRSVILGDSTVSGVAAGVPRVVFLFPGQGTQWAGMGADLLESSPVFAARMRQCAAELSKYTDWDLF
TALSDPALLDRVDVVQPVSWALMVSLAALWQHCGVQPDAVIGHSQGEIAAACVAGALTLQDGARLITGRSALIAHLS
GRGTMASIALPADDLTLPDDVCIAAVNGPATTIIAGPTPAIEHLLATYEASNIHTRRIPVDYPSHTPHVEDLHDPLL
AITTHLTPHTPTTPWLSTVDNTWIHTPPHPDYWYRNLRHPVQLAPAITTLTHPHPTHLIEISTHPVLLPAIDTTTTL
TTTATLRRNHGTPHQLLTSLAHAHTHGATINWPALLGNPPTATTADLPTYPFQRRRFWAERISGLEPAERGQALLEI
VREAAAVVLGHTDAREIAPTTAFRDLGIDSLTAIELRNRVATETGLRLPATLVFDHPTPTTLATWI
SEQ ID NO: 52
EPLAIVGMACRLPGGISSPEDLWQLVQSGGDAISDFPTDRGWDLTHLYDNDAPPVYRGGFLTDAGDFDAAFFGISPR
EALAMDPQQRLILETSWEVLERAGIEPGTLRGSETGVFVGGFTQGYGTGADLGGFGMTSGHSSVLSGRVSYFFGFEG
PAVTVDTACSSSLVALHQASSALRQGECSLALVGGVTVMASPQGFTEFSRQGGLSPDGRCKAFADAADGTGWAEGVG
VLLVERLSDAQRNGHTVLAVVRGSAVNQDGASNGLSAPNGPSQQRVIRQALANARLTPADIDVVEAHGTGTTLGDPI
EAQAILATYGQDRDQPLLLGSLKSNIGHTQAAAGVAGVIKMVMAMRHGTAPRTLHIDEPSRHIDWTTGSVALSTENQ
PWPETGHPRRAGVSAFGVSGTNAHVVLEGVPVAGPPEEDVEPGVVPLLISAKSRPALMEQEQRLRTYLDGSQTDIRA
VAATLAHARSVFEHRSVLVGDTVVSGTAADARLVLVFSGQGSQRAGMGEELAARFPVFAEIHQRVWDLLDVGPGLDV
DDTGYAQPALFALQVALFGLLESWGVRPDVLIGHSIGELAAACVSGVWSLQDACALVSARARLMQALPAGGVMAAVP
VSEAEAEAVLREGVEIAAVNGPASIVLSGDEDAVLQAAASLGRFTRLSTSHAFHCARMDPMLDEFRQVAESIAYQPP
RIAMAAGDQVITPDYWVRQVREPVRFGDQVAAHADAVFLEIGPDRTLARLIDGVPLLSKEDEVQAALVALAELHVRG
VPLEWSTVIGGMTSIVDLPTYPFRRKRYWIESAERLTTQSSVEQHRLMLDLVTSHAAAVLGHSSAAAITTDTPFRDL
GFDSLTAVELRNRVAADTGLRLPATLVFNHPNAGDLARHL
SEQ ID NO: 53
EPLAIVGMACRLPGGISSPEDLWQLVQSGGDAITDLPTDRGWDLTHLYDNDAPPVYRGGFLTDAGDFDAAFFGISPR
EALAMDPQQRILLETSWEAFERGGINPEAIRGSNTGVFIGGFSYGYGTGADLGGFGATSTQTSVLSGRLSYFYGFEG
PAVTVDTACSSSLVALHQASSALRQGECSLALAGGVTVMATPAGFEEFARQGGLAADGRCKAFADTADGTGWAEGVG
VLLVERLSDAQRNGHTVLAVVRGSAVNQDGASNGLSAPNGPSQQRVIRQALANARLTPADIDVVEAHGTGTTLGDPI
EAQAILATYGQDRDQPLLLGSLKSNIGHTQAAAGVAGIIKTVMAMRHGTAPRTLHADEPSRHVDWSAGAVELLSENR
LWPETDRPRRAAVSSFGVSGTNAHVILESAPAESAEGPAGMGSESMGSESGPVVVVLSAKSASALAGQEERLRAYLA
SGADVRAVAAGLARRSVFEHRSVILGDSTVSGVAAGVPRVVFLFPGQGTQWAGMGADLLESSPVFAARMRQCAAELS
KYTDWDLFTALSDPALLDRVDVVQPVSWALMVSLAALWQHCGVQPDAVIGHSQGEIAAACVAGALTLQDGARLITGR
SALIAHLSGRGTMASIALPADDLTLPDDVCIAAVNGPATTIIAGPTPAIEHLLATYEASNIHTRRIPVDYPSHTPHV
EDLHDPLLAITTHLTPHTPTTPWLSTVDNTWIHTPPHPDYWYRNLRHPVQLAPAITTLTHPHPTHLIEISTHPVLLP
AIDTTTTLTTTATLRRNHGTPHQLLTSLAHAHTHGATINWPALLGNPPTATTADLPTYPFQHKRYWLQDTRLSALAP
AEREQALVKAVCETAAMVLGHADTREIAATTAFKELGLDSLTAVQLRDRLAAETGRKLPATLVFDYPSPQALAAWL
SEQ ID NO: 54
EPLAIVGMACRLPGGVASPEDLWRLVESGTDAITDFPTDRGWDLDEVADQSYCLQGGFLDNAAGFDAAFFGISPREA
LAMDPQQRLVLEASWEAFERAGIKPGSLRGSDTGVFMGAYPGGYGTGADLGGFGATAGAVSVLSGRISYFFGFEGPA
MTVDTACSSSLVALHQAGYALRQGECSIALVGGVTVMATPQSFIEFSRQRGLAADGRCKTFADAADGTGWAEGVGVL
LVERLSDARAKGHQILAVVRGSAVNQDGASNGLSAPNGPSQQRVIRQALVNAGLSPADVDVVEAHGTGTTLGDPIEA
QALLTTYGQGRSVPLLLGSVKSNLGHTQAAAGVTGVIKMVMALRHGVVPRTLHVDEPSRHVDWSAGAVELVTSNREW
PVVDRPGRAGVSSFGISGTNAHVILEAVPSDTPASTSTDAVLPLVVSARTAPAAEDLTARLRAYLSAAPETDQRAAA
ATLALTRSVFEHRAVVLGDELVSGQAVRDPRVVFVFSGQGSQRAGMGEQLAAVFPVFAEIHERVWALLDVPDGLDVD
DTGHAQPALFALQVALSGLLESWGVRPAAVIGHSIGELAAAYVSGVWSLEDACALVSARARLMQALPPGGVMVAVPV
PEAEARAVLRDGVEIAAVNGPSSVVLSGDEDAVLQAVSGFAKWTRLKTSHAFHSAHMDPMLDEFRAVAERLTYRRPS
VEMAAGDRVTTAEYWVRQVREAVRFGDQTTAYEDAVFVEIGPGRTLARLIDGITMLHGDTEREAALTGLSQLFVRGV
DVDWATVIEDTTARILDLPTYAFQHENYWLHWLSGLTPAEREQALLTAVRENAAAVLGHADARTVPVNSAFRDLGFD
SLTAIELRNSLAKATGLSLPATMAFDYPTPAVLATRL
SEQ ID NO: 55
EPLAIVGMACRLPGGVSSPEELWRLVESGVDAISGFPVDRGWDVENLFDPDPDAAGKSYCVQGGFLDSAAEFDAAFF
GISPREALAMDPQQRLVLETSWEAFERAGIEPGSIKGSDTGVFMGAYQGGYGSGADLGGFGATAGATSVLSGRVSYF
FGFEGPAMTVDTACSSSLVALHQAGYALRQGECSLALVGGVTVMATPQSFVEFSRQRGLAVDGRSKAFADAADGTGW
AEGVGVLLVERLSDAQAKGHQILAVVRGSAVNQDGASNGLSAPNGPSQQRVIRQALANASLTPADVDVVEAHGTGTT
LGDPIEAQAVIATYGQDRSTPLLLGSLKSNIGHTQAAAGVSGVIKMVMALRHGVVPQTLHVDQPSRHVDWSAGAVEL
LTSNQPWPSSERARRAGVSAFGVSGTNAHVILESAPAEPVVAEAGPVPVVSDVLPLVLSAKSAPALRALEQRLRAYD
GAAGRALATARATFDHRAVLIGDDTVTGVAVPDPRVVFVFPGQGWQWLGMGRELRGSSVVFAERMAECAAALSEFVD
WDLFAALDDPAVVDRVDVVQPVCWAVMVSLAAVWQAAGVNPDAVVGHSQGEIAAAVVAGSLSLRDGARVVALRSQLI
KGLAGRGAMASIALPADQIGLVEGAWIAALNGPSSTVIAGTPEAVEQVLAAQDARVRRIAVDYASHTPQVEAIRDEL
LELTAGVSSQPPTVPWLSTVDNTWVEGPLPADYWFRNLREQVGFAQAVVTLGDAVFVEVSGSPVLMQSMGDAVTVAS
FRRDDGSATRMVTSLAEAYVQGVNVNWAAVLGAGTERALDLPTYPFQRQHYWISLAALPPAERERALLKVVRDSAAV
VLGHADGRTVPATAAFKDLGLDSLTAVELRNSLRKATGLQLPATLVFDYPSPVALAARL
SEQ ID NO: 56
EPLAIVGMSCRLPGGVSSPEDLWRLVESGVDAISGFPVDRGWDAEGLFDPDPDAAGKTYCVQGGFLEAAGEFDTAFF
GISPREALAMDPQQRVLLEASWEAFERAGIGADTVRGTDTGVFIGAYPVAYGAGVDREGYGATAAPNVLSGRLSYFF
GLEGPAITVDTACSSSLVALHLAASALRNGECSLALAGGVTVMATPEVFTEFARQRGLAFDGRSKSFADAADGAGFS
EGAGLLVLERLSDARRNGHQVLAVIRGSAVNQDGASNGFTAPNGPSQQRVIEAALGNAGLTTAEVDVVEAHGTGTKL
GDPIEAQAVLATYGQDRDLPLLLGSLKSNIGHTQAASGVAGVIKMVMALRHGVVPQTLHVDEPSRHVDWSAGAVELV
TSNQPWPSSERPRRAGVSAFGVSGTNAHVILESAPVEPVVAEAGPVPVVGDVLPLVVSAKSAPALTVLEQRLRAYEA
ADEKAVAATLAAARATFGHRAVLLGGDTVTGVAVPDPRVVFVFPGQGWQWLGMGRELRGSSVVFAERMAECAAALSE
FVDWDLFTALDDPAVVDRVDVVQPVCWAVMVSLAAVWQASGVNPDAVVGHSQGEIAAAVVAGSLSLRDGARVVALRS
QLIKGLAGRGAMASIALPAAEIDLVEGSWIAALNGPSSTVIAGTPEAVEQVLAVQDARVRRIAVDYASHTPQVEAIR
DELLELTGEVVSRKPDVPWLSTVDNAWIEGPLGADYWFRNLREQVGFAQAVVTLGDAVFVEVSASPVLMQSMGDAVC
VPSLRRDDGTATRMVTSLAEAYVQGVQVNWAAVLGAGTERALDLPTYPFQRQHYWALHWLARLSPAEREQALLKLVC
ESASVVLGHADAGAIPVTAAFKDLGVDSLTAVELRNSLATATGQRLPATAVFDYPTPAVLAARL
SEQ ID NO: 57
EPLAIVGMACRLPGGVSSPEGLWRLVVSGSDVISGFPADRGWGVEGLRGGFLPGAADFDAGFFGISPREALAMDPQQ
RLVLEASWEVLERAGIAPGSLRGSDTGVFMGAYPGGYGIGADLGGFGATAGAVSVLSGRVSYFFGFEGPAMTVDTAC
SSSLVALHQAGHALRNSECSLALVGGVTVMASPQTFVEFERQGGLAADGRSKAFSDGADGAGFSEGVGVLLVERLSD
ARAKGHQILALVRGSAVNQDGASNGLTAPNGPSQQRVIRQALANASLTVADVDVVEAHGTGTTLGDPIEAQALLATY
GQDRDRPLLLGSVKSNLGHTQAAAGVTGVIKMVMALRHGVVPRTLHVDEPSRHVDWSAGAVELVTSNREWPVTDRPG
RAGVSSFGISGTNAHVILEAVPVVSAVSTGGEVQPLVVSARTAPAAEDLTARLRTYLADTPDTDQRAAATTLALTRS
VFEHRAVLLGDDTITGAAVPDPRVVFVFSGQGSQRAGMGEQLAAAFPVFAEIHERVWALLDVPDGLDVDDTGHAQPA
LFALQVALSGLLESWGVRPAAVIGHSIGELAAAYVSGVWSLEDACVLVSARARLMQALPPGGVMVAVPVPEAEARAV
LRDGVEIAAVNGPSSVVLSGDEDAVLQAVAGFAKWTRLKTSHAFHSAHMDPMLDEFRAVAERLTYRRPSVEMAAGHG
VTTAEYWVRQVREAVRFGDQTTAYEDAVFVEIGPGRTLARLIDGITMLHGDTEREAALTGLSQLFVRGVDVDWPAVI
EDTTARILDLPTYPFQRQRYWLTPRWLAGMSPEDRRQALLRVVRDSAAVVLGHAEAGTIPPNAAFKDLGIDSLTAVE
LRNSLATATGLRLPATLVFDYPAPETLAARLD
SEQ ID NO: 58
EPLAIVGMACRLPGGVASPEDLWRLVASGTDAISGFPTDRGWDVEGLFDPDPDVAGKTYCVQGGFLDTAARFDAAFF
GISPREALAMDPQQRLVLEASWEAFERAGIEPGSLRGSDTGVFMGAFPGGYGLGADLEGYGVTGGPNAVSGRLSYFF
GLEGPAFTVDTACSSSLVALHQAGYALRQGECSLALVGGVTVMGTPQTFVEFSRQRGLAVDGRSKSFSDQADGTGWS
EGVGVLVVERLSDARAKGHQILAVVRGSAVNQDGASNGLTAPNGPSQQRVIRQALANASLTVADVDVVEAHGTGTTL
GDPIEAQALLATYGQDRDRPLLLGSVKSNLGHTQAAAGVAGVIKMVMALQHGIVPQTLHVSEPSRHVDWTAGAVELV
TSNQPWPSSGRPGRAGVSAFGVSGTNAHVILEGVPSNTPVSTAAGDVLPLVVSARTAPAVEDLTARLRTYLADTPGT
DQRAAATTLALTRSVFEHRAVLLGEDTITGVAVPDSRVVFVFSGQGSQRAGMGEQLAAAFPVFAAIHERVWALLDVP
DGLDVDDTGHAQPALFALQVALSGLLESWGVRPDAVIGHSIGELAAAYVSGVWSLEDACALVSARARLMQALPSGGV
MVAVPVPEAEARAVLRDGVEIAAVNGPSSVVLSGDEDAVLQAVAGFAKWTRLKTSHAFHSAHMDPMLDEFRAVAERL
TYRRPSVEMAAGDRVTTAEYWVRQVREAVRFGDQTTAYEDAVFVEIGPGRTLARLIDGITMLHGENEGHAALAALSH
LFVQGVRVDWPAVLGTTAERVDLPTYPFQHEHYWARAEHWLAGLPADEREKALLKIVRDSAAAVLGHADGRTVASGA
VFKELGLDSLTAVELRNSLGKATGLRLPSTAAFDYPTPAALATRL
SEQ ID NO: 59
EPLAIVGMACRLPGGVSSPEDLWRLVESGSDAISGFPTDRGWDVDGLFDPDPDAAGKSYCVQGGFLDSAAEFDAAFF
GISPREALAMDPQQRLLLETSWEAFERAGIDPGSVRGSDTGVFVGAFPGGYGAGADIEGYGATAGPSVLSGRLSYFF
GLEGPAFTVDTACSSSLVALHQAGHALRQGECSLALVGGVTVMASPVTFVEFSRQRGLAADGRCKAFGDGADGTGWS
EGVGVLLVERLSDAQAKGHQILAVVRGSAVNQDGASNGLTAPNGPSQQRVIQAALASAGLVTSDVDVVEAHGTGTTL
GDPIEAQAVLATYGQDRSTPLLLGSLKSNIGHTQAAAGVSGVIKMVMALRHGVVPQTLHADEPSRHVDWSAGAVELL
TSNRSWPSSERARRAGVSAFGVSGTNAHVILESAPVEPVVAVAGPVPVVSDVLPLVLSAKSAPALTALEQRLRVYDG
AAGRALATARATFDHRAVLIGDDTVTGVAVPDPRVVFVFPGQGWQWLGMGRELRDSSVVFASRMAECAAALSEFVDW
DLFTALDDPAVVDRVDVVQPVCWAVMVSLAAVWQASGVNPDAVVGHSQGEIAAAVVAGSLSLRDGARVVALRSQLIK
GLAGRGAMASIALPAAEIDLVEGSWIAALNGPSSTVIAGTPEAVEQVLAVQDARVRRIAVDYASHTPQVEAIRDELL
ELTAEVESRRPDVPWLSTVDNTWVEGPLSADYWFRNLREQVGFAQAVVTLGDAVFVEVSASPVLMQSMGDAVTVATL
RRDDGSALRMVTSLAEAYVQGVNVNWAAVLGAGTERALDLPTYPFQRQHYWVTAQSLAGLPAEDREKALLKIVRDSA
AQVLGHPDGRAVPAGAAFIELGVDSLTGVEMRNRLGGITGLRLPATMVFDYPTPAALAGRL
SEQ ID NO: 60
EPLAIVGMACRLPGGVSSPEELWRLVESGVDAISGFPVDRGWDVENLFDPDPDAAGKSYCVQGGFLDTAAEFDAAFF
GISPREALAMDPQQRLVLETSWEAFERAGIEPGSLKGSDTGVYMGAFSGGYAADLEGFGATAGATSVLSGRVSYFFG
FEGPAMTVDTACSSSLVALHQAGYALRQGECSLALVGGVTVMASPQSFVEFSRQRGLAADGRSKAFADAADGTGWAE
GVGVLLVERLSDAQAKGHQILAVVRGSAVNQDGASNGLSAPNGPSQQRVIRQALANASLTPADIDVVEAHGTGTTLG
DPIEAQAVIATYGQDRSTPLLLGSLKSNIGHTQAAAGVSGVIKMVMALRHGVVPQTLHVDQPSRHVDWSAGAVELVT
SNQPWPSSERPRRAGVSAFGVSGTNAHVILESAPAEPVVAEVGLVPVVSDVLPLVLSAKSAPALTVLEQRLRAYEAA
DERTVAATLATARATFDHRAVLIGTETVTGPLMTDPRVVFVFPGQGWQWLGMGRELRGSSVVFAERMAECAAALSDF
VDWDLFTALDDPAVVDRVDVVQPVCWAVMVSLAAVWQAAGVNPDAVVGHSQGEIAAAVVAGSLSLRDGALVVALRSQ
LIKGLAGRGAMASIALPADQIGLVEGAWIAALNGPSSTVIAGSPEAVEQVLAAQDARVRRIAVDYASHTPQVEAIRD
ELLELTAGVSSQPPTVPWLSTVDNTWVEGPLPADYWFRNLREQVGFAQAVVTLGDAVFVEVSASPVLMQSMGDAVCV
PSLRRDDGSATRMVTSLAEAYVQGVNVNWAAVLGAGTERALDLPTYPFQRQRYWAGHWLARLAPGERETALLKLVSE
SAAAVLGHADARSIPATAVFRDLGMDSLTAVEVRNSLAKTTGLRLPATLAFDYPTPAVLAARL
SEQ ID NO: 61
EPLAIVGMACRLPGGVSSPEGLWRLVVSGSDVISGFPADRGWGVEGLRGGFLPGAADFDAGFFGISPREALAMDPQQ
RLVLEASWEVLERAGIAPGSLRGSDTGVFMGAYPGGYGIGADLGGFGATAGAVSVLSGRVSYFFGFEGPAMTVDTAC
SSSLVALHQAGHALRNSECSLALVGGVTVMASPQTFVEFERQGGLAADGRSKAFSDGADGAGFSEGVGVLLVERLSD
ARAKGHQILALVRGSAVNQDGASNGLTAPNGPSQQRVIRQALANASLTVADVDVVEAHGTGTTLGDPIEAQALLATY
GQGRSVPLLLGSVKSNLGHTQAAAGVTGVIKMVMALRHGVVPRTLHVDEPSRHVDWSAGAVELVTSNREWPVVDRPG
RAGVSSFGISGTNAHVILEGIPSNTPVSTAAGAVLPLVVSARTAPAAEDLTARLRAYLSAAPETDQRAAAATLALTR
SVFEHRTVLLGDDTITGAAMPDPRVVFVFSGQGSQRAGMGEQLAAVFPVFAEIHERVWALLDVPDGLDIDDTGHAQP
ALFALQVALSGLLESWGVRPDAVIGHSIGELAAAYVSGVWSLEDACALVSARARLMQALPPGGVMVAVPVSEAEART
VLRDGVEIAAVNGPSSVVLSGDEDAVLQAVSGFAKWTRLKTSHAFHSAHMDPMLDEFRAVAERLTYRRPSVEMAAGH
GVTTAEYWVRQVREAVRFGDQTTAYEDAVFVEIGPGRNLARLIDGITMLHGDTEREAALTGLSQLFVRGVDVDWATV
IEDTTARILDLPTYPFQHERYWLSWLVGLPPAERAKALLKTVRDSAAVVLGHQGTRAIPVDGAFRELGMDSLTAVEL
RNSLAKATGLSLSATLVFDYPTPKVLADHLD
SEQ ID NO: 62
EPLAIVGMACRLPGGVSSPEELWRLVESGSDAISGFPVDRGWDADGLFDPDPDAAGKSYCVQGGFLDTAAEFDAAFF
GISPREALAMDPQQRLVLETSWEAFERAGIEPGSIKGSDTGVFIGAYPGGYGSGVELGGFGATSGAGSVLSGRVSYF
FGFEGPAMTVDTACSSSLVALHQAGYALRQGDCSMALVGGVTVMSTPHIFVEFSRQRGLAADGRCKAFGDGADGTGW
SEGVGVLLVERLSDARAKGHQVLAVVRGSAVNQDGASNGLTAPNGPSQQRVIHAALASAGLVTSDVDVVEAHGTGTT
LGDPIEAQAVIATYGQDRSTPLLLGSLKSNIGHTQAAAGVSGVIKMVMALRHGVVPQTLHVDQPSRHVDWSAGAVEL
LTSNQPWPSSERARRAGVSAFGVSGTNAHVILESAPVEPVVAEAGPVPVVSDVLPLVLSAKSAPALRALEQRLRVYD
GAAGRALATARATFDHRAVLIGDDTVTGVAVPDPRVVFVFPGQGWQWLGMGRELRGSSVVFAERMAECAAALSEFVD
WDLFAALDDPAVVDRVDVVQPVCWAVMVSLAAVWQAAGVNPDAVVGHSQGEIAAAVVAGSLSLRDGALVVALRSQLI
KGLAGRGAMASIALPATEISLVEGAWIAALNGPSSTVIAGSPEAVEQVLAVQDARVRRIAVDYASHTPQVEAIRDEL
LELTAGVSSQLPTVPWLSTVDNTWVEGPLPADYWFRNLREQVGFAAAVQELGESVFVEVSGSPVLIQSMGDAVTVAT
LRRDDGSATRMVTSLAEAYVQGVQVNWAAVLGAGSERALDLPTYPFQRDHFWVLSLAALPSAEREKALVKIVCESAA
AVLGHTDTSAVPAAAAFKELGLDSLTAVDLRNRLRRATGLQLPATLVFDYPTPTAMAARL
SEQ ID NO: 63
EPLAIVGMSCRLPGGVSSPEDLWRLVESGSDAISGFPTDRGWDVDGLFDPDPDAAGKTYCVQGGFLEAAGEFDAAFF
GISPREALTMDPQQRVLLEASWEAFERAGIAPTSVRGTDTGVFIGAFPVGYGAGADHEGYTATAGVGSVLSGRLSYF
FGLEGPAMTMDTACSSSLVALHLAASALRNGECSLALAGGVTVMATPEVFTEFARQRGLAADGRCKPFADAADGAGF
SEGAGLLVLERLSDARRNGHQVLAVIRGSAVNQDGASNGLTAPNGPAQQRVIRQALANAGLNSSDVDVLEAHGTGTT
LGDPIEAQAVLATYGQDRSTPLLLGSLKSNIGHTQAASGVAGVIKMVMALRNGLVPRSLHLDEPSRHVDWSAGAVEL
LTSNQPWPSSDRPRRAGVSAFGVSGTNVHVILESAPAEPVGAEAGPLPVVGDVLPLVVSAKSAPALTALEQRLRAHV
AADERAAAATLATARATFDHRAVLIGAETVTGVAAVDPRVVFVFPGQGWQWLGMGRELRGSSVVFAERMAECAAALS
EFVDWDLFTALDDPAVVDRVDVVQPVCWAVMVSLAAVWQAVGVNPDAVVGHSQGEIAAAVVAGSLSLRDGALVVALR
SQLIAGLAGRGAMASIALPADQISLVEGAWIAALNGPSSTVIAGTPEAVEQVLAAQDARVRRIAVDYASHTPQVEAI
RDELLELTGEVVSRKPDVPWLSTVDNAWIEGPLGADYWFRNLREQVGFAQAVVTLGDAVFVEVSASPVLMQSMGDAV
CVPSLRRDDGTATRMVTSLAEAYVQGVQVNWAAVLGAGTERALDLPTYPFQRERFWVLWLAGLAPQERETALLKLVC
DSAAVVLGHGDGQAIPDTTAFKDLGVDSLTAVEVRNRLAAATGLRLPATMVFDYPTPTALAARL
SEQ ID NO: 64
EPLAIVGMACRLPGGVASPEDLWRLVESGTDAITGFPADRGWTTEPGQGGFLADAAGFDAAFFGISPREALAMDPQQ
RLLLETSWEAFERAGIAPLSLRGSDTGVYIGAYPDGYGIGADLGGFGTTAGSPSVLSGRVSYFFGLEGPAITVDTAC
SSSLVALHQAGYALRNNECSLALVGGVTVMATPEVFSAFALQDGLAADGRSKAFSDGADGAGFSEGVGVLLVERLSD
AQANGHQILALVRGSAVNQDGASNGLTAPNGPSQQRVIRQALANASLTPADVDVIEAHGTGTTLGDPIEAQALLATY
GQGRSVPLLLGSVKSNLGHTQAAAGVTGVIKMVMALRHGVVPRTLHVDEPSRHVDWSAGAVELVTSNREWPVTDRPG
RAGVSSFGISGTNAHVILEAVPVVSAVSTGGEVQPLVVSARTAPAAEDLASRLRTYLADTPDTDQRAAAATLALTRS
VFEHRTVLLGDDTITGAAMPDPRVVFVFSGQGSQRAGMGEQLAAVFPVFAEIHERVWALLDVPDGLDIDDTGHAQPA
LFALQVALSGLLESWGVRPDAVIGHSIGELAAAYVSGVWSLEDACALVSARARLMQALPPGGVMVAVPVPEAEARTV
LRDGVEIAAVNGPSSVVLSGDEDAVLQAVSGFAKWTRLKTSHAFHSAHMDPMLDEFRTVAERLTYRRPSVEMAAGHG
VTTAEYWVRQVREAVRFGDQTTAYEDAVFVEIGPGRTLARLIDGITMLHGDTEREAALTGLSQLFVRGVDVDWATVI
EDTTARILDLPTYPFQHERYWAGRWLAGLAPDKRDAALLTMVRDSAARVLGHADGSAISPTATFRDLGVDSLTAVEL
RNRLARTAGLRLATTIVFDYPTPTALAAHL
SEQ ID NO: 65
EPLAIVGMACRLPGGVASPEDLWRLVESGTDAITGLPTDRGWDLGAVAAESYCVEGGFLDGVAGFDAAFFGISPREA
LAMDPQQRLLLETSWESLERAGIAPLSLRGSDTGVFMGAYPGGYGAGADLGGFGTTSGAASVLSGRISYFFGLEGPA
MTVDTACSSSLVALHLAGQALRNGECSLALVGGVTVMAAPDIFPEFARQRGLASDGRSKAFADSADGTGWSEGVGVL
LVERLSDAQANGHQILALVRGSAVNQDGASNGLTAPNGPSQQRVIRQALANASLTPADVDVIEAHGTGTTLGDPIEA
QALLATYGQGRSVPLLLGSVKSNLGHTQAAAGVTGVIKMVMALRHGVVPQTLHVDEPSRHVDWSAGAVELVTSNREW
PVTDRPGRAGVSSFGISGTNAHVILEAVPSDTPAPTTTDAVLPLVVSTRTAPAAEDLTARLRAYLSAAPETDQRAAA
ATLALTRSVFEHRAVVLGEDTITGVAVPDPRVVFVFSGQGSQRAGMGEQLAAAYPVFAAIHERVWALLDVPDGLDVD
DTGHAQPALFALQVALSGLLESWGVRPAAVIGHSIGELAAAYVSGVWSLEDACVLVSARARLMQALPPGGVMVAVPV
PEAEARAVLRDGVEIAAVNGPSSVVLSGDEDAVLQAVSGFAKWTRLKTSHAFHSAHMDPMLDEFRAVAERLTYRRPS
VEMAAGDRVTTAEYWVRQVREAVRFGDQTTAYEDAVFVEIGPGRTLARLIDGITMLHGDTEREAALTGLSQLFVRGV
DVDWATVIEDTTARILDLPTYPFQHEHYWLRRAARTPAERAQELLKLVRDNAAAVLGHADGRTVPAAAAFRDLGVDS
LIAVELRNNLALATGLQLPTTIVFDYPTASSLAERL
SEQ ID NO: 66
EPLAIVGMACRLPGGVESPEDLWRLVESGADAISGFPTDRGWDADGLFDPDLAVGKTYCVQGGFLQTAAEFDPAFFG
ISPREALAMDPQQRLVLETSWEAFERAGIEPGSLKGSDTGVFMGAYPGGYGMGADLGGFAATAGAGSVLSGRVSYFF
GFEGPAMTVDTACSSSLVALHQAGYALRQGECSLALVGGVTVMASPQSFVEFSRQRGLAADGRSKAFADAADGTGWA
EGVGVLLVERLSDAQAKGHRILAVVRGSAVNQDGASNGLSAPNGPSQQRVIRQALANASLTPADIDVVEAHGTGTTL
GDPIEAQAVIATYGQDRSTPLLLGSLKSNIGHTQAAAGVSGVIKMVMALRHGVVPQTLHVDQPSRHVDWSAGAVELV
TSNQPWPSSERPRRAGVSAFGVSGTNAHVILESAPVEPVGAEAGLVPVVADVLPWVVSAKSAPALRALEQRLRAYEA
ADERTVVATLATARATFDHRAVLIGTETVTGPLMTDPRVVFVFPGQGWQWLGMGRELRGSSVVFAERMAECAAALSE
FVDWDLFTALDDPAVVDRVDVVQPVCWAVMVSLAAVWQAAGVNPDAVVGHSQGEIAAAVVAGSLSLRDGALVVALRS
QLIKGLAGRGAMASIALPADQIDLVEGAWIAALNGPSSTVIAGTPEAVEQVLAAQDARVRRIAVDYASHTPQVEAIR
DELLELTAEVLSRKPDVPWLSTVDNTWVEGPLPADYWFRNLREQVGFAQAVVTLGDAVFVEVSASPVLIQSMGDAVT
VATLRRDDGSATRMVTSLAEAYVQGVQVNWGAVLGAGTERALDLPTYPFQRQHYWALERLGERAGTERHRLMLEVVL
GHAASVLGHSSAAALEPDRPFKDLGMDSLTAIELRNHLVAETGLRLPATMVFDFPTADALAGHL
SEQ ID NO: 67
EPIAVVSMACRLPGGVDTPEGLWRLVESGTDAISGFPTDRGWDLTDFYSADPQGGFLTGAAEFDAGFFGISPREALG
MDPQQRLLLETTWEAIERAQLDPRSLRGRDVGVYVGGAAQGYGVGFAGEPRDNAITASSISLLSGRVSYALGLQGPG
VTVDTACSSSLVALHLACQALRQRECSLALVGGVSVIATPDVFAEFSRQNGLAADGRCKSFGAAADGTGWSEGVGML
VLERLSEATRHGHRILAVVRGSAVNSDGASNGLTAPNGQSQQRVIRQALSNAGLAASDVDVVEAHGTGTRLGDPIEA
EAILATYGQDRAAPAWLGSLKSNIGHTMAASGVLGVIKMVEAMRHGTVPRTLHVDEPSPHVDWSAGRVALLTENQPW
PDGAKPRRAGVSSFGLSGTNAHVVLEQHPEPASPVPARETGPVPWVLSAQSPKALQEQAGRLHAALVSDPRWHPLDV
AFSLATTRSAFTHRTAVVASGRDLLEALSTLATSATATSTTARTRRVAFLFDGQGTQRAGMGRELYERHPAFARAWD
EVSAAFDKHLEHPLHAVYFGAGALDELVDDTGYAQAAIFTFEVALFELLHEWGVRPDFVAGHSIGEVAAAYVSGLFS
LADAAQLIVARGRALRSAPPGAMAALRAGETETREFLARTGTALDVAAVNSPEAVVVSGSPEAVAEFTAAWTASGRR
ARRLNVNRAFHSRHVDGLLDDFRAVLESLTCRTDTVLPMVSTVTGRLIDPAELRTPQYWLSQVRDTVRFQDAVAELA
ANGVGVFVEVGPSSSLASAGTETLGDEAHFQALQHSRTPADPALLTALAGLHSGGVGVDWEKVLVGGRAVELPVYPF
QHRAYWLAPASTQEPATMLELVRFEVAAVLGMPDPAAVFEETSFLELGFDSLSAVRLRNRLTRSTGVELPATLLFDH
PTPAELAAHL
SEQ ID NO: 68
EPLAIVGMACRLPGGVASPEDLWRLVESGTDAISDFPTDRGWDVEGLYDPDPDVPGKSYAVKGGFLDAAGFDAAFFG
ISPREAAAMDPQQRLVLEASWEAFERAGIEPGSVRGSDTGVFMGAMANGYGAGADLGGFGATAGAGSVLSGRISYFF
GLEGPAMTVDTACSSSLVALHQASFALRQGECSLALVGGVTVMPTPQLFVEFARQRGLAVDGRSKAFADAADGSGFS
EGVGVLVVERLSDAQAKGHRVLAVVRGSAVNQDGASNGLSAPNGPSQQRVIRQALDNAGLSSMDVDVVEGHGTGTRL
GDPIEAQAVISTYGQDRERPLLLGSLKSNIGHAQAAAGVSGVIKMVMALRHGVVPQTLHVDEPSRHVDWAAGAVELV
TENQPWPVAERARRAGVSSFGISGTNAHVILESAPAEAASASEPVTPPSEVSVPVVASDVVPLVVSAKTPGALTDIE
ERLRGYLAAAPEADMQAVASTLAATRSVFEHRAVLLGDDTITGIATPDPRVVFVFPGQGWQWLGMGSVLRETSPVFA
GRMAECAAALREFVDWDLFSVLDDPTVVDRVDVLQPACWAVMVSLAAVWQEAGVSPDAVIGHSQGEIAAACVAGAVS
LRDAARIVALRSKLIGARLGHGAMASIALPADAITLTDGAWIAAHNGPASTVIAGTPQAVDTVLAAYEAQGIRVRRI
TVDYASHSPQVEEIHTELLDATATVGSQTPAVPWLSTVDGAWVEGPLDADYWYRNLREPVRFDQAVTHLQTQGETVF
IEVSASPALTPAMNDDAITVATLRRDDDSPTRILTALAEAFVQGVGVDWPAVTGATTARVLDLPTYAFQRQRYWTLS
GLAAAERRQALAKLVRESAAVVLGHADPDSVPAAAAFKDLGVDSLTAVELRNSLGRSTGLRLPATMVFDYPTPDALA
ARLD
SEQ ID NO: 69
EPLAIVGMACRLPGGVDSPEDLWRLVESGTDAISGFPTDRGWDLDSLYDPILGASGEFYSAQGGFLDRAADFDASFF
GISPREALAMDPQQRLVLEVSWEALERAGIEASSVRGSDTGVFMGAMANGYGIGADFGAFGMTASAGSVLSGRVSYF
FGLEGPAMTVDTACSSSLVALHQAGYSLRQGECSMALVGGVTVMPTPQTFVEFARERGLAVDGRSKAFADAADGSGF
SEGVGVLVVERLSDARARGHRVLAVVRGSAVNQDGASNGLSAPNGPSQQRVIRQALANAGLASADVDVVEAHGTGTR
LGDPIEAQAVIETYGQDRERPLLLGSLKSNIGHTQAAAGVSAVIKMVMALRHGVVPQTLHVDEPSRHVDWTAGAVEL
ATEKLPWPASDRVRRAGVSSFGISGTNAHVILESVPAEVVSPSESSGPNLASDVVPLVVSAKTSGALVDIEERLRGY
LAAVPGVDLGAVASVLAGSRSVFGHRGVLVGGELVSGVALSGPRVVFVFSGQGSQCVGMGERLAGVFPVFAEVYGRV
WDLLDVPGSGLGVDDTGFVQPALFALQVGLFGLLESWGVRPEVVVGHSVGEVAAGYVAGLWSLEDACVLVSARARLM
QGLPGGGVMVSVSVSEERARAALVEGVEIAAVNGPSSVVLSGDEAAVVGVAEGLGGRWRRLATSHAFHSARMDPMLD
EFRVVAEGLEYREPRIVMAGGAGVVSPEYWVRQVRDTVRFGDQVAAYQGDAVFVEVGPGGSLARLIDGVAVGDGEDE
VRAAVMAVAELFVRGVDVDWPAVVGTTATPVDLPTYPFQRQRYWTASWLVALEPEERGQALLRMVREGASVVLGHAD
ARAVEVDRAFRDLGVDSLTAVQVRNNLAKATGLRLPATMVFDYPTPAALAARLD
SEQ ID NO: 70
EPLAIVGMACRLPGGVASPEDLWRLVESGTDVISGFPTDRGWDLDNLYDPDPAVGKSYCVQGYFLDDVADFDASFFG
ISPREALAMDPQQRLILEASWEAFERAGIEPGSVRGSDTGVFMGAFSSGYGIGADHSGFGMTAGAGSVLSGRISYLF
GLEGPAMTVDTACSSSLVALHQASSALRQGECSLALVGGVTVMPTPQTFLEFARQRGLAADGRSKAFSDAADGSGFS
EGVGVLVVERLSDARARGHRVLAVVRGSAVNQDGASNGLSAPNGPSQQRVIRQALANAGLSSADVDVVEAHGTGTRL
GDPIEAQAVIETYGQDRERPLLLGSLKSNIGHTQAAAGVSAVIKMVMALRHGVVPQTLHVDEPSRHVDWTAGAVQLA
TEKQPWPASDRARRAGVSSFGISGTNAHVILESAPVHSVETDETAPMALASDVVPLVVSAKTSGALVDIEERLRGYL
AVAGSEVDLGAVASVLAGSRSVFGHRGVLVGGELVSGVALSGPRVVFVFSGQGSQCVGMGERLAGVFPVFAEVYGRV
WDLLDVPGSGLGVDDTGFVQPALFALQVGLFGLLESWGVRPEVVVGHSVGEVAAGYVAGLWSLEDACVLVSARARLM
QGLPGGGVMVSVSVSEERARAALVEGVEIAAVNGPSSVVLSGDEAAVVGVAEGLGGRWRRLATSHAFHSARMDPMLD
EFRVVAEGLEYREPRIVMAGGAGVVSPEYWVRQVRDTVRFGDQVAAYQGDAVFVEVGPGGSLARLIDGVAVGDGEDE
VRAAVMAVAELFVRGVDVDWPAVVGTTAAPVDLPTYPFQRQRYWTQTWLTGLASEDRRQALLKVVRDSAATVLGHAD
AGMIPATAAFKDLGLDSLTAVELRNSLGKSTGLSLPATMVFDYPTPDALADRLD
SEQ ID NO: 71
EPLAIVGMACRLPGGVASPEDLWRLVESGTDAISDFPTDRGWDVEGLYDPDPDAPGKSYAVKGGFLDAAGFDAAFFG
ISPREALAMDPQQRLVLEASWEAFERAGIEPGSVRGSDTGVFMGAFPAGYGGDREGFGATAGAGSVLSGRVSYFFGL
EGPAITVDTACSSSLVALHQAGYSLRQGECSLALVGGATVMATPQTFVEFSRQRGLSVDGRSKAFADAADGTGWAEG
VGVLVVERLSDAQAKGHQVLAVVRGSAVNQDGASNGLTAPNGPSQQRVIRQALANAGLSSADVDVVEAHGTGTKLGD
PIEAQAVIATYGQDRERPLLLGSLKSNIGHTQAAAGVSGVIKMVMALQHGVVPQTLHVDEPSRHVDWAAGAVELVTE
NQPWPVAERARRAGVSSFGISGTNAHVILESAPAEAASASEPVTPPSEASVPVVASDVVPLVVSAKTPGALTDIEER
LRGYLAAASDVDMAAAASTLAATRSVFEHRAVLLGDDTITGIATPDPRVVFVFPGQGWQWLGMGSVLRETSPVFAGR
MAECAAALGEFVDWDLFSVLDDPTVVDRVDVLQPACWAVMVSLAAVWQEAGVSPDAVIGHSQGEIAAACVAGAVSLR
DAARIVALRSKLIGARLGHGAMASIALPAGDVALVDGAWIAAHNGPASTVIAGTPQAVDTVLAAHEAQGIRVRRITV
DYASHSPQVEEIHAELLDATAAVGSQAPAVPWLSTVDGAWVEGPLDADYWYRNLREPVRFDQAVTLLQTQGETVFIE
VSASPALTPAMNDDAITVATLRRDDDSPARILTALAEAFVQGVGVDWPAVTGATTSRVDLPTYPFQHQRYWAWLAGL
APEARGQALLKVVRESAAVVLGHTGADTVPVTAAFKDLGLDSLTAVELRNSLGRSTGLRLPVTAVFDYPTPAALAAR
LD
SEQ ID NO: 72
EPLAIVGMACRLPGGVASPEDLWRLVESGRDVISDFPVDRGWDLDNLYDPDPAVGKTYCKRGGFLDAAAEFDAAFFG
ISPREAAAMDPQQRLVLEASWEAFERAGIEPGSVRGSDTGVFMGAFANEYGAGADFGAFGMTAGAGSVLSGRVSYLF
GLEGPAMTVDTACSSSLVALHQAGSALRQGECSLALVGGVTVMPTPQLFVGFARERGLAVDGRSKAFSDAADGAGWA
EGVGVLVVERLSDAQARGHRVLAVVRGSAVNQDGASNGLTAPNGPSQQRVIRQALANAGLSSADVDVVEAHGTGTRL
GDPIEAQAVIATYGQDRERPLLLGSLKSNIGHTQAAAGVSGVIKMVMALRHSVVPRTLHVDEPSRHVDWAAGAVELV
TEKQPWPTSDRARRAGVSSFGISGTNAHVILESAPAQPLETDEALVPVVASDVMPLVVSAKTPDALTDIEDRLRAHL
AAAPEADMQAVASTLAATRSVFEHRAVLLGDDTITGVAASGPRVVFVFPGQGWQWLGMGSVLRETSPVFAGRMAECA
AALREFVDWDLFSVLDDPTVVDRVDVLQPACWAVMVSLAAVWQEAGVSPDAVIGHSQGEIAAACVAGAVSLQDAARI
VALRSKLIAHLAGHGAMASIALPADAITLTDGAWIAAHNGTASTVIAGTPQAVDTVLATHEAQGIRVRRITVDYASH
SPQVEEIHTELLDATATVGSQTPAVPWLSTVDNTWISRPLDTDYWYRNLREPVRFDQAVTLLQTQGETVFIEVSASP
ALTPAMNDDAVTVATLRRDDDSPTRILTALAEAFVQGVGVDWPAVTGATTTPVDLPTYPFQRQRYWTASWLAGLAPE
ARGQALLKVVRESTAVVLGHVDTETVPATAPFKDLGLDSLTAVQVRNGLAKATGLRLPATMVFDYPTPAALAARLD
SEQ ID NO: 73
EPLAIVGMACRLPGGVASPEDLWRLVESGTDAISDFPTDRGWDVEGLYDPDPDVPGKSYAVKGGFLDAAGFDAAFFG
ISPREALAMDPQQRLVLEASWEAFERAGIEPGSVRGSDIGVFMGAMANEYGAGADFGAFGMTAGAGSVLSGRVSYFF
GLEGPAMTVDTACSSSLVALHQAGSALRQGECSMALVGGVTVMPTPQTFVEFARQRGLATDGRSKAFADAADGSGFS
EGVGVLVVERLSDARARGHRVLAVVRGSAVNQDGASNGLSAPNGPSQQRVIRQALANAGLASADVDVVEAHGTGTRL
GDPIEAQAVIETYGQDRERPLLLGSLKSNIGHTQAAAGVSAVIKMVMALRHGVVPQTLHVDEPSRHVDWTAGAVQLA
TEKQPWPASDRARRAGVSSFGISGTNAHVILESAPVHSVETDETAPMALASDVVPLVVSAKTSGALVDIEERLRGYL
AAVPGVDLGAVASVLAGSRSVFGHRGVLVGGELVSGVALSGPRVVFVFSGQGSQCVGMGERLAGVFPVFAEVYGRVW
DLLDVPGSGLGVDDTGFVQPALFALQVGLFGLLESWGVRPEVVVGHSVGEVAAGYVAGLWSLEDACVLVSARARLMQ
GLPGGGVMVSVSVSEDRARAALVEGVEIAAVNGPSSVVLSGDEAAVVGVAEGLGGRWRRLATSHAFHSARMDPMLDE
FRVVAEGLEYREPRIVMAGGAGVVSPEYWVRQVRDTVRFGDQVAAYQGDAVFVEVGPGGSLARLIDGVAVGDGEDEV
RAAVMAVAELFVRGVDVDWPAVVGTTATPVDLPTYPFQRQRYWAWLTGLASEDRRQALLKVVRDSAATVLGHADARA
VEVDRAFRDLGVDSLTAVQVRNNLAKATGLRLPATMVFDYPTPAALAARLD
SEQ ID NO: 74
EPLAIVGMACRLPGGVASPEDLWRLVESGTDAISDFPTDRGWDVEGLYDPDPDVPGKSYAVKGGFLDAAGFDAAFFG
ISPREALAMDPQQRLVLEASWEAFERAGIEPGSVRGSDTGVFMGAFTNGYGTGADLDGFGATAGTGSVLSGRVSYFF
GLEGPAMTVDTACSSSLVALHQAGYSLRQGECSMALVGGVTVMPTPQTFVEFARQRGLATDGRSKAFADAADGSGFS
EGVGVLVVERLSDAQARGHRVLAVVRGSAVNQDGASNGLTAPNGPSQQRVIRQALANAGLASADVDVVEAHGTGTRL
GDPIEAQAVIETYGQDRERPLLLGSLKSNIGHTQAAAGVSGVIKMVMALQHGVVPQTLHVDEPSRHVDWSAGAVELA
RERQPWPVAGRARRAGVSSFGISGTNAHVILESAPVHSVETDETAPMALASDVVPLVVSAKTSGALVDIEERLRGYL
AAVPGVDLGAVASVLAGSRSVFGHRGVLVGGELVSGVALSGPRVVFVFSGQGSQCVGMGERLAGVFPVFAEVYGRVW
DLLDVPGSGLGVDDTGFVQPALFALQVGLFGLLESWGVRPEVVVGHSVGEVAAGYVAGLWSLEDACVLVSARARLMQ
GLPGGGVMVSVSVSEERARAALVEGVEIAAVNGPSSVVLSGDEAAVVGVAEGLGGRWRRLATSHAFHSARMDPMLDE
FRVVAEGLEYREPRIVMAGGAGVVSPEYWVRQVRDTVRFGDQVAAYQGDAVFVEVGPGGSLARLIDGVAVGDGEDEV
RAAVMAVAELFVRGVDVDWPAVVGTTATPVDLPTYPFQRQRYWAWLTGLASEDRRQALLKVVRDSAATVLGHADARA
VEVDRAFRDLGVDSLTAVQVRNNLAKATGLRLPATMVFDYPTPAALAARLD
SEQ ID NO: 75
EPLAIVGMACRLPGGVDSPEDLWRLVESGTDVISGFPTDRGWDLDNLYDPDPAVGKSYCVQGYFLDDVADFDASFFG
ISPREALAMDPQQRLILEASWEAFERAGIEPGSVRGSDTGVFMGAFSSGYGIGADHSGFGMTAGAGSVLSGRVSYLF
GLEGPAMTVDTACSSSLVALHQAGSALRQGECSLALVGGATVMPTPQTFVEFARQRGLATDGRSKAFADAADGAGWA
EGVGVLVVERLSDAQARGHRVLAVVRGSAVNQDGASNGLTAPNGPSQQRVIRQALANAGLSSADVDVVEAHGTGTRL
GDPIEAQAVIETYGQDRERPLLLGSLKSNIGHTQAAAGVSAVIKMVMALRHGVVPQTLHVDEPSRHVDWTAGAVQLA
TEKQPWPASDRARRAGVSSFGISGTNAHVILESAPAQPLETDEPSAPIVASDVVPLVVSAKTLDALTDIEDRLRGYL
AAASDVDMAAVASTLAATRSIFEHRAVLLGDDTITGIATPGPRVVFVFPGQGWQWLGMGSTLRETSPVFAARMAECA
AALREFVDWDLFSILDDPTVVDRVDVLQPACWAVMVSLAAVWQEAGVSPDAVIGHSQGEIAAACVAGAVSLQDAARI
VALRSKLIAHLAGHGAMASIALPADAITLTDGAWIAAHNGTASTVIAGTPQAVDTVLATHEAQGIRVRRITVDYASH
SPQVEEIHTELLDATTTINPRTPAVPWLSTVDNTWISRPLDTDYWYRNLREPVRFDQAVTLLQTRGETVFIEVSASP
ALTPAMNDDAITVATLRRDDDSPARILTALAEAFVQGVGVDWPAVTGATTARVLDLPTYAFQHQRYWATAWLAGLAP
AERGEALLKVVSDTVARVLGHADGRTIPATAAFKELGVDSLTAVELRNRLSAATGLRLPATMVFDYPSPGALAGWL
SEQ ID NO: 76
QPLAIVGMACRLPGGVASPEDLWRLVESGTDAISDFPVDRGWDLEGLYDPASDEPGVLYCDQGGFLDAAAGFDAAFF
GISPREAAAMDPQQRLVLEASWEAFERAGIEPGSVRGSDTGVFMGAMANGYGAGADLGGFGATAGAGSVLSGRISYF
FGLEGPAMTVDTACSSSLVALHQASFALRQGECSLALVGGVTVMPTPQTFVEFARQRGLAADGRSKAFSDAADGAGW
AEGVGVLVVERLSDAQAKGHQVLAVVRGSAVNQDGASNGLTAPNGPSQQRVIRQALANAGLSSAEVDVVEAHGTGTR
LGDPIEAQAVIATYGQDRERPLLLGSLKSNIGHAQAAAGVSGVIKMVMALRHGVVPQTLHVDEPSRHVDWAAGAVEL
VTENQPWPVAERARRAGVSSFGISGTNAHVILESAPAEAASASEPVTPPSEASVPVVASDVVPLVVSAKTPGALTDI
EERLRGYLAAAPEADMQAVASTLAATRSVFEHRAVLLGDDTITGVAASGPRVVFVFPGQGWQWLGMGSVLRETSPVF
AGQMAECAAALREFVDWDLFSVLDDPTVVDRVDVLQPACWAVMVSLAAVWQEAGVSPDAVIGHSQGEIAAACVAGAV
SLRDAARIVALRSKLIGARLGHGAMASIALPAGAITLTDGAWIAAHNGPASTVIAGTPQAVDAVLAAYEAQGIRVRR
ITVDYASHSPQVEEIRAELLDATATVGSQAPVVPWLSTVDGAWVEGPLDADYWYRNLREPVRFDQAVTLLQTQGETV
FIEVSASPALTPAMNDDAITVATLRRDDDSPARILTALAEAFVQGVGVDWPAATGATTSRVDLPTYPFQHQRYWTQT
LSGLAAAERRQALAKLVRESAAVVLGHADPDSVPAAAAFKDLGVDSLTAVELRNSLGRSTGLRLPATMVFDYPTPDA
LAARLD
SEQ ID NO: 77
EPLAIVGMACRLPGGVDSPEDLWRLVESGTDAISGFPTDRGWDLDSLYDPILGASGEFYSAQGGFLDRAADFDASFF
GISPREALAMDPQQRLVLEVSWEALERAGIEASSVRGSDTGVFMGAFSSGYGTGSDFGAFGATSSAGSVLSGRISYF
FGLEGPAMTVDTACSSSLVALHQAGSALRQGECSLALVGGITVMSTPLTFAEFARQRGLAPDGRSKAFSDAADGAGF
SEGVGVLVVERLSDAQAKGHRVLAVVRGSAVNQDGASNGLTAPNGPSQQRVIRQALANAGLASADVDVVEAHGTGTR
LGDPIEAQAVIATYGQDRERPLLLGSLKSNIGHAQAAAGVSGVIKMVMALRHGVVPQTLHVDEPSRHVDWAAGAVEL
VTENQPWPVAERARRAGVSSFGISGTNAHVILESAPAEAASASEPVTPPSEASVPVVASDVVPLVVSAKTPGALTDI
EERLRGYLAAASDVDMAVVASTLAATRSVFEHRAVLLGDDTITGVAASGPRVVFVFPGQGWQWLGMGSVLRETSPVF
AGRMAECAAALREFVDWDLFSVLDDPTVVDRVDVLQPACWAVMVSLAAVWQEAGVSPDAVIGHSQGEIAAACVAGAV
SLRDAARIVALRSKLIGARLGHGAMASIALPADAITLTDGAWIAAHNGPASTVIAGTPQAVDAVLAAYEAQGIRVRR
ITVDYASHSPQVEEIRAELLDATATVGSQAPVVPWLSTVDNTWISRPLDTDYWYRNLREPVRFDQAVTLLQTQGETV
FIEVSASPALTPAMNDDAITVATLRRDDDSPARILTALAEAFVQGVGVDWPAVTGATTARVLDLPTYPFQRQRYWAW
LTGLASEDRRQALLKVVRDSAATVLGHADARAVEVDRAFRDLGVDSLTAVQVRNNLAKATGLRLPATMVFDYPTPAA
LAARLD
SEQ ID NO: 78
EPLAIVGMACRLPGGVASPEDLWRLVESGTDVISGFPTDRGWDLDNLYDPDPAVGKSYCVQGYFLDDVADFDASFFG
ISPREALAMDPQQRLILEASWEAFERAGIEPGSVRGSDTGVFMGAFSSGYGIGADHSGFGMTAGAGSVLSGRISYLF
GLEGPAMTVDTACSSSLVALHQASSALRQGECSLALVGGATVLATPYGFVEISRQRGLAADGRSKAFSDAADGMSFS
EGAGVLVLERLSDAQAKGHRVLAVVRGSAVNQDGASNGLTAPNGPSQQRVIQQALANAGLASADVDVVEAHGTGTRL
GDPIEAQAVIATYGQNRERPVLLGSLKSNIGHTHAAAGVSGVIKMVMALQHGVVPRTLHVDAPSRHVDWAAGAVELV
TENQPWPVAERARRAGVSSFGISGTNAHVILESAPAQPLETGEPSAPIVASDVVPLVVSAKTPDALTDIEDRLRAHL
AAAPEADMQAVASTLAATRSIFEHRAVLLGDDTITGIATPDPRVVFVFPGQGWQWLGMGSTLRETSPVFAARMAECA
TALREFVDWDLFSILDDPTVVDRVDVLQPACWAVMVSLAAVWQEAGVSPDAVIGHSQGEIAAACVAGAVSLQDAARI
VALRSKLIAHLAGHGAMASIALPADAITLTDGAWIAAHNGPASTVIAGTPQAVDTVLATHEAQGIRVRRITVDYASH
SPQVEEIRAELLDATATVGSQAPVVPWLSTVDGAWVEGPLDADYWYRNLREPVRFDQAVTLLQTQGETVFIEVSASP
ALTPAMNDDAVTVATLRRDDDSPTRILTALAEAFVQGVGVDWPAVTGATTTPVDLPTYPFQRQRYWTASDRLSGRTS
GDQHRIMLELVLGHAASVLGHGAADAVAADKPFKDLGMDSLTAIELRNHLVAETGLRLPATTAFDHPTADDLARRL
SEQ ID NO: 79
EPIAIVSMACRAPGGVDSPDGLWRLVESGTDAISGFPTDRGWDVADLYSPDPAGYKSYCVQGGFLDTAADFDAAFFG
ISPREALGMDPQQRLLLEASWEAIERARIDPRSLRGRSVGVFVGGASQGYGAGADDQQQSNAITGGSISLLSGRVSY
ALGLEGPGVTVDTACSSSLVALHLASQALRQRECSLALVSGVSVMATPDVFVEFSRQRGLAPDGRCKSFSAAADGTG
WSEGVGVLVLERLSEATRLGHRVHAVVRGSAVNSDGASNGLTAPNGASQQKVIRQALANAGLAASEVDAVEAHGTGT
KLGDPIEAEAILATYGQDRAAPVWLGSLKSNIGHTMAASGVLGVIKMVESMRHGLLPRTLNVDEPSPHVDWASGDVA
LLTENQPWPADVGPRRAAVSSFGISGSNAHVVLEQYGEPAGPDLSDLTNTRAVNAADAPDRRQPVPLMLSARSQRAL
REQAGRLHAALAGAPDWRPLDIGYSLATTRSHFTHRAVAVGSGRELLRALSKLADGADWPALTTRIAKSRRVAFLFD
GQGTQRLGMGSGLYAGFPVFAGVWDQVSAAFDKHLDHSLTDVFLGRDDRPAAAELVDDTLYAQAGLFTLEVALFRLL
EEWGVRPDFLAGHSIGEAAAAYAGGMFSLEDVTALIVARGEALRLAPPGAMLALRASEEEVREFLGRTGAELDLAAV
NGPASVVVSGASEAVADFRARWTAAGRKARELNVSRAFHSRHVEAGLGRFREVLESLTFGTPVLPIVSTVTGQLVDP
VEMSTPEYWLRQVRQPVLFQDALRELSGQGVNTFVEIGPSGTLASAGLECLGGDASFHAVQQPRSPQDVGLMTAVAE
LHAGGTAVDWAKALAGGRATDLPVYPFQHESYWLAPADYAYPEEPGTMLELVRLEAAKVLGITEPDTILEETSFLDL
GFDSLGTMRLRNRLSEVTELDLPATLLFDNPSPAELAAYLD
SEQ ID NO: 80
EPLAIVGMACRLPGGVSSPEDLWRLVESGTDAISHFPTDRGWDLDNLYDPDPDAPGKGYRVQGGFLDAAGFDAAFFG
ISPREAQAMDPQQRLVLEASWEAFERAGIDPGAMRGSHTGVFMGAMANGYGAGADLGGFGATAGAVSVLSGRVSYLF
GLEGPAMTVDTACSSSLVALHQAAYSLRQGECSLALVGGVTVMPTPQMFVEFARQRGLAADGRSKAFADAADGAGFS
EGVGVLVVERLSDAQAKGHRVLAVVRGSAVNQDGASNGLSAPNGPSQQRVIRQALANARLAPNEIDVVEAHGTGTTL
GDPIEAQALIAAYGQDREQPVLLGSLKSNIGHTQAAAGVSGVIKMVMALQHGVVPRTLHVDSPSRHVDWTAGAVELV
TENQPWPAIDRARRAGVSSFGISGTNAHVILESAPAQPVVETEEVPAPPVVASDMMPLVISAKTPSALVEFEGRLRA
YLTSTPGVDMRAVASTLAGTRSVFEHRAVLLGDETVTGPGTGAGSGVAVSDPRVVFVFPGQGSQRAGMGEQLAAVFP
VFAEIHQQVWDLLDVPDPGLDTDETGYAQPSLFALQVALFGLLESWGVRPQAVIGHSVGEIAAGYVAGLWSLRDACT
LVSARARLMQTLPTGGAMVAVPVSEKQAQAALTDGVEIAAVNGPSSVVLTGDETAVLETAAALGRSTRLTTSHAFHS
ARMEPVLDEFRTVAETLDYRTPHIPMAAGDAVVTPEYWVRQIRDTVRFGDQVAAHENAVFVEIGPDRTLSRLTDGIA
MLHGDNETQTAITALATLHTHGVNIHWPAVIGATTARVLDMPTYAFQHQRYWTTWLAGLAPEERKQALLKVVRDSAA
AVLGHAGADTVPVTAAFKDLGLDSLTAVELRNSLGKSTGLRLPATMVFDYPNPTTLAARLD
SEQ ID NO: 81
EPLAIVGMACRLPGGVSSPEDLWRLVESGTDAISGFPTDRGWDIENLYDPDPDAPGKGYRVQGGFLDRAAEFDASFF
GISPREAQAMDPQQRLVLETSWEAFERAGIEPGAMRGSDTGVFMGAMANGYGTGADLGAFGMTSAAVSVLSGRVSYL
FGLEGPAMTVDTACSSSLVALHQAAYSLRQGECSLALVGGVTVMPTPQTFVEFARQRGLAADGRSKAFSDAADGAGF
SEGVGVLVVERLSDARAKGHHVLAVVRGSAVNQDGASNGLSAPNGPSQQRVIRQALDNAGLSSTDVDVVEAHGTGTT
LGDPIEAQALIAAYGQDREQPVLLGSLKSNIGHTQAAAGVSGVIKMVMALQHGVVPRTLHVDSPSRHVDWTAGAVEL
VTENQPWPAIDRARRAGVSSFGISGTNAHVILESAPDLPVVETEETPAPVVTSDMMPLVISAKTPAALADMEGRLRS
YLTSMPGVDMRAVASTLAGTRSVFEHRAVLLGDETVTGPGTGVAVSGPRVVFVFPGQGSQRAGMGEQLAAVFPVFAE
IHQQVWDLLDVPDPGLGADETGFAQPSLFALQVALFGLLESWGVRPQAVIGHSVGEIAAGYVAGLWSLRDACTLVSA
RARLMQTLPTGGAMVAVPVSEKQAQAALTDGVEIAAVNGPSSVVLTGDETAVLETAAALGKSTRLTTSHAFHSARME
PVLDQFRTVAETLDYRTPHIPMAAGDAVVTPEYWVRQIRDTVRFGDQVAAHENAVFVEIGPDRTLSRLTDGIAMLHG
DNETQTAITALATLHTHGVNIHWPAVIGATTTPVDLPTYAFERQRYWAWLAGLAPEERKQALLKTVRDNAAKVLGHA
DARDIAVNTAFRDLGLDSLTAVQVRNSLAKATGLRLPTTTVFDYPNPTALATHLD
SEQ ID NO: 82
EPLAIVGMACRLPGGVSSPEDLWRLVESGTDAISDFPTDRGWDLDNLYDPDPDTPGKAHNVQGGFLDAAGFDASFFG
ISPREAQAMDPQQRLVLETSWEAFERAGIDPASVRGSDTGVFMGAFGSGYGTGADLGGFGATAGAVSVLSGRVSYLF
GLEGPAMTVDTACSSSLVALHQASSALRQDECSLALVGGVTVMPTPQTFVEFARQRGLAADGRSKAFADAADGAGFS
EGVGVLVVERLSDARAKGHRVLAVVRGSAVNQDGASNGLTAPNGPSQQRVIQSALDNAGLSSTDVDVVEAHGTGTTL
GDPIEAQALIAAYGQDREQPVLLGSLKSNIGHTQAAAGVSGVIKMVMALQHGVVPRTLHVDEPSRHVDWTAGAVELV
TENQPWPAIDRARRAGVSSFGISGTNAHVILESAPDLPVVETEEVPAPPVVASDLMPLVISAKTPSALADMEGRLRA
YLTATPGVDMRAVASTLAGTRSVFEHRAVLLGDDTVTGPGVAVSGPRVVFVFPGQGSQRAGMGEQLAAVFPVFAEIH
QQVWDLLDVPDPGLDTDETGFAQPSLFALQVALFGLLESWGVRPQAVIGHSVGEIAAGYVAGLWSLRDACTLVSARA
RLMQTLPTGGAMVAVPVSEKQAQAALTDGVEIAAVNGPSSVVLTGDETAVLETAAALGKSTRLTTSHAFHSARMEPV
LDQFRTVAETLDYRTPHIPMAAGDAVVTPEYWVRQIRDTVRFGDQVAAHENAVFVEIGPDRTLSRLTDGIAMLHGDN
ETQTAITALATLHTHGVNIHWPTIVGTTTPVLDLPTYAFQHQRYWTSWLAGLAPEERKQALLKTVRDSAAAVLGHVG
TDTVPATAAFKDLGLDSLTAVELRNSLGKSTGLRLPATMVFDYPNPTALAARLD
SEQ ID NO: 83
EPLAIVGMACRLPGGVSSPEDLWRLVESGTDAISGFPTDRGWDIENLYDPDPALGRTYTVQGGFLDIAGFDAAFFGI
SPREAQAMDPQQRLVLEASWEAFERAGIEPGSMRGSDTGVFMGAFSSGYGAEHEGFGATAGAVSVLSGRVSYFFGLE
GPALTVDTACSSSLVALHQAGYSLRQGECSLALVGGVTVMPTPQTFVEFSRQRGMAVDGRSKAFADAADGAGWAEGV
GVLVVERLSDARAKGHRVLAVVRGSAVNQDGASNGLSAPNGPSQQRVIRQALANAGLSSADVDVVEAHGTGTRLGDP
IEAQAVLATYGQDREQPLLLGSLKSNIGHAQAAAGVSGVIKMVMALQHGVVPRTLHVDSPSRHVDWTAGSVELVTEN
QPWPVLERARRVGVSSFGISGTNAHVILESAPDPDVAVVEVEETPAPPVVVISAKTPSALADMEGRLRAYLAARPGV
DVRAVASTLAGTRSVFEHRAVLLGDDTVTGTSTGTGSGAAVSGVAVSGPRVVFVFPGQGWQWLGMGCGLRETSAVFA
GRLAECAAALSEFVDWDLLTVLDDPSVVDRVDVLQPACWAVMVSLAAVWQEAGVVPDAVIGHSQGEIAAACVAGALT
LRDAARIVALRSRLIARLAGQGAMASIALPAHEIVLGDGAVVAARNGPAATVVAGTARAVERVLAVHEKEGARVRRI
TVDYASHSPQVEEIRTELLDILATTGSRTPVVPWLSTVDGTWTEQPLDPDYWYRNLREPVGFHPAVDTLRSMGETVF
IEISASPTLTPAMDDATTVATLRRDNDTPQQILTALAEAHTHGVNIHWPAIIGTTTTPARVDLPTYAFQHQRYWTSW
LAGLAPEERKQALLKMVRDSAAAVLGHAGADTVPVTAAFKDLGLDSLTAVELRNSLGKSTGLRLPVTMVFDYPNPTT
LAARLD
SEQ ID NO: 84
EPLAIVGMACRLPGGVSSPEDLWRLVESGTDAISGFPTDRGWDIENLYDPDPDAAGTTYTVQGGFLDIAGFDASFFG
ISPREALAMDPQQRLVLETSWEAFERAGIEPSSMRGSDTGVFMGAFTNGYGAGVDFGAFGGASAAVSVLSGRVSYFF
GLEGPAITVDTACSSSLVALHQAGSALRQGECSLALVGGVTVMPTPQLFVDFSRQRGLAADGRSKAFADPADGAGFS
EGVGVLVVERLSDAQAKGHRVLAVVRGSAVNQDGASNGLSAPNGPSQQRVIRQALDNARLAPNEIDAVEAHGTGTTL
GDPIEAQALIATYGQDREQPVLLGSLKSNIGHTQAAAGVSGVIKMVMALQHGVVPRTLHVDSPSRHVDWTAGAVELV
TENQPWPAIDRARRAGVSSFGISGTNAHVILESAPDPDVPVVETEETPPPPVVVISAKTPSALADMEGRLRAYLAAT
PGVDVRAVASTLAGTRSVFEHRAVLLGDDTVTGTSTGTGSGAAVSGVVVSGPRVVFVFPGQGWQWLGMGCGLRETSA
VFAGRLAECAAALSEFVDWDLLTVLDDPSVVDRVDVLQPACWAVMVSLAAVWQEAGVVPDAVIGHSQGEIAAACVAG
ALTLRDAARIVALRSRLIARLAGQGAMASIALPAHEIALGDGAVVAARNGRAATVIAGTARAVDRVLAVHEKEGARV
RRITVDYASHSPQVEEIRTELLDILATTGSRTPVVPWLSTVDGTWTEQPLDPDYWYRNLREPVGFHPAVDTLRSMGE
TVFIEISASPTLTPAMDDATTVATLRRDNDTPQQILTALAEAHTHGVNIHWPAIMGATTTRVDLPTYAFQHQRYWTS
WLAGLAPEERKQALLKVVRDSAAKVLGHAGADTVPVTAAFKDLGLDSLTAVELRNSLGKSTGLRLPATMVFDYPNPT
TLAARLD
SEQ ID NO: 85
EPLAIVGMACRLPGGVSSPEDLWRLVESGTDAISGFPTDRGWDIENLYDPDPDAPGKGYRVQGGFLDRAAEFDASFF
GISPREAQAMDPQQRLVLETSWEAFERAGIEPGAMRGSDTGVFMGAMANGYGTGADLGAFGMTSAAVSVLSGRVSYL
FGLEGPAMTVDTACSSSLVALHQAAYSLRQGECSLALVGGVTVMPTPQTFVEFARQRGLAADGRSKAFSDAADGAGF
SEGVGVLVVERLSDARAKGHHVLAVVRGSAVNQDGASNGLSAPNGPSQQRVIRQALDNAGLSSTDVDVVEAHGTGTT
LGDPIEAQALIATYGQDREQPVLLGSLKSNIGHTQAAAGVSGVIKMVMALQHGVVPRTLHVDSPSRHVDWTAGAVEL
VTENQPWPAIDRARRVGVSSFGISGTNAHVILESAPAQPVVETEEVPAPPVVASDMMPLVISAKTPSALVEFEGRLR
AYLTSTPGVDMRAVASTLAGTRSVFEHRAVLLGDDTVTGPDTGTGAGSGVAVSDPRVVFVFPGQGSQRAGMGEQLAA
VFPVFAEIHQQVWDLLDVPDPGLGADETGFAQPSLFALQVALFGLLESWGVRPQAVIGHSVGEIAAGYVAGLWSLRD
ACTLVSARARLMQTLPTGGAMVAVPVSEKQAQAALTDGVEIAAVNGPSSVVLTGDETAVLETAAALGKSTRLTTSHA
FHSARMEPVLDQFRTVAETLDYRTPHIPMAAGDAVVTPEYWVRQIRDTVRFGDQVAAHENAVFVEIGPDRTLSRLTD
GIAMLHGDNETQTAITALATLHTHGVNIHWPAVIGATTARVLDLPTYAFERQRYWAWLAGLAPEERKQALLKVVRDS
AAAVLGHADARDIAVNTAFRDLGLDSLTAVQVRNSLAKATGLRLPTTTVFDYPNPTALATHLD
SEQ ID NO: 86
EPLAIVGMACRLPGGVSSPEDLWRLVESGTDAISDFPTDRGWDLDNLYDPDPDTPGKAHNVQGGFLDAAGFDAAFFG
ISPREALAMDPQQRLVLETSWEAFERAGIDPASVRGSDTGVFMGAFGSGYGTGADLGGFGATAGAVSVLSGRVSYLF
GLEGPAMTVDTACSSSLVALHQASSALRQDECSLALVGGVTVMPTPQTFVEFARQRGLAADGRSKAFADAADGAGFS
EGVGVLVVERLSDARAKGHRVLAVVRGSAVNQDGASNGLTAPNGPSQQRVIRQALDNAGLSSTDVDVVEAHGTGTRL
GDPIEAQALIATYGQDREQPVLLGSLKSNIGHTQAAAGVSGVIKMVMALQHGVVPRTLHVDSPSRHVDWTAGAVELV
TENQPWPAIDRARRVGVSSFGISGTNAHVILESAPAQPVVETEEVPAPPVVASDMMPLVISAKTPSALVEFEGRLRA
YLTSTPGVDMRAVASTLAGTRSVFEHRAVLLGDDTVTGPDTGTGAGSGVAVSDPRVVFVFPGQGSQRAGMGEQLAAV
FPVFAEIHQQVWDLLDVPDPGLDTDETGYAQPSLFALQVALFGLLESWGVRPQAVIGHSVGEIAAGYVAGLWSLRDA
CTLVSARARLMQTLPTGGAMVAVPVSEKQAQAALTDGVEIAAVNGPSSVVLTGDETAVLETAAALGRSTRLTTSHAF
HSARMEPVLDEFRTVAETLDYRTPHIPMAAGDAVVTPEYWVRQIRDTVRFGDQVAAHENAVFVEIGPDRTLSRLTDG
IAMLHGDNETQTAITALATLHTHGVNIHWPAVIGATTARVLDMPTYAFQHQRYWTTWLAGLTPEERKQALLKTVRDS
AAAVLGHADARDIAVNTAFRDLGLDSLTAVQVRNSLAKATGLRLPTTTVFDYPNPTALATHLD
SEQ ID NO: 87
EPLAIVGMACRLPGGVSSPEDLWQLVESGTDAISHFPTDRGWDIDNLYDPDPDTPGKTYCVQGYFLDGIAEFDASFF
GTSPREALAMDPQQRLVLETSWEAFERAGIDPASVRGSDTGVFMGAFSSGYGTGADLGGFGATAGAGSVLSGRVSYL
FGLEGPAMTVDTACSSSLVALHQAGYSLRQGECSLALVGGVTVMPTPQAFVEFSRQRGLAADGRSKAFADAADGAGW
AEGVGVLVVERLSDARAKGHRVLAVVRGSAVNQDGASNGLTAPNGPSQQRVIRSALDNARLAPNEIDVVEAHGTGTR
LGDPIEAQALIAAYGQDREQPVLLGSLKSNIGHTQAAAGVSGVIKMVMALQHGVVPRTLHVDEPSRHVDWTAGAVEL
VTENQPWPAIDRARRAGVSSFGISGTNAHVILESPPAQPVVETEEVPAPPVVASDMMPLVISAKTPSALADMEGRLR
AYLAARPGVDVRAVASTLAGTRSVFEHRAVLLGDDTVTGTSTGTGSGAAVSGVVVSGPRVVFVFPGQGWQWLGMGCG
LRETSAVFAGRLAECAAALSEFVDWDLLTVLDDPSVVDRVDVLQPACWAVMVSLAAVWQEAGVVPDAVIGHSQGEIA
AACVAGALTLRDAARIVALRSRLIARLAGQGAMASIALPAHEIALGDGAVVAARNGPAATVIAGTPRAVDRVLAVHE
KEGARVRRITVDYASHSPQVEEIRTELLDILATTGSRTPVVPWLSTVDGTWTEQPLDPDYWYRNLREPVGFHPAVDT
LRSMGETVFIEISASPTLTPAMDDATTVATLRRDNDTPRQILTALAEAHTHGVNIHWPTVIGTTTTPARVDLPTYAF
QHQRYWTSWLAGLAPAERDEALLKMVRDSAALVLGHAGGRTIPVAAAFKDLGVDSLTAVELRNRLSAATGLRLPATL
VFDYPNPAALAGWL
SEQ ID NO: 88
QPLAIVGMACRLPGGVSSPEDLWRLVESGTDAISDFPTDRGWDIENLYDPDSPGEGEAYSAQGGFLDAAGFDAAFFG
ISPREAQAMDPQQRLVLEASWEAFERAGIDPGAMRGSHTGVFMGAMANGYGAGADLGGFGATAGAGSVLSGRISYLF
GLEGPAMTVDTACSSSLVALHQAAYSLRQGECSLALVGGVTVMATTQTFVEFARQRGLAADGRSKAFADAADGAGWA
EGVGVLVVERLSDARAKGHRVLAVVRGSAVNQDGASNGLTAPNGPSQQRVIRSALDNAGLSSADVDVVEAHGTGTTL
GDPIEAQALIAAYGQDREQPVLLGSLKSNIGHAQAAAGVSGVIKMVMALQHGVVPRTLHVDSPSRHVDWTAGAVELV
TENQPWPVTERARRAGVSSFGISGTNAHVILESAPDPDVPVVETEKVPAPPVVVISAKTPSALVEFEGRLRAYLAAR
PGVDVRAVASTLAGTRSVFGHRAVLLGDDTVTGTSTGTGSGAAVSGVVVSGPRVVFVFPGQGWQWLGMGCGLRETSA
VFAGRLAECAAALSEFVDWDLLTVLDDPSVVDRVDVLQPACWAVMVSLAAVWQEAGVVPDAVIGHSQGEIAAACVAG
ALTLRDAARIVALRSRLIARLAGQGAMASIALPAHEIALGDGAVVAALNGPAATVIAGTPRAVDRVLAVHEKEGARV
RRITVDYASHSPQVEEIRTELLDILATTGSRTPVVPWLSTVDGTWTEQPLDPDYWYRNLREPVGFHPAVDTLRSMGE
TVFIEISASPTLTPAMDDATTVATLRRDNDTPQQILTALAEAHTHGVNIHWPTVMGATTTRVDLPTYAFQHQRYWTS
WLAGLAPEERKQALLKVVRDSAAAVLGHAGTDTVPVTAAFKDLGLDSLTAVELRNSLGKSTGLRLPATLVFDYPNPT
TLAARLD
SEQ ID NO: 89
EPLAIVGMACRLPGGVSSPEDLWRLVESGTDAISGFPTDRGWDIENLYDPDPDAPGTGYRVQGGFLDRAAEFDASFF
GISPREALAMDPQQRLVLETSWEAFERAGIEPGSVRGSDTGVFMGAFSSGYGTGADFGAFGATSAAVSVLSGRVSYF
FGLEGPAMTVDTACSSSLVALHQAGSALRQGECSLALVGGVTVMSTPLTFAEFARQRGLAADGRSKAFADAADGAGF
SEGVGVLVVERLSDARAKGHRVLAVVRGSAVNQDGASNGLSAPNGPSQQRVIRQALANAGLSSADVDVVEAHGTGTR
LGDPIEAQAVLATYGQDREQPLLLGSLKSNIGHTQAAAGVSGVIKMVMALQHGVVPRTLHVDSPSRHVDWTAGAVEL
VTENQPWPVLERARRAGVSSFGISGTNAHVILESAPDPDLPVVEVEETPAPVVAVISAKTPSALVEFEGRLRTYLTA
RPGVDVRAVASTLAGTRSVFGHRAVLLGDDTVTGTGPGAAVSGVVVSGPRVVFVFPGQGWQWLGMGCGLRETSAVFA
GRLAECAAALSEFVDWDLLTVLDDPSVVDRVDVLQPACWAVMVSLAAVWQEAGVVPDAVIGHSQGEIAAACVAGALT
LRDAARIVALRSRLIARLAGQGAMASIALPAHEIALGDGAVVAARNGPAATVIAGTPRAVDRVLAVHEKQGARVRRI
TVDYASHSPQVEEIRTELLDILATTGSRTPVVPWLSTVDGTWTEQPLDPDYWYRNLREPVGFHPAVDTLRSMGETVF
IEISASPTLTPAMDDATTVATLRRDNDTPQQILTALAEAHTHGVNIHWPTVMGATTTPVRVDLPTYAFERQRYWAWL
AGLTPEERKQALLKTVRDSAAAVLGHTDARDIAMNTAFRDLGLDSLTAVQVRNSLAKATGLRLPTTTVFDYPNPTAL
ATHLD
SEQ ID NO: 90
EPLAIVGMACRLPGGVSSPEDLWQLVESGTDAISHFPTDRGWDIDNLYDPDPDTPGKTYCVQGYFLDGIAEFDASFF
GISPREAQAMDPQQRLVLETSWEAFERAGIDPASVRGSDTGVFMGAFGSGYGTGADLGGFGMTAGAGSVLSGRVSYF
FGLEGPAMTVDTACSSSLVALHQASSALRQGECSLALVGGTTVLATPYGLVEISRQRGLAADGRSKAFSDAADGMGF
SEGVGVLVVERLSDARAKGHRVLAVVRGSAVNQDGASNGLTAPNGPSQQRVIRSALDNAGLSSADVDVVEAHGTGTR
LGDPIEAQAVLATYGQDREQPLLLGSLKSNIGHTHAAAGVSGVIKMVMALQHGVVPRTLHVDEPSRHVDWTAGAVEL
VTENQPWPAIDRARRAGVSSFGISGTNAHVILESPPAQPVVEVEETPAPPVVASDMMPLVISAKTPSALADMEGRLR
AYLAARPGVDVRAVASTLAGTRSVFEHRAVLLGDDTVTGTSTGTGSGAAVSGVVVSGPRVVFVFPGQGWQWLGMGCG
LRETSAVFAGRLAECAAALSEFVDWDLLTVLDDPSVVDRVDVLQPACWAVMVSLAAVWQEAGVVPDAVIGHSQGEIA
AACVAGALTLRDAARIVALRSRLIARLAGQGAMASIALPAHEIALGDGAVVAARNGRAATVIAGTARAVDRVLAVHE
KEGARVRRIAVDYASHSPQVEEIRTELLDILATTGSRTPVVPWLSTVDGTWTEQPLDPDYWYRNLREPVGFHPAVDT
LRSMGETVFIEISASPTLTPAMDDATTVATLRRDNDTPRQILTALAEAHTHGVNIHWPTVIGTTTTPARVDLPTYAF
QHQRYWTSDRLNGRTGLEQHRVMLELVLGHAASVLGHSAPDAIAADRPFKDLGMDSLTAIELRNHLVAETGLRLPAT
TAFDHPTADDLAKRL
SEQ ID NO: 91
EPIAIVSMACRVPGGVDSPEGLWHLVESGTDAISDFPTNRGWDVANLYSPDPAGYTSYCVQGGFLDSAADFDATFFG
ISPREALGMDPQQRLVLEASWEAIERAQIDPRSLRGSNVGVFVGGASQGYGASANEQQQSNAITGGSSSLLSGRVTY
ALGLEGPAVTVDTACSSSLVALHLASQSLRQRECSLALVSGVSVMATPDVFVEFSRQRGLAPDGRCKSFSASADGTG
WSEGVGVLVLERLSEATRLGHRVLAVVRGSAVNSDGASNGLTAPNGASQQRVIRQALANAGLTASQVDAVEAHGTGT
TLGDPIEAEALLATYGQDRSTPAWLGSLKSNIGHTMAASGVLGVIKMVEAMRHGLLPRTLHVDEPSPHVDWASGDIA
LLSESRPWPDGSTPRRAGVSSFGISGTNAHVVLEQYRDPAGPDTPTGSDTQTGPETTTEHGPLPLMLSARSPKALRE
QAGRLHAALVEAPRWRPLDIGYSLATTRSSFAHRAVAVGSDRELLRALSQLADGGTSPALVTATAKAGRVAFLFDGQ
GTQRLGMGSGLYERFPAFARTWDLVSAAFDKHLNHSLTDVFLGRSGSVTAELVDDTLYAQAGIFTMEVALFELLDEW
GIRPDFLTGHSIGEAAAAYGAGMLSLEDVTTLIVARGQALRLSPPGAMVALRASEEEVREFLDRTGAALDLAAVNSP
TSVVVSGAPDAVSDFRTAWTESGREARALNVRHAFHSRHVEAGLGRFREVLDSLTFRAPVLPVVSTVTGRLVEPAEM
STPEYWLRQVRQTVRFHDALRELSGRGVGTFVEIGPSGTLASAGLECLGGDAAFHAVQRPRSAEDVCLMTAVAELHA
GGTAVDWTKVLAGGRRTDLPVYPFQHEAYWLTPAEPSYAEEPLTTLELVCSEAANVLGITEPGILLEDSSFLDLGLD
SLGAMRLRNRLSELTELDLPATLLFDNPNPTDLAAYLD
SEQ ID NO: 92
EPLAIVGMAARFPGGVASADDLWRLVVSGGDAIGGFPTDRGWDLDELYDPDPAATGRSYVREGGFLSDATTFDASFF
RIGPREAKAMDPQQRLLLETSWEAFEHAGIRPETLRGTATAVFAGISLQDYGVLAGSDPELEGYAGTGNAPSVLSGR
LSYFYGLEGPAVTIDTACSSSLVALHLAGQSLRRDECTLAVVGGVTVMPSPNVFVEFSRQRGLAPDGRCKPFAAAAD
GTGWSEGAGVLVVERLSDARRNNRRILAVVRGSAVNQDGASSGLTAPHGPSQQRVIRAALAAAGLTAGDVDVVEAHG
TGTTLGDPIEAQGVLATYGDRKGAPVRLGSVKSNLGHTQAAAGVAGVIKMVQALRHGVMPRSLHIDEPSPHVDWTAG
RVELLTSNLPWPASERPRRAAVSSFGISGTNAHVILEQAFPATEPEPPFTPVVSGPELPLIFSAKDPDALAAQTRVT
DGPGVAYALATSRSMFDHRTVRLGDMTVTGIAVTDPEVVFVFPGQGTQWAGMGRDLMEASPVFAERMNECAAALEPY
LDLWAAIDAPDHVETLQPASWAMMVSLAAVWQAAGVQPAAVIGHSQGEIAAACVSGAISLQDAAAVVALRSKAIAAS
LGKGAMASIPLPADAIELTGEVWVAALNGPSSTVVAGVPEAVELVRARYEGRRIAVDYASHTPHVEALRGQVVSVPS
QAPVIPWFSTVDSGWVEGPLDDDYWFRNLRQPVQFGPAAAGFDNAVFIEVSARPVLIPALEASVTVPSLRRDDGGPE
RMLASLAQAFVAGVPVDWTTIVAPAPFVELPTYPFQGERYWIDPRTLDEVLAVVRDSAATVLGHTDPTAITPDRSFK
DLGFDSLAAVQLRNHLLTATGVRLSATAVFDFPTPVVLAGEV
SEQ ID NO: 93
EPIAVVGMACRLPGDVSSPEDLWRLVSEGRDAVGPFPADRGWEPGDAAYARVGGFVTGATGFDAGFFGISPREAQAM
DPQQRLLLEVAWEAFERAGIAPDELRGSDTGVFVGTYGQGYGELAVDGDAEGYVGIGNSGSVVSGRVSYFFGLEGPA
VTVDTACSSSLVALHQAAQALRQGECSLALVGGVTVMSSPLIFQEFARQGGLAADGRCKAFADGADGTGWGEGVGVL
VVERLSEAQRRGHTVLAVVRGSAVNQDGASNGLTAPNGPSQQRVIRAALASAGLGVGDVDLVEAHGTGTALGDPIEA
QALLATYGSDGSPVWLGSLKSNIGHTQAAAGVAGVIKAVEAMRHGVMPRTLHVDQPSSHVDWSAGAVELLTANRPWD
SGGRPRRAAVSSFGISGTNAHVILEESPSAPVPPEPGTAPLLLSARSPAALAQFESRTAGLRPSRDLASTLSRRALF
DHRAVVLPDGDTVRGGVGDAPLVFVFAGQGSQRADMGSRLAEEFPVFAAAYERVWSLLDVDESLEVDHTGFAQPALF
AFEVALAELLGVRPDAVIGHSVGELAAAYVAGAMSLEDACRLVSARARLMQALPSGGVMVSVRVSEEAARTVLRDGV
EIAAVNGPQAVVLSGDEDAVLAAAAELGEFKRLRTSHAFHSARMEPMLEEFRAVASTVAFDEPQIALSFVPSAEYFV
RQVRETVRFGEQVAAFAPGTLFVEVGPDGSLSRLTGGVSAAEPMKALAYLWVRGVGVDWTPYIGDGRLDDAPTYPFQ
PERYWPEQRRRARHGDFLALVTATAAVVLGHPEGTDIPADTPFQSLGLDSLSAVDLRNQLAQATGVRLSPTAVFDYP
TPRALAERL
SEQ ID NO: 94
DPIAIVGMACRYPGGVATADDLWDLVAEGGDAVGPFPVDRGWDLAALYDPDPEAAGKSYVREGGFLGGAADFDAAFF
GISPREALAMDPQQRLLLETAWEAFEHAGIDPLDLRRSDTGVFVGTMAQEYGGLVTDSAHGLEGWIGTGNSQSVMSG
RLSYFFGLQGPAVTVDTACSSSLVALHQAAQALRNGECALAVVGGVTVMSSPRTFQEFSRQRGMAPDGRCKPFAAAA
DGTGWSEGVGVLVVERLSEARRNGHAVLAVVRGTAVNQDGTSNGLTAPNGPAQQQVIRAALERAGLGVGDVDVVEAH
GTGTALGDPIEAQAILDTYGSRTGGEPVRLGSVKSNLGHTQAAAGVAGVIKMVQAMRHATMPRSLHIDEPSPHVDWA
SGAVELLTAERGWPATDRPRRAAVSSFGISGTNAHVIVEGVADPELSREASPGGPLPFVLSAPTAEALSAQETRLRR
FRVERPDVDERDIAITLAGRTGFAHRTVLIGDLTVSGVAVADRRVVFVFPGQGTQWAGMGRDLMAASPVFAERMNEC
AAALEPYLDLWEAIDSPDRVETLQPASWAVMVSLAAVWQAAGVQPAAVIGHSQGEIAAACVSGAISLQDAAAVVALR
SKAIGASLGKGAMASIPLPADAIELIDEVWIAALNGPSSTVVAGAPEAVEQIRARYDGRRIAVDYASHTPHVEALRG
QVVSVPSRAPAIPWFSTVDSAWVEDPLDEDYWFRNLRQPVQFGPAAAGFDNAVFIEVSARPVLIPALEDAVTVPTLR
RDDGGIDRLHASVAQAWTAGADVDWAALLPAGGRRIALPPYAFTHERFWPRRPTAAGQDLLTVVRTAAATVLGHRDA
ARVPADRAFKELGFDSLSAVQLRNELLTATGVRLSATAVFDHPTAAALAEAL
SEQ ID NO: 95
EPIAIVGMACRLPGDVSSPDELWELVEAGRDAVGPFPADRGWNLSTLFDPDPDAPGKSYVREGGFLTGAGLFDADFF
GISPREALAMDPQQRLLLEMAWEAFERAGIAPDELRGSDTGVYVGTYAQGYGELAAATAGEGFVGIGNSGSVVSGRV
SYFLGLEGPAVTVDTACSSSLVALHQAAQALRLGECSMALVGGVTVMASPLMFQEFSRQRGLSPDGRCKAFAESADG
TGWGEGVGVLVVERLSEARRRGHTVLAVVRGSAVNQDGASNGLTAPNGPSQQRVIRAALASAGLTVGDVDLVEGHGT
GTALGDPIEAQALLATYGSAGSPVWLGSLKSNIGHTQAAAGVAGVIKMVQAMRHGVMPRTLHVDQPSSHVDWSAGAV
ELLTANRPWDSGGRPRRAAVSSFGISGTNAHVILEGVPAPEPAAGDAETAPLVLSARTAPALTDLEARVSARPSSPD
LAATLAGRASFDHRAVVLPDGEVVRGRAGAAPVVLVFAGQGSQRADMASRLAGEFPVFAAAYERVWSLLDVDEALDT
DQTGFAQPALFAYEVALAELLNVRPDAVIGHSIGELAAAYTSGSLSLEDACRLVSARARLMQALPPGGAMVSVRVSE
EVAREVLRDGVEIAAVNGPQAVVLSGDEDAVLAAAAKLGEFKRLRTSHAFHSARMEPMLEEFRAVALTVEFREPEVA
LSFVPSAEYFVGQVRETVRFGEQVASFEPGTLFVEVGPDGSLSRLTGGVSAAEPLTALAYLWVHGVAIDWVPYLGGG
RLDLGAPTYPFQHERYWPARALAQLPPARRGRALLDLVQNRVAKTLGLVRPADPGRAFTDLGFTSLTALELRNSIAE
ETGLPMPASLVFDHPNARSLAGYLD
SEQ ID NO: 96
EPLAIVGMACRLPGGISSAEELWRLVAEGGDAIGPFPGDRGWDVDALYDPDPAAGHTYTRSGGFLPGATDFDAAFFG
ISPREAQAMDPQHRQLLETSWEALEHAGIDPAGLRGRDVGVFAGFSGQDYIAEMGVGPAEAGGYQVTGRAASVLSGR
LSYFYGLEGPAVTVDTACSSSLVALHLAGQSLRDGESSLALVGGVTVMSSPGLFVEFSRQRGLAPDGRCKAFSADAD
GTGWSEGVGVLVVERLSDARRNGHRILAVVRGSAVNQDGASNGLTAPNGPSQQRVIRQALAQSGLSVADVDVVEAHG
TGTALGDPIEAQAVLATYGGRAGGEPVRLGSLKSNIGHTQAAAGIASLIKMVQAIRYGVMPRTLHVSEPSPLVDWAS
GRVELLTSDIPWPDGVRRAAVSAFGISGTNAHVILEEAPAPAAVPSIRPVVSGPALPLVFSARDPSALAAQTRVTDG
PGVAYALATSRTMLDHRTVRLNDVTVTGIAVTDPEVVFVFPGQGSQWAGMGRDLMGSSPVFAERMNECAAALEPYLD
LWAAIDAPDRVETLQPASWAVMVSLAAVWQAAGVQPAAVIGHSQGEIAAACVSGAISLQDAAAVVALRSRAIAASLG
KGAMASIPLPADAIELADEVWVAALNGPSSTVVAGALEAVEQVRARYEGRRIAVDYASHTPHVEALRGQVVSVPSQA
PAIPWFSTVDSGWIEGPLDDDYWFRNLRQQVQFGPAAAGFDNAVFIEVSARPVLIPALDASVTVPSLRRDDGGPERM
LASLAQAFVAGVPVDWTTIVPPAPHVELPSYPFQRQRHWIDMERLGQLPPGDRDRFLLDLVRDAAAAVLGHGSRETV
PASAAFKELGFDSLIAVQLRNAVSAATGVRLPATVTFDHPTPQALAALL
SEQ ID NO: 97
EPLAIVGMACKFPGGVDSPERLWEMLEAGEDVIGPFPDDRGWDVDGGYDPDPEKAGSWYARAGGFLAGAADFDAAFF
GINPREALAMDPQQRLLLEVAWEAFERSGIAPDSLRGTDTGVFVGTFGQGYGRLVSAGAPGLEAYSGTGNTGSVASG
RLSYVFGLEGPAVTVDTACSSSLVALHQASQSLHRGECSLALVAGVTVMSTPDSFVEFSRQRGLSPDGRCKAFAAAA
DGTGFSEGAGVLVVERLSDAQRNGHQILAVVRGSAVNQDGASNGLTAPHGPSQQRVINTALTDADLTTTDIDLVEAH
GTGTTLGDPIEAQAILATYGNRTTGNPVHLGSVKSNLGHTQAAAGVAGVIKVIQAMRHATMPKSLHIDQPSPHVDWT
AGRVELLTGNRPWPATDRPRRAAVSSFGVSGTNAHVILEERAAAEEQPPAVDGPVPLVLSARTPEALTAQEEAVRGL
STDDRHRVAPALALGRAALPHRAVLLGDSVIRGTASADDGRPVFLFPGQGAQWAGMGRELMAASPVFAERMRECAVA
LAGFVDWDLFAVLDDAEALRRTEIVQPASWAVMVSLAALWESWGVHPAAVVGHSQGETAAAVVAGAIGLRDGARLSA
TRSRVLALLAGHGALASIALPAAEVEVVDGVSVAAVNGPRATLISGDPAGVEAVTARYEASGVRVRRIPADVASHSP
HVERAEETLLTALAGIEARVPGVPWLSTATGDWITEPVDERYWYRNLRSPVLFHPAITTLRDRGHRLFLEISTHPQL
LPAMEDDLLTVGSLRRDDGGPDRMHTALAEAWAGGADVDWPAVLGAGPVRALDLPTYPFQRRRFWPEAALPPVERDR
ALVEIVRDQAAAVLGHPDAGALTPGTAFRDLGFDSLTAVQLRNHLATATGLTLPATVIFDHPTPRALATFLD
SEQ ID NO: 98
EPLAVVGMACRLPGGVASPDQLWDLVVSGGDGIGPFPADRGWPTDDIFDPDPDAPGKTYVREGGFLDGAGEFDAAFF
GISPREALAMDPQQRLLLETSWEAFEHAGIDPAGLRGGDTGVFVGGFTQAYGVGTADLEGYAATGTVGSVLSGRLSY
FYGFEGPAVTVDTACSSSLVALHQAGQALRQGECTLAVVGGVTVMPTPVVFQEFSRQRGLATDGRCKAFADEADGTG
FAEGAGVLLVCRLSDARRDGRRILAVVRGSAVNQDGASNGLTAPHGPSQQRVIRAALANARLGPGDVDLIEGHGTGT
TLGDPIEAQALLATHGSGASPVRLGSLKSNIGHTQAAAGVAGVIKVIQALRNGLMPRTLHAGTPSSRVDWSAGNVEL
LTSNLPWPAADRPRRAAVSSFGISGTNAHVILEEAPAAAAVPTISPVVSGPALPLVFSARDPSALAAQTRVTEGPGV
AFALATTRSMFEHRAVRIGDFSVSGAAVADRRVVFVFPGQGTQWAGMGRDLMSASPVFAERMNECAAALEPYLDLWE
AIDSPDRVETLQPASWAVMVSLAAVWQAAGVQPAAVIGHSQGEIAAACVAGSITLQDAAAVVALRSRAIAASLGKGA
MASIPLPAEQIELAGEVWVAALNGPSSTVVAGLPEAVEQVRARYEGRRIAVDYASHTPHVEALRGQVVSVPSRAPAI
PWFSTVDSGWIEGPLDEDYWFRNLRQPVQFGPAAGRFDDAVFIEVSARPVLIPALEDAATVPSLRRDDGGGDRMLAS
LAQAFVAGVPVDWTTIIPPAPFVELPSYPFQHRRYWIDSSEDALRDLVREQAAAVLGYPDPSRITPGVAFRDLGFDS
LTAVQLRNALSAATGLRLSATVAFDHPTPAALAAAL
SEQ ID NO: 99
EPIAIVGMACRLPGGVSSPDELWELVESGRDAIGPFPADRGWNLDELYDPDPDAAGRSYVREGGFLTGAADFDAGFF
GINPREALAMDPQQRLVLEVAWEAFERAGIAPDSLRGTDTGVFLGAFAGGYLTLVNGAADLEGYAGTGNSVSVLSGR
LSYVLGLEGPAVTVDTACSSSLVALHQAAQALRLGECSLAVVGGVTVMSTPDSHVEFSRQRALSPDGRCKAFADGAD
GTGWAEGAGVLVVERLSEARRRGHTVLAVVRGSAVNQDGASNGLTAPNGPSQQRVIRAALASAGLGVGDVDLVEGHG
TGTALGDPIEAQALLATYGSDGSPVWLGSLKSNIGHTQAAAGVAGVIKAVESMRRGVMPQTLHVGTPSSHVDWAAGA
VELLTANRAWDSVERPRRAAVSSFGISGTNAHVILEGVPAPEPAAGSAESAPLLLSARSAAALAQFESLTSGLRPSR
DLASTLSRRAFFDHRAVVLPGGDVVRGRVGDAPVVLVFAGQGSQRADMASRLTAEFPVFAAAHERVWSLLDVDEGLG
IDQTGFAQPALFAYEVALAELLDVRPDAVIGHSIGELAAAYVAGAVSLEDACRLVSARARLMQALPPGGAMVSVRVS
EEAARAVLRDGVEIAAVNGPQAVVLSGDEDAVLAAAAELGEFKRLRTSHAFHSARMEPMLDEFRAVALTVEFREPEV
ALSFVPSAEYFVRQVRETVRFGEQVAAFAPGTLFVEVGPDGSLSRLTGGVSAAEPLTALAYLWVRGVGVEWTPYVGG
GILDQGAPTYPFQRERYWVRPRLAGRTTDERDALLIDLVRDDVASVLGHSGRRRLETDRPLLELGFDSLTALRLRNR
LAAATDVALPATLIFDYPNIQAIAVHL
SEQ ID NO: 100
EPLAVVGMACRYPGGVASADDLWRLVAAGGDAVGPFPDDRGWELESLVDPDPEAVGRSTTGQGGFLADAAGFDAAFF
GISPREATAMDPQQRLLLEVSWAALEHAGLRADALRGSATGVFMGSNGQDYAGLLAGAPELEGWIGTGVSASVVSGR
LSYFYGFEGPAVTVDTACSSSLVALHLAAQSLRTGESSLALVGGVTVMTSPTVFRSFSRQRGLAPDGRCKAFSAGAD
GTGWSEGVGVLVVERLSDAQRNGHQILAVVRGSAVNQDGASNGLTAPHGPSQQRVINTALTDADLTTTDIDLIEAHG
TGTTLGDPIEAQAILATYGNRTTGNPVHLGSVKSNLGHTQAAAGIAGIIKAIQAMRHATMPRTLHIDEPSPHVDWTA
GRVELLTSNLPWPATGRPRRAAVSSFGVSGTNAHVILEEAPAPAAVPSIRPVVSGPALPLVFSAKDPDALAEFQSHT
PAGEGVAYALATSRSTLDHRSVRIGDVTVTGIAVTDPEVVFVFPGQGTQWAGMGRDLMSASPVFAERMNECAAALEP
YLDLWAAIDAPDRVETLQPASWAMMVSLAAVWQAAGVQPAAVIGHSQGEIAAACVSGAISLQDAAAVVALRSKAIAA
SLGKGAMASIPLPADAIELTGEVWVAALNGPSSTVVAGVPEAVELVRARYEGRRIAVDYASHTPHVEALRGQVVSVP
SQAPVIPWFSTVDSGWVEGPLDDDYWFRNLRQPVQFGPAAAGFDNAVFIEVSARPVLIPALDASVTVPSLRRDDGGP
ERMLASLAQAFVAGVPVDWTTIVPPAPHVDLPSYPFQHQRFWIEGRVTAAAGAERLRIMLEVVLAETATVLGHGGAA
AIGPGRAFQDLGFDSLTAVELRNRLAAATGLTLPTTLVFNHPTPEALAAHL
SEQ ID NO: 101
EPVAIVGMACRLPGDVESPEDLWRLVAEGRDAVGPFPSDRGWNLGTLDDPDAAGRSYVKEGGFLAGAAHFDPAFFGI
GPREALGMDPQQRILLEIAWEALEHARIAPGDLRGSETGVYVGAAAQGYGVDAPLEGNLLTGGSTSAMSGRVAYALG
LHGPAVTVDTACSSSLVALHLAAQALRHGECTLALAGGVAVMASPVLFTEFSRQRGLAPDGRCKAFAAAADGTGWSE
GAGLVVLERLSDAERHGHRVLAVIRGSAVNSDGASNGLTAPNGTAQRRVIRSALRAAGLGAGDVDVVEAHGTGTTLG
DPVEADALIATYGQRSDTPPVRIGSLKSNIGHTVAAAGVAGVIKMVEAMRHGTMPRTLHVDRPTPHVDWSAGAAELL
TGELPWPRGDRPRRAAVSAFGLSGTNAHLILEDVAAAAEPPAGDDSGSGSETVPLLLSADDLPAVRDQAARLRAHLL
AHPELRMRDVAYALATTRTARPHRAAVTATERELLRELALLAAGDQGPGTQLGEAVPHRRVAFLFDGQGTQRHGMGR
ALHQRHPVFAAAWDEVCAALDPLLDRGVAEVYFAEAGRDLADDPLYTQAGLFALEVALYRLLTSWGVTPDAVAGHSV
GEVAVAHVAGVLSLPDAASLLAARGAALRQLPPGAMAAIRASEDDTRGVLPPDLDVAAVNGPEMTVVSGAEEAVDRF
VAEQAGAGRQVRRLRVGRAYHSRHVDAVLAEFGATLSALTFHEPTLPVVSTVTGRPAGAGDLTTPEYWLRHARRPVR
FGAALASLSELGMDSFVEVGPSGSLSSMAGETVAGTFHPLLDRRVPDEIGAAAAAGELFTAGMALDWTAVLAGGRPI
DLPVYPFRREFYWLGARYDLMAAAVRRDALLDLVRVQVALLLGRADAIGVRDNTSFLDVGLDSLGASRLRNRLAAAT
GLTLPGGLAFDHPTPARLADHLD
SEQ ID NO: 102
EPLAIVGMACRLPGGVWSPEDLWHLVASGTDAISDFPADRGWDVEKLFDPDPDAPGKTYCVQGGFLEATAAFDAAFF
GISPREALAMDPQQRLMLEVSWEAFERAGIEPGSVRGSDTGVFLGAYPGGYGAGAGADLGGFGATGGAGSVLSGRVS
YFFGLEGPAMTVDTACSSSLVALHQAAYSLRQRECSLALVGGVTVMGTPHMFVDFSRQRGLSVDGRCKAFADAADGT
SWSEGVGVLLVERLSDAQAKGHRVLAVVKGSAVNQDGASNGLTAPNGPSQQRVIRQALANADLAPHEVDVVEAHGTG
TTLGDPIEAQALLATYGQDREQPLLLGSLKSNVGHTQAAAGVSGVIKMVMALQHGVVPRTLHVDEPSRHVDWSAGAV
ELVTQNQPWPSSDRPRRAGVSSFGVSGTNAHIILESAPAQPLAPSTPITGLVPLVISAKTAPALTAFEARLRSYVTA
DADLTAIAATLATTRSTFEHRAVLLGDDTVTGIATPDPRVVFVFPGQGWQWLGMGSALRETSVVFKERMAECAAALS
EFVDWDLFSVLDDPAVVDRVDVVQPACWAVMVSLAATWQAAGARPDAVVGHSQGEIAAACVAGAISLQDGARVVALR
SQLIARLAGHGAMASIALPADQITLTDGVWIAARNGPAATVIAGAPEAVDSVLAAHQDARVRRITVDYASHTPHVEK
IRDELLPMLADIDSQTPLVPWLSTVDGLFIEGPLKADYWYRNLREQVGFDTAVNQLPDSIFIEVSASPVLLPGMGDA
LTVATLRRDEGGQERLVTALAEAYVQGVAVDWAAVIYNTTALVDLPTYPFQHEHYWLDSTRLMGLAAEERDKALVAV
VRESAAVVLGHADARAIPATAAFRELGVDSLTAVQLRNSLAKATGLRLPTTLAFDYPTPAVLAARL
SEQ ID NO: 103
EPLAIVGMACRLPGGVSSPEDLWRLVESGTDAISDLPTDRGWDLDNLYDPDPGAPGKSYCVQGGFLDTVADFDPAFF
GISPREALAMDPQQRLVLEVSWEAFERAGIEPGSVRGSDTGVFMGAFGNGYGIDTDGGGFGATAGTGSVLSGRVSYF
LGLEGPAMTVDTGCSSALVALHQARYALRQGDCSLALVGGVTVMASPYTFVEFSRQRGMAADGRCKAFADAADGTGW
AEGVGVLLVERLSDAEAKGHQVLAVVRGSALNQDGASNGLTAPNGPSQQRVIQAALANAGLVSADVDVVEAHGTGTT
LGDPIEAQAVLATYGQDRERPLLLGSLKSNIGHTQAAAGVSGVIKMVMALQHGVVPRTLHVDEPSRHVDWSAGAVEL
VTENQPWPSVDRPPRAGVSAFGISGTNAHVILEAVPAPPFEPPTPVTGPVPLVISAKTRPALTAFEARLRAYVTADA
DLTAIASTLATTRSIFEHRAVLLGDDTVTGIAVPDPRVVFVFPGQGWQWLGMGSALRESSVVFAERMAECAAALSDY
VDWDLFSVLDDPAVVDRVDVVQPACWAVMVSLAATWQAAGVRPDAVIGHSQGEIAAACVAGAISLRDAAQIVALRSQ
LIAGLAGHGAMASIALSADQITLTDGAWIAARNGPAATVIAGAPAAVDSVLAAHEDARTRRITVDYASHTPHVEQIR
TELLDLTTDLDSRAPVIPWLSTVDVTWVEGPLDADYWYRNLREPVGFDTAVENLPDSVFIEVSANPVLLPAMGDALT
VATLRRDAGGQTRLLTALAEAYVQGVAVDWVTVIGATPARVDLPTYAFQHQRYWVADRLHDRPSAEQHRLMRELVQR
HAATVLGHASPDTIAADRPFKDLGLDSLTAVELRNHLVAETGLRLSATTAFDHPTADDLAGHL
SEQ ID NO: 104
EPIAIVAMACRAPGGVSSPEGLWRLVESGTDATSGFPTDRGWDVDNLFDPDPDAAGKTYSVRGGFLETAADFDAAFF
GISPREALGMDPQQRLLLETSWEAIERAQIDPKSLRGRDVGVYVGGAAQGYGIGATDQQQENLITGSSISLLSGRVS
YALGLEGPGVTVDTACSSSLVALHLASQALRQRECSLALVSGVSVMATPDVFVEFSRQRGLAADGRCKSFSAAADGT
TWSEGVGVLVLQRLSEAVREGHRVLAVVRGSAVNSDGASNGLTAPNGVSQQRVIRQALAGAGLTASEVDVVEAHGTG
TKLGDPIEAEAILATYGQDRDAPAWLGSLKSNIGHTMAASGVLGVIKMVQAMRHGLLPRTLHVDEPSPHVDWARGDI
ALLTENQPWPDGTRPRRAGVSSFGLSGTNAHVVLEEYPAPVAAAPPVTPARGGPLPWVLSAQSPNALREQAARLYAA
LAEDPDWHPLDIGYSLATTRPGFPHRAVAVGSDREDFQRALSKLADGAGWPGLITATAAKDRRVAFLFDGQGTQRLG
MGRGLHRRFPVFARAWDAVSAAFAKHLDHSLTDIYLGESSPTNTDLADDTLYAQAGIFTLEVALVELLQDWGVRPDF
VTGHSIGEAAAAYVAGVLSLEDVTALIVARGKALRLTPPGDMVALRAGEADVRDFLNRTGAALDLAAVNSPEAVVVS
GTPDAVADFRAAWTASGGQARNLTVRHAFHSRHVESALDEFRTTLETLTFRAPKVPLVSTATGRLVGPAELGAPEYW
LRQVRQTVRFEDALRDLSGRGVGTFVEIGPSGSLATAGLECLGDDASFHAVQRPRSPEDVCLMTAVAELHAGGTTVD
WAKVLAGGRTVDLPVYPFQHRPYWIAPASYPDEPRTMRELVRLEVAGILGLSDPSVILDDSSFLELGFDSLSSLRLG
NRLATVTGLDLPSTLLFDYATPAALATHLD
SEQ ID NO: 105
EPLAIVGMACRLPGGVSTPEDLWRLVESGVDAISDFPADRGWDVANLFDPDPDAPGKTYSVRGGFLDTAADFDAAFF
GISPREALAMDPQQRLVLEASWEAFERAGIEPSSVRGSDTGVFMGAFSAGYGTELEGFGATAGAVSVLSGRVSYFFG
LEGPAMTVDTACSSSLVALHQAGYALRQGECSLALVGGVTVMASPQSFVEFSRQRGLSVDGRCKAFADAADGTGWAE
GVGVLVVERLSDAEAKGHRIQAMVRSSAVNQDGASNGLSAPNGPSQQRVIRQALANAGLTGADVDVVEAHGTGTTLG
DPIEAQALLATYGQDREQPLLLGSLKSNVGHTQAAAGVSGVIKMVMALQHGVVPRTLHVDEPSRHVDWSAGAVELVT
QNQPWPSFDRPRRAGVSSFGVSGTNAHIILESAPAQPLAPSTPIPGLVPLVISAKTAPALTAFEARLRDYLTADADL
TAIAATLATTRSTFEHRAVLLGDDTVTGIAAPDPRVVFVFPGQGWQWLGMGSALCESSVVFASRMAECAAALSEFVD
WDLFSVLDDPAVVDRVDVVQPACWAVMVSLAATWQAAGVHPDAVVGHSQGEIAAACLAGAISLQDGARVVALRSQLI
ARLAGHGAMASIALPADQIALTDGAWIAARNGPAATVIAGAPEAVDSVLAAHGDARVRRITVDYASHTPHVEQIRAE
LLAILADIDSRPPSIPWLSTVDDALVEGPLKADYWYRNLREPVGFDTAVSALQDAVFIEVSANPVLLPAMGDAATVA
TLRRDDGGQDRLLTAVAEAYVQGVAVDWAAVIGATGARVLDLPTYAFQHQRFWARAASAAGLAPEALLKVVQDSAAQ
VLGYADPGAIAVTAAFKDLGIDSLTAVEMRNTLAKKTGLRLPATLVFDYPTPGVLAGRL
SEQ ID NO: 106
EPLAIVGMACRLPGGVSSPEDLWRLVESGGDAISDFPADRGWDIENLFDPDPDAAGKTYSVRGGFLDAAAGFDAAFF
GISPREALAMDPQQRLVLEASWEAFERAGIEPGSVRGSDTGVFMGAYPGGYGTGADVGGFGATAGAVSVLSGRVSYF
FGLEGPAMTVDTACSSSLVALHQAGHALRQGECSLALVGGVTVMATPHTFIEFSRQQGLASDGRSKAFADAADGAGF
SEGVGVLVVERLSDARAKGHRVLAVVRSSAVNQDGASNGLTAPNGPSQQRVIRQALANAGLTGADVDVVEAHGTGTT
LGDPIEAQAVLATYGQDRQKPLLLGSLKSNVGHTQAAAGVSGVIKMVMALQHGVVPRTLHVDEPSRHVDWSAGAVEL
VTENQPWPSVDRPRRAGVSSFGISGTNAHVILESVPVQLPVPSAGLAPLMISAKTAPALGDAEARLRGYLTADADLP
AIASTLATTRSMFEHRAVLLGDTTITGTAAADPKVVFVFSGQGSQRAGMGEQLAFPVFADIHRRVWDLLDVPDLDVD
QTGYAQPALFALQVALAGLLESWGVRPQAVIGHSVGELAAGYVAGLWSLEDACTLVSARARLMQALPPGGVMVAVPV
SEDQARAALLEGVEIAAVNGPSSVVLSGDETAVLQVAAGLGKWTRLSTSHAFHSARMEPMLEEFRAVAEQVTYRTPV
ITMAAGAATPDYWVRQVRDTVRFGDQVAAFEGATFVEIGPDRTLARLVDGIAMLHGDDEVEAALTGLARLFVQGVPV
AWDNGARVLDLPTYPFQHQRYWLDARRAASAGGDLLKMVRDNAALILGHTNPGAISETTAFRDLGVDSLTAVQLRNS
LAKATGLRLPATLVFDYPTPSVLAGRL
SEQ ID NO: 107
EPLAIVGMACRLPGGVSSPEDLWRLVESGTDAISDFPADRGWDVDNLFDPDPDAPGKTYSVQGGFLDAAAEFDAAFF
GISPREALAMDPQQRLVLEASWEAFERAGIEPGSVRGSDTGVFMGAYSGGYGAGADLDGFGATAGAGSVLSGRISYF
FGLEGPAMTVDTACSSSLVALHQAGSALRQGECSLAVVGGVTVMATPDLFVEFSRQRGLAADGRCKAFGDAADGTGW
AEGVGVLLVERLSDAEAKGHRVLAVVRSSAVNQDGASNGLTAPNGPSQQRVIRSALATAGLAPQDVDVVEAHGTGTT
LGDPIEAQAVLATYGQDRERPLLLGSLKSNVGHTQAAAGVSGVIKMVMALQHGVVPRTLHVDEPSRHVDWSAGAVEL
VTQNQPWPDVDRPRRAAVSAFGVSGTNAHVILESVPAVPPVPSAGPAPLMISAKTAPALGDAEARLRDYLTADADLT
AIASTLATTRSTFEHRSVIFENHTITGTAAPDPRVVFVFSGQGSQRAGMGEQLAATFPVFAEIHRRVWDLLDGPDLD
VDQTGYAQPALFALQVALVGLLESWGVRPEAVIGHSVGELAAGYVSGLWSLEDACTLVSARARLMQALPPGGVMVAV
PVPEDQARAALVEGVEIAAVNGPSSVVLSGDEAAVLQVAAGLGKWTRLATSHAFHSARMEPMLEEFRGVAEQLTYRT
PVISMAAEVATPDYWVRQVRDTVRFGDQVAEFEGATFVEIGPDRTLARLIDGIAMLHGDDEVEAALNGLARLFVQGV
PVAWDNGGRVLDLPTYPFQRQRYWAVSPEALLKAVRDSAAMILGHADPSAISETAAFRDLGVDSLTAVELRNSLAKA
TGLRLPATLVFDYPTPAVLAARL
SEQ ID NO: 108
EPLAIVGMACRLPGGVSSPEDLWRLVESGTDAISDFPTDRGWDAASLFDPDPDAAGKTYSVQGGFLDAAADFDAAFF
GISPREALAMDPQQRLVLEASWEAFERAGIEPGSVRGTDTGVFMGAFSAGYGARLEGFGATAGAVSVLSGRVSYLFG
LEGPAMTVDTACSSSLVALHQAAYSLRQGECSLALVGGVTVMATPQIFVDFSRQRGLAPDGRCKAFGDNADGTGWAE
GVGVLVVERLSDAQAKGHRVLAVVRSSAVNQDGASNGLTAPNGPSQQRVIRQALANAGLTSADVDVVEAHGTGTTLG
DPIEAQAVLATYGQDRDRPLLLGSLKSNIGHTQAASGVSGVIKMVMALQHGVVPPTLHADQPSQHVDWSTGAVELVT
QSQPWPSVDRPRRAGVSSFGISGTNAHVILESVPAQPPVPSAGPAPLMISAKTAPALGEAEARLRDYLTADADLPAI
ASTLATTRSIFEHRAVLLGDTTITGTAAADPKVVFVFSGQGSQRAGMGEQLAFPVFADIHRRVWDLLDVPDLDVDQT
GYAQPALFALQVALAGLLESWGVRPQAVIGHSVGELAAGYVAGLWSLEDACTLVSARARLMQALPPGGVMVAVPVSE
EQARAALTEGVEIAAVNGPSSVVLSGDETAVLQVAAGLGKWTRLSTSHAFHSARMEPMLEEFRAVAEQLTYRTPTIT
MTEEVTTPDYWVRQVRDTVRFGDQVAAFEGATFVEIGPDRTLARLIDGIAMLHGDDETEAALTGLARLFVQGVPVTW
DNKARVLDLPTYPFQRQRYWAGWLAGLAAEERDKALVTVVRDSVAAVLGYADSRKIPVSAAFKDLGVDSLTAVELRN
SLAKTTGLRLPATLVFDHPTLATLAARL
SEQ ID NO: 109
EPLAIVGVACRLPGGVSSPEALWQLVESGTDAISGFPADRGWDVDNLFDPDPEASGKTYCVQGGFLDAVAEFDASFF
GISPREALAMDPQQRLILEVSWEAFERAGIEPGSVRGSNTGVFMGAFGSGYGSDLEGFSATAGAGSVLSGRISYFFG
LEGPAMTIDTACSSSLVALHQAGYALRQGECSLALVGGATVMATPQTFIEFSRQRGLAADGRCKSFGDNADGTGWSE
GVGALLVERLSDAQAKGHRVLAVVRGSAVNQDGASNGLTAPNGPSQQRVIRQALANAGLAPQDVDVVEAHGTGTRLG
DPIEAQAVLATYGQDRERPLLLGSLKSNVGHTQAAAGVSGVIKMVMALQHGVVPQTLHVDEPSRQVDWSAGAVELVT
ENQPWPDVDRPRRAAVSAFGVSGTNAHVILESAPAQPVAPSTPATGLTPLVISAKTAPALTASEARLRDYLTADADL
TAIAATLAATRSAFEHRAVLLGDDTVTGIAAPDPRVVFVFPGQGWQWLGMGSALRDSSVVFAERMAECAAALSDYVD
WDLFSVLDDPAVVDRVDVVQPACWAVMVSLAATWQAAGVRPDAVVGHSQGEIAAACVAGAISLRDGAKIVALRSQLI
ARLAGHGAMASIALPADQITLTDGVWIAARNGPAATVIAGAPEAVDSVLSAHGDARVRRIAVDYASHTPHVEQIRTE
LLPILADIDSQTPRIPWLSTVDDTWIEGPLGADYWYRNLREQVGFDTAVEHLQDSVFIEVSASPVLLPAMGDAITVA
TLRRDEGGQDRLVTALAEAYVQGVPVDWAAVIDNTTARVLDLPTYAFQHQRFWVANLTPEALLKAVRDSAATVLGHA
DPGTIPETAAFKDLGIDSLTAVELRNSLAKTTGLRLPATLVFDYPTPGVLAARL
SEQ ID NO: 110
EPLAIVGMACRLPGGVSSPEDLWRLVESGGDAISDFPVDRGWDVDNLFDPDPDAAGKTYSVQGGFLDTAAEFDAAFF
GISPREALAMDPQQRLVLEASWEVFERAGIEPGSVRGSDTGVFMGAYPGYYGIGADLDGFGATAGAGSVLSGRVSYF
FGLEGPAMTVDTACSSSLVALHQAGSALRQGECSLALVGGVTVMATPQTYVEFSRQRGLASDGRSKAFADAADGAGF
SEGVGVLLVERLSDARRHGHRVLAVVRSSAVNQDGASNGLTAPNGPSQQRVIRQALATAGLSPHEVDVVEAHGTGTT
LGDPIEAQAVLATYGQDRDRPLLLGSVKSNIGHTQAAAGVSGVIKMVMALQHGVVPPTLHVDEPSRHVDWSAGAVDL
VTENRPWPDLDRPRRAGVSSFGISGTNAHVILESVPAVPPVPSAGPAPLMISAKTAPALGEAEARLRDYLTADADLP
AIASTLASTRSTFEHRAVIFQNHTITGTAAADPRVVFVFSGQGSQRAGMGEQLAATFPVFKDIHRRVWDLLDVPDLD
VDQTGYAQPALFALQVALFGLLESWGVRPEAVIGHSVGELAAGYVAGLWSLEDACTLVSARARLMQALPPGGVMVAV
AVSEEHAQAALIKGVEIAAVNGPSSVVLSGDETAVLQVAAGLGKWTRLSTSHAFHSARMEPMLEKFRAVAEQLTYRT
PVITMAAEVTTPDYWVRQVRDTVRFGDQVAAFEGATFVEIGPDRTLARLVDGIAMLHGDDEVEAALTGLARLFVQGV
PVTWDNGGRVLDLPTYAFQRQRYWATSTRWLAGLTPQERENALLKVVRDNAAVVLGHAGAGAIPATAAFRDLGVDSL
TAVELRNSLATTTGLRLPATMVFDYPTPAAVAARL
SEQ ID NO: 111
EPLAIVGMACRLPGGVSSPEDLWRLVESGTDAISDFPTDRGWDVESLFDPDPDAAGKTYSVRGGFLDAAASFDAAFF
GISPREALAMDPQQRLVLEASWEAFERAGIEPSSVRGSDTGVFMGAFSAGYGTELEGFGVTAGAVSVLSGRVSYFFG
LEGPAMTVDTACSSSLVALHQAGYALRQGECSLALVGGVTVMASPQSFVEFSRQRGLSVDGRCKAFADAADGTGWAE
GVGVLVVERLSDAQAKGHRVLAVVRSSAVNQDGASNGLSAPNGPSQQRVIRQALANAGLTGADVDVVEAHGTGTTLG
DPIEAQAVLATYGQDREQPLLLGSLKSNIGHTQAAAGVSGVIKMVMALQHGVVPRTLHIDEPSQHVDWSAGAVELVT
QNQPWPGNDRPRRAGVSSFGVSGTNAHVILESAPTQPALPSVTATGPVPLVISAKTAPALTAFEARLRDYLTADADL
TAIAATLATTRATFDHRAVLLGDDTVTGVAVPEPRVVFVFPGQGWQWLGMGSALSESSVVFAERMAECATALDEFVD
WDLFSVLDDPAVVDRVDVVQPACWAVMVSLAATWQAAGVHPDAVIGHSQGEIAAACVAGAISLRDGARIVALRSQLI
ARLAGHGAMASIALPADQITLTDGVWIAARNGPAATVIAGDPAAVDSVLAAHQDARVRRITVDYASHTPHVEQIRAE
LLAILSDIGSQTPVIPWLSTVDGEWVEGPLGNDYWYRNLRETVGFDTAVGLLPDSVFIEVSASPVLLPAMGDAVTVA
TLRRDDGGLTRLLTALAEAWVQGVAVDWAIGATTARVLDLPTYAFQHQHYWAVTGTGLTPEALLKVVQDSTAQVLGY
TDAAAIAVTAAFKDLGIDSLTAVEMRNTLAKATGLRLPATLVFDYPTPSLLAGRL
SEQ ID NO: 112
EPLAIVGMACRLPGGVSSPEDLWRLVESGTDAISDFPTDRGWDVESLFDPDPDAAGKTYSVRGGFLDAAAGFDAAFF
GISPREALAMDPQQRLVLEASWEAFERAGIEPGSVRGSDTGVFMGAYPGGYGTGVDVGGFGATAGAVSVLSGRVSYF
FGLEGPAMTVDTACSSSLVALHQAGHALRQGECSLALVGGVTVMATPHTFIEFSRQQGLASDGRSKAFADAADGAGF
SEGVGVLVVERLSDAQAKGHRVLAVVRSSAVNQDGASNGLTAPNGPSQQRVIRQALADAGLVSADVDVVEAHGTGTT
LGDPIEAQAVLATYGQDREHPLLLGSLKSNVGHTQAAAGVSGVIKMVMALQHGVVPRTLHVDEPSRHVDWSAGAVNL
VTENLPWPSLDRPRRAGVSSFGISGTNAHVILESVPAQPPVSSTGPAPLVISAKTGPALTAFEARLRTYLAAASEVD
LGAVAATLATTRSVFEHRAVLLGEETIAGTAAVDPRVVFVFSGQGSQRAGMGEQLADAFPVFADIHRRVWDLLDVPD
LDVNQTGYAQPALFALQVALFGLLESWGVRPAAVIGHSIGELAAGYVSGLWSLEDACTLVSARARLMQALPPGGVMV
AVPVSEEQARGVLVEGVEIAAVNGPSSVVLSGDEAVVLQVASGLGKWTRLSTSHAFHSARMEPMLEEFQAVAEQLTY
RTPAIEMAAGEEVTTPDYWVRQVRDTVRFGEQVAAFSDAVFVEVGPDRTLARLIDGVAMLHGDDEPSAALTGLATLF
VQGVPVDWSAVVSGTEARVLDLPTYAFQHQRYWLDRKAARRAASAGGDLLKMVRGNAALILGHADPSAIAATTAFRE
LGVDSLTAVQLRNSLAKATGLRLPATLVFDYPTPAVLAGRL
SEQ ID NO: 113
EPLAIVGMACRLPGGVSSPEDLWRLVESGTDAISDFPPDRGWDVENLFDPDPDAPGKTYSIHGGFLDTAAEFDAAFF
GISPREALAMDPQQRLVLEASWEAFERAGIEPGSVQGSDTGVFMGAYSAGYGAGADLDGFGATAGAGSVLSGRISYF
FGLEGPAMTVDTACSSSLVALHQAGSALRQGECSLALVGGVTVMATPDLFVEFSRQRGLATDGRCKAFADTADGTGW
AEGVGVLLVERLSDAQAKGHRVLAVVRGSAVNQDGASNGLTAPNGPSQQRVIRQALANAGLAPHEVDVVEAHGTGTT
LGDPIEAQAVLATYGQDREQPLLLGSLKSNVGHTQAAAGVSGVIKMVMALQQGVVPQTLHVDEPSQHVDWSAGAVNL
VTQNQPWPDIDRPRRAAVSAFGVSGTNAHVILESVPASPPVPSTGPAPLVISAKTVPALTAFEARLRTYLAAVPEVD
LGAVAATLATTRATFEHRAVLLGEETIAGTAAVDPRVVFVFSGQGSQRAGMGEQLAAAFPVFADIHHRVWELLDIPD
LDVDQTGYAQPALFALQVALFGLLESWGVRPAAVIGHSVGELAAGYVSGLWSLEDACTLVSARARLMQALPPGGVMV
AVPVSEEQARAVLVEGVEIAAVNGPSSVVLSGDEAVVLQVASGLGKWTRLSTSHAFHSARVEPMLEEFRVIAGQLTY
RTPVIEMAAGEQVTSPDYWVRQVRDTVRFGEQVAAFSDAVFVEIGPDRTLARLIDGVALLHGDDETEAAMAGLARLF
VQGVPVDWSAVLGGTEARVLDLPTYAFQHQRYWAALTPEALLKVVRDSAAMVLGHADPSAISGTAAFRDLGLDSLTA
VELRNSLAKATGLRLPATLVFDYPTPSVLAGRL
SEQ ID NO: 114
EPLAIVGMACRLPGGVSSPEDLWRLVESGTDAISDFPPDRGWDTASLFDPDPDAAGKTYSVQGGFLDAVAEFDAGFF
GISPREALAMDPQQRLVLEASWEAFERAGIEPGSVRGTDTGVFMGAFSAGYGAHLEGFGATAGAVSVLSGRVSYLFG
LEGPAMTVDTACSSSLVALHQAAYSLRQGECSLALVGGVTVMATPQIFVDFSRQRGLAADGRCKAFADDADGTGWAE
GVGVLVVERLSDAQAKGHRVLAVVRSSAVNQDGASNGLTAPNGPSQQRVIRQALANAGLTSADVDVVEAHGTGTTLG
DPIEAQAVLATYGQDREQPLLLGSLKSNLGHTQAAAGVSGVIKMVMALQHGIVPRTLHVDQPSQHVDWSAGAVELVT
ENQPWPSLDRPRRAGVSSFGISGTNAHVILESVPASPPVPSTGPAPLVISAKTGPALTAFEARLRTYLAATPDADLP
TIASTLATTRSVFEHRAVLLGEETIAGTAAVDPRVVFVFSGQGSQRAGMGEQLADAFPVFADIHRRVWDLLDVPDLD
VNQTGYAQPALFALQVALFGLLESWGVRPAAVIGHSIGELAAGYVSGLWSLEDACTLVSARARLMQALPPGGVMVAV
PVSEEQARGVLVEGVEIAAVNGPSSVVLSGDEAVVLQVASGLGKWTRLSTSHAFHSARMEPMLEEFQAVAEQLTYRT
PAIEMAAGEEVTTPDYWVRQVRDTVRFGEQVAAFSDAVFVEVGPDRTLARLIDGVAMLHGDDEPSAAGTALARLHVQ
GVPVDWSAVLGGTGARVLDLPTYAFQRQRYWAGWLAGLAAEERDKALVTVVRDSVAAVLGYADSRKIALSASFKELG
VDSLTAVELRNNLAKTTGLRLPATLVFDHPTLAAMAARL
SEQ ID NO: 115
EPLAIVGVACRLPGGVSSPEALWRLVESGTDAISGFPADRGWDVDNLFDPDPEASGKTYCVQGGFLDTVADFDASFF
GISPREALAMDPQQRLILEVCWEAFERAGIEPGSVRGSDTGVFMGAFGSGYGSDLEGFSATAGAGSVLSGRISYFFG
LEGPAMTVDTACSSSLVALHQAGYALRQGECSLALVGGATVMATPQTFIEFSRQRGLAADGRCKSFGDNADGTGWSE
GVGALLVERLSDAQAKGHRVLAVVRGSAVNQDGASNGLTAPNGPSQQRVIRQALANAGLAPHEVDVVEAHGTGTRLG
DPIEAQAVLATYGQDRERPLLLGSLKSNVGHTQAAAGVSGVIKMVMALQHSVVPQTLHVDAPSRQVDWSAGAVELVT
QNQPWPETGRARRAAVSAFGVSGTNAHVILESAPAQPPAPSTPVTGPVPLVISAKTASALGQAEARLRTYLADKPDA
DLAAIAATLATTRSTFEHRAVLLGDETIRGVAVPDPRVVFVFPGQGWQWLGMGSALRESSVVFAERMAECAAALSDY
VDWDLFSVLDDLAVVDRVDVVQPACWAVMVSLAATWQAAGVRPDAVIGHSQGEIAAACVAGAISLRDAAQIVALRSQ
LIAGLAGQGAMASIALPADQITLTDGVWIAARNGLAATVIAGDPAAVDGVLAAHQDARVRRITVDYASHTPHVEQIR
TELLDLTTDISSRTPAIPWLSTVDSTWIEGPLDTDYWYRNLREPVGFDTAVNLLPDSVFIEVSASPVLLPAMGDAAT
VATLRRDDGSQTRLLTALAEAYVQGVAIDWTIGATTARVLDLPTYAFQHQRFWVANALTPEALLKVVRDSAATVLGH
ADPGTIPETAAFKDLGVDSLTAVELRNSLAKATGLRLPATLVFDYPTPSVLAGRL
SEQ ID NO: 116
EPLAIVGMACRLPGGVSSPEDLWRLVESGTDAISDFPADRGWDIENLFDPDPDAPGKTYSVQGGFLDTAAEFDAGFF
GISPREALAMDPQQRLVLEASWEVFERAGIEPGSVRGSDTGVFMGAYPGYYGIGADLDGFGATAGAGSVLSGRVSYF
FGLEGPAMTIDTACSSSLVALHQAGSALRQGECSLALVGGVTVMATPQTYVEFSRQRGLASDGRSKAFADAADGAGF
SEGVGVLLVERLSDARRHGHRVLAVVRSSAVNQDGASNGLTAPNGPSQQRVIGSALANAGLAPHDVDVVEAHGTGTA
LGDPIEAQAVLATYGQDREQPLLLGSVKSNLGHTQAAAGVSGVIKMVMALQHGIVPRTLHVDEPSRHVDWSAGAVEL
VTENQPWPEHDRPRRAGVSSFGISGTNAHVILESVPAQPPVSSTGPAPLVISAKTASALGQAEARLRTYLTVDADLP
AIAATLATTRAVFEHRAVLLGDTTITGVAADPRVVFVFSGQGSQRAGMGEQLAAAFPVFADTHRRVWDLLDVPDLDV
DQTGYAQPALFALQVALFGLLESWGVRPEAVIGHSVGELAAGYVSGLWSLEDACALVSARARLMQALPPGGVMVAVA
VSEEQARTALVEGVEIAAVNGPGSVVLSGDEAVVLQVASGLGKWTRLATSHAFHSARMEPMLEEFRAVAEQLTYRTP
AIEMAAGEQVTTPDYWVRQVRDTVRFGEQVAAFGDAVFVEIGPDRTLARLIDGVAMLHGDDETEAAMAGLAKLFVEG
IPVDWSAVLGGNAARVDLPTYAFQRQRYWAASLLAGLTPEERGNALLKVVRDNAAVILGHAGAAAIPATAAFRDLGV
DSLTAVELRNSLATSTGLRLPATMVFDYPTPAAMAARLD
SEQ ID NO: 117
EPLAIVGMACRLPGGVFSPEDLWHLVESGTDAISGFPADRGWDVEKLFDPDPDAPGKTYCVQGGFLEATAAFDAAFF
GISPREALAMDPQQRLMLEVSWEAFERAGIEPGSVRGSDTGVFLGAYPGGYGAGAGTDLGGFGATGGAGSVLSGRVS
YFFGLEGPAMTVDTACSSSLVALHQAAYSLRQRECSLALVGGVTVMGTPHMFVDFSRQRGLSVDGRCKAFADAADGT
SWSEGVGVLLVERLSDAQAKGHRVLAVVRGSAVNQDGASNGLTAPNGPSQQRVIRQALANAGLAPHEVDVVEAHGTG
TTLGDPIEAQAVLATYGQDREQPLLLGSLKSNVGHTQAAAGVSGVIKMVMALQRGVVPQTLHVDQPSRHVDWSAGAV
DLTTENRPWPDTDRPRRAGVSSFGVSGTNAHVILESAPAQPPTPSTPVTGPVPLVISAKTASALGQAEARLRDYLTA
DADLTAIAATLAITRSTFEHRAVLLGDDTITGVATPDPRVVFVFPGQGWQWLGMGSALRESSVVFAERMAECAAALD
EFVDWDLFSVLDDPAVVDRVDVVQPACWAVMVSLAATWQAAGVRPDAVIGHSQGEIAAACVAGAISLRDGAKIVALR
SQLIAGLAGQGAMASIALPADQITLTDGVWIAARNGPAATVIAGTPSAVDSVLAAHQDARVRRITVDYASHTPHVEQ
IRTELLGILADIDSQTPLIPWLSTMEGTWVEGPLHSDYWYRNLREPVGFDTAVSLLPDSVFIEVSASPVLLPAMGDA
LTVATLRRDEGGQNRMFTALAEAYVQGVAVDWAAVIGATTARVLDLPTYAFQHEDYWLDSTRLMGLAAEERDKALVT
VVRESAAVVLGHADARAIPVTAAFRELGVDSLTAVQLRNSLAKATGLRLPTTLAFDYPTPAVLAARL
SEQ ID NO: 118
EPLAIVGMACRLPGGVLSPEDLWRLVESGTDAISGLPTDRGWDIDNLYDPEPGAPGKSYCVQGGFLDTVADFDPAFF
GISPREALAMDPQQRLVLEVSWEAFERAGIEPGSVRGSDTGVFMGAFGNGYGIDTDGGGFGATAGTGSVLSGRVSYF
LGLEGPAMTVDTGCSSALVALHQARYALRQGDCSLALVGGVTVMASPYTFVEFSRQRGMAANGRCKAFADAADGTGW
AEGVGVLLVERLSDAEAKGHRVLAVVRGSALNQDGASNGLTAPNGPSQQRVIQAALANAGLVSADVDVVEAHGTGTT
LGDPIEAQAVLATYGQDREHPLLLGSLKSNIGHTQAAAGVSGLIKMVMALQHGVVPQTLHVDEPSRHVDWSAGAVEL
VTENRPWPSVDRPRRAGVSAFGISGTNAHVILESAPPSPAPSTPVTGLVPLVISAKTAPALGQAEARLRDYLTADVD
LTAIAATLVTTRSTFEHRAVLLGDDTVTGVAVPDPRVVFVFPGQGWQWLGMGSALRESSVVFAERMAECASALSDYV
DWDLFTVLDDPAVVDRVDVVQPACWAVMVSLAATWQAAGVRPDAVIGHSQGEIAAACVAGAISLRDAAQIVALRSQL
IAGLAGHGAMASIALPADQITLTDGVWIAARNGPTATVIAGNPQAVDSVLAAHQDARVRRITVDYASHTPHVEQIRT
ELLDLTTDVGSRTPAIPWLSTVDGEWVEGPLDTDYWYRNLREPVGFDTAVGMLPDSVFIEVSASPVLLPAMGDAATV
ATLRRDDGGQTRLLTALAEAYVQGVAVDWAVGATTARVLDLPTYAFQHQRYWVADRLHDRPGVEQHRLMRELVLRHA
ATVLGHDSPDAIAADHPFKDLGLDSLTAVELRNHLVAETGLRLSATTAFDHPTADDLARHL
SEQ ID NO: 119
EPIAIVSMACRAPGGVSSPEGLWRLVESGTDATSGFPTDRGWDVENLFDPDPDAAGKTYSMRGGFLETAADFDAPFF
GISPREALGMDPQQRLLLETAWEAIERAQIDPKSLRGQDVGVYVGGAAQGYGIGATDQQQENLITGSSISLLSGRVS
YALGLEGPGVTVDTACSSSLVALHLAGQALRQRECSLALVSGVSVMATPDVFVEFSRQRGLAADGRCKSFAASADGT
TWSEGVGVLVLQRLSEAVRQGHRVLAVVRGSAVNSDGASNGLTAPNGVSQRRVIRQALASAGLAASEVDVVEAHGTG
TKLGDPIEAEAILATYGQDRAAPAWLGSLKSNIGHTMAASGVLGVIKMVEAMRHGLLPRTLHVDEPSSHVDWERGDV
ALLTENQPWPDSTRPRRAGVSSFGLSGTNAHVVLEEYPAPAAADPPVTPAGGGPLPWVLSAQSPNALREQAARLYAA
LAEDPDWRPLDIGYSLATTRAGFPHRAVAVGSDREEFQRALSKLADGTGWPGLITATAAKDRRMAFLFDGQGTQRLG
MGKGLHRRFPVFARAWDAVSAAFAKHLDHSLTDIYLGPSSPASAELADDTLYAQAGIFTMEVALVELLEDWGVRPDF
VAGHSIGEAAAAYTAGMFSLEDVTALIVARGRALRLTPPGEMVALRGGEADVRELLQRTGAALDLAAVNSPEAVVVS
GAPDAVAEFRAAWTASGRRARDLTVRHAFHSRHVESVLDEFRATLAALTFRAPALPVVSTMTGRLADPAEMGTPEYW
LRQVRQTVRFEEAVRELSGQGVGTFVEIGPSGALATAGLECLGGDATFHAVQRPRAPEDVCLMTAVAELHAGGTAVD
WTKILAGGRPVDLPVYPFQHRPYWIAPAPSYPDEPRTMRELVRLEVAGILGLSDPSVILDDSSFLELGFDSLSSMRL
GNRLATVTGLDLPSTLLFEYATPAALATHLD
SEQ ID NO: 120
EPLAIVGMACRLPGGVESPDDLWRLVASGTDAISGFPRDRGWDVDNLYDPDPDAPGKTYTVLGGFLDSVAGFDASFF
GISPREALAMDPQQRLVLEVAWEAFEHAGIAPRSVRGTDTGVFMGAFSSGYDAELEEFGMTGDAVSVLSGRVSYFFG
LEGPAMTVDTACSSSLVALHQASSALRQGECSLALVGGVTVLATPKTFVEFSRQRGLAGDGRSKAFADAADGAGWSE
GVGVLLVERLSDARAKGHHVLGVVRGSAVNQDGASNGLSAPNGPSQQRVIRQALAGAGLSPHEVDVVEAHGTGTKLG
DPIEAQAVIATYGQDRDQPALLGSLKSNVGHTQAAAGVAGVIKMVMALQHATVPATLHVDAPTRHVDWTAGAVELVT
ENRPWPETGRARRAAVSSFGISGTNAHVILESAPAAAPEETEPVAPVVASDRVPLVISAKTPAALTSTEDRLRAYLA
AHPGTDPRAVASTLATTRSVFEHRAVLLGENTVTGSVAGADPRVVFVFPGQGWQRLGMGRELLAASPVFAGRMAECA
TVLREFVDWDLFTMLDDPAVVDRIEVVQPVCWAIMVSLAAVWQAAGVRPDAVIGHSQGEIAAACVSGAVSLRDAARI
VTFRSDMIARMTGHGVMASVALHADDIPLVEGAWVAARNGPAATVVAGTPEAVDQVLAACEERGARVRRITAGVASH
TPLAEHVRGELLDATGGLPSRVPDIPWLSTVDGTWVEKPLDPAYWFRNMREPVGFAPAVDLLRAQGDHVFLEISASP
VLLPSMDDAVTVATLRRDDGSADRMLAALAEAHTHGVVVDWPRVLGTAGRVRGLPTYAFQHQRYWAVSRPAVLTPDA
LLKVVRDSAATVLGYTDADSITVTTAFRDLGVDSLSAVELRNNLAKSTGLRLPATLVFDYPTPADLATHL
SEQ ID NO: 121
EPLAIIGMACRLPGGITSPEDLWRLVASGSDAISDFPDDRGWDVGNLYDPDPDAPGRSTTVRGGFLDEVAGFDASFF
GISPREALAMDPQQRLVLEVSWEAFERAGIEPGTVRGSDTGVFMGAYPGGYGVGADLGGFGTTAVSGSVLSGRVSYF
FGLEGPAMTVDTACSSSLVALHQAGHALRQGECSLALVGGVTVMPTPNIFVEFSRQRGLAADGRCKPFADAADGTGF
SEGAGVLLVERLSDAQTNGHHILAVVRASAVNQDGASNGLTAPNGPSQQRVIRSALANAGLTTADVDVVEAHGTGTT
LGDPIEAQAVIATYGQDRAQPVLLGSLKSNIGHTQAAAGVSGVIKMVMALRNGTVPRTLHVDEPSRHIDWTAGAVEL
ATENRPWPETERPRRAGVSSFGISGTNAHVILESTPTQPVEPSTPAAHPLPLPISAKTPPALAALEARLRAYLTSET
DLAAVASTLASTRAVFEHRAVLLGDETIVGVAALDPRVVFVFSGQGSQRAGMGEQLAAVFPVFAQIHREVLDLLDIP
DLDIDQTGHAQPALFAFQVALAGLLDSWGVRPDAVIGHSIGELAAAYIAGLWSLEDACTLVSARARLMQALPSGGAM
VAVQATEEQARAVLIDGVEIAAVNGPSSVVLSGDETAVLQVAAELGGKSARLKTSHAFHSARMEPMLDQFRQVAEQL
TYRSPVIEMAAGTTSDYWVRQVRDTVRFGDQVRVHQGSVLVEIGPDRTLARLIDGIATSHGDDEVRAVMTALAELHV
RGVAVDWPGTTSARVLDLPTYAFQHERYWLANTAAELTAADLLKAVRDSAAVVLGHADADSIPATTAFKDLGFDSLT
AIELRNRLAKDIGLRLPATMAFDYPTPAALAARL
SEQ ID NO: 122
EPLAIVGMACRLPGGVTSPEDLWRLVASGTDAITEFPTDRGWDVGNLYDPDPDAPGKSTTVHGGFLEGVAGFDASFF
GISPREALAMDPQQRLVLEVSWEAFERAGIEPGAVRGSDTGVFMGAYPGGYGVGADLGGFGTTAGAGSVLSGRVSYF
FGLEGPAMTVDTACSSSLVALHQAGHALRQGECSLALVGGVTVMPTPNIFVEFSRQRGLSADGRCKPFADAADGTGW
SEGVGVLVVERLSDARANGHRILAVVRGSAVNQDGASNGLTAPNGPSQQRVIRQALANAGLTTADVDVIEAHGTGTT
LGDPIEAQAVIATYGQDRTQPVLLGSLKSNIGHTQAAAGVSGVIKMVMALQHDTVPASLHVDEPSRHVDWTAGAVEL
ATESRPWPKTGRAHRAGVSSFGVSGTNAHVILESAPTQPEEPSTPAPHPLPLPVSAKTSAALTDLEDRIRAYLTPET
DLAAVASTLASTRAMFEHRAVLLGDETITGVAAPDPRLVFVFSGQGSQRAGMGEQLAAVFPVFAQIHREVLDLLDVP
DLDIDQTGHAQPALFAFQVALAGLLDSWGVRPDAVIGHSIGELAAAYVAGLWSLQDACALVSARARLMQALPPGGAM
VAVAVPEEQARAVLIDGVEIAAVNGPSSVVLSGDETAVLQVAAELGGKSTRLRTSHAFHSARMEPMLDQFRQVAEQL
TYRSPVIEMAAGTTPDYWVRQVRDTVRFGDQVRVHQGSVLVEIGPDRTLARLIDGIATSHGDDEVRAAMTALAELHV
RGVAVDWPGTTSARVLDLPTYAFQHRRYWVAPARRAAGRPADLTPEGLLTTVRDSAAVVLGHADASAIPATAAFQAL
GVDSLIAVELRNNLAKNTGLRLPATLIFDYPTPVDLATHL
SEQ ID NO: 123
EPLAIIGMSCRLPGGVTSPEDLWRLVASGTDAITGFPADRGWDLENLYDPDPDAPGRTTTVQGGFLDDVAGFDASFF
GISPREAVAMDPQQRLALEASWEAFERAGIEPGSVRGSDTGVFMGAYPGGYGIGADLGAFMLTGRAGSVLSGRLSYF
FGLEGPAMTVDTACSSSLVALHQASYALRQGECSMALVGGVTVMPTPVMFVEFSRQRNLADDGRCKAFADGADGTGW
SEGVGVLLVERLSDALAKGHRIMAVVRGSAVNQDGASNGLTAPNGPSQQRVIQSALDSAGLTTADVDVIEAHGTGTT
LGDPIEAQAVIATYGQDRAQPVLLGSLKSNIGHTQAAAGVSGVIKMVMALQNGVVPRTLHVDEPSRHVDWTAGAVEL
ATENRPWPEVGRARRAAVSSFGFSGTNAHVILESAPAQPATPSAPVAHLLPLPISAKTPPALADLEARLRAYLTPEA
DLPAVASTLASTRAVFEHRAVLLGDETIVGIAALDPRVVFVFSGQGAQRAGMGEQLAAVFPVFAQIHREVLDLLDIP
DLDIDQTGHAQPALFAFQVALAGLLESWGVRPDAVIGHSIGELAAAYIAGLWSLEDACALVSARARLMQALPSGGAM
VAVQATEDQARAVLIDGVEIAAVNGPSSVVLSGDETAVLQVAAGLGGKSTRLRTSHAFHSARMEPMLDQFRQVAEQL
TYRSPVIEMAAGVTPDYWVRQVRDTVRFGDQVRVHQGSVLVEIGPDRTLARLIDGIATSHGDDEVQAAMTALAELHV
RGVAVDWPGTTSARVLDLPTYAFQHQRYWTVSWLAGLTPEEREGALVKVVRDSAAVVLGHADAGTIPVTAAFKDLGL
DSLTAVELRNSLARSTGLRLPATMVFDYPTLGALAARLD
SEQ ID NO: 124
EPLAIVGMACRLPGGVTSPEDLWRLVESGTDAVSAFPADRGWDADALYDPDPEAAGKTYCVRGGFLDGVAGFDASFF
GISPREALAMDPQQRLILEASWEAFERAGIEPGSVRGSDTGVFMGAFPGSYGVDADLGGFGMTGGAASVLSGRVSYF
FGLEGPAMTVDTVCSSSLVALHQAGHALRQGECSLALVGGVTVMSTPDTFVEFSRQRGLAADGRCKAFGDGADGTGW
AEGAGVLLVERLSDAQAKGHRILAVVRGSAVNQDGASNGLTAPNGPSQQRVIRSALANAGLSSADVDVVEAHGTGTK
LGDPIEAQAVLDTYGQDRERPLLLGSLKSNVGHTQAAAGVSGVIKMVMALQHGVVPRTLHADVPSRQVDWTAGAVEL
VTENRSWPEADRPRRAAVSSFGLSGTNAHVILESPPDQPTTASAPTTGPVPLPISAKTPAALADLETRLRAYLTPET
DLPAVAATLAVNRSLFEHRAVLIGDDTITGTASTEPRVVFVFPGQGWHWLGMGSALLASSAVFADRMAECNAALSEF
VDWDLFTALDDPAVFDRVDVVQPTCWAVMVSLAAVWQHAGVRPDAVLGHSQGEIAAACFAGAISLQDAARIVALRSR
LIGRLAGRGAMASVSLPPDEIPLIDGVTVAVLNGPSAVIAGAPDAVDAVLADCEARGARVRKINVDYASHTPHVEQI
RTELLDITAGITAETPTVPWLSTVDGTWIDRPLDTEYWYRNLREPVGFGATIELLQAQGDTIFIEVSASPVLLQAID
DSIAIPTLRRDDGTPTRLLTALAEAHVHGVTIDWAKLLGSTASPVNLPTYAFQRQRYWAASAAAGRPAELTPEHLLK
VVRDSAAVVLGHTDAGAIPATAAFQALGVDSLIAVELRNNLAKSTGLRLPATLIFDYPTPADLATHL
SEQ ID NO: 125
EPLAIIGMACRLPGGITSPEDLWRLVESGSDAISDFPDDRGWDVDRLFDPDPDAAGKTYTTQGGFLSEVAGFDASFF
GISPREAVAMDPQQRLVLEVAWEAFERAGIEPGTVRGSDTGVFMGAYPDGYGSGTDLAGFGVTAGAGSVLSGRVSYF
FGLEGPAMTVDTACSSSLVALHQAGSALRQGECSLALVGGVTVMPTPRTFVEFSRQRGLAADGRCKPFADAADGTGF
SEGAGMLVVERLSDAQTNGHHILAVVRASAVNQDGASNGLTAPNGPSQQRVIQSALAGAGLVSADIDVIEAHGTGTT
LGDPIEAQAVIATYGQDRSQPVLLGSLKSNIGHTQAAAGVSGVIKMVMALQHDTVPATLHVDEPSRHVDWTAGAVAL
VTENQPWPRNGHARRAGVSSFGVSGTNAHVIIEEAPAEPPVEPVPAADVVVPLVVSARDAIPLGDQAARLAALVEAP
DGPVLPALADALLTRRTTFAQRAVVVAGSRDDAAAGLRALATGTAHPALVTGAAGTSGRVVLMFPGQGSQWDGMGAQ
LIGASPVFAARIADCAAALQPWIDWDLQDVLRGNAPTDLLERVDVVQPASFAVMVGLAAVWESVGVRPDAVLGHSQG
EIAAAYVAGALTLADAAKVVAVRSRLIAARLGRGGMASVALSPQDAAARRGRAELAAVNSPASVVLAGASEALDETL
AALEADGVRVRRVAVDYASHTGHVEELEQDLAEALADVRSQAPLVGFRSTVTGEWVTEAGALDGGYWYRNLRQQVRF
GPAVAALAEDGYSVFVEASAHPVLVQPVTETLDRTDAVVTGSLRRQDGGLSRLLTSVAEVFVGGVPVDWAGLLPAGA
GRSWVDLPTYAFDHQHYWLPAGGTRGRSEAELLELVRGRAAAVLGHTDAGSIPATAAFKDLGLDSLTAVELRNSLAK
STGLRLPATMVFDYPTPAAVAARL
SEQ ID NO: 126
EPLAIVGMACRLPGGITSPEDLWRLVASGSDAISDLPVDRGWTVDGHFQGGFLDEVAGFDASFFGISPREAVAMDPQ
QRLVLEVAWEAFERAGIEPGSVRGTDAGVFMGAYADGYGMGTDLGGFGMTSVAVSVLAGRISYFFGLEGPAMTVDTA
CSSSLVALHQAGHALRQGECSLALVGGVTVMPTPQTFVEFDRQRGLAADGRCKAFADAADGTSFSEGAGMLVVERLS
DALANGHHILAVVRGSAVNQDGASNGLTAPNGPSQQRVIRSALANAGLTTADVDVVEAHGTGTTLGDPIEAQAVIAT
YGQNRQRPLLLGSLKSNIGHTQAAAGVSGVIKMVMALRNGTVPATLHVDEPSRHIDWTAGAVALVTENQPWPETERP
RRAGVSSFGISGTNAHVILESTPTPPATLSAQVAHPLPLPISAKTPPALADLEARLRAYLTPEADLAAVASTLASTR
AVFEHRAVLLGDETIVGVAALDPRVVFVFSGQGSQRAGMGEQLAAVFPVFAQIHREVLDLLDIPDLDIDQTGHAQPA
LFAFQVALAGLLDSWGVRPDAVIGHSIGELAAAYVAGLWSLQDACALVSARARLMQALPSGGAMVAVAVPEDEARAV
LIDGVEIAAVNGPSSVVLSGDETAVLQVAESLGGKSARLKTSHAFHSARMEPMLDQFRQVAEQLTYRSPVIEMTAGV
TPDYWVRQVRDTVRFGDQVRVHQGSVLVEIGPDRTLARLIDGIATSHGDDEVQAVMTALAELHVRGVAVDWPGTTSA
RVLDLPTYAFQHDHYWAHPVDRTPEALLALVRDSAAVALGHAGAATVPATAAFQSLGMDSLIAVELRNNLARSTGLR
LPATLVFDYPTPAALATRL
SEQ ID NO: 127
EPLAIVGMACRLPGGVTSPEDLWRLVASGTDAITGLPTDRGWEEDDRFRGGFLAGVAGFDASFFGISPREAVAMDPQ
QRLVLEASWEAFERAGIEPGSVRGSDTGVFMGAYPGGYGFGADLGGFALTSGSGSVLSGRVSYFFGLEGPAMTVDTA
CSSSLVALHQAGYALRQGECSLALVGGVTIMPTPQTFIEFERQRGLAADGRSKAFADSADGTGWSEGVGVLVVERLS
DAQANGHHILAVVRGSAINQDGASNGLTAPNGPSQQRVIRSALANAGLTTADIDVIEAHGTGTTLGDPIEAQAVIAT
YGQDRSQPVLLGSLKSNIGHTQAAAGVSGVIKMVMALQHDTVPATLHVDRPSRHVDWAAGAVELVTENRPWPENGRV
RRAGVSAFGVSGTNAHVILESPPDQPVKPSAPAAGPVPLPISAKTPAALAALENRLRAYLTPETDLPAVASTLATTR
AMFEHRAVLLGDDTITGTASTEPRVVFVFPGQGWHWLGMGSALLASSAVFADRMAECNAALHEFVDWDLFTALDDPA
VFDRVDIVQPTCWAVMMSLAALWQHAGVRPDAVLGHSQGEIAAACFAGAISLQDAARIVALRSQLIGRLAGRGAMAS
VSLPPDEIPLIDGVTVAVLNGPSAVIAGSPEAVDAVLADCEARGARVRKINVDYASHTPHVEQIRTELLHITAAITA
ETPTVPWLSTVDGTWIDHPLDTEYWYRNLREPVGFGATIELLQTQGDTIFIEVSASPVLLQAIDDSIAIPTLRRDDG
TPTRLLTALAEAHVNGVTIDWATVLGATGSPVDLPTYAFQHQRFWVGDRLHGRTSAEQHRIMLDLVLGHATSVLGHQ
TPDAVASDRAFKDLGMDSLTAVELRNHLVAETGLRLPATTAFDHPTADDLARRL
SEQ ID NO: 128
EPIAIVSMACRAPGCVTSPEGLWRLVESGTDAIADFPADRGWDLATLYSPDPIGYTSYCLQGGFLDAAADFDAAFFG
ISPREALGMDPQQRLLLETSWEAIERARIDPRSLRGRDVGVYVGGATQGYGVGAVDQQRDNVITGSSISLLSGRLSY
ALGLEGPGVTVDTACSSSLVALHLASQALRQRECSMALVSGVSVIPTPDVFVEFSRQRGLASDGRCKSFSAAADGTI
WAEGVGVLVLERLSEATRLGHEVLAVIRGSAVNSDGASNGLTAPNGASQQRVIRQALASAGLNAADVDTVEAHGTGT
KLGDPIEAEAILATYGQDRSSPVWLGSLKSNIGHSMAASGVLGVIKMVEAMRHARLPRTLHVDEPSPHVDWASGDVA
LLTENQPWPDGARPRRAGVSSFGLSGTNAHVVLEQHRAPAVPVAAETVADDVPLPLLLSARHPKALRDQAARLHAAL
AEAPGWRPLDVGYSLATTRSAFAHRAVAVGSGRELLRALAKLAEGAAWPALVTGTAKAGRVAFLFDGQGTQRLGMGR
VLHDRFPVFARAWDTVSARFDQHLDHSLTDVYLGRDTSAAALADDTLYAQAGIFTMEVALFELLAEWGVRPDLVSGH
SIGEVAAAYAAGLFSLEDAATLIVARGRALRQMPPGAMLALRASEDQVRELLDRTGADLDVAAVNSPVSVVVSGDPD
AVAAFRAEWEASERDARALNVHHAFHSRRVDAVLDEFRAVLGTLTFRTPALPVVSTVTGRLAGPAEMSTPEYWLRQI
RRTVRFQDAVRELSGQGAGTFVEIGPSGALAAAGLECVDASFHAVQRPRSPEDACLLTAVAELHAGGTAVDWAKVLA
GGRATDLPVYPFQHETYWIPPASPPADTRTMLEVVHEEAALVLGVTDPRVILDDSSFLDLGFDSLSAMRLGNQLSAV
TGLDLPPSLLFEHPTVGELAAHLD
SEQ ID NO: 129
EPLAIVGMAARFPGGVASADDLWRLVVSGGDAIGGFPADRGWDLEELYDPDPAATGRSYVREGGFLNDATTFDASFF
RIGPREAKAMDPQQRLLLETSWEAFEHAGIRPETLRGTATGVFAGISLQDYGVLAGSDPELEGYAGTGNAPSVLSGR
LSYFYGLEGPAVTIDTACSSSLVALHLAGQSLRRDECTLAVVGGVTVMPSPNVFVEFSRQRGLAPDGRCKPFAAAAD
GTGWSEGVGVLVVERLSDARRNKRRILAVVRGSAVNQDGASSGLTAPNGPSQQRVIRSALAAAGLTAGDVDVVEAHG
TGTTLGDPIEAQGVLATYGDRSGAPVRLGSVKSNLGHTQAAAGIAGVIKMVQALRHGVMPRSLHIDEPSPHVDWTAG
RVELLTSNLPWPTSERPRRAAVSSFGISGTNAHVILEQAFPATEPEPSFTPVVSGPALPLVFSARDSGALATRTHLS
DGPGVAYALATSRSMFDHRSVRIGDMTVTGVATTDPEVVFVFPGQGTQWAGMGRALMDASPVFAERMNECAAALEPY
LDLWEAIDTPDQVETLQPASWAVMVSLAAVWQAAGVRPAAVIGHSQGEIAAACVAGSLSLADAAAVVALRSKAIAAS
LGKGAMASIPLPVEEIELIDEVWVAALNGPSSTVVAGAPDAVEQVRARYDGRRIAVDYASHTPHVEALRGQVVSVPS
QAPDIPWFSTVDSEWVEGPLDDDYWFRNLRQPVQFGPAAARFDDAVFVEVSARPVLIPALDASVTVPSLRRDDGGPE
RMLASLAQAFVAGVAVDWTTIVPPAPFVDLPTYPFQGERFWIDLDDVLAVVRDCAATVLGHTDPAAIAPDRPFKDLG
FDSLAAVQLRNHLLTVTGVRLSATAVFDFPTPAVLAGEV
SEQ ID NO: 130
EPIAVVGMACRLPGDVASPEDLWRLVAEGRDAVGPFPADRGWELGEAAYARVGGFVTGATGFDAGFFGISPREALAM
DPQQRLLLEVAWEAFERAGIAPDALRGSDTGVFVGTYGQGYGELAVDGDAEGYVGIGNSGSVVSGRVSYFFGLEGPA
VTVDTACSSSLVALHQAAQALRQGECSLALVGGVTVMSSPLIFQEFARQGGLAADGRCKAFADGADGTGWGEGVGVL
VVERLSEAQRRGHTVLAVVRGSAVNQDGASNGLTAPNGPSQQRVIRSALASAGLGFGDVDLVEAHGTGTALGDPIEA
QALLATYGSAGTPVWLGSLKSNIGHTQAAAGVAGVIKAVEAMRHGVLPQTLHADQPSSHVDWTAGAVELLTANRPWD
SAGRPRRAAVSSFGISGTNAHVILEEFSSAPVSPEPGAGAAPLLLSARSAAALAEFESRVAALRPSRDLAATLAGRV
FFDHRAVVLPGGEVVRGRVGDAPVVFVFAGQGSQRSDMASRLAGEFPLFAAAHERVWSLLDVDESLDVDQTGFAQPA
LFAYEVALAELLGVRPDAVIGHSVGELAAAYVAGALSLEDACRLVSARARLMQALPPGGVMVSVRVSEEAARAVLRD
GVELAAVNGPRAVVLSGDEGAVLAAAAELGEFRRLRTSHAFHSALMEPMLEEFRAVASSVEFGEPEIALSFVPSADY
FVRQVRETVRFGEQVAAFEPGTLFVEVGPDGSLSRLTGGVNAAEPLTALAHLWAHGAVVDWTPYTSDGRLDTAPTYP
FQPERYWPEQRRRRARRGDSLALVIATAAAVLGHPEGTDIPADTPFQSLGFDSLSAVDLRNQLAHATGVRLSPTAVF
DHPTPRALAERL
SEQ ID NO: 131
DPIAIVGMACRYPGGVATADDLWDLVAEGGDAVGPFPADRGWDLAGLYDPDPEAAGKSYVREGGFLGGAADFDAAFF
GISPREALAMDPQQRLLLETAWEAFEHAGIDPLDLRRSDTGVFVGTMAQEYGGLVTDSAHGLEGWIGTGNSQSVMSG
RLSYFFGLQGPAVTIDTACSSSLVALHQAAQALRSGECSLAVVGGVTVMSSPRTFQEFSRQRGMAPDGRCKPFAAAA
DGTGWSEGVGVLVVERLSEARRNGHAVLAVVRGTAVNQDGTSNGLTAPNGPAQQQVIRAALERAGLGVGDVDVVEAH
GTGTALGDPIEAQAILDTYGSRTTGEPVRLGSVKSNLGHTQAAAGVAGVIKMVQAMRHATMPRSLHIDEPSPHVDWA
SGAVELLTAERGWPATDRPRRAAVSSFGISGTNAHVIVEGVTEPEPSREAAPSGPLPLMLSAPTAEALAEQETRLRR
FRADRPDADERDIAVTLAGRTGFAHRTVLIGELSVSGVAVADRRVVFVFPGQGTQWAGMGRDLMDASPVFAERMNEC
AAALEPYLDLWEAIDTPDRVETLQPASWAVMVSLAAVWQAAGVRPAAVIGHSQGEIAAACVAGSLSLADAAAVVALR
SKAIAASLGKGAMASIPLPAEEIELIDEVWVAALNGPSSTVVAGAPDAVEQVRARYDGRRIAVDYASHTPHVEALRG
QVVSVPSQAPDIPWFSTVDSGWVEGPLDDDYWFRNLRQPVQFGPAAARFDDAVFIEVSARPVLIPVLEDAVTVPTLR
RDDGGIGRLHASVAQAWTAGADVDWAALLPAGGRRIALPPYAFTHERFWPRRPAAAGQDLLTVVRTAAATVLGHRDA
ARVPADRAFKELGFDSLSAVQLRNELLTATGVRLSATAVFDHPTAAALAEAL
SEQ ID NO: 132
EPIAIVGMACRLPGDVSSPDELWELVESGRDAIGPFPADRGWNLSTLFDPDPDAPGKSYVREGGFLTGAGLFDADFF
GISPREALAMDPQQRLLLEVAWEAFERAGIAPDALRGSDTGVYVGTYAQGYGELAAATAGEGFVGIGNSGSVVSGRV
SYFLGLEGPAVTVDTACSSSLVALHQAAQALRLGECSLALVGGVTVMASPLMFQEFSRQRGLSPDGRCKAFAEGADG
TGWGEGAGVLVVERLSEARRRGHTVLAVVRGSAVNQDGASNGLTAPNGPSQQRVIRAALAAAGLTFGDVDVVEGHGT
GTALGDPIEAQALLATYGAAGSPVRLGSLKSNIGHTQAAAGVAGVIKMVQAMRHGVMPRTLHVDQPSSHVDWSAGAV
ELLTANRTWEAPGRPRRAAVSSFGISGTNAHVILEGVPAPEPAAGSAETAPLLLSARTVPALNDFEARVSARPSSPD
LAATLSRRVFFDHRAVVLPGGEVVRGRVGDAPVVFVFAGQGSQRADMASRLAGEFPVFAAAHERVWSLLDVDEGLAV
DQTGLAQPALFAYEVALAELLGVRPDAVIGHSVGELAAAYVAGALSLEDACRLVSARARLMQALPPGGVMVSVRVSE
EAARAVLRDGVEIAAVNGPRAVVLSGDEDAVLAAAAELGEFRRLRTSHAFHSARMEPMLEEFRAVASSVVFGEPEIA
MSFVPSADYFVRQVRETVRFGEQVASFDPGSLFVEVGPDGSLSRLTGGVSAAEPMKALAYLWVRGVGVDWAPYVGGG
RLDLGAPTYPFQREGFWPTREALAQLPPARRGRALLDLVQNRVAKTLGLVRPADPGRAFTDLGFTSLTALELRNSIA
EETGLPLPASLVFDHPNARALAAYLD
SEQ ID NO: 133
EPLAIVGMACRLPGGISSAEELWRLVAEGGDAIGPFPGDRGWDIDALYDPDPDAAGRTYTRSGGFLPGAGDFDAAFF
GISPREAQAMDPQHRQLLETSWEALEHAGIDPAGLRGRDVGVFAGFSGQDYIAEMGVGPAEAGGYQVTGRAASVLSG
RLSYFYGFEGPAVTVDTACSSSLVALHLAGQSLRDGESSLALVGGVTVMSSPGLFVEFSRQRGLAPDGRCKAFSVDA
DGTGWSEGVGVLVVERLSDARRNNHQILAVVRGSAVNQDGASNGLTAPNGPSQQRVIRQALAQSGLSVGDVDVVEAH
GTGTALGDPIEAQAVLATYGSRTGGEPVRLGSLKSNIGHTQAAAGIASLIKMVQSIRYGVMPRTLHVSEPSPLVDWA
AGRVELLTSDVPWPEGVRRAAVSAFGISGTNAHVILEEAPAPAEAVPSIRPVVSGPELPLVFSARDADALAAQSRLT
DGPGVAHALVTARTVFDHRSVRMGDVTVTGVATPDPEVVFVFPGQGTQWPGMGRDLMAASPVFADRMNECALALSPY
LDLWAAIDAPDRVETLQPASWAVMVSLAAVWQAAGVQPAAVIGHSQGEIAAACVAGSLSLADAAAVVALRSRAIASL
AGKGAMASIPLPAEEIELVDEVWVAALNGPSSTVVAGTPDAVEQIRSRYDGRRIAVDYASHTPHVEALRGQVVSVPS
QSPAVPWFSTVDSAWVEGPLDEDYWFRNLRQPVQFGPAAAGFDNAVFVEVSARPVLIPALDASVTVPSLRRDDGGPE
RMLASLAQAFVAGVAVDWTTIVPPAPHVDLPTYPFRRQRHWIDMERLGQLPPGDRDRFLLDLVRDAAAAVLGHGSRE
TVPASAAFKELGFDSLIAVQLRNAVAAATGVSLPATVTFDHPTPQALAVLL
SEQ ID NO: 134
EPLAIVGMACKFPGGVDSPERLWEMVEAGEDVIGPFPDDRGWDVDGGYDPDPEKAGSWYARAGGFLAGAADFDAAFF
GINPREALAMDPQQRLLLEVAWEAFERSGIAPDSLRGTDTGVFVGTFGQGYGRLVAAGAPGLEAYSGTGNTGSVASG
RLSYVFGLEGPAVTVDTACSSSLVALHQAGRSLQSGECSLALVAGVTVMSTPDSFVEFSRQRGLSPDGRCKAFAAAA
DGTGFSEGAGVLVVERLSDARRNNHQILALVRGSAVNQDGASNGLTAPNGPSQQRVITAALTDARLTTTDIDLVEAH
GTGTTLGDPIEAQAILATYGNRTTGNPVHLGSVKSNLGHTQAAAGIAGVIKAIQAIRHTTMPKSLHIDQPSPHVDWT
SGRVELLTSNQPWPATDRPRRAAVSSFGVSGTNAHVILEEQTPVEEPPPASAGPVPLALSARTPEALTAQEKAVRGL
PDGDRRRAAPALALGRAALPHRAVLLGDSVIRGTASADDGRPVFLFPGQGAQWAGMGRELMAASPVFAERMRECAVA
LAGFVDWDLFAVLDDAEALRRTEIVQPASWAMMVSLAALWESWGVRPAAVVGHSQGETAAAVVAGAIGLRDGARLSA
TRSRVLALLAGHGALASIALPAGEVEVVDGVSVAAVNGPRATLISGDPAGVEAVTARYEASGVRVRRIPADVASHSP
HVERAEETLLAALAGIEARVPGVPWLSTATGDWITEPVDERYWYRNLRSPVLFHPAITTLRDRGHRLFLEISTHPQL
LPAMEDDLLTVGSLRRDDGDLDRMHAALAEAWAAGADVDWRAFLGSGPVRALDLPTYPFQRRRFWPEAGALPPAERE
RALVEIVRDQAAAVLGDPDAGALTPGTAFRDLGFDSLTAVQLRNHLATATGLTLPATVIFDHPTPRALATFLD
SEQ ID NO: 135
EPLAVVGMACRLPGGVSSPDQLWDLVVSGGDGIGPFPGDRGWATDEIYDPDPDASGKTYVREGGFLDSAGDFDAAFF
GISPREALAMDPQQRLLLETSWEAFEHAGIDPAGLRGGDTGVFVGGFTQAYGVGTADLEGYAATGTVGSVLSGRLSY
FYGFEGPAVTIDTACSSSLVALHQAGQALRQGECTLAVVGGVTVMPTPVVFQEFSRQRGLAADGRCKAFADEADGTG
FAEGAGVLLVCRLSDARRDGRRILAVVRGSAVNQDGASNGLTAPNGPSQQRVIRAALASARLGPGDVDLIEGHGTGT
TLGDPIEAQALLATHGSGASPVRLGSLKSNIGHTQAAAGVAGVIKVIQALRHGLMPRTLHVGTPSSQVDWSAGNVEL
LTSNLPWPATDRPRRAAVSSFGISGTNAHVILEEAPAPAAVPSITPVVSGPALPLVFSARDSGALAARTRLTDGPGV
AFALATSRSMFDHRAVRIGDLSVSGVAVADRRVVFVFPGQGTQWAGMGRALMDASPVFAERMNECAAALSPYLDLWE
AIDAPDRVETLQPASWAVMVSLAAVWQAVGVEPAAVIGHSQGEIAAACVAGSISLPDAAAVVALRSKAIASLAGKGA
MASIPLPPDQIDLIDQVWIAALNGPSSTVVAGSPEAVEQVRARYDGRRIAVDYASHTPHVEALRGQVVSVPSQAPDI
PWFSTVDSAWVEKPLDGDYWFRNLRQPVQFGPAAARFDDAVFIEVSARPVLIPALDTSVTVPSLRRDDGGPERMLAS
LAQAFVAGVAVDWTTIVPPAPFVELPTYPFQRRRYWIDSSEEALRDLVREQAAAVLGYPDPSRITPGVAFRDLGFDS
LTAVQLRNALSAATGLRLSATVAFDHPTPAALAAAL
SEQ ID NO: 136
EPIAIVGMACRLPGDVSSPDELWDLVESGRDAIGPFPADRGWNLDELYDPDPDATGRSYVREGGFLAGAADFDAEFF
GINPREALAMDPQQRLVLEVAWEAFERAGIAPDSLRGTDTGVFLGAFAGGYLTLVNGAADLEGYAGTGNSVSVLSGR
LSYVLGLEGPAVTVDTACSSSLVALHQAAQALRQGECSLAVAGGVTVMSTPDSHVEFSRQRALSPDGRCRAFADGAD
GTGWSEGAGVLVVERLSEARRRGHTVLAVVRGSAVNQDGASNGLTAPNGPSQQRVIRSALASAGLGFGDVDLVEGHG
TGTALGDPIEAQALLATYGSAGTPVWLGSLKSNIGHTQAAAGVAGVIKAVEAMRRGVMPRTLHVDAPSSHVEWSSGS
VELLTANRPWDGVGRPRRAAVSSFGISGTNAHVILEGVPAPEPAGTGQAPLLLSARSVSALAEFESRIAGLVPSRDL
AATLAGRAFFDHRAVILPDGDVVRGRAGGAPLVFVFAGQGSQRADMASRLAEEFPAFAAAHERVWSLLDVDEGLDVD
QTGLAQPALFAYEVALAELLGVRPDAVIGHSIGELAAAYVAGALSLEDACRLVSARARLMQDLPSGGAMVSVRVSEE
AARAVLRDGVEIAAVNGPQAIVLSGDEDAVLAAAAELGEFRRLRTSHAFHSGRMEPMLEEFRLVASSVVFREPEIAM
SFVPSADYFVRQVRETVRFGEQVASFDAGAVFVEVGPDGSLSRLTGGVSAAEPLTALAYLWVRGVGVDWAPYVGGGR
LDLGAPTYPFQRERYWVRPRLAGRTTDERDALLISLVRDDVASVLGHPDRRRLATDRPLLELGFDSLTALRLRNRLA
AATDIALPATLIFDYPNIQAIAVHL
SEQ ID NO: 137
EPLAVVGMACRYPGGVASADDLWRLVTAGGDAIGPFPDDRGWELESLVDPDPEAVGRSTTGQGGFLADAAGFDAAFF
GISPREATAMDPQQRLLLEVSWAALEHAGLRADALRGSATGVFMGSNGQDYAGLLAGAPELEGWIGTGVSASVVSGR
LSYFYGFEGPAVTVDTACSSSLVALHLAAQSLRTGESSLALVGGVTVMTSPTVFRSFSRQRGLAPDGRCKAFSAGAD
GTGWSEGVGVLVVERLSDARRNNHQILALVRGSAVNQDGASNGLTAPNGPSQQRVITAALTDARLTTTDIDLVEAHG
TGTTLGDPIEAQAILATYGNRTTGNPVHLGSVKSNLGHTQAAAGIAGVIKAIQAIRHTTMPKSLHIDQPSPHVDWTS
GRVELLTSNQPWPATDRPRRAAVSSFGVSGTNAHVILEEAPAPAEAVPPIRPVVSGPALPLVFSARDSGALATRTHL
SDGPGVAYALATSRSMFDHRSVRIGDMTVTGVATTDPEVVFVFPGQGTQWAGMGRALMDASPVFAERMNECAAALEP
YLDLWAAIDAPDQVETLQPASWAVMVSLAAVWQAAGVRPAAVIGHSQGEIAAACVAGSITLQDAAAVVALRSKAIAA
SLGKGAMASIPLPVEEIELIDEVWVAALNGPSSTVVAGAPDAVEQVRARYDGRRIAVDYASHTPHVEALRGQVVSVP
SQTPAVPWFSTVDSEWVEGQLDDDYWFRNLRQPVQFGPAAARFDDAVFIEVSARPVLIPALDASVTVPSLRRDDGGP
ERMLASLAQAFVAGVAVDWTTIVPPAPFVDLPTYPFQHERFWIEGRVAAATGAERPRILLEVVLAETATVLGHGGAA
AIGPDRAFQDLGFDSLTAVELRNRLAAATALTLPTTLVFNHPTPEALAAHL
SEQ ID NO: 138
ELVAIVGMACRLPGDVASPEDLWRLVAEGRDAVGPFPADRGWNLGTLDDPDAAGRSYVKEGGFLAGAAHFDPGFFGI
GPREALGMDPQQRILLEIAWESLERARIAPGSLRGSETGVYVGAAAQGYGVDAPLEGNLLTGGSTSAMSGRVAYSLG
LHGPAVTIDTACSSSLVALHLAAQALRNGECTLALAGGVAVMASPVLFTEFSRQRGLAPDGRCKAFAAAADGTGWSE
GAGLVVLERLSDAERHGHPVLAVIRGSAVNSDGASNGLTAPNGTAQRRVIRSALRAAGLGAGDVDVVEAHGTGTTLG
DPVEADALIATYGQRPGMPPVRIGSLKSNIGHTVAAAGVAGVIKMVEAMRHDTMPRTLHVDRPTPHVDWSAGAAELL
TGEQPWPRGDRPRRAAVSAFGLSGTNAHLILEDVAPGAASGAEPPGAADETVPLLLSADDLPAVRDQAARLRAYLLA
RPELRMRDVAYALATTRTARPHRAAVAATEREFLRELALLAAGDQGPGTQLGEAVPHRRVAFLFDGQGTQRHGMGRA
LHQRHPVFAAAWDEVCAALDPLLGRGVADVYFAEAGRDLADDPLYTQAGLFALEVALYRLLTSWGVTPDAVAGHSVG
EVAAAHVAGVLSLPDAAALLAARGAALRRLPAGAMAAIRASEADTRAVLPPDLDVAAVNGPEMTVVSGAPDAVDRFI
AEQAGAGRQVRRLRVGRAYHSRHVDAVLAEFGATLSALTFHEPVLPVVSTVTGRPAGAGDLTTPEYWLRHARRPVRF
GAALAALSELGMDSFVEVGPSGSLSSMAGETVAGTFHPMLDRRVPDEIAVAAGELFTAGMVLDWAAVLAGGRTIDLP
VYPFRREFYWLGARRYDLMAAAERRDALLDLVRVQVALLLGRADAIGVRDNTSFLDVGLDSLGASRLRNRLAAATGL
TLPGGVAFDHPTPARLADHL
SEQ ID NO: 139
EPLAIVGMACRLPGGVSSPEDLWRLVESGTDATSGFPVDRGWADSSMRGGFLDAAADFDAAFFGISPREALAMDPQQ
RLVLEASWEAFERAGIEPGSVRGSDTGVFMGAFSGGYGAGADLGGFGVTAGAVSVLSGRVSYFFGLEGPAVTVDTAC
SSSLVALHQAGHALRQGECSLALVGGVTVMSTPDIFAEFSRQGGLASDGRCKAFADTADGTSWSEGVGVLVVERLSD
ARAKGHQVLAVVRGSAVNQDGASNGLTAPNGPSQQRVIQAALTHAGLTTAEVDVVEAHGTGTTLGDPIEAQAVIATY
GRDRERPVLLGSLKSNVGHTQAAAGVSGVIKMVMALQHGVVPRTLHVDEPSRHVDWTAGAVRLATESQPWPDTGRPR
RAAVSSFGVSGTNAHVILEGVAEEPAQSEESSELVPLVISAKTPAALTRLEERLRAYLTAESNLSAVASTLAETRSL
FEHRAVLLGDDTIKGTAQPNPRVVFVFSGQGSQRAGMGDELAAAFPVFAKIRQQVWDLLDVPDLEVNDTGHAQPALF
ALQVALFGLLESWGVRPQALIGHSIGELAAGYVSGIWSLEDACTLVSARARLMQSLPPGGAMVAVPVSEQQARAVLT
DGVEIAAVNGPSSVVLSGDEEAVLRAAAALDGRSKRLVTSHAFHSARMEPMLDEFRAVAEQLTYRAPRIPMAVGEGP
EYWVRQVRETVRFGEQVAAHDGAVFVELGPEGTLARLIDGVAVLDREDEPRAALTALGKLHVRGVRVDWPLTSGRRV
DLPTYAFQRERYWATALTPAEREQALLKLVRDSAAVVLGYTDAVPVSGSFKDLGIDSLTAVELRNSLATTTGLRLPA
TLVFDYPTPATLAARL
SEQ ID NO: 140
EPLAIVGMACRLPGGVSSPEDLWRLVESGTDAIAPFPTDRGWDVEALFDPDPDAAGKSYCVRGGFLDGVADFDASFF
GISPREALAMDPQQRLILEASWEAFERAGIDPADARGSDTGVFMGAFTSGYGADLEGFGGTAGALSVLSGRVSYFFG
LEGPAVTVDTACSSSLFALHQAGYALRQGECSMALVGGVTVMATPRTFVEFSRQRGLASDGRCKAFGDTADGTGWAE
GVGVLVVERLSDAQAKGHRVLAVVRGSAVNQDGASNGLTAPNGPSQQRVIQAALHNARLTPADVDVVEAHGTGTTLG
DPIEAQAVIAAYGQGRDEPLLLGSLKSNVGHTQAAAGVSGVIKMVMALQHGVVPRTLHVDEPSRHVDWTAGEVRLVT
ENQSWPDTGRPRRAGVSAFGVSGTNAHVILEGPPTQPPATAPQEPAPLVISAKTPAALADYEGRLRAYLAATPGTDA
RALAVTRSLFEHRAVLLGDDTISGAAVTDPRVVFVFPGQGWQWLGMGVALRDSSVVFAERMTECAAALSEFVDWDLF
AVLDDPAVVDRVDVVQPACWAVMVSLAAVWQAAGVHPDAVVGHSQGEIAAACVAGAISLRDAARVVALRSRLIGERL
GQGAMASVTLPADEISLVDGVWIAAYNGPASTVIAGSPDAVDQMVGDRVRRIAVDYASHSPQVEQIKDELLDITADV
GSRTPTVPWFSTVDGSWIEGPLDADYWYRNLRQPVGFHPAVEALRALGETVFVEVSASPVLLPAMDDALTVATLRRD
DGTIARMHTALAEAHVHGVNVDWAAVLGVAARHVDLPTYAFQRQRFWADERELASLGPAEREQALRKLVSDTAAGVL
GYADPGAVPIKAAFRELGVDSLTAVELRNGLAKATGMRLPATMVFDYPTPHALAARL
SEQ ID NO: 141
EPLAIVGMACRLPGGVSSPEDLWRLVESGTDAISEFPADRGWDVENLYDPDPDAAGKSYCVRGGFLDAAAEFDASFF
GISPREALAMDPQQRLILEASWEAFERAGIEPGSVRGSDTGVFMGAFSGGYGADVEGFGATAGAGSVLSGRVSYFFG
LEGPAITVDTACSSSLVALHQAGYSLRQGECSLALVGGATVMAKPQSFVEFSRQRGLAADGRCKAFADAADGTGWAE
GVGVLLVERLSDAERNGHQVLAVVRSSAVNQDGASNGLSAPNGPSQQRVIRQALANARLTAADVDVVEAHGTGTTLG
DPIEAQALIAAYGQDREWPLLLGSLKSNVGHTQAAAGVSGVIKMVMALQHGVVPRTLHVDEPSRHVDWTAGAVRLVT
DNQPWPETGRPRRAGVSSFGVSGTNAHVILESPPTQPSGTFKKPAHEPQPLIISAKTPAALADYEDRLGAYLTAAPG
VDVPAVAATLAVTRSLFEHRAVLLGDNTVTGTAITDPRVVFVFPGQGWQWLGMGAALRGSSVVFAERMTECAAALSE
FVDWDLFAVLDDPAVVDRVDVVQPACWAVMVSLAAVWQAAGVHPDAVVGHSQGEIAAACVASAVSLRDAARVVALRS
RLISERLGQGAMASVALPADQIVLADGVWIAAHNGPTSTVVAGSPDAVEQMLGDRVRKIAVDYASHTPHVEQIKTEL
LGITAGIGSRTPTVPWFSTVDGSWIEGPLDADYWYRNLRQPVGFDAAVGRLRALGATVFVEVSASPVLLPAMDDAVT
IATLRRDEGSITRMHTALAEAHVLGVNVDWPTLLGDTDRRALDLPTYAFQRQRYWGDAAGLAPAEREQALLKLVRDS
AALVLGYAGGDAVPATDAFKDLGIDSLTAVELRNGLAKATGLRLPATLVFDYPTPQVLAARL
SEQ ID NO: 142
EPLAIVGMACRLPGGVSSPEDLWRLVESGTDATSGFPVDRGWADSSMRGGFLDAAADFDAAFFGISPREALAMDPQQ
RLVLEASWEAFERAGIEPGSVRGSDIGVFMGAYPGGYGIGADLAGFGATAGAGSVLSGRVSYFFGLEGPAVTVDTAC
SSSLVALHQAGHALRLGECSLALVGGVTVMATPDTFVEFARQGGLASDGRSKAFADSADGAGFSEGVGVLLVERLSD
AQRHGHRVLAVVRGSAVNQDGASNGLTAPNGPSQQRVIQAALAHAGLAPHEVDVVEAHGTGTTLGDPIEAQAVIATY
GQDRDEPLLLGSVKSNVGHTQAAAGVAGVIKMVMALRHGVVPQTLHVDEPSRHVDWTAGAVRLLTEKQPWPSTDRPR
RAGVSSFGISGTNAHVILEGVAEEPAQSEDSSELVPLVISAKTPAALTQVEERLRAYLTAESNLSAVASTLAETRSL
FEHRAVLLDGHAVRGVAESNPRVVFVFSGQGSQRAGMGDELAAAFPVFAKIRGQVWDLLDVPDLDVNDTGHAQPALF
ALQVALFGLLESWGVRPHALIGHSIGELAAGYVSGIWSLEDACALVSARARLMQALPPGGAMVAVPVSEQQARAVLT
DGVEVAAVNGPSSAVLSGDEEAVLRAAAALGGRWKRLATSHAFHSARMEPMLDEFRAVAEQLTYRAPRIPMAVGEGP
EYWVRQVRETVRFGEQVAAHDGAVFVELGPDGSLARLIDGIATLDRDDEPRVALTALAELHVRGVDVDWPLTSGRRV
DLPTYAFQRQRYWIDRAGRTPAEREQALLKVVRDSAATVLGHADGGSVGAAAAFKDLGVDSLTAVELRNSLAKATGL
RLPATLVFDYPTPAAVAVRL
SEQ ID NO: 143
EPLAIVGMACRLPGGVSSPEDLWRLVESGTDATSGFPTDRGWADSSMRGGFLVAAADFDAAFFGISPREALAMDPQQ
RLVLEASWEAFERAGIEPGTVRGSDTGVFMGAYPGGYGIGADLAGFGATAGAGSVLSGRVSYFFGLEGPAVTVDTAC
SSSLVALHQAGHALRQGECSLALVGGVTVMATPDLFVEFARQGGLASDGRCKAFGDTADGTGWAEGVGVLLVERLSD
AQAKGHRVLAVVRGSAVNQDGASNGLTAPNGPSQQRVIQAALHNARLTPADVDVVEAHGTGTTLGDPIEAQAVIAAY
GQGRDEPLLLGSLKSNVGHTQAAAGVSGVIKMVMALQHGVVPRTLHVDEPSSHVDWTAGAVRLVTENQSWPDTGRPR
RAAVSAFGVSGTNAHVILESSAAPSPTIPQPPSAEPMPLVISAKTPAALADYEGRLRAYLTAPGVDVPAVAATLAVT
RSLFEHRAVLLGGNTVTGTAVADPRVVFVFPGQGWQWLGMGAALRGSSVVFAERMTECAAALSEFVDWDLFAVLDDP
AVVDRVDVVQPACWAVMVSLAAVWQAAGVHPDAVVGHSQGEIAAACVAGALSLRDAARVVALRSRLIGERLGRGAMA
SVSLPADQIVLADGVWIAAHNGPASTVIAGGAGAVDQMVGERVRRIAVDYASHTPDVEQIQTELLDITADVGSQAPV
VPWFSTVDGVWVDGPLDRDYWYRNLRQPVGFHPAVEALQALGETVFVEVSASPVLLPAMDDAVTVATLRRDEGSITR
MHTALAEAHVLGVNVDWPTVVGDTDRRTLDLPTYAFQHHRYWISAAARLDGLTAAEKHSLLLDIVLANAATVLGHHT
VDTIAPDKPFKDLGIDSLTAVELRNGLAKATGLRLPATLVFDYPTPDMAAARL
SEQ ID NO: 144
EPLAIVGMACRLPGGVSSPEDLWRLVESGTDAISDFPTDRGWADAAGAPYSPQGGFVDAAADFDAAFFGISPREALA
MDPQQRLVLEASWEAFERAGIEPGTVRGSDTGVFMGAYPGGYGIGADQAGFGTTAGAGSVLSGRVSYFFGLEGPAVT
VDTACSSSLVALHQAGHALRQGECSLALVGGVTVMGTPDIFAEFSRQGGLASDGRCKAFGDDADGTGWGEGVGILLV
ERLSDAQRHSHRVLAVVRGSAVNQDGASNGLTAPNGPSQQRVIQAALAHAGLAPHEVDVVEAHGTGTTLGDPIEAQA
VIATYGQDRDEPLLLGSVKSNVGHTQAAAGVAGVIKMVMALRHGVVPRTLHADQPSRHVDWTAGAVRLATENQPWPA
IDRPRRAGVSSFGISGTNAHVILEGVAEEPAQSEESSPLMPLVISAKTPAALTRLEERLRAYLAAKPETSLGAVAST
LAETRSLFEHRAVLLNGDVVRGVAEPNPRVVFVFSGQGSQRAGMGDEVAAAFPVFAKIRRQVWDLLDVPDLDVNDTG
HAQPALFALQVALFGLLESWGVRPDALIGHSIGELAAGYVSGIWSLEDACALVSARARLMQALPAGGAMVAVPVSEQ
QARAVLTDGVEIAAVNGPSSVVLSGDEEAVLRAAAGLGSRWKRLATSHAFHSARMEPMLDEFRVVAEQLSYKTPRIP
VAVGEGPEYWVRQVRETVRFGEHVAAHDGAVFVELGPDGSLARLIDGIATLDRDDEPRAALTALAELHVRGVDVDWP
LTSGRRVDLPTYAFQRQRYWTTAGLTRAEREQALLKLVRDTAAVVLGYGDGNAVPVTAAFKDLGVDSLTAVELRNGL
AEAIGLRLPATLVFDYPTPATLAVRL
SEQ ID NO: 145
EPLAIVGMACRLPGGVESPDDLWRLVESGTDAITGFPTDRGWPDVTGTSHSQHGGFLHTAADFDAAFFGISPREALA
MDPQQRLILEASWEAFERAGINPADAHGTDTGVFMGAFSAGYDADRDDSPATAGAVSVLSGRISYFFGLEGPAMTVD
TACSSSLVALHQAGYSLRQGECSMALVGGVTVMATPRTFVEFSRQGGLASDGRCKAFGDTADGTGWSEGVGVLVVER
LSDARAKGHRVLAVVRSSAVNQDGASNGLTAPNGPSQQRVIQAALHNAHLTPADVDVVEAHGTGTTLGDPIEAQAVI
AAYGQDRDEPLLLGSIKSNVGHTQAAAGVSGVIKMVMALRHGVVPRTLHADQPSRHVDWNAGAVQLVTENQSWPETG
RPRRAAVSSFGISGTNAHVILEGVPEQPAQPEPPSERVPLMISAKSTSALSQLEDRLRAYLAARPEASLGAVASTLA
TRSLFEHRAVLLDGQVVKGVAEPNPRVVFVFSGQGSQRAGMGDELAAAFPVFAKIRGQVWDLLDVPDLDVNDTGHAQ
PALFALQVALFGLLESWGVRPDALIGHSIGELAAGYVSGIWSLEDACTLVSARARLMQALPAGGAMVAVPVSEQQAR
AVLTGGVEIAAVNGPSSVVLSGDEGAVLRAAAALGGRWKRLATSHAFHSARMEPMLDEFRAAAEQLTYQTPRIPMVV
GDGPDYWVRQVRETVRFGEQVAAHDGAVFVELGPDRSLARLIDGIATLDRDDEPRAALTALAELHVRGVDVDWPHDG
QLVDLPTYAFQRERYWATALAALPLAEREQALLAVVSDNAAVVLGYAEGRDVTQTAAFKDLGVDSLTAVELRNTLAK
ATGLRLPATIVFDYPTPDTLAARL
SEQ ID NO: 146
EPLAIVGMACRLPGGVSSPEDLWRLVESGTDAISRFPDDRGWDVEGLFDPDPDAPGKSYSVEGGFLDAVADFDAAFF
GISPREALAMDPQQRLVLEASWEAFERAGIEPGSVRGSDTGVFMGAYSGGYGIGADLPGLGVTAGAVSVVSGRVSYF
FGLEGPAVTVDTACSSSLVALHQAGHALRRRECSLALVGGVTVMATPFGFVEFSRQRGLAADGRCKAFADTADGTSW
SEGVGVLVVERLSDARANGHQVLAVVRGSAVNQDGASNGLTAPNGPSQQRVIQAALAHADLAPHEVDVVEAHGTGTR
LGDPIEAQAVIATYGQGRDEPLLLGSIKSNVGHTQAAAGVSGVIKMVMALRHGVVPQTLHVDEPTQHVDWTAGAVRL
ATENQPWPDTGRPRRAGVSSFGVSGTNAHVILEGVAEEPAQSEESSELVPLVISAKTPAALTRLEERLRAYLSAESN
LSAVASTLAETRSLFEHRAVLLGDDTIKGTAQPNPRVVFVFSGQGSQRAGMGDELAAAFPVFARIRRQVWDLLDVPD
VSVDDTGFAQPALFALQVALFGLLESWGVRPDALIGHSIGELAAGYVSGIWSLEDACTLVSARARLMQALPAGGAMV
AVPVSEQQARAVLTGGVEIAAVNGPSSVVLSGDEEAVLRAAAALGGRSKRLVTSHAFHSARMEPMLDEFQAVAEQLT
YQAPRIPMAVGDGPDYWVRQVRETVRFGDQVAAQDGAVFVELGPDRSLARLIDGIATLDRDDEPRAALTALAELHVR
GVDVDWPLTSGRRVDLPTYAFQRQRYWIDSALTPAEREQALLKVVRDSAAVVLGYTDAVPVSGSFKDLGIDSLTAVE
LRNSLAKVTGLRLPATLVFDYPTPATLAARL
SEQ ID NO: 147
EPLAIVGMACRLPGGVESPDDLWRLVESGTDAITGFPTDRGWPDVTGTSHSQHGGFLHTAADFDAAFFGISPREALA
MDPQQRLILEASWEAFERAGINPADAHGTDTGVFMGAYSGGYGIGADLAGFGATSGATSVLSGRVSYFFGLEGPAIT
VDTACSSSLVALHQAGHALRQGECAMALVGGVTVMATPDIFVEFSRQRGLAADGRCKAFADAADGTGWAEGVGVLLV
ERLSDAERNGHRVLAVVRSSAVNQDGASNGLTAPNGPSQQRVIQAALHNARLTPADVDVVEAHGTGTTLGDPIEAQA
VIAAYGQGRDEPLLLGSLKSNVGHTQAAAGVSGVIKMVMALQHGVVPRTLHVDEPSSHVDWTAGAVRLATENQSWPD
TGRPRRAAVSAFGVSGTNAHVILESSAAPSPTIPQPPSAEPMPLVISAKTPAALADYEDRLRAYLTNPGVDVPAVAA
TLAMTRSLFEHRAVLLGGNTVTGTAVADPRVVFVFPGQGWQWLGMGAALRGSSVVFAERMTECAAALSEFVDWDLFA
VLDDPAVVDRVDVVQPACWAVMVSLAAVWQAAGVHPDAVLGHSQGEIAAACVAGAISLQDAARVVALRSQAISGLSG
KGAMASIALPADQIALPDGAWIAAHNGPASTVVAGSPDAVEQMLGDRVRKIAVDYASHTPHVEQIQTELLDITAGIG
SRTPTIPWFSTVDGMWVDGPLDRDYWYRNLRQPVGFHPAVEALQALGETVFVEVSASPVLLPAMDDAVTVATLRRDE
GSITRMHTALAEAHVLGVNVDWPTLLGDTGRRTLDLPTYAFQHHRYWINGSRLIGRTTAEQHRLMLAFVLGNVASVL
GHGSADAIAADKPFKDLGMDSLTSVELRNSLAKATELRLPATIVFDHPTADALAAHL
SEQ ID NO: 148
EPIAIVSMACRVPGGVTSPEGLWRLVESGTDAISAFPGDRGWDIANLYSPDPDAPGKSYSVQGGFLDGAAAFDASFF
GISPREALGMDPQQRVLLETAWEAVERARIDPRSLRGRDVGVYVGGAAQGYGLGAAEAHRDNLITGGSISLLSGRLS
YALGLEGPGLTVDTACSSSLVALHLAAQALRQGECSLALVSGVSVMPTPDVFVEFSRQRGLASDGRCKSFAASADGT
SWSEGVGVLVLERLSEARRLGHQVLAVVRGTAVNSDGASNGLTAPNGAAQQRVIRQALANAGLSTADVDAVEAHGTG
TTLGDPIEAEAILATYGKDRSTPVWLGSLKSNIGHTMAASGVLGVIKMVEAMRHGVLPRTLHVDEPSPHVDWAAGEV
ALLTENQTWPGDVRPRRAGVSSFGLSGTNAHVVLEQDEAPAAPVTTKESGPLPWVLSAQSPKALRQRAGQLATALAE
DSTWHPLDVAYSLATTRSDFAHRAVVVGADRELLRTLGKVADGAGWPGLTTGTAKARRVAFLFDGQGTQRLTMGQGL
YGSFPAFARAWDTVSAEFGKHLDHPLADVYFDGSGGAATADLVDDPLYAQAGIFAVEVALVELLAEWGVRPDVVTGH
SIGEAAAAYTAGMLSLSDVTTLIVARGAALRSAPPGAMLALRAGEQEVRNFLDGTGAALDLAAVNGPAAVVVSGAPD
AVTDFASAWTASGREARRLKVRRAFHSRHVEGVLDDFRTALESLSFRTPLLPVVSTVTGRLIDPAEMGTPEYWLDQV
RQPVRFQEAVQELAGQGVGTFVEVGPSGTLASAGMECLDGDASFHALLRPRSAEDVGVLTALAELHAGGTAIDWPTV
LAGGRPMDLPVYPFQHQSYWLVSTDEPRTTLELVHLEVARVLGITDPDTVLDDASFLELGFDSLGGVRLRNRLAQVT
GLTLPPTLLFDHVTPAALAAELD
SEQ ID NO: 149
EPLAIVGMACRLPGGVSSPEDLWRLVESGTDAISGFPADRGWDVENLYDPDPEAAGKSYCVQGGFLDSAGGFDASFF
GISPREALAMDPQQRLVLEASWEAFERAGIEPGSLRGSDTGVFMGAYPGGYGVGADLGGFGATAGAVSVLSGRVSYF
FGLEGPAVTVDTACSSSLVALHQAGYALRQGECSLALVGGVTVMATPQSFVEFSRQRGLASDGRCKAFADAADGTGW
AEGAGVLLVERLSDAQAKGHQVLAVVRGSAVNQDGASNGLSAPNGPSQQRVIRAALSNAGLSTAEVDVVEAHGTGTT
LGDPIEAQALLATYGQDREQPLLLGSVKSNLGHTQAAAGVSGVIKMVMALQHGLVPRTLHVDEPSRHVDWTDGAVAL
VTENQPWPDMGRPRRAGVSSFGISGTNAHVILESAPPTQAVDDVPPAEAPVVASELVPLVISARTLPALVEYEDRLR
AYLAASPGVDVRGVASTLAVTRSVFEHRAVLLGDDTVTGTTVSDPRVVFVFPGQGSQRAGMGEELAAAFPVFARIHQ
QVWGLLDVPDLEVNETGYAQPALFALQVALFGLLESWGVRPDAVVGHSVGELAAGYVSGLWSLEDACTLVSARARLM
QALPPGGVMVAVPVSEDEARAVLGEGVEIAAVNGPSSVVLSGDETAVLQAAAALGKSTRLATSHAFHSARMEPMLEE
FRTVAERLTYQTPRLAMAAGDRVTTAEYWVRQVRDTVRFGEQVASYEDAVFIELGADRSLARLVDGVAMLHTDHEAQ
AAISALAHLYVNGVTVDWTALLGDAPATRVDLPTYAFQHQRYWLEGWLAALAPEERAKALLKVVRDTAATVLGHADA
RTIPVTGAFRDLGIDSLTAVELRNGLAKVTGLRLPATLVFDYPTPAVLAARL
SEQ ID NO: 150
EPLAIVGMACRLPGGVSSPEDLWRLVESGTDAISGFPADRGWDAESLFDPDPAVGKSYCVEGGFLDSAASFDAGFFG
ISPREALAMDPQQRLIMEVSWEAFERAGIEPGSVRGSDTGVFMGAYAGGYGAGADLGGFAATASATSVLSGRVSYFF
GLEGPAITVDTACSSSLVALHQAGYALRQGECSLALVGGVTVMATPQSFVEFSRQRGLASDGRCKAFADSADGTGWA
EGVGVLLVERLSDAQAKGHQVLAVVRSSAVNQDGASNGLSAPNGPSQQGVIQAALSNAGLAAHEVDVVEAHGTGTTL
GDPIEAQAVIATYGQDRERPLLLGSLKSNIGHAQAASGVSGVIKMVMALQHNTVPRTLHVDEPSRHVDWAAGAVELV
RENQPWPGTDRPRRAGVSSFGVSGTNAHVILESAPPAQPAEEAQPVETPVVASDVLPLVISAKTQPALTEHEDRLRA
YLAASPGVDTRAVASTLAVTRSVFEHRAVLLGDDTVTGTAVSDPRVVFVFPGQGWQWLGMGSALRDSSIVFAERMAE
CAAALREFVDWDLFTVLDDPAVVDRVDVVQPASWAMMVSLAAVWQAAGVRPDAVIGHSQGEIAAACVAGAVSMRDAA
RIVTLRSQAIAGLAGRGAMASVALPAQDVELVDGAWIAAHNGPASTVIAGTPEAVDHVLTAHEARGVRVRRITVDYA
SHTPHVELIRDELLDITSDSSSQAPVVPWLSTVDGSWVDSPLDVEYWYRNLREPVGFHPAVGQLQAQGDTVFVEVSA
SPVLLQAMDDDVVTVATLRRDDGDATRMLTALAQAYVHGVTVDWPAILGTTTTRVDLPTYAFQHQRYWVEWLAALAP
AEREKALLKVVCDSAAVVLGHADARTIPVTGAFKDLGVDSLTAVELRNSLVKATGLRLPATMVFDYPTPTALAARLD
SEQ ID NO: 151
EPLAIVGMACRLPGGVSSPEDLWRLVESGTDAVSGFPTDRGWDVEGLFDPDPDAAGKSYRAEGGFLDTAAGFDAGFF
GISPREALAMDPQQRLLLEVSWEAFERAGIEPGSVRGSDTGVFIGAFPVGYGAGAAREGYGATAAPNVLSGRLSYFF
GLEGPAITMDTACSSSLVALHLAAQALRNGECSMALAGGVTVMATPEVFTEFARQRGLASDGRCKAFADSADGAGFS
EGAGLLLVERLSDARRNGHQVLAVVRGSAVNQDGASNGFTAPNGPAQQRVIRQALANAGLTTAEVDVVEAHGTGTTL
GDPIEAQAVIATYGQDREQPLLLGTLKSNVGHTQAAAGVSGVIKMVMALQHSTVPRTLHVNEPSRHVDWSAGAVELV
TENQSWPVTGRPRRAGVSAFGVSGTNAHVVLESAPPAQSVNNAQPVATPVVASELVPLVISAKTLPALTEHEDRLRA
YLAASPGADMRAVGSTLALTRSVFEHRAVLLGHDTVTVTGTGTAVSNPRVVFVFPGQGWQWLGMGSALRGSSVVFAE
RMAECAAALSEFVDWDLFAVLDDPAVVDRVDVVQPASWAVMVSLAAVWQAAGVRPDAVIGHSQGEIAAACVAGAVSL
RDAARIVTLRSQAIAGLAGRGAMASVALPAHEIELVDGAWIAAHNGPASTVVAGAPEAVDRVLAVHEARGVRVRRIA
VDYASHTPHVELIRDELLDITAGIGSQAPVVPWLSTVDGTWVEGPLDVEYWYRNLREPVGFDSAVGQLRAEGDTVFV
EVSASPVLLQAMDDDVVTVATLRRDDGDATRMLTALAQAFVEGVTVDWPAILGTATTRVDLPTYAFQHQRFWAEGWL
ARLAPVEREKALLKLVCDGAATVLGHADASTIPATAAFKDLGIDSLTAVELRNSLTKATGLRLPATLVFDYPTPTAL
AARL
SEQ ID NO: 152
EPLAIVGMACRLPGGVSSPEDLWRLLESGTDAVSGFPTDRGWDVENLFGPAAGDSYRLQGGFLDAAAGFDASFFGIS
PREALAMDPQQRLVLEVSWEAFERAGIEPGSVRGTDTGVFMGAYPGGYGIGADLGGFGATASAVSVLSGRVSYFFGL
EGPAITVDTACSSSLVALHQAGYALRQGECSLALVGGVTVMATPQTFVEFARQGGLAGDGRSKAFADSADGAGFSEG
VGVLLVERLSDAQAKGHQVLAMLRSSAVNQDGASNGLTAPNGPSQQRVIQAALSNAGLAAHEVDVVEAHGTGTTLGD
PIEAQALLATYGQDREQPLLLGSVKSNLGHTQAAAGVSGVIKMVMALQRGFVPRTLHVDEPSRHVDWSAGAVALVTE
NQPWPDMGRARRAGVSSFGISGTNAHVILESAPPTQPADNAVIERAPEWLPMVISARTQSALTEHEGRLRAYLAASP
GVDMRAVASTLAMTRSVFEHRAVLLGDDTVTGTAATDPRVVFVFPGQGSQRAGMGEELAAAFPVFARIHQQVWDLLD
VPDLEVNETGYAQPALFALQVALFGLLESWGVRPDAVVGHSVGELAAGYVSGLWSLEDACTLVSARARLMQALPAGG
VMVAVPVSEDEARAVLGEGVEIAAVNGPSSVVLSGDEAAVLQAAEGLGKWTRLATSHAFHSARMEPMLEEFRTVAEG
LTYRTPQVSMAAGDQVTTTEYWVRQVRDTVRFGEQVASYEDAVFVELGADRSLARLVDGVAMLHGDHEAQAAVSALA
HLYVNGVTVDWPALLGDAPATRVDLPTYAFQHQRYWLEGRWLAALAPEERAKALVKVVCDSAATVLGHADVDSIPVT
AAFRDLGVDSLTAVELRNSLTKATGLRLPATLVFDYPTPGALAARL
SEQ ID NO: 153
EPLAIVGMACRLPGGVSSPEDLWRLVESGTDAISGFPTDRGWDVENLSDPDAAGKSYCVEGGFLATAANFDASFFGI
SPREALAMDPQQRLVLEVSWEAFERAGIEPGSVRGSDTGVFMGAFPGGYGIGADLEGYGATAGLNVLSGRLSYFFGL
EGPAVTVDTACSSSLVALHQAGYALRQGECSLALIGGVTVMATPHTFVEFSRQRGLASDGRCKAFADSADGTGWSEG
VGVLLVERLSDAQAKGHQVLAVVRSSAVNQDGASNGLSAPNGPSQQRVIRQALANAGLTTAEVDVVEAHGTGTTLGD
PIEAQAVIATYGQDRDQPVLLGSVKSNVGHTQAAAGVSGVIKMVMALQHGLVPRTLHVDEPSRHVDWTDGAVELVTE
NQSWPEAGRPRRAGVSSFGVSGTNAHVILESAPPTQAVDDVRPADAPVVASVMASELVPLVISAKTQSALAEYEGRL
RAYLAASPGVDMRAVASTLAMTRSVFEHRAVIVGDDTVSGTAATDPRVVFVFPGQGSQRAGMGAELAAAFPVFARIH
QQVWDLLDVPDLEVNETGYAQPALFALQVALFGLLESWGVRPDAVIGHSVGELAAAYVSGLWSLEDACTLVSARARL
MQALPAGGVMVAVPVSEDEARAVLGEGVEIAAVNGPSSVVLSGDEAAVLQAAEGLGKWTRLATSHAFHSARMEPMLE
EFRAVAQGLTYHAPGVVMAAGDRVMTAEYWVRQVRDTVRFGEQVASYEDAVFVELGADRSLARLVDGVAMLHGDHET
QAAIGALAHLYVNGVTVDWTALLGDVPVTRVDLPTYAFQQQRYWAERWLAALAPAEREKALLKLVSDGAATVLGHAD
TSTIPATTAFKDLGIDSLTAVELRNSLAKATELRLPATLVFDYPTPTALAARLD
SEQ ID NO: 154
EPLAIVGMACRLPGGVSSPEDLWRLVESGTDAISGFPTDRGWDVENLYDPDPDAPGKSYSVQGGFLDAAAGFDASFF
GISPREALAMDPQQRLMLEVSWEAFERAGIEPGSVRGSDTGVFIGAYPGGYGIGADLGGFGTTAGAASVLSGRVSYF
FGLEGPAFTVDTACSSSLVALHQAGYALRQGECSLALVGGVTVMATPQTFVEFSRQRGLSADGRCKAFADAADGTGW
AEGVGVLLVERLSDAQANGHQILAVVRSSAVNQDGASNGLSAPNGPSQQRVIRAALSNAGLAPHEVDVVEAHGTGTT
LGDPIEAQAVIATYGQGRGEPLLLGSLKSNVGHTQAAAGVSGVIKMVMALQHSMVPRTLHVDEPSRHVDWSAGAVEL
VAENQPWPETGRPRRAGVSSFGISGTNAHVILESAPAQSVGDTAGSTPVLVSELVPLVISAKTQPALTEHEDRLRAY
LAASPGVDIRAVASTLAVTRSVFEHRAVLLGDETVTGTAVSDPRIVFVFPGQGWQWLGMGSALRDSSVVFAERMAEC
AAALSEFVDWDLFAVLDDPAVVDRVDVVQPASWAVMVSLAAVWQAAGVRPDAVIGHSQGEIAAACVAGAVSMRDAAR
IVTLRSQAIAGLAGRGAMASVALPAQDVELVDGAWIAAHNGPASTVIAGTPEAVDHVLTAHEAQGVRVRRITVDYAS
HTPHVELIRDELLDITSDSSSQTPLVPWLSTVDGTWVDSPLDGEYWYRNLREPVGFHPAVSQLQAQGDTVFVEVSAS
PVLLQAMDDDVVTVATLRRDDGDATRMLTALAQAYVHGVTVDWRAVLGDVPATRVDLPTYAFQHQRYWAEAWLVGLA
PEERAKALLKVVRDSAATVLGHADARSIPATGAFKDLGVDSLTAVELRNSLTKATGLRLPATMVFDYPTPADLAARL
SEQ ID NO: 155
EPLAIVGMACRLPGGVSSPEDLWRLLESGTDAVSGFPTDRGWDVENLYDMAGKSHRAEGGFLDAAAGFDAGFFGISP
REALAMDPQQRLVLEVSWEAFERAGIEPGSVRGSDTGVFMGAYPGGYGAGADLGGFAATASATSVLSGRVSYFFGLE
GPAFTVDTACSSSLVALHQAGYALRQGECSLALVGGVTVMATPELFTEFSRQRGLASDGRCKAFADSADGTGWAEGV
GVLLVERLSDAQAKGHQVLAVVRSSAVNQDGASNGLTAPNGPSQQRVIQAALSNAGLAAHEVDVVEAHGTGTTLGDP
IEAQAVIATYGQDRERPLLLGSLKSNIGHAQAASGVSGVIKMVMALQHNTVPRTLHVDEPSRHVDWAAGAVELVREN
QPWPGTDRPRRAGVSSFGVSGTNAHVILESAPPAQPAEEAQPVETPVVASDVLPLVISAKTQPALTEHEDRLRAYLA
ASPGVDTRAVASTLAVTRSVFEHRAVLLGDDTVTGTAVSDPRVVFVFPGQGWQWLGMGSALRDSSVVFAERMAECAA
ALSEFVDWDLFTVLDDPAVVDRVDVVQPASWAVMVSLAAVWQAAGVRPDAVIGHSQGEIAAACVAGAVSLRDAARIV
TLRSQAIAGLAGRGAMASVALPAQDVELVDGAWIAAHNGPASTVIAGTPEAVDHVLTAHEARGVRVRRITVDYASHT
PHVELIRDELLDITSDSSSQAPLVPWLSTVDGSWVDSPLDGEYWYRNLREPVGFHPAVGQLQAEGDTVFVEVSASPV
LLQAMDDDVVTVATLRRDDGDATRMLTALAQAYVHGVTVDWPAILGTATTRVDLPTYAFQHQRYWLRSWLAALAPAE
REKALLKLVCDSAAMVLGHADARSIPAAGAFKDLGVDSLMAVELRNGLVKATGLRLPATLVFDYPTPTVLAARLD
SEQ ID NO: 156
EPLAIVGMACRLPGGVSSPEDLWRLVESGTDAVSGFPTDRGWDVENLYDSDPEAAGKSYCVQGGFLDTAAGFDAGFF
GISPREALAMDPQQRLLLEVSWEAFERAGIEPGSVRGSDTGVFIGAFPVGYGAGFDREGYGATSGPSVLSGRVSYVF
GLEGPAITMDTACSSSLVALHLAAQALRNGECSMALAGGVTVMATPEVFTEFARQRGLASDGRCKAFADSADGAGFS
EGAGLLLVERLSDARRNGHQVLAVVRGSAVNQDGASNGLTAPNGPSQQRVIRAALSNAGLSTADVDVVEAHGTGTTL
GDPIEAQALLATYGQDREQPLLLGSLKSNIGHTQAASGVSGVIKMVMALRHGFVPRTLHVDEPSRHVDWAAGAVELV
RENQPWPGTDRPRRAGVSSFGVSGTNAHVVLESAPPAQPAEEEQPVETPVVASDVLPLVISAKTQPALTEHEDRLRA
YLAASPGADTRAVASTLAVTRSVFEHRAVLLGDDAVTGTAVTDPRVVFVFPGQGWQWLGMGSALRDSSVVFAERMAE
CAAALSEFVDWDLFAVLDDPAVVDRVDVVQPASWAVMVSLAAVWQAAGVRPDAVIGHSQGEIAAACVAGAVSLRDAA
RIVTLRSQAIAGLAGRGAMASVALPAHEIELVDGAWIAAHNGPASTVIAGTPEAVDHVLTAHEARGVRVRRITVDYA
SHTPHVELIRDELLGITAGIGSQPPVVPWLSTVDGSWVDSPLDGEYWYRNLREPVGFHPAVSQLQAQGDAVFVEVSA
SPVLLQAMDDDVVTVATLRRDDGDATRMLTALAQAYVHGVTVDWPAILGTTTARVLDLPTYAFQHQRYWVKSWLAAL
APEERAKALLRVVCDSAATVLGHADIDSIPVTAAFKDLGVDSLTAVELRNSLAKATGLRLPATLVFDYPTPTALAAR
LD
SEQ ID NO: 157
EPLAIVGMACRLPGGVSSPEDLWRLVESGTDAISDFPADRGWDVENLYDPDPDASGKSYCVQGGFLDSAGGFDASFF
GISPREALAMDPQQRLVLEVSWEAFERAGIEPGSLRGSDTGVFIGAYPGGYGAGAGADLEGYGTTSGPSVLSGRVSY
FFGLEGPAITVDTACSSSLVALHQAGYALRQGECSLALVGGVTVMATPDVFTEFARQRGLATDGRSKAFADSADGAG
FSEGIGVLLVERLSDAEAKGHQVLAVVRSSAVNQDGASNGLTAPNGPSQQRVIQTALSNAGLTTAEVDVVEGHGTGT
TLGDPIEAQAVIATYGQDREQPLLLGSLKSNIGHTQAAAGVSGVIKMVMALRHALVPRTLHVDEPSRHVDWTAGAVE
LVTENQPWPEIGRPRRAGVSSFGVSGTNAHVILESAPPTQAEDAAQPVEAPVMGSEPVPLVISAKTLPALNAHEDRL
RAYLAASPGVDMRAVASTLAMTRSMFEHRGVLLGDGTVSGTAVSDPRVVFVFPGQGSQRAGMGEELAAAFPVFARIH
QQVWDLLDVPDLDVNETGYAQPALFALQVALFGLLESWGVRPDAVIGHSVGELAAAYVSGVWSLEDACTLVSARARL
MQALPAGGVMVAVPVSEDEARAVLGEGVEIAAVNGPSSVVLSGDEAAVLQAAEGLGKWTRLATSHAFHSARMEPMLE
EFRAVAEGLTYRTPQVAMAAGDQVMTAEYWVRQVRDTVRFGEQVASFEDAVFVELGADRSLARLVDGVAMLHGDHEA
QAAVGALAHLYVNGVSVEWSAVLGDVPVTRVDLPTYAFQHQRYWLEGRWLAALAPAEREKALLKLVSDGAATVLGHA
DTSTIPATTAFKDLGINSLTAVELRNSLAKATELRLPATLVFDYPTPAALAARLD
SEQ ID NO: 158
EPLAIVGMACRLPGGVSSPEDLWRLLESGTDAVSGFPTDRGWDVENLYDMAGKSHRAEGGFLDAAAGFDAGFFGISP
REALAMDPQQRLVLEVSWEAFERAGIEPGSVRGSDTGVFMGAYPGGYGIGADLGGFGATASSVSVLSGRVSYFFGLE
GPAFTVDTACSSSLVALHQAGYALRQGECSLALVGGVTVMATPQTFVEFSRQGGLASDGRCKAFADAADGTGWAEGV
GVLLVERLSDARRNGHQVLAVVRGSAVNQDGASNGLTAPNGPSQQRVIRAALSNAGLSTAEVDVVEAHGTGTTLGDP
IEAQALIATYGQDRDQPVLLGSVKSNLGHTQAAAGVSGVIKMVMALQHGLVPRTLHVDEPSRHVDWSAGAVQLVTEN
QPWPDMGRARRAGVSSFGISGTNAHVILESAPPTQPADNAVIERAPEWVPLVISARTQSALTEHEGRLRAYLAASPG
VDMRAVASTLAMTRSVFEHRAVLLGDDTVTGTAVSDPRAVFVFPGQGSQRAGMGEELAAAFPVFARIHQQVWDLLDV
PDLEVNETGYAQPALFAMQVALFGLLESWGVRPDAVIGHSVGELAAAYVSGVWSLEDACTLVSARARLMQALPAGGV
MVAVPVSEDEARAVLGEGVEIAAVNGPSSVVLSGDEAAVLQAAEGLGKWTRLATSHAFHSARMEPMLEEFRAVAEGL
TYRTPQVSMAVGDQVTTAEYWVRQVRDTVRFGEQVASYEDAVFVELGADRSLARLVDGVAMLHGDHEIQAAIGALAH
LYVNGVTVDWPALLGDAPATRVDLPTYAFQHQRYWLEGRWLAALAPAEREDALLKLVRDSAALVLGHADASTIPAAA
AFKDLGIDSLTAVELRNSLAKATGLRLPNTTVFDYPTPAILATRL
SEQ ID NO: 159
EPLAVVGMACRLPGGVSSPEDLWRLVESGTDAISGFPADRGWDAESLFDPDPAVGKSYCVEGGFLDSAASFDAGFFG
ISPREALAMDPQQRLIMEVSWEAFERAGIEPGSVRGSDTGVFMGAYAGGYGAGADLGGFAATASATSVLSGRVSYFF
GLEGPAITVDTACSSSLVALHQAGYALRQGECSLALVGGVTVMATPQSFVEFSRQRGLASDGRCKAFADSADGTGWA
EGVGVLLVERLSDAQAKGHQVLAVVRSSAVNQDGASNGLTAPNGPSQQRVIQAALSNAGLAAHEVDVVEAHGTGTTL
GDPIEAQALIATYGQDRERPLLLGSLKSNIGHAQAASGVSGVIKMVMALQHNTVPRTLHVDEPSRHVDWAAGAVELV
RENQPWPGTDRPRRAGVSSFGVSGTNAHVILESAPPAQPAEEAQPVETPVVASDVLPLVISAKTQPALTEHEDRLRA
YLAASPGADIRAVASTLAVTRSVFEHRAVLLGDDTVTGTAVTDPRIVFVFPGQGWQWLGMGSALRDSSVVFAERMAE
CAAALREFVDWDLFTVLDDPAVVDRVDVVQPASWAMMVSLAAVWQAAGVRPDAVIGHSQGEIAAACVAGAVSLRDAA
RIVTLRSQAIAGLAGRGAMASVALPAQDVELVDGAWIAAHNGPASTVIAGTPEAVDHVLTAHEAQGVRVRRITVDYA
SHTPHVELIRDELLDITSDSSSQTPLVPWLSTVDGTWVDSPLDGEYWYRNLREPVGFHPAVSQLQAQGDTVFVEVSA
SPVLLQAMDDDVVTVATLRRDDGDATRMLTALAQAYVHGVTVDWPAILGTTTTRVDLPTYAFQHQRYWLKSRLTGRT
SVEQHRIMLELVLGEAASVLGHSSADAIATDTSFKDLGMDSLTAIELRNRLVAETGLQLPATMVFDYPTANALAAHL
SEQ ID NO: 160
EPIAIVAMACRLPGGVSSPEGLWHLVESGTDAISGFPTDRGWDVEGLFDPDPDAAGKSYCVQGGFLDTAADFDAPFF
GISPREALGMDPQQRLLLETTWEAIERAQIDPKSLRGRDVGVYVGGAAQGYGVGVDQQHDNGITGSSVSLLSGRVSY
ALGLEGPGVTVDTACSSSLVALHLASQALRQRECSLALVSGVSVMSSPAMFVEFSRQRGLSSDGRCKSFAASADGTI
WSEGVGVLVVERLSDARRLGHRVLATVRGSAVNSDGASNGLTAPNGTSQQRVIRQALANAGLTASDVDVVEAHGTGT
KLGDPIEAEAILATYGQERSAPAWLGSLKSNIGHAMAASGVLSVIKMVEAMGHGSLPRTLHVDAPSPHVDWTSGSVA
LLTEHQPWPDDTKLRRAGVSSFGLSGTNAHVVLEQYQAPAPPVTPVTPAPPTPVTPVTPNEPGPLPWVLSAQSPKAL
REQAGRLYASLAGDSEWNSLDIGYSLATTRSDFAHRAVAVGSGREFLRALSKLADGAPWPGLTTATATAKARRVAFL
FDGQGTQRLGMGKELYDSYPAFARAWDTVSAGFDKHLDHSLTDVCFGEGGSTTAGLVDDTLYAQAGIFAMEAALFGL
LEDWGVRPDFVAGHSIGEATAAYASGMLSLENVTTLIVARGRALRTTPPGAMVALRAGEEEVREFLSRTGAALDLAA
VNSPEAVVVSGEPEPVADFEAAWTASGREARKLKVRHAFHSRHVEAVLDEFRTALESLKFRAPALPVVSTVTGRLID
QDEMGTPEYWLRQVRRPVRFQDAVRELAEQGVGTFVEVGPSGALASAGVECLGGDASFHAVLRPRSPEDVCLMTAIA
ELHAGGTAIDWAKVLSGGRAVDLPVYPFQHQSYWLAPAEPSYADEPRTMLELVHMEVASLLGMADPGVILDDSSFLE
LGFDSLSAVRLRNRLSKATGLDLPSTLLFEHPTSAE
LAAHLD
SEQ ID NO: 170
MSRAELVRPIYDLLRANAERLGDKMAYVDSRLALTHAELAARTGRIAGHLVDMGVDRGDRVAILLGNRVENIESYLA
IARASAVAVPLNPDATEAEVAHFLSDSGAVVVITDSAHLDDVRRTAPAVTIVLVGEERIPPGVRSFAELATAEPQQS
ARDDLGLDEAAWMLYTSGTTGTPKGVLSTQGSGLWSAAYCDIPAWELTENDVLLWPAPLFHSLALHLCVLATTAVGA
TARIMNGFVASEVLEELTEHPCTVLVGVPTMFRYLLGAADTFEPRTSSLKMGLVAGSVAPASLIEGFEDVFGVPLLD
TYGCTETSGSLTVNWLSGERVPGSCGLPVPGLSLRFVDPISGADVADGEEGELWASGPSIMIGYHEQPEATAEVLSD
GWYRTGDLARRSETGHVTITGRIKELIIRSGENIHPHEIEAVALDVPGVKDAAAAGKRHPVLGEVPVLYVVPETGGV
DADMVLAVCRERLSYFKVPEEIYRVDAIPRTASGKVKRSSLTEEPAELLAGASGGETLHRLEWIPLELPEQAAPDGH
VVVRVDSLASDDSDLADADLADAVRDLARSWLADKRRADSTLVFVTRRAVHTGPSDIPSPEHAAVWDAIRREQTENP
GVFVVIDVDDDDDDDDDVNDREDDDTLLPALAGLGEPQVALRDGNPLVPRLAHANTPDSGSLTIPEDRAWLLEHSRS
GTLRDLALVPADAAERPLHPGEVRISVRAAGLNFRDVLIALGTYPGEGLMGGEAAGVVLEVGSEVSDLAPGDRVFGL
VGSAFGTVAIADRRLLGAIPDTWSFATAASIPIVFATAYYGLVDLAGLSAGESVLIHAAAGGVGMAATQIARHLGAR
IFATASVGKQHILSEAGLEDTRIAGSRTLAFREAFLNTTDGQGVDVVLNSLSGDFVDASLDLLPRGGRFLEMGKTDI
RDADRITADRPGTTYQAFDLLDAGPDRLREIIAELLPLFAQGVLRPLPLLTWDIRKARDAFSWMSRARHTGKITFTI
PRQLDPGGTVLIADGSGVLTGTVARHLVAEQGVRHLLLLSRSTPDEALINELIESGARVDTAVRDVSDRAGLEQALA
GISPEHPLTAVIHTGGPAVAHESHQLHGLTKRLDLAAFVVFSQDAPASVDALARRRRAEGLPTTTIAWGIPEAEAVV
VQGPLLGRAMASADSAHIVTRLNTVGLRALAAADTLPPLLRNLVGAQTDNTQQQAWSRQLLAAEAAREQALRDLVRS
CVMDILGLSAADRYAPDKTFREMGIDSLTAVELRNSLAKATDLRLPATMVFDYPTPAMLVVRLGE
SEQ ID NO: 171
MSREEFIQPIHDLLRVNAERLGDKIAYADSRRELTHAELRTRTGRIAGHLVDLAVERGDRVAILLGNRVETIESYLA
IARAGAIAVPLNPDATGAEVAHFLADSGAVLVITDSAHLDDVRRAAAAVTVVLVDEGPLPAGTRSFAELATAEPPTP
ARDDLGLDEAAWMLYTSGTTGTPKGVVSTQGSGLWSAANCDVPAWELTENDVLLWPAPLFHSLAHHLCLLATTAVGA
TARIMSGFVAGEVLHELEEHACTVLVGVPTMYHYLLGAVGEAGPRLPSLKMGLVAGAVSPPALIEGFERVFGVPLLD
TYGCTETTGSLTVNRLSGPRMPGSCGQAVPGISLRFVDPHTGAEVAEGEEGELWASGPSLMIGYHGRPDATREVLSD
GWYRTGDLARRSETGHVTITGRVKELIIRGGENIHPRDIEAVALELPGVRDAAAAGKQHPVLGEIPALYLVPDADGV
DAEAVLAACREKLSYFKVPEEIYRVDAIPRTLSGKVKRAALTEAPAELLSAASGNGSLYRLEWVPAETPPAGTGGPV
AVHVTRRAVATGPADLPDQEQAATWDALRGEQTGPGGPVLIDLDGADIDDARLSALASLGEPQIVVRDDTPLVARLA
REKSPALTIPGERAWVLEPDHSGVLQELALVAADTDVRPLRPGEVRIEVRAAGLNFRDVLVALGTDLGDGVFGAEGA
GVVLETGSDVRDLRPGDRVFGLLEGGHGSIAIADRRMLAVIPEGWSFATAASVPEVFVIAYYGLVDLAGLRAGESVL
IHAATGGVGMAATQIARHLGAQVYATAGVGKQHILRDAGLGDDRIADSRTTDFREAFRDSTQGRGVDVVLNSLKGDF
VDASLDLLADGGRFLELGQTDIRDAGEIAAERPGTTYHSFTRMNAGPDRLREIIAELLALFEQGVLRPSPVHTWDIR
HAREAFSWMSGGRHTGKMVLTMPQRIDPGGTVLIAGDSEALARIAARHLGVRHLLLDRGVADAAPDAVVCDVSDHDA
LERVLADLSPEHPLTAVIHTGGAAVTDEIRRLHDLTESLDLTDFVVFSQDAPAAVEAFARSRRAHGLPVRTIAWGIP
EADPVVADEHLLGRALASAEQAQIVARVNTAGLRALTAANALPTLLRNLIRAEPEETGQSAWPHRFEAAGADREEAL
LDLIRANVVDILSLPTADRYAPDRTFREMGIDSLTAVGLRNSLAKATGLPLPTTMVFDYPTP
SEQ ID NO: 172
MSHAKLIQPIYDLLRVNAERLGDKIAYADSRHALTYTELEARTGRLAGHLADLGVERGDRVAILLDNRVETIESYLA
IARASAIAVPLNPAAAGDELAHFLSDSGSVLVITDSAHLDDVRLVAPAVTVVRVDEDPVPPGVRSFAELVAVEPRTQ
ARDDLGLDEAAWMLYTSGTTGTPKGVVSTQGSGLWSAAFCDVPAWELTEEDVLLWPAPLFHSLAHHLCLLATITAGA
TARIMNGFVASEVLNELEKHACTVLVCVPTMYHYLLGAVGEGESRTFSLKLGVVAGSVSPPALIEGFEKAFGAPLLD
TYGCTETTGSLTVNWLNGPRVPGSCGTAVPGVTLRFVDPSTGADVADGEEGELWASAPSVMTGYHGQPEATREVLTD
GWYHTGDLARRSETGHVTITGRIKELIIRGGENIHPQEIEAAVLGLPGVRDAAAAGRPHPVLGDVPALYIVPDADGV
DADAVLAACRERLSYFKVPEEIYRVDAIPRTMSGKVKRTSLTEAPAELLAGASGSDALYRLKWVPAETPGPAATGGH
VIVRVASLRADGTELAGAARDLARSWLSDERRAGATLVFVTGRAVSAGPSDVPVPEHAAVWDAIRDEQTENPGAFVL
IDLEEAETEEPESAAPEAGDPQADTPGADDTRLSTLVALGEPQIALRDSTPLVPRLAPESSTALTTPAARAWVLEPA
RSGTLRELSLVAADTDARPLRPGEVRVDVRAAGLNFRDVLIALGTYPGDGVMGGEAAGVVLEVGPEVNDLSVGDRVF
GLVTDGFGPVTITDRRLLAAMPQDWSFTTAASAAMAFATAHYGLVELAGLKAGESVLIHAATGGVGMAATQIAHHLG
AHIYATASSGKQHLLRAAGIDDDRIANSRTTGFRDAFLDSTGGRGVDVVLNSLSGEFVDSSLDLLAHGGRFIEMSTD
IRDAGRIAAERPGTTYQAFHLVDADPDRLREILTELLALFDQGILDPLPVQAWDIRQAREAFSWMSRARHTGKLVLT
IPQHIDPDGTVLITGGSGGLAGVVARHLVADKGARRLLLLSCDTLDATLAAELTESGARVDTAVCDVSDRAALAQVL
AGVSPEHPLTAIVHAGGAAVADESRQLHHLTKNRDLAAFVVFSQDAPAATEAFAGIRQAEGLPVTTIAWGIPEAEPV
VVGQHLLDRAMASADRAHVAARVNTAGLRALAAANALPPVLKNLVGAETDGTGHQDWSRRFMVAEAARQQELLDLIR
TTVMEILSLPTTARYFPDRTFRENGIDSLTAVELVNSLAKTTGLRLSATMVFDYPTPTALAGRMREL
SEQ ID NO: 173
MSRLDLIRPLSESLCASAASFGDKVAYTDSRRSVTYAELQIRTGRLAGHLAEHGVARGDRVAILLGNRVEIIESYLA
VARASAVAVPLNPDAMDAELAHFLRDSGAVVVITDLAHLEQTRGVAPAMTVVLIGDGRTVPGTSSFAELADTEPASP
ARDDLRLDEPAWMLYTSGTTGTPKGVVSAQRSGLWSAASCDVPAWDLSDEDLLLWPAPLFHSLGHHLCLLAVVAVGA
SARIMSGFAADEVLDALREHPCTVLVGVPTMYRYLLAAVGESGADAPALKMALIAGSVTPASLVEAFERSFGVPLID
TYGCTETTGSVTANRLHGERVPGSCGVPVPGVEIRLVDPVTGADVPLGAEGELWAKTPSVMIGYHGQPEATGEVLVD
GWYRSGDLARRQESGHITITGRVKELIIRSGENIHPREIETVALEVPGVEDAAAAGKPHRVLGEVPVLYVVPAEAGV
DVTAVFAACREQLSYFKVPEEIYQVESIPRTPSGKVKRGLLTEQPAELLAAADGGGSLYRVEWRPAVPPGAGDTGGD
SPVVVRVDSLPADEQELLGAVRDLIHDRIADPRRTTAPLVFVTRHAVLSRNPAHAHAAVWDLVSRAQADNPGLFVLV
DADGDDAPLPSAVGLGEPRVAWRDGGLLVPRLAHPGTEALIAPESGSWLLAETGGGTLRDLALVGTDTADRVLLPGE
VRIAVRAAGLNFRDVTVALGVVSDDRLMGGEAAGVVLDVAPDVTDLEPGDRVFGLVEGAFASVAVTDRRLLGRIPAG
WSFATAASVPVVFSTAYHALTDLVDLRPGEAILIHAAAGGVGMAATQLARHIGAKIYATASPAKQHALLGVDQVANS
RTTEFRGTFLEATGGRGVDVVLNSLAGEHIDASLELLPRGGRFLELGKTDLRDPRHLPAGVSYQVLNRLDSSPDRVR
EILAELLVMFERGVLRPLPVRTWDIREAPEAFSWMSRGRHLGKIVLTIPRDLDPDGTVLVTGASADHMARYLSAERG
HAHVLVSDDPAAVPATHPLTAVVHTGGDEVVSESTRLHQLTRELDLAAFAVFSQSAPASVEALVRHRRTEGLPATAV
SWGLPEAEPAPVQGALLDRTIASVEPAHVVTRVNSAGLRALANSGELPSVLRDVTPALSAKWPRPGTPRPGTPRPGT
PHPAALDQAALLDLVRESLTTVLGLPGVESCAPDRPFRETGLDSLTTIGLANTLSARLGRKLPATMIFDHPTPRTLA
TRLAEEL
SEQ ID NO: 174
MTPAYDVRPLPELLIANAERLGDKPAYTGLHRTVGHAELADRTRRAAGHLAGFAARGDRIALLLGNRVEMVEGYLAV
ARAAAVAVPLNPQASDAELAHFLTDSEAVAVLADAEHTEQVRRVAPGLRLVPIGEWETLATTEPDRPARDDLGLDEV
AWMLYTSGTTGAPKGVLSTQGSGLWSAYHCDVPALGLTDADVLLWPAPLFHSLAHHLAVLAATVSGATVRLLSGFAA
DEVLRELREEGCTLLAGVPTMYHYLLGAAGPDDEVRAPALRGAVVAGAVTPAALITAFGERFGAPLLDTYGCTETTG
SLTINRLDGPRVPGSCGVAVPGVRLRLVDPRTGDDAPEGGEGEIWASGPSLMRGYHRRPDATAEVLADGWYRTGDLA
VRAATGHITITGRVKELIIRGGENIHPREIEAVLAEVPGVADVAVAGRSHAVLGDLPVAYLVTEAGLDPAALFAACR
ERLSSFKVPEEVYRVAAVPRTPSGKIKRRELVAGPAELLATAGGAETLLRTRWTAVDVLDPASLDGWRVVHADQEVD
LGGRLDDDGPAIVVTTRAVRTSADERPSASAAAAWDLVTAAQARRPGRYLLVDTDGVPGGLGAALATGEPQVAVRED
VVLVPRLEAAGETGAPVRLDGTVLVTGEHTERVARHLRARGVTVTDDPAARPLHAVVHVGGTSGLAELAELTGCPER
AAFVVCTEDSRATADALVRAIPGGVAVGVGLPGIEPAALLPELLDRLTADGPYVVARPGSTGLRALATAGRLPAGLG
ALVDTGAAPDPDAAVRRDLVRRLIALPRRARDQALVELVWDAVRATLGAGATPGGPGQAFSEVGFDSLTSVQLRNRL
VAATGVRLSATAVFDFPTPRALADELGRVLI

In another aspect, the disclosure provides a chimeric polyketide synthase, wherein at least one module of the chimeric polyketide synthase has been modified as compared to a polyketide synthase having the sequence of SEQ ID NO: 175-176.

SEQ ID NO: 175

MSREEFIQPIHDLLRVNAERLGDKIAYADSRRELTHAELRTRTGRIAGHL
VDLAVERGDRVAILLGNRVETIESYLAIARAGAIAVPLNPDATGAEVAHF
LADSGAVLVITDSAHLDDVRRAAAAVTVVLVDEGPLPAGTRSFAELATAE
PPTPARDDLGLDEAAWMLYTSGTTGTPKGVVSTQGSGLWSAANCDVPAWE
LTENDVLLWPAPLFHSLAHHLCLLATTAVGATARIMSGFVAGEVLHELEE
HACTVLVGVPTMYHYLLGAVGEAGPRLPSLKMGLVAGAVSPPALIEGFER
VFGVPLLDTYGCTETTGSLTVNRLSGPRMPGSCGQAVPGISLRFVDPHTG
AEVAEGEEGELWASGPSLMIGYHGRPDATREVLSDGWYRTGDLARRSETG
HVTITGRVKELIIRGGENIHPRDIEAVALELPGVRDAAAAGKQHPVLGEI
PALYLVPDADGVDAEAVLAACREKLSYFKVPEEIYRVDAIPRTLSGKVKR
AALTEAPAELLSAASGNGSLYRLEWVPAETPPAGTGGPVAVHVTRRAVAT
GPADLPDQEQAATWDALRGEQTGPGGPVLIDLDGADIDDARLSALASLGE
PQIVVRDDTPLVARLAREKSPALTIPGERAWVLEPDHSGVLQELALVAAD
TDVRPLRPGEVRIEVRAAGLNFRDVLVALGTDLGDGVFGAEGAGVVLETG
SDVRDLRPGDRVFGLLEGGHGSIAIADRRMLAVIPEGWSFATAASVPEVF
VIAYYGLVDLAGLRAGESVLIHAATGGVGMAATQIARHLGAQVYATAGVG
KQHILRDAGLGDDRIADSRTTDFREAFRDSTQGRGVDVVLNSLKGDFVDA
SLDLLADGGRFLELGQTDIRDAGEIAAERPGTTYHSFTRMNAGPDRLREI
IAELLALFEQGVLRPSPVHTWDIRHAREAFSWMSGGRHTGKMVLTMPQRI
DPGGTVLIAGDSEALARIAARHLGVRHLLLDRGVADAAPDAVVCDVSDHD
ALERVLADLSPEHPLTAVIHTGGAAVTDEIRRLHDLTESLDLTDFVVFSQ
DAPAAVEAFARSRRAHGLPVRTIAWGIPEADPVVADEHLLGRALASAEQA
QIVARVNTAGLRALTAANALPTLLRNLIRAEPEETGQSAWPHRFEAAGAD
REEALLDLIRANVVDILSLPTADRYAPDRTFREMGIDSLTAVGLRNSLAK
ATGLPLPTTMVFDYPTPAVLTARMRELLAGESPAPARTAARAVAQDEPLA
IVGMACRLPGGVSSPDDLWRLVAAGTDAISEFPADRGWDVDNLYDPDPDA
PGKTYTVLGGFLDGVAGFDASFFGISPREALAMDPQQRLMLEVSWEAFEH
AGIPPRSVRGSDAGVFMGAFPSGYDAGLEEFGMTGDAVSVLSGRVSYFFG
LEGPAITVDTACSSSLVALHQASSALRQGECSLALVGGVTVLATPQTFVE
FSRQRGLALDGRSKAFADAADGAGWAEGVGVLVVERLSDARAKGHQIWGV
IRGSAVNQDGASNGLSAPNGPSQQRVIRQALANAGLAPHEVDVVEAHGTG
TTLGDPIEAQAVIATYGQDREQPLLLGSLKSNVGHTQAAAGVSGVIKMVM
ALQHDTVPATLHVDAPSRHVDWTAGAVELVTENRPWPETGRVRRAGVSSF
GISGTNAHVILESAPEQPVSPPEAVAPVVASDRVPLVISAKTPAALAEME
NRLRAYLAAAPGADPRAVASTLATARSVFEHRAVLLGENTITGTVAGADP
RVVFVFPGQGWQQLGMGRALRESSPVFAARMAECAAALSEFVDWDLFTML
DDPAVIDRIDVLQPACWAVMMSLAAVWQAAGVRPDAVIGHSQGEIAAACV
AGALSLRDAARIVALRSQLLAREMVGHGVMAAVALPADDIPLVDGVWIGA
CNGPSSTVISGTPEAVEVVVAACEERGARVRRITAAVASHSPLGEKIRTE
LLGISASIPSRTPVVPWLSTADGIWIEAPLDPAYWWRNLREPVGFGPAVD
LLQARGENVFLEMSASPVLLPAMNDAVTVATLRRDDGTPDRMLTALAEAH
AHGVIVDWPRVFGSTTRVLDLPTYAFEHQRYWAVSADRPSDAGHPMVETV
VPLPASGGVALTGRVSLATHAWLADHAVRGTALLPGTAFVELVTRAATEV
DCPVIDELVIEAPLPLTQTGAVQLSTTVGEADESGRRPVTVFSQADGTDA
WTRHVTATIGRAASLPDPVAWPPAQAEPVDVTGFYDELAAAGYEYGPAFQ
GLRAAWSDGDTVYAEVVLAEEQAHEVDRYAVHPALLDAALQAGMVNTAGT
GQGVRLPFSWNGIQVHSTGATTLRVAATPLADGWSVRAAADNGRPVATIG
SLVTRPVTTDMLGSTTDDLFAVVWTEITAPEPGDPSDVGVFTALPEAGGD
PLTQTRALTAQVLQTVQQWLAGEDRPLVVRTGTDLASAAVSGLVRSAQSE
HPGRLILVESDDELTPEQLAGTAGLDEPRIRIDGGHYEVPRLAREDASLT
VPEDRAWLLELPGSGTLRDLRVIPTDTAERPLRWGEVRVGVRAGGLNFRD
VVVALGMVTDPRPAGGEAAGVVLETGPGVEDLSPGDRVFGILDGGFGSVA
IADRRLLAVIPDGWSFTTAASIPVVFATAYYGLVDLAGLRAGESVLIHAA
TGGVGMAATQIARHLGAEIYGTAGIAKQHVLRDAGLGDDRIADSRTTGFR
ETFRDSTQGRGVDVVLNSLSGDFVDASLDVLAEGGRFIEMGKTDIRDAEQ
ITHATYRAFDLMDAGPDRVREIIAELLGLFEQGVLRPLPVQAWDIRQARD
AFTWMSRARHIGKIVLTIPQQLDPDGTVLISGGSGVLAGILARHLVAERG
VRHLLLVSRSAPSEALISELTALGAQVETVACDVSDRVALEQVLDGVPLT
AVFHTAAALDDGVVESLTPQRVDTVLRPKADAAWYLHELTRDADLAAFVM
YSSVAGIMGAAGQGNYAAANAFLDALAAHRRREGLPALSLAWGLWEDASG
LSAGLTETDHDRIRRGGLEAIAAEHGMRLFDTATRQGEPVLLASPLNLTR
QGEVPALLRTLHRPVARRAATANGRPADLTPEALLKLVCGRAAAVLGHVD
ADAVPVAVAFRDLGVDSLTAVELRNSLAKATGLRLPATLVFDYPTPTVLA
GRLGELLAGGTAPVRAAVVRRAAASDEPLAIVGMACRLPGGVLSPEDLWR
LVESGGDAISGFPVDRGWDVENLFDPDPDAAGRTYAVRGGFLDGAAGFDA
SFFGISPREAQAMDPQQRLVLEVSWEAFERAGIEPGSVRGSDTGVFMGAY
PGGYGVGTDLGGFGMTSVAVSVLAGRVSYFFGLEGPAMTVDTACSSSLVA
LHQAGSALRQGECSLALVGGVTVMPTPQTFVEFSRQRGLAADGRCKAFAD
AADGTGFSEGVGVLLVERLSDAQARGHNILAVVRGSAVNQDGASNGLTAP
NGPSQQRVIRQALANAGLAGAEVDVVEAHGTGTTLGDPIEAQAVIATYGQ
DRDQPVLLGSLKSNLGHTQAAAGVSGVIKMVMALRHDTVPATLHIDEPSR
HIDWTAGAVELVTENQSWPETGRARRAAVSSFGISGTNAHVILESAPAQP
VPLVDTPVSAVTAGVVPLPISARTVPALADLEDRLRAYLTTTPETDLPAV
ASTLAVTRSVFEHRAVLLGEETVTGIAVSDPRVVFVFSGQGSQRVGMGEE
LAAAFPLFARLHRQVWDLLDVPDLEVDDTGYVQPALFALQVALFGLLESW
GVRPEAVIGHSVGEVAAGYVAGVWSLEDACTLVSARARLMQALPAGGAMV
AVPVSEERARAVLVDGVEIAAVNGPASVVLSGDESAVLRVAEGLGRWTRL
SASHAFHSVRMEPMLEEFRQVASELTYREPRIVMAAGEQVTTPEYWVRQV
RDTVRFGDQVAAFGDAVFLEIGPDRTLSRLIDGIPTLHGDDEQHAVVAAL
AELHVQGVPIDWSSILGANPARVLDLPTYAFQHERYWMVSTGRVGGEGHP
LLGWGVPVAEAGGRLYTGRVARQDGPVLSVAAFVEMAFAAAGGRPIRELS
VDALLYIPDDGTAELQTWVSEHRLTIHARYRDTEPWTRLATAALDTTAPA
TTHTPHPGLITTALTLTGDEAPAIWHDLTLHTSNATELHTHITPGDDGTL
TITATDTTGQPVLTAHTATPTTIPVHTPTTPADDLLTLTWTQIPTPGPGD
PTDIAVCTALPDPDGDPLAQTRTLTAQVLQSIQTTLTGEDRPLVVHTGTG
LASAAVSGLVRSAQSEHPDRFILVESDDSLPQAQLAAVAGLDEPWLRITG
SCYEVPRLTKTTTATATAVSEPVWNPDGTVLITGGSGALAGILARHLVTE
RGVRHLLLISRSTPSTTLTDELRELGAHVDVAACDVSDRDALARVLDGVD
LTAVFHTAGALDDGVVESLTPQRLDTVLTPKADGAWHLHELTRDRDLTAF
VMYSSAAGVMGAAGQGNYAAANAFLDALAEHRHADGLPALSLAWGMWDDT
DGMTASLSGTDHRRIRRSGQRAITAEHGMRLLDKASGRSEPVLVATAMNP
IPDTDLPALLRSLYPKTARKSQPIQELSPEALLKIVRDSAALMLGHPNTD
AIAATTAFRDLGVDSLIAVELRNSLAKATGLRLPATLVFDYPTPTVLAGR
LGELLAGVTPQRHATVRTGTASDEPLAIVGMACRLPGGVSSPEDLWRLVE
SGTDAITDFPTDRGWDTDDLFDPDPDTAGKTYTVHGGFLDDVAGFDASFF
GISPREAQAMDPQQRLVLEAAWEAFERAGIEPGSVRGSDTGVFMGAYPGG
YGIGADLGGFGATAGAGSVLSGRLSYFFGLEGPAMTVDTACSSSLVALHQ
AGSALRQGECSLALVGGVTVIANPQIFVEFSRQRGLAADGRCKAFADSAD
GTGWSEGVGVLLVERLSDAQARGHNILAVVRGSAVNQDGASNGLTAPNGP
SQQRVIRQALANAGLAGAEVDVVEAHGTGTTLGDPIEAQAVIATYGQDRD
QSVLLGSLKSNLGHTQAAAGVSGVIKMVMALQNGVVPRTLHADQPSRHID
WTAGAVELVTENQPWPELDRPRRAAVSAFGVSGTNAHVILESAPDQPVPL
VDTPVSAVTAGVVPLPISARTVPALADLEDQLRAYLTTAPETDLPAVAST
LATTRSVFEHRAVLLGEDTVTGTAIPDPRIVFVFSGQGSQRAGMGEELAA
AFPLFARLHRQVWDLLDVPDLDVDDTGYVQPALFALQVALFGLLESWGVR
PRAVIGHSVGEVAAGYVAGVWSLEDACALVSARARLMQALPAGGAMVAVP
VSEERARAVLVDGVEIAAVNGPASVVLSGDEAAVLRVAEGLGRWTRLSAS
HAFHSVRMEPMLEEFRQVVSRLTYREPRIVMAAGEQVTTPEYWVRQVRET
VRFGDQVAAFGDAVFLEIGPDRTLSRLIDGIAMLDGDDEVRAAVAALAMM
HVQGVGVDWPAILGTTTGRVLDLPTYAFQHERYWMANADEGHPLLGKVEH
PLLGSVMALPNSDGVVLTGRISLATHAWLADHVVRGTVLLPGTGFVEMVA
RAAAEVGCGVIDELLIEAPLLLPEHGGVHLSVSVGEADGAGRRPVTVFAQ
ADDAEVWVRQVTATISPAGPAVSLPELEVWPPVQAEPVDVSTFYERLARA
DWQWGPAFQGLRAAWRDGDTIYAEIVLADEEAREADQFLVHPALLDAALQ
TSVLKTPDDLRLPFSWNQIEFHATGAAILRVAVTPVADRWIVHAADSTGR
PVATIGALVSRPVTAETLGSNTDDLFALTWTEIPTPGPGDPADVAVCTAL
PEPDSDPLTQTRTLTAQVLQSIQTSLTGEDRPLVVHTGTGLASAAVSGLV
RSAQSEHPDRFILVECDDETLTPDQLAATAGLDEPWLRITGGHYEVPRLT
KTTTAAATTVSEPVWDPDGTVLITGGSGALAGILARHLVTERSVRHLLLI
SRSTPSTTLINELRELGAHIETAACDVSDRDALARVLDGVDLTAVFHTAG
ALDDGVVESLTPQRLDTVLMPKADAAWHLHELTRDRDLAAFVMYSSAAGV
MGAAGQGNYAAANAFLDALAEHRRADGLPALSLAWGMWDDADGMTASLSG
TDHRRIRRSGQRAITAEHGMRLLDKASGRSEPVLVATAMNPAGEGEVPAL
LRTLHRPVARRAATTNGRPADLTPEALLKVVRDSAAVVLGHASADTVPAA
TAFQELGLDSLIAVELRNSLAKATGLRLPATMVFDYPTPAALAGRLGELL
AGETTPATAAVVRRATASDEPLAIVGMACRLPGGVSSPEDLWRLVESGFD
AITGFPTDRGWDVDNLYDPDPDAPGKSTTLHGGFLDDVAGFDASFFGISP
REAVAMDPQQRLAMEVSWEAFERAGIEPGSVRGSDTGVFMGAYPGGYGIG
AELGGFMLTGRAGSVLAGRVSYFFGLEGPAMTVDTACSSSLVALHQAAYA
LRQGECSLALVGGVTVMPTPVMFVEFSQQQNLADDGRCKAFADSADGTGW
SEGVGVLLVERLSDAQARGHNILAVVRGSAVNQDGASNGLTAPNGPSQQR
VIRSALTSAGLTTADVDVVEAHGTGTTLGDPIEAQAVLATYGQDRDQPVL
LGSLKSNLGHTQAAAGVSGVIKMVMALQNGVVPRTLHVEEPSRHVDWTAG
AVELVTENQSWPETGRARRAAVSSFGFSGTNAHVILESAPAQPVPPMDTP
APAVTTGVVPLPISAKSLPALADLEDQLRAYLTATPETDLPAVASTLAMT
RSVFEHRAVLLGEETVTGTAIPDPRIVFVFSGQGSQRVGMGEELAAAFPL
FARLHRQVWDLLDVPDLDVDDTGYVQPALFALQVALFGLLESWGVRPRAV
IGHSVGEVAAGYVAGVWSLEDACALVSARARLMQALPAGGAMVAVPVSEE
RARVALVDGVEIAAVNGPASVVLSGDEAAVLQIAEGLGRWTRLSASHAFH
SVRMEPMLEEFGQVASELTYQEPRIVMAAGEQVTTPEYWVRQVRDTVRFG
DQVAAFGDAVFLEIGPDRTLSRLIDGIAMLDGDDEVRAAVAALAELHVQG
VPIDWPAVLGTTTGRVLDLPTYAFQHQRYWAASTDRPAGDGHPLLDTVVA
LPGADGVVLTGRISLATHAWLADHAVRGTVLLPGTGFVEMVARAAAEVGC
AVVDELVIEAPLLLPASGGVQLSVSVGEADDAGHRPVTVHSQADETEAWV
RHVTATISPSGPIVSPPEFEVWPPAQAEPVEVARFYDELAAAGYEYGAAF
QGLRAAWRAGETIYAEVVLAEDQTLEAARFTVHPALLDAALQANILNASG
DLRLPFSWGQVQFHTTGAATLRVAVTPVADGWTIQATDDAGRPVATVGSV
VARPVAGLGATAEDLFALTWNEIPAPGQGGRTVGRFEDLADDGPVPELVV
FTALPDVDADPLVRTRALTARVLEAIQRWLGEPRFADSTLVVRTGTDLAS
AAVSGLVRSAQSEHPDRFILVEGDSSPVEIGLDEPWLRVDGGRYEVPRLI
RLSAEPVQEAAWNPDGMVLITGGTGALAGILARHLVAENKARRLLLVSRS
VPDDALISELTELGAEVGTAVCDVSDRAALARVLAGVPSLTAVIHTAGVL
DDGVMESLTPQRLDTVLRAKADGAWHLHELTRDRDLAAFVMYSSAAGLMG
SPGQGNYAAANAFLDALAVERRAEGLPALSLAWGFWEETTGLTANLTGAD
RDRIRRGGLQTITAERGMRMFDTATQHGEPVLLAAPISPVRDGEVPALLR
SLHRRGTRRGTTADASAQWLAGLAPEEREGALIKVVRDTAAVVLGHADAG
TIPVTAAFKDLGLDSLTAVELRNSLAKSTGLRLPATMVFDYPTPASLAAR
LDDLMNPRVSSTALLAELDRIEGMFDSVTFDEKQASLVKDRLSAALGKWQ
QISRSADVATVALANADAGEILDFIDREFGNPTI

SEQ ID NO: 176

MPDHDKLVEYLRWATAELHTTRAKLQAATEAGTQPLAIVGMACRLPGGVS
SPEDLWRLVESGTDAISGFPVDRGWDVDGLYDPDPDVPGKSYTVEGGFLD
AVTGFDAPFFGISPREALAMDPQQRLVLEASWEAFERAGIEPGSVRGSDT
GVFMGAFPGGYGTGADLGGFGMTGGAASVLSGRVSYFFGLEGPAMTVDTV
CSSSLVALHQAGYALRHGECSLALVGGVTVMSTPQTFVEFSRQRGLAADG
RCKAFADNADGTGWSEGVGVLLVERLSDAQARGHNILAVVRGSAVNQDGA
SNGLTAPNGPSQQRVIRQALANAGLTGADVDVVEAHGTGTTLGDPIEAQA
VIATYGRDRDQPVLLGSLKSNLGHTQAAAGVSGVIKMVMALQNGVVPRTL
HIEEPSRHVDWTAGAVQLVTENRPWPELGRARRAAVSSFGLSGTNAHVIL
ESAPDQPPAPTTDTPVSAVTAGVVPLPISAKTVPALADLEDRLRTYLTTT
PDTDLPAVASTLATTRSLFEHRAVLLGEDTVTGTAIPDPRVVFVFPGQGW
QWQGMGSALLTSSTVFAERMAECAAALSEFVDWDLLTVLDDPSVVDRVDV
VQPACWAVMISLAAVWQAAGIHPDIVLGHSQGEIAAACLAGAISLPDAAR
IVAQRSQLIAHQLTGHGAMASISLPADDIPTTDKVWIAAHNGTSTVIAGD
PQAVEAVLATCETRGARVRKINVDYASHTPHVEQIRTELLDITTGIEAHT
PAVPWLSTTDNTWIDQPLDPTYWYRNLREPVRFGPAIDLLQTQDNNLFIE
ISASPVLLQTMDNAATVATLRRDEDTTQRLLTAFAEAHVHGATIDWPTVL
DTTTTPVLDLPTYPFQRQRYWATSNGRSTGQGHPLLETVVALPGTDGVAL
TGRISLATHPWLTDHTVRGTVLLPGTAFVELVTRAATEVNCQIIDELIIE
APLPLPQTDGVQLSVTVGEADEAGHRPVTVYSQTDESDDWIQHVTATIGP
GASLPETAAWPPAHAEPVNVTGLYDNLAAAGYEYGPAFQGLQAAWRAGDT
VYAEVTLAEEQAQETARFTMHPALLDAALHTIALHDTGDLHLPFSWTRVQ
FHGTGAATLRVAVTPAADGWNIRATDDTGRAVATIGSLVTRPMAAETTDD
LLALTWTEIPAPEPVDPTDVVVFTALPDTVEDVPAQTRALTTRVLHTIQE
WLADDDRTLIVRTGTDLASAAVSGLVRSAQSEHPGRFILVESADEALTQE
QLAATAGLDEPRLRITGGRYEVPRLTREDTALAVPTDRAWLLEQPRSGSL
EDLALLPTDAAERPLQAGEVRIGVRAAGMNFRDVVVALGMVTDTRLAGGE
AAGVVLEVGTDVNDFRPGDRVFGILEGGFGSVAICDHRTLAVIPDGWSFT
TAASVPIAFATAYYGLVDLAGLRAGESVLIHAATGGVGIAATQIARHLGA
EIYGTASVGKQHVLRDAGLADDRIADSRTTDFRDTFRDGTQGRGVDVVLN
SLRGEFIDASLDLLVDGGRFIEMGKTDIRDAAQIPDATYHAFDLMDAGHD
RLREIMTELLALFEQGVLHPMPVHAFDIRQAREAFSWMSRARHIGKLVLT
IPQPIDPDGTVLITGGSGVLAGIVARYLVTENRARHLLLLSRSAPSASLI
DELTALGAHVDVAACDVADRAALAEILDGVDLTAVIHTAGALDDGVVESL
TPQRLDTVLTPKADGAWHLHELTRDRDLAAFIVYSSAAGVLGAAGQGNYA
AANAFLDALAVHRRLEGLPGLSLAWGLWEDASGLTADLTDADRDRIRRSG
QRAITAAYGMRMLDAATRQSEAILLAAPISPIQDGDVPAILRSLHRRVGR
RASVAHGHPADLTPEALLKVVRDSAAMVLGHTNADTVPTATAFQELGLDS
LTAVELRNSLTKATGLRLPATMAFDYPTPDALAARLGELLAGEAAPKAAA
AVRRATASDEPLAIVGMACRLPGGVSSPEDLWRLVESGTDAITDFPTDRG
WDTDTLFDPDPDTPGKTYTVHGGFLNDVAGFDAPFFGISPREAVAMDPQQ
RLVLESSWEAFERAGIQPDSIRGSDTGVFMGAYPDGYGIGADLAGFGVTA
GAGSVLSGRVSYFFGLEGPAMTVDTACSSSLVALHQAAYALRQGECSLAL
VGGVTVMPSPRTFIEFSRQRGLAADGRSKAFADAADGTGFSEGVGVLLVE
RLSDAQAKGHNILALVRSSAVNQDGASNGLTAPNGPSQQRVIQSALAGAG
LTSADVDVVEAHGTGTTLGDPIEAQAVLATYGQDRDQPVLLGSLKSNLGH
TQAAAGVSGVIKMVMALQHNTVPATLHVDAPSRHVDWTAGAVRLATENQP
WPETNRPRRAGVSSFGVSGTNAHVILEQAPAASPVEPVDTTDVVIPLVVS
ARSSGSLSDQADRLAALVGSPDAPALTSLADALLTRRTVFSQRAVVVAGS
HEQAAAGLRALASGDSHPALVTGAAGPARGVVLVFPGQGSQWAGMGAELL
DTSPVFAARIAECAEALRPWVDWSLDEVLRGDASADVLGRVDVVQPASFA
VMVGLAAVWESAGVRPDAVLGHSQGEIAAAYVAGALSLTDAAKIVAVRSR
LIAARLAGRGGMASVALAPDEAAAKLGRTELAAVNGPASVVIAGDAEALD
ETLAMLEGEAVRVRRVAVDYASHTPHVEELEQSMAEALADVRSRQPRVGF
LSTVTGDWVTEAGALDGGYWYRNLRQPVRFGPAVASLAEAGYTVFVEASA
HPVLVQPVAETLDRTDAVVTGTLRRQDGGLPRLLTSMAELFVGGVPVNWP
VLLPAGAVRGWVDLPTYAFDHQRYWLENRVATDAAALGLAGADHPLLGAI
VAVPQSGGVAMTSRLSPRNHPWLAEHTLGGVPTVPTSVLVELAVRAGDEV
GCGVVEELTVDAPLLLPERGGVRVQVIVGATDANGQRGLDIFSAPEDTGQ
EAWTRHATGTLAPGGDIAADVDLSAWPPANAQPVDVTDGYDLLERAGYGY
GPAFQGVRAIWRRGEELFAEVALEPELTDTAARFGLHPALLDAAWHPELR
DEVAETSPDGRRWWSQPSRWAGLRLHTAGATVLRVRLAPVDADSMSLQAA
DETGDPVLTVDSLSLCAVSADQLTTAESSDDALFRLEWTPLSKAPTAARS
WVPVETGADVAALDGQAVVDAVMLEAAGTGDALELTCRVLEVVQAWLTLP
GWDESRLVVVTRGAVGAVGDPAGSAVWGLVRAAQAENPDRIALLDLDGGR
PVEPLLAESEPQLAIRGAEALVPRLIRAAAATDAPALFDESQTVLITGGT
GSLGGLLARHLVGRYGLRRLVLVSRRGPDAPGAYELAAELAAHGAEAALV
ACDLTDRDAVARLLTEHHPTAVVHAAGVSDDGVIGTLTSDRLAYVFGPKA
TAARHLDELTRELLPDLAAFVTYSSISAVFLGAGSGGYAAANAYLDGLMA
RRHAEGLPGLSLAWGLWDQEADGGGMAAGLQDITRNRMRRRGGVLSFTPA
EGMALFDAAMATDEALVVPVRLDLPALRAEAVAEGRSAPVLLRGLVRPGR
RLARTVSGGTGVLADLTPEALLKLVRGRAAAVLGHVDADAVPVAAAFKDL
GVDSLTAVELRNSLAKATGLRLPATLVFDYPTPTVLAGRLGELLAGGTAP
VRAAVVRRAAASDEPLAIVGMACRLPGGVLSPEDLWRLVESGGDAISGFP
VDRGWDVENLFDPDPDAAGRTYAVRGGFLDGAAGFDASFFGISPREAQAM
DPQQRLVLEVSWEAFERAGIEPGSVRGSDTGVFMGAYPGGYGMGTDLGGF
GMTSVAVSVLAGRVSYFFGLEGPAMTVDTACSSSLVALHQAGSALRQGEC
SLALVGGVTVMPTPQTFVEFSRQRGLAADGRCKAFADAADGTGFSEGVGV
LLVERLSDAQARGHNILAVVRGSAVNQDGASNGLTAPNGPAQQRVIQSAL
AGAGLASADVDVVEAHGTGTTLGDPIEAQAVIATYGQDRDQPVLLGSLKS
NLGHTQAAAGVSGVIKMVMALQNGVVPRTLHIDEPSRHIDWTAGAVELVT
ENQSWPETGRARRAAVSSFGISGTNAHVILESAPAQPVPLVDTPVSDVTA
GVVPLPISARTVPALADLEDQLRAYLTTAPETDLPAVASTLAMTRSVFEH
RAVLLGEETVTGIAVSDPRVVFVFSGQGSQRVGMGEELAAAFPLFARLHR
QVWDLLDVPDLEVDDTGYVQPALFALQVALFGLLESWGVRPRAVIGHSVG
EVAAGYVAGVWSLEDACTLVSARARLMQALPAGGAMVAVPVSEERARAVL
VDGVEIAAVNGPASVVLSGDESAVLRVAEGLGRWTRLSASHAFHSVRMEP
MLEEFRQVASELTYREPRIVMAAGEQVTTPEYWVRQVRDTVRFGDQVAAF
GDAVFLEIGPDRTLSRLIDGIAMLDGDDEVRAAVAALAMMHVQGVGVDWP
AVLGTTTGRVLDLPTYAFQHERYWMVSTGRPGGEGHPLLGWGVPVAEADG
RLYTGRVARQDGPVLPVAAFVEMAFAAAGGRPIRELSVDALLYIPDDGTA
ELQTWVSEHRLTIHARYRDTEPWTRLATATLDTTEPATTHTPHPGLITTA
LTLTGDEAPAIWHDLTLHTSNATELHTHITPGDDGTLTITATDATGQPVL
TAHAATPTTIPVHTPTTPADDLLTLTWTQIPTPGPGDGADIAVCTALPDP
DSDPLAQTRTLTAQVLHSIQASLTGEDRPLVVHTGTGLASAAVSGLVRSA
QSEHPDRFILVESDETLTPDQLAAVAGLDEPWLRITDGRYEVPRLTKTTT
TATATAVSEPVWDPDGTVLITGGSGALAGILARHLVTERGVRHLLLVSRS
TPSTTLIDELRELGAHVDVAACDVSDRAALARVLDGVDLTAVFHTAGALD
DGVVESLTPQRVDAVLRPKADGAWHLHELTRDRDLTAFVMYSSAAGVMGA
AGQGNYAAANAFLDALAEHRRADGLPALSLAWGMWDDADGMTASLSGTDH
RRIRRSGQRAITAEHGMRLLDKASGRSEPVLVATAMNPIPDTDLPALLRS
LYPKTARKSQPIQELSPEALLKIVRDSAAMVLGHANADTVPTATALQELG
LDSLTAVELRNSLTKATGLRLPATMAFDYPTPAALAGRLGELLAGDTTPA
TAAVVRRATASDEPLAIVGMACRLPGGVSTPEDLWRLVESGTDAITDFPT
DRGWDTDDLFDPDPDTPGKTYTVHGGFLDDVAGFDASFFGISPREALAMD
SQQRLVLEAAWEAFERAGIEPGSVRGSDTGVFMGAYPDGYGIGADLGGFG
ATAGAGSVLSGRLSYFFGLEGPAMTVDTACSSSLVALHQAGSALRQGECS
LALVGGVTVIANPQIFVEFSRQRGLAADGRCKAFADNADGTGFSEGVGVL
LVERLSDAQAKGHNILALVRSSAVNQDGASNGLTAPNGPSQQRVIRQALA
NAGLTGAEVDVVEAHGTGTTLGDPIEAQAVLATYGQDRDQPVLLGSLKSN
LGHTQAAAGVSGVIKMVMALRHDTVPATLHIDEPSRHIDWTAGAVELVTE
NQPWPVLGRPRRAAVSAFGVSGTNAHVILESAPDQPPAPATDTPAPAATA
GVVPLPISAKTVPALADLEDRLRTYLTTTPETDLPAVASTLATTRSLFEH
RAVLLGEDTVTGTTIPDPRIVFVFPGQGWQWQGMGSALLTSSTVFAERMA
ECAAALSEFVDWDLLTVLDDPSIVDRVDVVQPACWAVMISLAAVWQAAGI
HPDIVLGHSQGEIAAACLAGAISLPDAARIVAQRSQLIAHQLTGHGAMAS
ISLPADDIPTTDKVWIAAHNGTSTVIAGDPQALDTVLATCETHGARVRKI
NVDYASHTPHVEQIRTELLDITTDIEAHTPTVPWLSTTDNTWIDQPLDPT
YWYRNLREPVRFGPAIDLLQTQDNNLFIEISASPVLLQTMDNATTVATLR
RDEDTTQRLLTAFAEAHVHGATIDWPTVLDTTTTPVLDLPTYPFQRQRYW
ATSNGRPTSQGHPLLETVVALPGTHGVALTGRISLATHPWLTDHTVRGTV
LLPGTAFVELVTHAATEVNCQVIDELIIEAPLPLPQNGGVQLSVTVGEAD
EAGHRPVTVYSQTDESDDWVQHVTATIAPGVSSSESAAWPPAQAEPVNVT
GLYDNLAAAGYEYGPAFQGLQTAWRDGSTVYAEVTLAEEQAQETARFTMH
PALLDAALHTIALHDTADLQLPFSWRQVQFHGSGAATLRVAVTPAADGWN
IRATDDTGQTVATIGSLVTRPMAAETTNDLLALTWTEIPAPEPVDPADVV
VFTALPEPGSDPLAQTRALTTRVLHTIQEWLADDDRTLIVRTGTDLASAA
VSGLVRSAQSEHPGRFILVESDDETLTHEQLAATAGLDEPRLRITDGRYE
VPRLTREDTALAVPEGGAWMLDQPSRSGTLQDLRLVPTDAAERPLRPGEV
RVGVRAAGLNFRDVAVALGMVTDTRLIGGEGAGVVLEAGPGVEDLRPGDR
VFGLLEGGFGPVAVADRRALALIPDGWSFTTAASVPIAFATAYYGLLDLA
GLRAGESVLIHAATGGVGMAATQIARHLGADVYATASTGKQHVLRDAGLS
DDRIADSRTTGFRETFRDSTDGRGVDVVLNSLKGDFVDASLDLLVDGGRF
IEMGKTDIRDAAQIPDATYRAFDLMDAGPERLREIITELLALFEQGVLRP
LPVHAFDIRQARDAFGWMSRARHIGKLVLTIPQPIDPDGTVLITGGSGVL
AGIVARHLVIAEGLRNLLLLSRSAPSEALIGELTALGAQVETAACDIADR
AALARVLDGVPLTAVIHTAGALDDGVVESLDPQRLDSVLTPKADGAWHLH
ELTRDRDLAAFIMYSSAAGVLGAAGQGNYAAANAFVDALAVHRRFMGLPA
LSLAWGLWDDTSALTAGLTDSDHDRIRRSGARTITAEHGMRMFDAATRQS
EAVLLAAPMGPIRGEDVPALLRGLATVRQPRTRAKRDMGPERLRDRLNGR
TSVEQHRIMVELVLAHATSVLGHESPDAIAPDRAFKDLGMDSLTAIELRN
HLVAETGVRLPATTAFDHPTADDLAKRLLAEVGLTPAPQRTEADIREEVV
VREPAGDDSWTSEPIAIVSMSCRAPGGVDSPESLWRLVESGTDAITDFPG
DRGWDVAGLYSPDPDTGYKTYCVQGGFLDAAADFDAAFFGISPREALGMD
PQQRLLLETSWEAIERARIDPRSLRGRNVGVYVGGAAQGYGVGAIDQQRD
NVITGSSISLLSGRLSYALGLEGPGVTVDTACSSSLVALHLACQALRQRE
CSMALVSGVSVIPTPDVFVEFSRQRGLAADGRCKSFSASADGTIWAEGVG
VLVLERLSEATRLGHRVLAVVRGSAVNSDGASNGLTAPNGVSQQRVIRQA
LTGAGLTAADVDVVEAHGTGTKLGDPIEAEAILATYGQDRSTPVCLGSLK
SNIGHAMAASGVLAVIKMVEAMRHGLIPRTLHVEEPSPHVDWASGDVALL
TENQPWPDDAKLRRAGVSSFGLSGTNAHVVLEQYRAPAAPDITTTEHEPL
AWTLSARDPKALREQAGRLHAALTESPQWRPLDIGYSLATTRSNFAHRAV
AVGSDREDLLRALSKLADGSAWPALVTATAKDRRVAYLFDGQGSQRPDMG
SGLYERFPAFARAWDRISAEFGKHLDHSLTDVYLGRGDAATADLVDDTLY
AQAGLFTMEIALFELLAEWGVRPDFVSGHSIGETAAAYAAGVLSLEDVTT
LIVARGRALRQVPPGAMVALRAGEDEAREFLGRTGAALDLAAVNSPTSVV
VSGASEAVAGFRARWTESGREARTLNVRHAFHSRHVEAVLGEFREVLESL
TFRTPALPVVSTVTGRLIEPTELSTSEYWLRQVRQTVRFHDAVRELSGQG
VGTFVEIGPSGALASAGLECLGDEASFHAVQRPGSPGDVCLMTAVAELHA
GGTTVDWATVLAGGRATDLPVYPFQHGSYWLAPVTRAADGAPSAGVPAPG
EYARPSAPEEPRTMLELVRLEAAIALSITDPGLIADDSSFLDLGFDSISA
LRLSNRLAAVTGLDLPPSLLFDHPTPAELAARLDELSAADLDGAGVYALL
EEIDELDDEDLDMTEEEQTAISELLTKLSAKWSR

In some embodiments, the disclosure provides a chimeric polyketide synthase where at least one module includes a portion having at least 90% identity to any one of SEQ ID NO: 1-174.

In another aspect, the disclosure provides a nucleic acid encoding any one of the above described polyketide synthases.

In some embodiments of any of the above described aspects, the nucleic acid encoding any one of the above described polyketide synthases further encodes an LAL in which the sequence encoding the LAL is operatively linked to the sequence encoding the polyketide synthase.

In some embodiments, the LAL may be a heterologous LAL.

In some embodiments, the LAL may include a portion having at least 80% (e.g., at least 80%, at least 85%, at least 90%, at least 95%, at least 99%) sequence identity to SEQ ID NO: 177. In some embodiments, the LAL may include a portion having the sequence of SEQ ID NO: 177. In some embodiments, the disclosure provides a nucleic in which the LAL has the sequence of SEQ ID NO: 177. In some embodiments, the LAL lacks a TTA inhibitory codon in an open reading frame.

SEQ ID NO 177:
MPAVESYELDARDDELRRLEEAVGQAGNGRGVVVTITGPIACGKTELLDA
AAAKSDAITLRAVCSEEERALPYALIGQLIDNPAVASQLPDPVSMALPGE
HLSPEAENRLRGDLTRTLLALAAERPVLIGIDDMHHADTASLNCLLHLAR
RVGPARIAMVLTELRRLTPAHSQFHAELLSLGHHREIALRPLGPKHIAEL
ARAGLGPDVDEDVLTGLYRATGGNLNLGHGLIKDVREAWATGGTGINAGR
AYRLAYLGSLYRCGPVPLRVARVAAVLGQSANTTLVRWISGLNADAVGEA
TEILTEGGLLHDLRFPHPAARSVVLNDLSARERRRLHRSALEVLDDVPVE
VVAHHQAGAGFIHGPKAAEIFAKAGQELHVRGELDAASDYLQLAHHASDD
AVTRAALRVEAVAIERRRNPLASSRHLDELTVAARAGLLSLEHAALMIRW
LALGGRSGEAAEVLAAQRPRAVTDQDRAHLRAAEVSLALVSPGASGVSPG
ASGPDRRPRPLPPDELANLPKAARLCAIADNAVISALHGRPELASAEAEN
VLKQADSAADGATALSALTALLYAENTDTAQLWADKLVSETGASNEEEGA
GYAGPRAETALRRGDLAAAVEAGSAILDHRRGSLLGITAALPLSSAVAAA
IRLGETERAEKWLAEPLPEAIRDSLFGLHLLSARGQYCLATGRHESAYTA
FRTCGERMRNWGVDVPGLSLWRVDAAEALLHGRDRDEGRRLIDEQLTHAM
GPRSRALTLRVQAAYSPQAQRVDLLEEAADLLLSCNDQYERARVLADLSE
AFSALRHHSRARGLLRQARHLAAQCGATPLLRRLGAKPGGPGWLEESGLP
QRIKSLTDAERRVASLAAGGQTNRVIADQLFVTASTVEQHLTNVFRKLGV
KGRQHLPAELANAE

In some embodiments of any of the foregoing nucleic acids, the nucleic acid includes an LAL binding site, in which the sequence encoding the LAL binding site is operatively linked to the sequence encoding the polyketide synthase.

In some embodiments, the LAL binding site includes a portion having at least 80% (e.g., at least 80%, at least 85%, at least 90%, at least 95%, at least 99%) sequence identity to the sequence of SEQ ID NO: 178 (CTAGGGGGTTGC). In some embodiments, the LAL binding site includes a portion having the sequence of SEQ ID NO: 178 (CTAGGGGGTTGC). In some embodiments, the LAL binding site has of the sequence of SEQ ID NO: 178 (CTAGGGGGTTGC). In some embodiments of the above described aspect, the LAL binding site has the sequence of SEQ ID NO: 179 (GGGGGT).

In some embodiments of any of the foregoing nucleic acids, the binding of an LAL to the LAL binding site promotes expression of the polyketide synthase.

In some embodiments of any of the foregoing nucleic acids, the nucleic acid encoding any one of the above described polyketide synthases, further encodes a nonribosomal peptide synthase.

In some embodiments of any of the foregoing nucleic acids, the nucleic acid encoding any one of the above described polyketide synthases further encodes a P450 enzyme.

In some embodiments of any of the foregoing nucleic acids, the nucleic acid encoding any one of the above described polyketides and a first P450 enzyme, further encodes a second P450 enzyme.

In another aspect, the disclosure provides an expression vector including any of the foregoing nucleic acids. In some embodiments, the expression vector may be an artificial chromosome, e.g., a bacterial artificial chromosome.

In another aspect, the disclosure provides a host cell including any of the above described expression vectors.

In another aspect, the disclosure provides a host cell including any of the foregoing polyketide synthases, in which the polyketide synthase is heterologous to the host cell.

In some embodiments of any of the foregoing host cells, the host cell naturally lacks an LAL and/or an LAL binding site.

In some embodiments of any of the foregoing host cells, the host cell includes an LAL capable of binding to an LAL binding site and regulating expression of a polyketide synthase. In some embodiments, the LAL and/or LAL binding site may be heterologous to the cell. In some embodiments, the host cell includes an LAL with a portion having at least 80% (e.g., at least 80%, at least 85%, at least 90%, at least 95%, at least 99%) sequence identity to the sequence of SEQ ID NO: 177.

In some embodiments of any of the foregoing host cells, t he host cell is a bacterium, e.g., an actinobacterium, such as an actinobacterium selected from the group consisting of Streptomyces ambofaciens, Streptomyces hygroscopicus, or Streptomyces malayensis. In some embodiments in which the host cells is an actinobacterium, the actinobacterium is S1391, S1496, or S2441.

In some embodiments of any of the foregoing host cells, the host cell has been modified to enhance expression of a polyketide synthase. For example, the host cell has been modified to enhance expression of a compound-producing protein by (i) deletion of an endogenous gene cluster which expresses a compound-producing protein; (ii) insertion of a heterologous gene cluster which expresses a compound-producing protein; (iii) exposure of the host cell to an antibiotic challenge; and/or (iv) introduction of a heterologous promoter that results in an at least 2-fold increase in expression of a compound compared to the homologous promoter.

In another aspect, the disclosure provides a method of producing a polyketide by culturing any of the foregoing host cells under suitable conditions.

In another aspect, the disclosure provides a method of producing a polyketide by culturing a host cell engineered to express any of the foregoing polyketide synthases under conditions suitable for the polyketide synthase to produce a polyketide.

In another aspect, the disclosure provides a method of producing a compound, the method including: (a) providing a parent polyketide synthase sequence capable of producing a compound; (b) determining the compatibility of at least one module of a second polyketide synthase with at least two modules of the parent polyketide synthase; (c) producing a nucleic acid encoding a modified polyketide synthase, wherein the modified polyketide synthase includes at least one module of a second polyketide synthase which has been determined to be compatible with the at least two modules of the parent polyketide synthase.

In another aspect, the disclosure provides a method of producing a compound, the method including: (a) providing a parent nucleic acid encoding a parent polyketide synthase; (b) modifying the parent nucleic acid to create a modified nucleic acid encoding a modified polyketide synthase capable of producing a compound, wherein the modification produces a modified polyketide synthase including at least one heterologous module.

In another aspect, the disclosure provides a method of producing a compound, the method including: (a) providing a parent polynucleotide sequence capable of producing a compound; (b) identifying one or more heterologous modules suitable for replacement of one or more modules in the parent polynucleotide sequence; (c) producing a nucleic acid encoding a modified polyketide synthase, wherein the modified polyketide synthase includes at least one heterologous module identified in step (b).

In another aspect, the disclosure provides a method of producing a plurality of engineered polyketide synthases, wherein each of the plurality of polynucleotides corresponds to an engineered polyketide synthase, and wherein each of the plurality of polynucleotides includes one or more heterologous modules with altered enzymatic activity relative to a reference polyketide. The method includes the steps of: (a) providing a parent polynucleotide sequence encoding a polyketide synthase; (b) identifying one or more modules for replacement in the parent polynucleotide sequence; (c) identifying two or more heterologous modules suitable for replacement for each of the modules identified in step (b); (d) generating a plurality of polynucleotides, wherein each of the plurality of polynucleotides corresponds to an engineered polyketide synthase, and wherein each of the plurality of polynucleotides includes a heterologous module selected from the two or more heterologous modules identified in step (c) in replacement of each of the one or more modules to be replaced identified in step (b).

### Definitions

A “polyketide synthase” refers to an enzyme belonging to the family of multi-domain enzymes capable of producing a polyketide. A polyketide synthase may be expressed naturally in bacteria, fungi, plants, or animals.

As used herein, the term “engineered polyketide synthase” is used to describe a non-natural polyketide synthase whose design and/or production involves action of the hand of man. For example, in some embodiments, an “engineered” polyketide synthase is prepared by production of a non-natural polynucleotide which encodes the polyketide synthase.

A cell that is “engineered to contain” and/or “engineered to express” refers to a cell that has been modified to contain and/or express a protein that does not naturally occur in the cell. A cell may be engineered to contain a protein, e.g., by introducing a nucleic acid encoding the protein by introduction of a vector including the nucleic acid.

The term “gene cluster that produces a small molecule” or “gene cluster that produces a compound,” as used herein, refers to a cluster of genes which encodes one or more compound-producing proteins.

The term “heterologous,” as used herein, refers to a relationship between two or more proteins, nucleic acids, compounds, and/or cell that is not present in nature. For example, the LAL having the sequence of SEQ ID NO: 177 is naturally occurring in the S18 Streptomyces strain and is thus homologous to that strain and would thus be heterologous to the S12 Streptomyces strain.

The terms “homologous” or “native,” as used interchangeably herein, refer to a relationship between two or more proteins, nucleic acids, compounds, and/or cells that is present naturally. For example, the LAL having the sequence of SEQ ID NO: 177 is naturally occurring in the S18 Streptomyces strain and is thus homologous to that strain.

The term “recombinant,” as used herein, refers to a protein that is produced using synthetic methods.

As used herein, the term “reference polyketide synthase” refers to a polyketide synthase that has a sequence having at least 80% identity (e.g., at least 80% identity, at least 85% identity, at least 90% identity, at least 95% identity, at least 99% identity, or 100% identity) to the sequence of an engineered polyketide synthase except to the sequence of the one or more modules which are modified.

As used here, the term “compatibility” refers to a measure of the likelihood of two adjacent modules to form a competent module-module junction, in which polyketide translocation is not substantially inhibited. A heterologous module may be considered compatible if it meets at least one of the following criteria: 1) the module is present in the same module clade as one or more adjacent modules of the reference PKS, as determined by the module-level phylogeny classification described in the detailed description of the invention; 2) the module is assigned a score of greater than or equal to 0.90 in the inter-module covariation analysis algorithm described in the detailed description of the invention; or 3) the module belongs to the same functional clade or sub-clade as one or more adjacent modules of the reference PKS, as determined by the evolutionary trace methodology outlined in the detailed description of the invention.

As used here, the term “linking sequence” refers to a sequence directly upstream or downstream of an inter-modular junction. For example, in a single module swap, the ACP for the upstream homologous module, the ACP and KS-AT didomain of the inserted heterologous module, and the KS of the downstream homologous module may all be considered linking sequences.

As used herein, the term “module” refers to a region of a polyketide synthase that includes multiple domains. Modules present in a polyketide synthase may include i) a loading module; ii) extending modules; and iii) releasing and/or cyclization modules, depending on whether the final polyketide is linear or cyclic. The domains which may be included in a given module include, but are not limited to, acyltransferase (AT), acyl carrier protein (ACP), keto-synthase (KS), ketoreductase (KR), dehydratase (DH), enoylreductase (ER), methyltransferase (MT), sulfhydrolase (SH), and thioesterase (TE).

As used here, the term “acceptor module” refers to a homologous module within a PKS cluster subject to engineering by module swapping. In the resulting engineered PKS cluster, the acceptor module is absent.

As used here, the term “donor module” refers to a heterologous module that is introduced into an engineered PKS cluster.

As used here, the term “module swapping” refers to the exchange of one or more heterologous donor modules for one or more homologous acceptor modules.

As used here, the term “does not substantially inhibit polyketide translocation” refers to the ability of a heterologous PKS module to function in a biosynthetic assembly line. For example, a heterologous loading module does not substantially inhibit polyketide translocation if the loading module is able to load a starter unit onto its ACP domain and pass the starter unit to the KS domain of the adjacent (n+1) extender module. A heterologous extender module does not substantially inhibit polyketide translocation if the extender module is able to receive a starter unit or polyketide chain from the previous (n−1) module, catalyze the addition of an extender unit, and pass the elongated polyketide chain to the adjacent (n+1) module. In some embodiments, a heterologous module does not substantially inhibit polyketide translocation if the engineered PKS that includes the heterologous module produces a compound in levels that are detectable by a highly sensitive detection method, e.g., LC-TOF mass spectrometry.

An extender unit, e.g., a malonyl-CoA, is loaded onto the acyl carrier protein domain of the current module catalyzed by another acyltransferase domain. The polyketide chain is then elongated by subsequent extender modules after being passed from the acyl carrier protein domain of module n to the ketosynthase domain of the n+1 module. The acyl carrier protein bound extender unit reacts with the polyketide chain bound to the ketosynthase domain with expulsion of CO₂ to produce an extended polyketide chain bound to the acyl carrier protein. Each added extender unit may then be modified by β-ketoprocessing domains, i.e., ketoreductase (which reduces the carbonyl of the elongation group to a hydroxy), dehydratase (which expels H₂O to produce an alkene), and enoylreductase (which reduces alkenes to produce saturated hydrocarbons).

### BRIEF DESCRIPTION OF THE FIGURES

FIGS. 1A and 1B are schematics illustrating the mechanisms by which PKS biosynthesis proceed. FIG. 1A depicts polyketide chain elongation and β-carbonyl processing within a module. FIG. 1B depicts translation between modules.

FIG. 2A is a diagram depicting complementary bioinformatics approaches to the prediction of functional protein-protein interactions at the module-module junction.

FIG. 2B is a phylogenetic tree resulting from multiple sequence alignments of complete FK-family modules.

FIGS. 2C-2E depict how inter-module residue covariation is used to generate an algorithm that ranks module-module junction compatibility. FIG. 2C is a diagram that illustrates the upstream and downstream module-module junctions used to determine the compatibility of a given heterologous module. FIG. 2D is a correlation map that depicts the alignment of the ACP domain of a given module and the KS-AT didomain of a second module. FIG. 2E depicts the compatibility score resulting from inter-domain residue covariation analysis for a series of heterologous modules. Scores are normalized to the homologous module for the polyketide synthase in question, which is given a score of 1.00.

FIGS. 2F and 2G depict how evolutionary trace analysis is used to predict module-module junction compatibility. FIG. 2F is a phylogenetic tree generated by multiple sequence alignments of FK-family KS and ACP domains, in which group-specific residues have been concatenated into functional clades or sub-clades. The distance between modules can be used to predict module-module junction compatibility. FIG. 2G is a schematic depicting the compatibility relationships predicted by evolutionary trace analysis between KS and ACP domains for the FK-family.

FIG. 3A is a schematic depicting a single module swap in which a donor module replaces either module 3 or module 4 of the PKS gene cluster that produces Compound 1.

FIG. 3B is an image of the engineered PKS that includes the heterologous module 3 from the S17 Streptomyces strain in place of the homologous module 3 in the PKS that produces Compound 1. The engineered PKS module 3 now includes an ER domain, and thus, the resulting compound produced by the engineered PKS, Compound 2, is reduced relative to Compound 1.

FIG. 3C is an image depicting compounds, e.g., Compound 2, Compound 3, Compound 4, and Compound 5, produced by single module swaps of either module 3 or module 4 in the PKS that produces Compound 1 with compatible heterologous modules.

FIG. 4A is a schematic depicting combinatorial swapping of a dimodule unit.

FIG. 4B is a schematic depicting the synthesis of dimodule units from exogenous donor modules by a first round of Gibson assembly. The dimodule product is shown as analyzed by DNA gel electrophoresis.

FIG. 4C is a schematic depicting dimodule capture, amplification, and enrichment in a shuttle vector. Dimodule units resulting from a first round of Gibson assembly are captured in a shuttle vector by a second round of Gibson assembly. This allows for the dimodule assembly to be amplified, enriched, and ligated into the intended PKS.

FIG. 4D is a schematic depicting the construction of dimodule libraries by combinatorial synthesis.

FIG. 4E is an image depicting the possible resulting compounds that may be generated by an exemplary dimodule library swapped into module 3 and module 4 of the PKS that produces Compound 1.

FIG. 4F depicts oversampling required for sufficient coverage of a large combinatorial dimodule library. FIG. 4F is a graphical representation of the oversampling required to achieve 90% or greater coverage of a 225 member dimodule combinatorial library. 18% of the 650 sampled clones were found to have produced polyketide compounds resulting from the engineered PKS cluster, as determined by LC-TOF mass spectrometry analysis.

FIG. 4G is a schematic depicting a method of preparing combinatorial dimodule libraries and characterizing the resulting libraries using NanoPore sequencing.

FIG. 4H is a schematic depicting the core informatics workflow for deconvoluting the sequences of combinatorial dimodule libraries by NanoPore sequencing.

FIGS. 5A and 5B depict the construction of trimodule libraries by combinatorial synthesis. FIG. 5A is a schematic illustrating a trimodule swap of modules 4, 5, and 6 of the PKS cluster that produces Compound 7, to produce a theoretical library size of 2,197 engineered polyketide synthases. FIG. 5b is an image of high efficiency trimodule assembly by Gibson assembly as analyzed by DNA gel electrophoresis.

FIG. 6A is a schematic illustrating a module swap that results in ring expansion by exchanging a single module acceptor for a dimodule donor. The resulting expanded ring compound produced by the engineered PKS, Compound 8, is also depicted.

FIG. 6B is a spectrogram that shows the production of an expanded ring compound, Compound 8, as analyzed by LC-TOF mass spectrometry.

FIG. 7A is schematic depicting the enzymatic domains of five PKS loading modules, including Rapamycin and novel PKS cluster, X23. Also shown is the starter unit associated with each loading module.

FIG. 7B depicts the compounds produced by engineered PKS clusters resulting from single module swaps in the X23 PKS cluster. The products include Compound 11 and 12, which are produced by an engineered PKS that contains a heterologous loading module.

### DETAILED DESCRIPTION OF THE INVENTION

The present invention describes compositions and methods for the production of polyketide compounds by an engineered polyketide synthase that includes one or more heterologous modules. The present invention also describes methods for predicting the compatibility of linking sequences of heterologous module-module junctions to produce an engineered polyketide synthase that does not substantially inhibit translocation during polyketide biosynthesis.

### Compounds

Compounds that may be produced with the methods of the invention include, but are not limited to, polyketides and polyketide macrolide antibiotics such as erythromycin; hybrid polyketides/non-ribosomal peptides such as rapamycin and FK506; carbohydrates including aminoglycoside antibiotics such as gentamicin, kanamycin, neomycin, tobramycin; benzofuranoids; benzopyranoids; flavonoids; glycopeptides including vancomycin; lipopeptides including daptomycin; tannins; lignans; polycyclic aromatic natural products, terpenoids, steroids, sterols, oxazolidinones including linezolid; amino acids, peptides and peptide antibiotics including polymyxins, non-ribosomal peptides, β-lactams antibiotics including carbapenems, cephalosporins, and penicillin; purines, pteridines, polypyrroles, tetracyclines, quinolones and fluoroquinolones; and sulfonamides.

### Proteins

### Polyketide Synthases

Polyketide synthases (PKSs) are a family of multi-domain enzymes that produce polyketides. Type I polyketide synthases are large, modular proteins which include several domains organized into modules. The modules generally present in a polyketide synthase include i) a loading module; ii) extending modules; and iii) releasing and/or cyclization modules depending on whether the final polyketide is linear or cyclic. The domains which generally are found in the modules are acyltransferase (AT), acyl carrier protein (ACP), keto-synthase (KS), ketoreductase (KR), dehydratase (DH), enoylreductase (ER), methyltransferase (MT), sulfhydrolase (SH), and thioesterase (TE).

A polyketide chain and the starter groups are generally bound to the thiol groups of the active site cysteines in the ketosynthase domain (the polyketide chain) and acyltransferase domain (the loading group and malonyl extender units) through a thioester linkage. Binding to acyl carrier protein (ACP) is mediated by the thiol of the phosphopantetheinyl group, which is bound to a serine hydroxyl of ACP, to form a thioester linkage to the growing polyketide chain. The growing polyketide chain is handed over from one thiol group to another by trans-acylations and is released after synthesis by hydrolysis or cyclization.

The synthesis of a polyketide begins by a starter unit, being loaded onto the acyl carrier protein domain of the PKS catalyzed by the acyltransferase in the loading module. An extender unit, e.g., a malonyl-CoA, is loaded onto the acyl carrier protein domain of the current module catalyzed by another acyltransferase domain. The polyketide chain is then elongated by subsequent extender modules after being passed from the acyl carrier protein domain of module n to the ketosynthase domain of the n+1 module. The acyl carrier protein bound extender unit reacts with the polyketide chain bound to the ketosynthase domain with expulsion of CO₂ to produce an extended polyketide chain bound to the acyl carrier protein. Each added extender unit may then be modified by β-ketoprocessing domains, i.e., ketoreductase (which reduces the carbonyl of the elongation group to a hydroxy), dehydratase (which expels H₂O to produce an alkene), and enoylreductase (which reduces alkenes to produce saturated hydrocarbons). Once the synthesis of the polyketide is complete, a thioesterase domain in the releasing modules hydrolyzes the completed polyketide chain from the acyl carrier protein of the last extending module. The compound released from the PKS may then be further modified by other proteins, e.g., nonribosomal peptide synthase. In some cases, the biosynthetic cluster harbors polyketide megasynthases and a non-ribosomal peptide synthase (NRPS). This hybrid architecture is referred to as hybrid PKS/NRPS.

### Polyketide Synthase Extender Modules

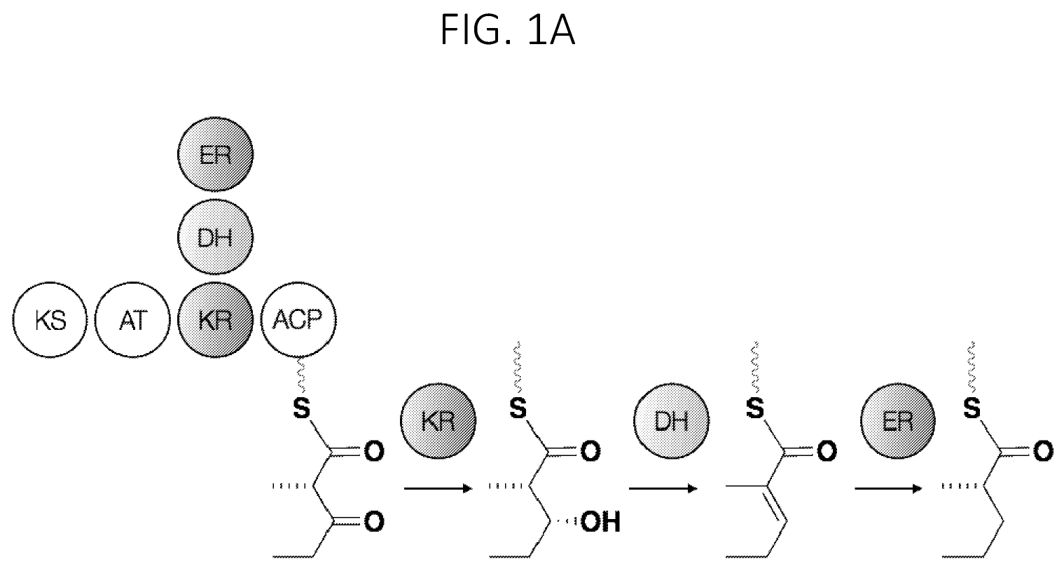

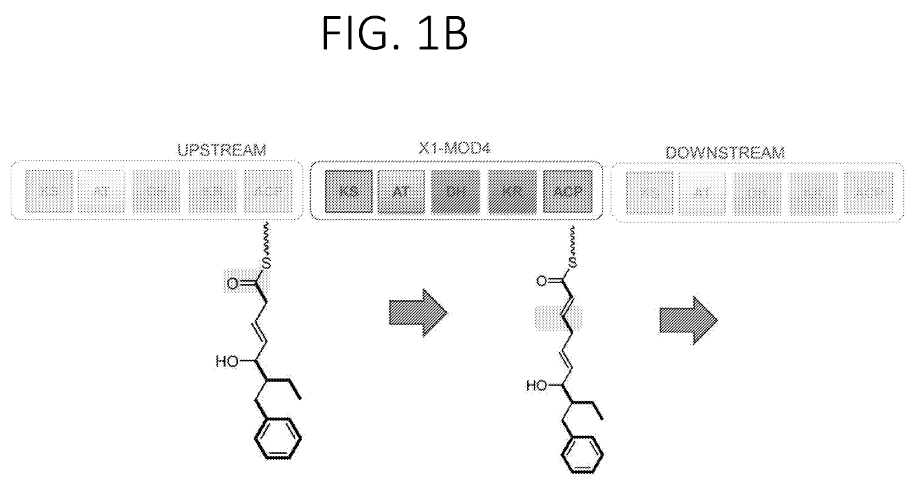

PKS biosynthesis proceeds by two key mechanisms: polyketide chain elongation within a module and translocation between modules (FIGS. 1A and 1B). The basic functional unit of polyketide synthase clusters is the extender module, which encodes a 2-carbon extender unit derived from malonyl-CoA. Within the extender module, the minimal domain architecture required for polyketide chain elongation includes the ketosynthase (KS), acyl-transferase (AT) and the ACP (acyl-carrier protein) domains, and the specific chemistry of each module is encoded by the AT domain and by the presence of the beta-carbonyl processing domains: ketoreductase (KR), dehydratase (DH), and enoylreductase (ER) domains. Productive chain elongation depends on the concerted function of numerous domains

### β-Ketone Processing Domains

β-ketone processing domains are the domains in a PKS which result in modification of the elongation groups added during the synthesis of a polyketide. Each β-ketone processing domain is capable of changing the oxidation state of an elongation group. The β-ketone processing domains include ketoreductase (which reduces the carbonyl of the elongation group to a hydroxy), dehydratase (which expels H₂O to produce an alkene), and enoylreductase (which reduces alkenes to produce saturated hydrocarbons).

### Module Swapping to Produce Engineered Polyketide Synthases

The present disclosure provides methods and compositions related to engineered polyketide synthases produced by swapping modules between related PKS clusters. Polyketide translocation is controlled by protein-protein interactions at the inter-modular junctions. In some embodiments, module swapping is guided by bioinformatic predictions to determine which modules have the highest probability of functioning in assembly-line polyketide biosynthesis. Multiple bioinformatics methods are used to determine the structural information in PKS sequence alignments to predict protein-protein interactions that mediate polyketide translocation at the inter-modular junction. The present disclosure includes a DNA assembly strategy to swap one or more heterologous donor modules for one or more acceptor modules to generate hybrid PKS clusters.

In some embodiments, module swapping is achieved by single, di- or tri-, or multi-module capture. In some embodiments, module swapping may be performed by exchange of the loading module. In some embodiments, module swapping may be performed by exchange of one or more extender modules. In some embodiments, module swapping may be performed by exchange of one or more releasing or cyclization modules. In some embodiments, two or more heterologous donor modules may replace a single acceptor module which may result in the production of a ring-expanded compound. In some embodiments, a single heterologous donor module may replace two or more acceptor modules which may result in a contracted ring compound. In some embodiments, the engineered polyketide synthases may produce novel compounds.

### Combinatorial Libraries of Engineered Polyketide Synthases

In some embodiments, the pooled capture and transfer of single, di- or tri-, or multi-module units enables the production of combinatorial libraries of engineered polyketide synthases. A dimodule unit, for example, consists of two heterologous modules, each of which may be independently selected from a pool of heterologous modules. A trimodule unit, example, consists of three heterologous modules, each of which may be independently selected from a pool of heterologous modules. One or more modules of a polyketide synthase may be replaced with a single, di-, tri-, or multi-module unit, where the single, di-, tri- or multi-module unit is selected from a pool of single- di-, tri- or multi-module units produced by combinatorial synthesis. Exemplary methods for the production of combinatorial libraries of engineered polyketide synthases (e.g., dimodule and trimodule combinatorial libraries) are provided in Examples 2 and 4.

### Characterization of Engineered PKS Libraries by Single-Molecule Long-Read Sequencing

In some embodiments of the invention, single-molecule long-read sequencing technology (e.g., Nanopore sequencing or SMRT sequencing) may be used to characterize libraries of engineered polyketide synthases which are produced by any of the methods described herein. In particular, single-molecule long-read sequencing (e.g., Nanopore sequencing or SMRT sequencing) may be used to characterize (e.g., deconvolute) combinatorial libraries of engineered polyketide synthases (e.g., combinatorial libraries of engineered polyketides synthases which are produced by pooled capture and transfer of single, di- or tri-, or multi-module units). Single-molecule long-read sequencing enables the identification of the module or modules which are incorporated into the combinatorial library. This further enables the prediction of the chemistry of the resulting plurality of engineered polyketide synthases. The predicted enzymatic chemistry can therefore be connected to the compounds produced by the engineered polyketide synthases. The resulting compounds may be identified by chemical methods of analysis known to one of skill in the art (e.g., mass spectrometry or high performance liquid chromatography). Furthermore, the predicted enzymatic chemistry can be connected to the function of the resulting compounds (e.g., binding to a target protein or inducing a phenotype, such as a cell based phenotype). Accordingly, long-read sequencing of a genetically encoded molecule may allow for genotypic-phenotypic linkage.

Single-molecule long-read sequencing technologies may be considered to include any sequencing technology which enables the sequencing of a single molecule of a biopolymer (e.g., a polynucleotide such as DNA or RNA), and which enables read lengths of greater than 2 kilobases (e.g., greater than 5 kilobases, greater than 10 kilobases, greater than 20 kilobases, greater than greater than 50 kilobases, or greater 100 kilobases). Single-molecule long-read sequencing technologies may enable the sequencing of multiple single molecules of DNA or RNA in parallel. Single-molecule long-read sequencing technologies may include sequencing technologies that rely on individual compartmentalization of each molecule of DNA or RNA being sequenced.

Nanopore sequencing is an exemplary single-molecule long-read sequencing technology that may be used to characterize libraries of engineered polyketide synthases that are prepared by any of the methods described herein. Nanopore sequencing enables the long-read sequencing of single molecules of of biopolymers (e.g., polynucleotides such as DNA or RNA). Nanopore sequencing relies on protein nanopores set in an electrically resistant polymer membrane. An ionic current is passed through the nanopores by setting a voltage across this membrane. If an analyte (e.g., a biopolymer such as DNA or RNA) passes through the pore or near its aperture, this event creates a characteristic disruption in current. The magnitude of the electric current density across a nanopore surface depends on the composition of DNA or RNA (e.g., the specific base) that is occupying the nanopore. Therefore, measurement of the current makes it possible to identify the sequence of the molecule in question. Exemplary methods for the use of Nanopore sequencing to characterize combinatorial libraries of engineered polyketide synthases are provided in Example 3.

Single molecule real-time (SMRT) sequencing (PacBio) is an exemplary single-molecule long-read sequencing technology that may be used to characterize libraries of engineered polyketide synthases that are prepared by any of the methods described herein. SMRT is a parallelized single molecule DNA sequencing method. SMRT utilizes a zero-mode waveguide (ZMW). A single DNA polymerase enzyme is affixed at the bottom of a ZMW with a single molecule of DNA as a template. The ZMW is a structure that creates an illuminated observation volume that is small enough to observe only a single nucleotide of DNA being incorporated by DNA polymerase. Each of the four DNA bases is attached to one of four different fluorescent dyes. When a nucleotide is incorporated by the DNA polymerase, the fluorescent tag is cleaved off and diffuses out of the observation area of the ZMW where its fluorescence is no longer observable. A detector detects the fluorescent signal of the nucleotide incorporation, and the base call is made according to the corresponding fluorescence of the dye.

### Computational Approaches for the Prediction of Functional Inter-Modular Junctions

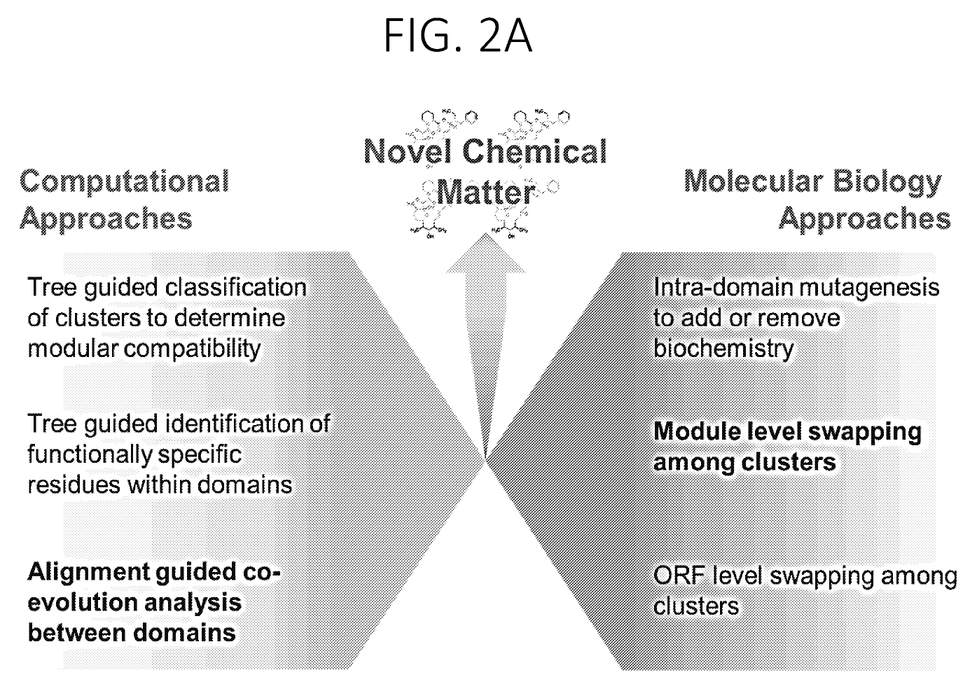

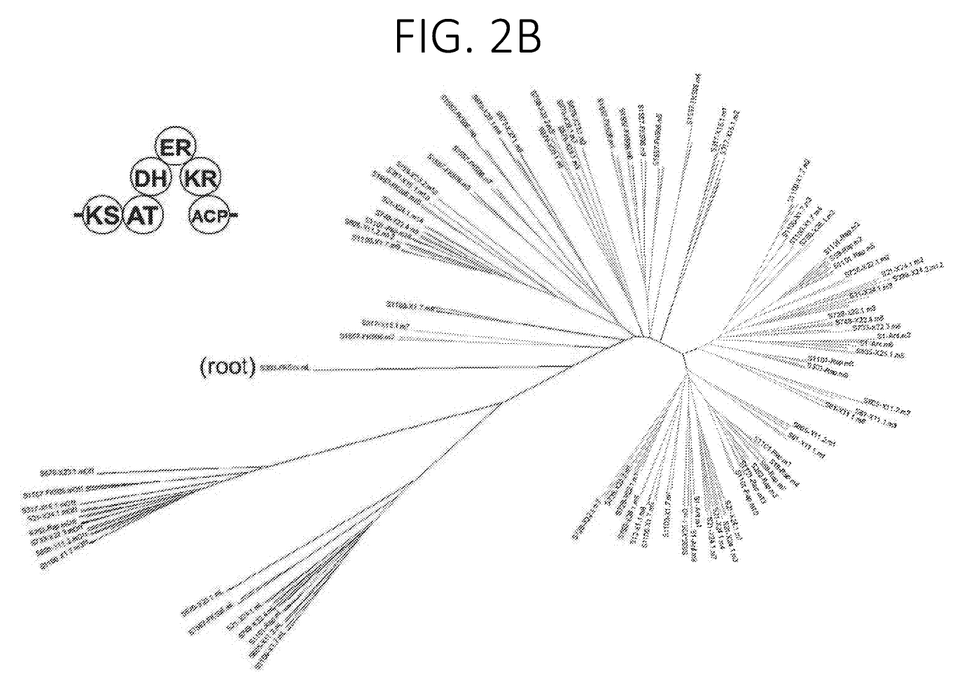

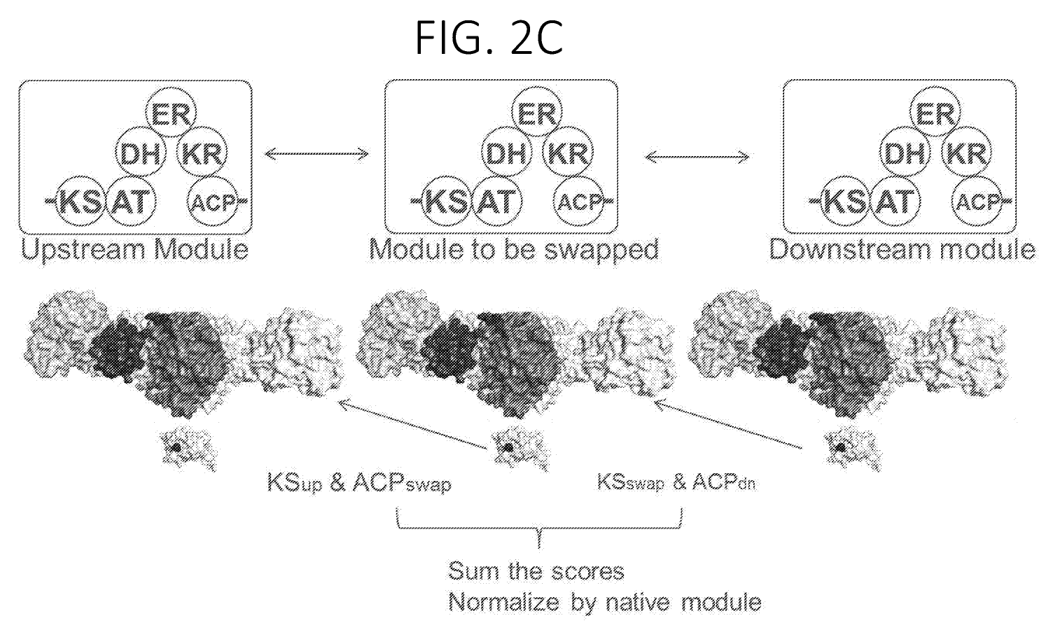

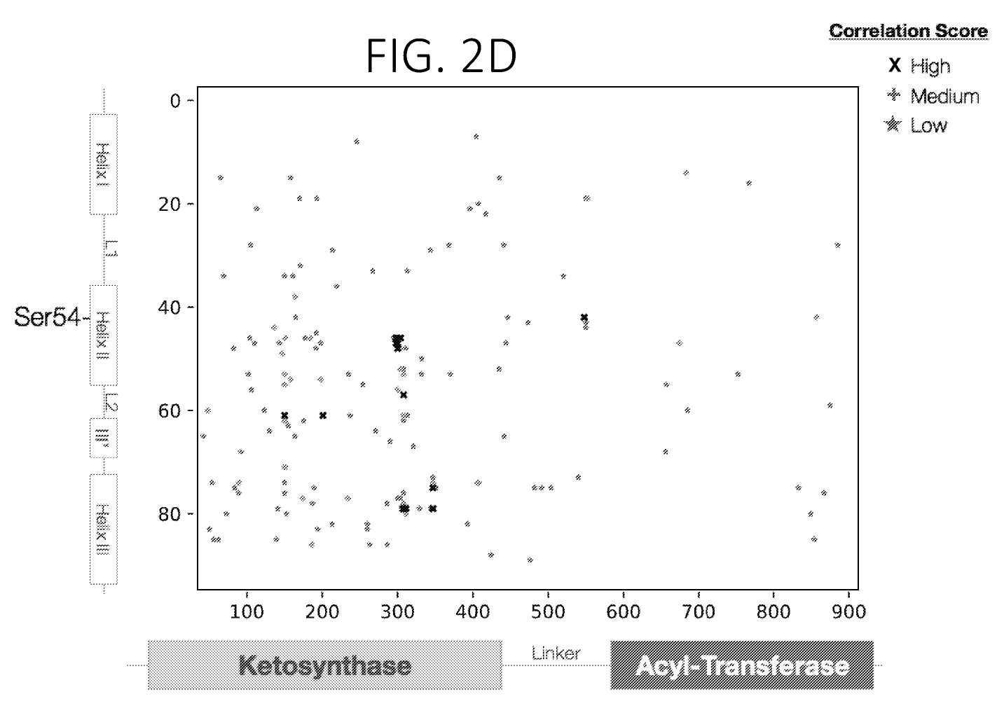

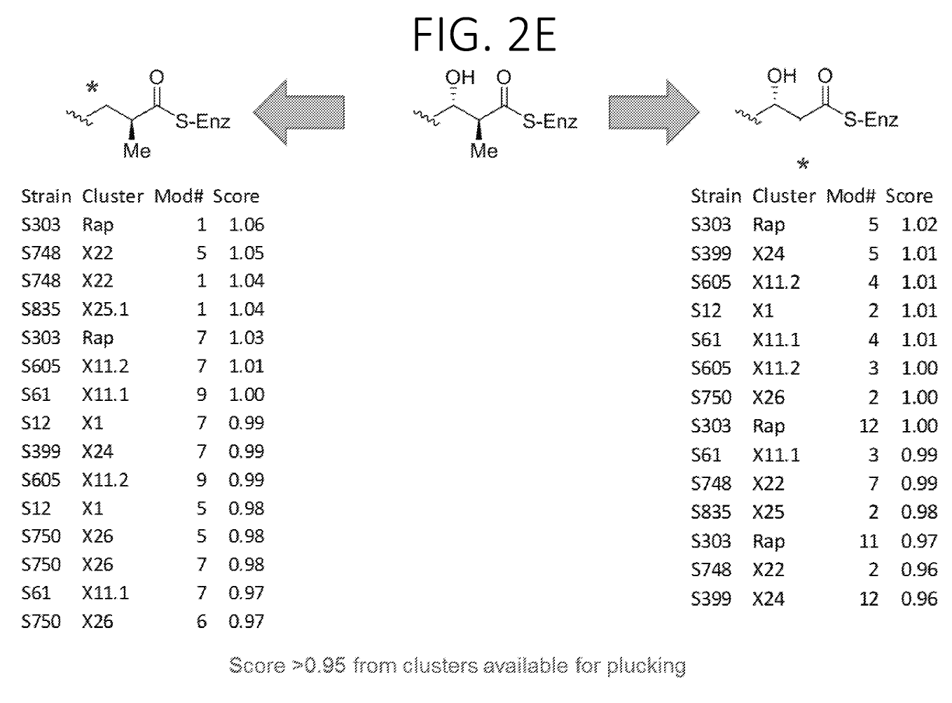

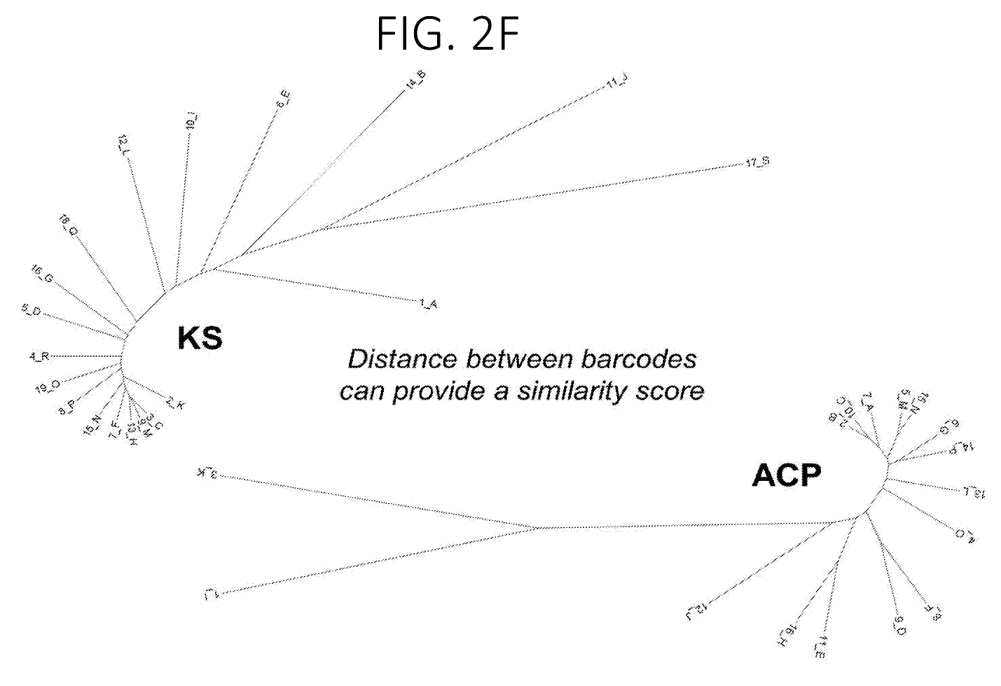

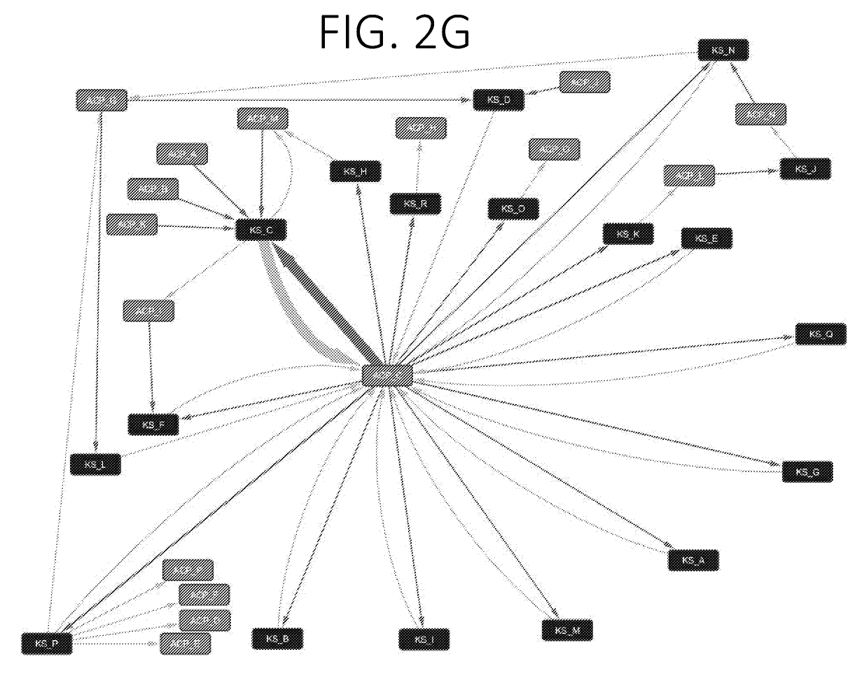

The present disclosure provides complementary bioinformatic approaches for the prediction of functional protein-protein interactions at the module-module junction (FIG. 2A). In some embodiments, these bioinformatic approaches serve as the predictive basis for the design of chimeric PKS proteins by module swapping.

### Module-Level Phylogeny

Sequence divergence between polyketide modules and inter-module linkers suggests importance in module-module compatibility. In some embodiments, a module-level phylogenic map may be constructed by multiple sequence alignment of PKS modules. For example, a module-level phylogenic map was generated by multiple sequence alignments of complete FK-family modules (FIG. 2B). This enabled the identification of 10 module clades including 8 elongation, 1 loading, and 1 off-loading. In some embodiments, a heterologous module is compatible if it is present in the same module clade as the adjacent modules.

### Inter-Module Residue Covariation

Inter-module residue covariation across the intermodular junction was computed to generate an algorithm to rank order intermodule compatibility (FIGS. 2C-2E). Type I polyketide synthase protein sequences were extracted from Genbank and an internal database using Hidden Markov Models trained on the ketosynthase (KS) and acyl carrier protein (ACP) domains. Shorter peptide sequences, starting with the ACP of a module and extending through the KS and acyl transferase (AT) of the following module, were extracted to generate a multiple alignment. Positions not aligning to an amino acid from PDB entry 2JU1 (for the ACP) or 2HG4 (for KS and AT and associated linkers) were removed to compress the multiple alignment. Evolutionary couplings were then calculated using the package FreeContact. These couplings take the form of a score matrix with two indices: the first amino acid position in the multiple alignment (I) and the second amino acid position in the multiple alignment (J, which is always greater than I) and the amino acid at position J. I,J pairs with a score above a specified cutoff and in which I is within the ACP and J within the KS-AT didomain are saved.

To generate a score for a potential single module substitution, the following alignments are retrieved from the original multiple alignment: the ACP for the upstream domain, the ACP and KS-AT didomain for the inserted module, and the KS for the downstream module. These are used to synthesize two rows compatible with the original multiple alignment: one with the ACP of the upstream module and KS-AT of the inserted module and a second with the ACP of the inserted module and KS-AT of the downstream module. For each I,J pair in the saved coupling matrix, the amino acids at position I and J in the synthesized alignment are retrieved (aaI, aaJ). The mutual information for this amino acid pair within the alignment is multiplied by the coupling score to generate a raw score. The raw scores are computed for each I,J pair in the saved coupling matrix and for each of the two synthesized alignments. The sum of the raw scores for the heterologous donor domain is divided by the sum of the raw scores for the homologous native domain to generate a normalized percentage score. Candidate swaps with the same chemistry are ranked by this score. In the case of multiple module swaps, the process is expanded, e.g., if N donor domains are to be swapped in, then one synthetic alignment is generated for the preceding module's ACP domain and the first donor module's KS-AT didomain, another for the first donor modules' ACP domain and the second donor module's KS-AT didomain and so forth, concluding with the final donor domain's ACP and the first module of the recipient synthase downstream of the breakpoint. Scores are computed and normalized in the same manner: the scores for the swapped modules are normalized for the score computed for the native modules. In some embodiments, a heterologous module is compatible if the module is assigned a score of greater than or equal to 0.90 in the inter-module covariation analysis algorithm described herein.

Evolutionary Trace Analysis to Identify Modules within Functional Clades or Sub-Clades

As an additional test of module compatibility, evolutionary trace analysis may be used to identify modules that belong to the same functional clade or sub-clade (FIGS. 2F-2G). For example, phylogenetic trees with uniform branch lengths were constructed based on multiple sequence alignments of FK-family KSs and ACPs. For every non-terminal node in a tree, a vertical cutoff was applied by which terminal nodes were partitioned into groups based on shared parental nodes at the cutoff. Residues globally conserved across all groups and residues locally conserved within groups, but specific to a given group, were identified as functional residues. Globally conserved residues suggest rules that likely must be observed for all members of the FK-family. Group-specific residues suggest guidelines that may provide predictive power for engineering within the FK class. For each tree, the earliest cutoff at which the number of group-specific residues exceeded the number of globally conserved residues was selected for further analysis. Group-specific residues were concatenated into functional clades and unrooted phylogenetic trees of the clades were constructed. Distances between terminal nodes in the phylogenetic tree were used to create an evolutionary distance score (EDS). The KS and ACP EDSs between a homologous acceptor module and a proposed heterologous donor module were calculated and used to predict engineering compatibility. KS and ACP clade classifications were then used to create network maps of neighboring KSs and ACPs weighted by the frequency a given KS-ACP or ACP-KS pair was observed in FK-family polyketides. Superimposing a proposed module swap onto the network map was used to predict engineering compatibility with upstream ACPs and downstream KSs. In some embodiments, a heterologous module is compatible if the module belongs to the same functional evolutionary clade or sub-clade as one or more adjacent modules in the reference PKS.

### Regulation of Polyketide Synthase Expression

The Large ATP-binding regulators of the LuxR family of transcriptional activators (LALs) are known transcriptional regulators of polyketides such as FK506 or rapamycin. The LAL family has been found to have an active role in the induction of expression of some types of natural product gene clusters, for example PikD for pikromycin production and RapH for rapamycin production. Binding of the LAL or multiple LALs in a complex to specific sites in the promoters of genes within a gene cluster that produces a small molecule (e.g., a polyketide synthase gene cluster) potentiates expression of the gene cluster and hence promotes production of the compound (e.g., a polyketide). In some embodiments, LALs may be used for the regulation of the expression of engineered PKS clusters.

### LALs

LALs include three domains, a nucleotide-binding domain, an inducer-binding domain, and a DNA-binding domain. A defining characteristic of the structural class of regulatory proteins that include the LALs is the presence of the AAA+ ATPase domain. Nucleotide hydrolysis is coupled to large conformational changes in the proteins and/or multimerization, and nucleotide binding and hydrolysis represents a “molecular timer” that controls the activity of the LAL (e.g., the duration of the activity of the LAL). The LAL is activated by binding of a small-molecule ligand to the inducer binding site. In most cases the allosteric inducer of the LAL is unknown. In the case of the related protein MalT, the allosteric inducer is maltotriose. Possible inducers for LAL proteins include small molecules found in the environment that trigger compound (e.g., polyketide) biosynthesis. The regulation of the LAL controls production of compound-producing proteins (e.g., polyketide synthases) resulting in activation of compound (e.g., polyketide) production in the presence of external environmental stimuli. Therefore, there are gene clusters that produce small molecules (e.g., PKS gene clusters) which, while present in a strain, do not produce compound either because (i) the LAL has not been activated, (ii) the strain has LAL binding sites that differ from consensus, (iii) the strain lacks an LAL regulator, or (iv) the LAL regulator may be poorly expressed or not expressed under laboratory conditions. Since the DNA binding region of the LALs of the known PKS LALs are highly conserved, the known LALs may be used interchangeably to activate PKS gene clusters other than those which they naturally regulate. In some embodiments, the LAL is a fusion protein.

In some embodiments, an LAL may be modified to include a non-LAL DNA-binding domain, thereby forming a fusion protein including an LAL nucleotide-binding domain and a non-LAL DNA-binding domain. In certain embodiments, the non-LAL DNA-binding domain is capable of binding to a promoter including a protein-binding site positioned such that binding of the DNA-binding domain to the protein-binding site of the promoter promotes expression of a gene of interest (e.g., a gene encoding a compound-producing protein, as described herein). The non-LAL DNA binding domain may include any DNA binding domain known in the art. In some instances, the non-LAL DNA binding domain is a transcription factor DNA binding domain. Examples of non-LAL DNA binding domains include, without limitation, a basic helix-loop-helix (bHLH) domain, leucine zipper domain (e.g., a basic leucine zipper domain), GCC box domain, helix-turn-helix domain, homeodomain, srf-like domain, paired box domain, winged helix domain, zinc finger domain, HMG-box domain, Wor3 domain, OB-fold domain, immunoglobulin domain, B3 domain, TAL effector domain, Cas9 DNA binding domain, GAL4 DNA binding domain, and any other DNA binding domain known in the art. In some instances, the promoter is positioned upstream to the gene of interest, such that the fusion protein may bind to the promoter and induce or inhibit expression of the gene of interest. In certain instances, the promoter is a heterologous promoter introduced to the nucleic acid (e.g., a chromosome, plasmid, fosmid, or any other nucleic acid construct known in the art) containing the gene of interest. In other instances, the promoter is a pre-existing promoter positioned upstream to the gene of interest. The protein-binding site within the promoter may, for example, be a non-LAL protein-binding site. In certain embodiments, the protein-binding site binds to the non-LAL DNA binding domain, thereby forming a cognate DNA binding domain/protein-binding site pair.

In some embodiments, the LAL is encoded by a nucleic acid having at least 70% (e.g., at least 70%, at least 75%, at least 80%, at least 85%, at least 90%, at least 95%, at least 99%) sequence identity to any one of SEQ ID Nos: 180-212 or has a sequences with at least 70% (e.g., at least 70%, at least 75%, at least 80%, at least 85%, at least 90%, at least 95%, at least 99%) sequence identity to any one of SEQ ID Nos: 180-212.

SEQ ID NO: 180

ATGCCTGCCGTGGAGTGCTATGAACTGGACGCCCGCGATGACGAGCTCAG
AAAACTGGAGGAGGTTGTGACCGGGCGGGCCAACGGCCGGGGTGTGGTGG
TCACCATCACCGGACCGATCGCCTGCGGCAAGACCGAACTGCTCGACGCA
GCCGCCGCGAAGGCCGACGCCATCACGTTACGAGCGGTCTGCTCCGCGGA
GGAACAGGCACTCCCGTACGCCCTGATCGGGCAGCTCATCGACAACCCGG
CGCTCGCCTCCCACGCGCTGGAGCCGGCCTGCCCGACCCTCCCGGGCGAG
CACCTGTCGCCGGAGGCCGAGAACCGGCTGCGCAGCGACCTCACCCGTAC
CCTGCTGGCGCTCGCCGCCGAACGGCCGGTGCTGATCGGCATCGACGAGT
CACACGCGAACGCTTTGTGTCTGCTCCACCTGGCCCGAAGGGTCGGCTCG
GCCCGGATCGCCATGGTCCTCACCGAGTTGCGCCGGCTCACCCCGGCCCA
CTCACAGTTCCAGGCCGAGCTGCTCAGCCTGGGGCACCACCGCGAGATCG
CGCTGCGCCCGCTCAGCCCGAAGCACACCGCCGAGCTGGTCCGCGCCGGT
CTCGGTCCCGACGTCGACGAGGACGTGCTCACGGGGTTGTACCGGGCGAC
CGGCGGCAACCTGAACCTCACCCGCGGACTGATCAACGATGTGCGGGAGG
CCTGGGAGACGGGAGGGACGGGCATCAGCGCGGGCCGCGCGTACCGGCTG
GCATACCTCGGTTCCCTCTACCGCTGCGGCCCGGTCCCGTTGCGGGTCGC
ACGGGTGGCCGCCGTGCTGGGCCAGAGCGCCAACACCACCCTGGTGCGCT
GGATCAGCGGGCTCAACGCGGACGCGGTGGGCGAGGCAACCGAGATCCTC
ACCGAAGGCGGCCTGCTGCACGACCTGCGGTTCCCGCACCCGGCGGCCCG
TTCGGTGGTACTCAACGACATGTCCGCCCAGGAACGACGCCGCCTGCACC
GGTCCGCTCTGGAAGTGCTGGACGACGTGCCCGTGGAAGTGGTCGCGCAC
CACCAGGTCGGCGCCGGTCTCCTGCACGGCCCGAAGGCCGCCGAGATATT
CGCCAAGGCCGGCCAGGAGCTGCATGTGCGCGGCGAGTTGGACACCGCGT
CCGACTATCTGCAACTGGCCCACCAGGCCTCCGACGACGCCGTCACCGGG
ATGCGGGCCGAGGCCGTGGCGATCGAGCGCCGCCGCAACCCGCTGGCCTC
GAGCCGGCACCTCGACGAGCTGACCGTCGTCGCCCGTGCCGGGCTGCTCT
TCCCCGAGCACACGGCGCTGATGATCCGCTGGCTGGGCGTCGGCGGGCGG
TCCGGCGAGGCAGCCGGGCTGCTGGCCTCGCAGCGCCCCCGTGCGGTCAC
CGACCAGGACAGGGCCCATATGCGGGCCGCCGAGGTATCGCTCGCGCTGG
TCAGCCCCGGCACGTCCGGCCCGGACCGGCGGCCGCGTCCGCTCACGCCG
GATGAGCTCGCGAACCTGCCGAAGGCGGCCCGGCTCTGCGCGATCGCCGA
CAATGCCGTCATGTCGGCCCTGCGCGGTCGTCCCGAGCTCGCCGCGGCCG
AGGCGGAGAACGTCCTGCAGCACGCCGACTCGGCGGCGGCCGGCACCACC
GCCCTCGCCGCGCTGACCGCCTTGCTGTACGCGGAGAACACCGACACCGC
TCAGCTCTGGGCCGACAAGCTGGTCTCCGAGACCGGGGCGTCGAACGAGG
AGGAGGCGGGCTACGCGGGGCCGCGCGCCGAAGCCGCGTTGCGTCGCGGC
GACCTGGCCGCGGCGGTCGAGGCAGGCAGCACCGTTCTGGACCACCGGCG
GCTCTCGACGCTCGGCATCACCGCCGCGCTACCGCTGAGCAGCGCGGTGG
CCGCCGCCATCCGGCTGGGCGAGACCGAGCGGGCGGAGAAGTGGCTCGCC
CAGCCGCTGCCGCAGGCCATCCAGGACGGCCTGTTCGGCCTGCACCTGCT
CTCGGCGCGCGGCCAGTACAGCCTCGCCACGGGCCAGCACGAGTCGGCGT
ACACGGCGTTTCGCACCTGCGGGGAACGTATGCGGAACTGGGGCGTTGAC
GTGCCGGGTCTGTCCCTGTGGCGCGTCGACGCCGCCGAGGCGCTGCTGCA
CGGCCGCGACCGGGACGAGGGCCGACGGCTCGTCGACGAGCAACTCACCC
GTGCGATGGGACCCCGTTCCCGCGCCTTGACGCTGCGGGTGCAGGCGGCG
TACAGCCCGCCGGCGAAGCGGGTCGACCTGCTCGATGAAGCGGCCGACCT
GCTGCTCTCCTGCAACGACCAGTACGAGCGGGCACGGGTGCTCGCCGACC
TGAGCGAGACGTTCAGCGCGCTCCGGCACCACAGCCGGGCGCGGGGACTG
CTTCGGCAGGCCCGGCACCTGGCCGCCCAGCGCGGCGCGATACCGCTGCT
GCGCCGACTCGGGGCCAAGCCCGGAGGCCCCGGCTGGCTGGAGGAATCCG
GCCTGCCGCAGCGGATCAAGTCGCTGACCGACGCGGAGCGGCGGGTGGCG
TCGCTGGCCGCCGGCGGACAGACCAACCGCGTGATCGCCGACCAGCTCTT
CGTCACGGCCAGCACGGTGGAGCAGCACCTCACGGACGTCTCCACTGGGT
CAAGGCCGCCAGCACCTGCCGCCGAACTCGTCTAG

SEQ ID NO: 181

ATGCCTGCCGTGGAGTGCTATGAACTGGACGCCCGCGATGACGAGCTCAG
AAAACTGGAGGAGGTTGTGACCGGGCGGGCCAACGGCCGGGGTGTGGTGG
TCACCATCACCGGACCGATCGCCTGCGGCAAGACCGAACTGCTCGACGCA
GCCGCCGCGAAGGCCGACGCCATCACGCTGCGAGCGGTCTGCTCCGCGGA
GGAACAGGCACTCCCGTACGCCCTGATCGGGCAGCTCATCGACAACCCGG
CGCTCGCCTCCCACGCGCTGGAGCCGGCCTGCCCGACCCTCCCGGGCGAG
CACCTGTCGCCGGAGGCCGAGAACCGGCTGCGCAGCGACCTCACCCGTAC
CCTGCTGGCGCTCGCCGCCGAACGGCCGGTGCTGATCGGCATCGACGAGT
CACACGCGAACGCTTTGTGTCTGCTCCACCTGGCCCGAAGGGTCGGCTCG
GCCCGGATCGCCATGGTCCTCACCGAGTTGCGCCGGCTCACCCCGGCCCA
CTCACAGTTCCAGGCCGAGCTGCTCAGCCTGGGGCACCACCGCGAGATCG
CGCTGCGCCCGCTCAGCCCGAAGCACACCGCCGAGCTGGTCCGCGCCGGT
CTCGGTCCCGACGTCGACGAGGACGTGCTCACGGGGTTGTACCGGGCGAC
CGGCGGCAACCTGAACCTCACCCGCGGACTGATCAACGATGTGCGGGAGG
CCTGGGAGACGGGAGGGACGGGCATCAGCGCGGGCCGCGCGTACCGGCTG
GCATACCTCGGTTCCCTCTACCGCTGCGGCCCGGTCCCGTTGCGGGTCGC
ACGGGTGGCCGCCGTGCTGGGCCAGAGCGCCAACACCACCCTGGTGCGCT
GGATCAGCGGGCTCAACGCGGACGCGGTGGGCGAGGCAACCGAGATCCTC
ACCGAAGGCGGCCTGCTGCACGACCTGCGGTTCCCGCACCCGGCGGCCCG
TTCGGTGGTACTCAACGACATGTCCGCCCAGGAACGACGCCGCCTGCACC
GGTCCGCTCTGGAAGTGCTGGACGACGTGCCCGTGGAAGTGGTCGCGCAC
CACCAGGTCGGCGCCGGTCTCCTGCACGGCCCGAAGGCCGCCGAGATATT
CGCCAAGGCCGGCCAGGAGCTGCATGTGCGCGGCGAGTTGGACACCGCGT
CCGACTATCTGCAACTGGCCCACCAGGCCTCCGACGACGCCGTCACCGGG
ATGCGGGCCGAGGCCGTGGCGATCGAGCGCCGCCGCAACCCGCTGGCCTC
GAGCCGGCACCTCGACGAGCTGACCGTCGTCGCCCGTGCCGGGCTGCTCT
TCCCCGAGCACACGGCGCTGATGATCCGCTGGCTGGGCGTCGGCGGGCGG
TCCGGCGAGGCAGCCGGGCTGCTGGCCTCGCAGCGCCCCCGTGCGGTCAC
CGACCAGGACAGGGCCCATATGCGGGCCGCCGAGGTATCGCTCGCGCTGG
TCAGCCCCGGCACGTCCGGCCCGGACCGGCGGCCGCGTCCGCTCACGCCG
GATGAGCTCGCGAACCTGCCGAAGGCGGCCCGGCTCTGCGCGATCGCCGA
CAATGCCGTCATGTCGGCCCTGCGCGGTCGTCCCGAGCTCGCCGCGGCCG
AGGCGGAGAACGTCCTGCAGCACGCCGACTCGGCGGCGGCCGGCACCACC
GCCCTCGCCGCGCTGACCGCCTTGCTGTACGCGGAGAACACCGACACCGC
TCAGCTCTGGGCCGACAAGCTGGTCTCCGAGACCGGGGCGTCGAACGAGG
AGGAGGCGGGCTACGCGGGGCCGCGCGCCGAAGCCGCGTTGCGTCGCGGC
GACCTGGCCGCGGCGGTCGAGGCAGGCAGCACCGTTCTGGACCACCGGCG
GCTCTCGACGCTCGGCATCACCGCCGCGCTACCGCTGAGCAGCGCGGTGG
CCGCCGCCATCCGGCTGGGCGAGACCGAGCGGGCGGAGAAGTGGCTCGCC
CAGCCGCTGCCGCAGGCCATCCAGGACGGCCTGTTCGGCCTGCACCTGCT
CTCGGCGCGCGGCCAGTACAGCCTCGCCACGGGCCAGCACGAGTCGGCGT
ACACGGCGTTTCGCACCTGCGGGGAACGTATGCGGAACTGGGGCGTTGAC
GTGCCGGGTCTGTCCCTGTGGCGCGTCGACGCCGCCGAGGCGCTGCTGCA
CGGCCGCGACCGGGACGAGGGCCGACGGCTCGTCGACGAGCAACTCACCC
GTGCGATGGGACCCCGTTCCCGCGCCTTGACGCTGCGGGTGCAGGCGGCG
TACAGCCCGCCGGCGAAGCGGGTCGACCTGCTCGATGAAGCGGCCGACCT
GCTGCTCTCCTGCAACGACCAGTACGAGCGGGCACGGGTGCTCGCCGACC
TGAGCGAGACGTTCAGCGCGCTCCGGCACCACAGCCGGGCGCGGGGACTG
CTTCGGCAGGCCCGGCACCTGGCCGCCCAGCGCGGCGCGATACCGCTGCT
GCGCCGACTCGGGGCCAAGCCCGGAGGCCCCGGCTGGCTGGAGGAATCCG
GCCTGCCGCAGCGGATCAAGTCGCTGACCGACGCGGAGCGGCGGGTGGCG
TCGCTGGCCGCCGGCGGACAGACCAACCGCGTGATCGCCGACCAGCTCTT
CGTCACGGCCAGCACGGTGGAGCAGCACCTCACGGACGTCTCCACTGGGT
CAAGGCCGCCAGCACCTGCCGCCGAACTCGTCTAG

SEQ ID NO: 182

GTGGTTCCTGAAGTGCGAGCAGCCCCCGACGAACTGATCGCCCGCGATGA
CGAGCTGAGCCGCCTCCAACGGGCACTCACCAGGGCGGGGAGCGGAAGGG
GCGGCGTCGTCGCCATCACCGGGCCCATCGCCAGCGGAAAGACGGCGCTG
CTCGACGCCGGAGCGGCCAAGTCCGGCTTCGTCGCACTCCGTGCGGTGTG
CTCCTGGGAAGAGCGCACTCTGCCGTACGGGATGCTGGGCCAGCTCTTCG
ACCATCCCGAACTGGCCGCCCAGGCGCCGGACCTTGCCCACTTCACGGCT
TCGTGCGAGAGCCCTCAGGCCGGTACCGACAACCGCCTGCGGGCCGAGTT
CACCCGCACCCTGCTGGCGCTCGCCGCGGACTGGCCCGTCCTGATCGGCA
TCGACGACGTGCACCACGCCGACGCGGAATCACTGCGCTGTCTGCTCCAC
CTCGCCCGCCGCATCGGCCCGGCCCGCATCGCGGTCGTACTGACCGAGCT
GCGCAGACCGACGCCCGCCGACTCCCGCTTCCAGGCGGAACTGCTGAGCC
TGCGCTCCTACCAGGAGATCGCGCTCAGACCGCTCACCGAGGCGCAGACC
GGCGAACTCGTACGTCGGCACCTCGGCGCGGAGACCCACGAGGACGTCTC
CGCCGATACGTTCCGGGCGACCGGCGGGAACCTGCTCCTCGGGCACGGTT
TGATCAATGACATCCGGGAGGCGCGGACAGCGGGACGGCCGGGGGTCGTC
GCGGGGCGGGCGTACCGGCTCGCGTACCTCAGCTCGCTCTACCGCTGCGG
CCCGAGCGCGCTGCGTGTCGCCCGGGCGTCCGCCGTGCTCGGCGCGAGCG
CCGAAGCCGTGCTCGTCCAGCGGATGACCGGACTGAACAAGGACGCGGTC
GAACAGGTCTATGAGCAGCTGAACGAGGGACGGCTGCTGCAGGGCGAGCG
GTTTCCGCACCCGGCGGCCCGCTCCATCGTCCTTGACGACCTGTCGGCCC
TGGAACGCAGAAACCTGCACGAGTCGGCGCTGGAGCTGCTGCGGGACCAC
GGCGTGGCCGGCAACGTGCTCGCCCGCCACCAGATCGGCGCCGGCCGGGT
GCACGGCGAGGAGGCCGTCGAGCTGTTCACCGGGGCCGCACGGGAGCACC
ACCTGCGCGGTGAACTGGACGACGCGGCCGGATACCTGGAACTCGCCCAC
CGTGCCTCCGACGACCCCGTCACGCGCGCCGCACTACGCGTCGGCGCCGC
CGCGATCGAGCGCCTCTGCAATCCGGTACGGGCAGGCCGGCATCTGCCCG
AGCTGCTCACCGCGTCGCGCGCGGGACTGCTCTCCAGCGAGCACGCCGTG
TCGCTCGCCGACTGGCTGGCGATGGGCGGGCGCCCGGGCGAGGCGGCCGA
GGTCCTCGCGACGCAGCGTCCCGCGGCCGACAGCGAGCAGCACCGCGCAC
TCCTGCGCAGCGGCGAGTTGTCCCTCGCGCTGGTCCACCCCGGCGCGTGG
GATCCGTTGCGCCGGACCGATCGGTTCGCCGCGGGCGGGCTCGGCTCGCT
TCCCGGACCCGCCCGGCACCGCGCGGTCGCCGACCAAGCCGTCATCGCGG
CGCTGCGTGGACGTCTCGACCGGGCGGACGCCAACGCGGAGAGCGTTCTC
CAGCACACCGACGCCACGGCGGACCGGACCACGGCCATCATGGCGTTGCT
GGCCCTGCTCTACGCGGAGAACACCGATGCTGTCCAGTTCTGGGTCGACA
AACTGGCCGGTGACGAGGGCACCAGGACACCGGCCGACGAGGCGGTCCAC
GCGGGGTTCAACGCCGAGATCGCGCTGCGCCGCGGCGACTTGATGAGAGC
CGTCGAGTACGGCGAGGCAGCGCTCGGCCACCGGCACCTGCCCACCTGGG
GAATGGCCGCCGCTCTGCCGCTGAGCAGCACCGTGGTTGCCGCGATCCGG
CTCGGCGACCTCGACAGGGCCGAGCGGTGGCTCGCCGAGCCGCTGCCGCA
GCAGACGCCGGAGAGCCTCTTCGGGCTGCACCTGCTCTGGGCCCGCGGGC
AGCACCACCTCGCGACCGGGCGGCACGGGGCGGCGTACACGGCGTTCAGG
GAATGCGGCGAGCGGATGCGGCGGTGGGCCGTCGACGTGCCGGGCCTGGC
CCTGTGGCGGGTCGACGCCGCCGAATCGCTGCTGCTGCTCGGCCGTGACC
GTGCCGAAGGACTGCGGCTCGTCTCCGAGCAGCTGTCCCGGCCGATGCGC
CCTCGCGCGCGCGTGCAGACGTTACGGGTACAGGCGGCCTACAGTCCGCC
GCCCCAACGGATCGACCTGCTCGAAGAGGCCGCCGACCTGCTGGTCACCT
GCAACGACCAGTACGAACTGGCAAACGTACTCAGCGACTTGGCAGAGGCC
TCCAGCATGGTCCGGCAGCACAGCAGGGCGCGGGGTCTGCTCCGCCGGGC
ACGGCACCTCGCCACCCAGTGCGGCGCCGTGCCGCTCCTGCGGCGGCTCG
GCGCGGAACCCTCGGACATCGGCGGAGCCTGGGACGCGACGCTGGGACAG
CGGATCGCGTCACTGACGGAGTCGGAGCGGCGGGTGGCCGCGCTCGCCGC
GGTCGGGCGTACGAACAGGGAGATCGCCGAGCAGCTGTTCGTCACGGCCA
GCACGGTGGAACAGCACCTCACGAACGTGTTCCGCAAACTGGCGGTGAAG
GGCCGCCAGCAGCTTCCGAAGGAACTGGCCGACGTCGGCGAGCCGGCGGA
CCGCGACCGCCGGTGCGGGTAG

SEQ ID NO: 183

ATGGTTCCTGAAGTGCGAGCAGCCCCCGACGAACTGATCGCCCGCGATGA
CGAGCTGAGCCGCCTCCAACGGGCACTCACCAGGGCGGGGAGCGGAAGGG
GCGGCGTCGTCGCCATCACCGGGCCCATCGCCAGCGGAAAGACGGCGCTG
CTCGACGCCGGAGCGGCCAAGTCCGGCTTCGTCGCACTCCGTGCGGTGTG
CTCCTGGGAAGAGCGCACTCTGCCGTACGGGATGCTGGGCCAGCTCTTCG
ACCATCCCGAACTGGCCGCCCAGGCGCCGGACCTTGCCCACTTCACGGCT
TCGTGCGAGAGCCCTCAGGCCGGTACCGACAACCGCCTGCGGGCCGAGTT
CACCCGCACCCTGCTGGCGCTCGCCGCGGACTGGCCCGTCCTGATCGGCA
TCGACGACGTGCACCACGCCGACGCGGAATCACTGCGCTGTCTGCTCCAC
CTCGCCCGCCGCATCGGCCCGGCCCGCATCGCGGTCGTACTGACCGAGCT
GCGCAGACCGACGCCCGCCGACTCCCGCTTCCAGGCGGAACTGCTGAGCC
TGCGCTCCTACCAGGAGATCGCGCTCAGACCGCTCACCGAGGCGCAGACC
GGCGAACTCGTACGTCGGCACCTCGGCGCGGAGACCCACGAGGACGTCTC
CGCCGATACGTTCCGGGCGACCGGCGGGAACCTGCTCCTCGGGCACGGTT
TGATCAATGACATCCGGGAGGCGCGGACAGCGGGACGGCCGGGGGTCGTC
GCGGGGCGGGCGTACCGGCTCGCGTACCTCAGCTCGCTCTACCGCTGCGG
CCCGAGCGCGCTGCGTGTCGCCCGGGCGTCCGCCGTGCTCGGCGCGAGCG
CCGAAGCCGTGCTCGTCCAGCGGATGACCGGACTGAACAAGGACGCGGTC
GAACAGGTCTATGAGCAGCTGAACGAGGGACGGCTGCTGCAGGGCGAGCG
GTTTCCGCACCCGGCGGCCCGCTCCATCGTCCTTGACGACCTGTCGGCCC
TGGAACGCAGAAACCTGCACGAGTCGGCGCTGGAGCTGCTGCGGGACCAC
GGCGTGGCCGGCAACGTGCTCGCCCGCCACCAGATCGGCGCCGGCCGGGT
GCACGGCGAGGAGGCCGTCGAGCTGTTCACCGGGGCCGCACGGGAGCACC
ACCTGCGCGGTGAACTGGACGACGCGGCCGGATACCTGGAACTCGCCCAC
CGTGCCTCCGACGACCCCGTCACGCGCGCCGCACTACGCGTCGGCGCCGC
CGCGATCGAGCGCCTCTGCAATCCGGTACGGGCAGGCCGGCATCTGCCCG
AGCTGCTCACCGCGTCGCGCGCGGGACTGCTCTCCAGCGAGCACGCCGTG
TCGCTCGCCGACTGGCTGGCGATGGGCGGGCGCCCGGGCGAGGCGGCCGA
GGTCCTCGCGACGCAGCGTCCCGCGGCCGACAGCGAGCAGCACCGCGCAC
TCCTGCGCAGCGGCGAGTTGTCCCTCGCGCTGGTCCACCCCGGCGCGTGG
GATCCGTTGCGCCGGACCGATCGGTTCGCCGCGGGCGGGCTCGGCTCGCT
TCCCGGACCCGCCCGGCACCGCGCGGTCGCCGACCAAGCCGTCATCGCGG
CGCTGCGTGGACGTCTCGACCGGGCGGACGCCAACGCGGAGAGCGTTCTC
CAGCACACCGACGCCACGGCGGACCGGACCACGGCCATCATGGCGTTGCT
GGCCCTGCTCTACGCGGAGAACACCGATGCTGTCCAGTTCTGGGTCGACA
AACTGGCCGGTGACGAGGGCACCAGGACACCGGCCGACGAGGCGGTCCAC
GCGGGGTTCAACGCCGAGATCGCGCTGCGCCGCGGCGACTTGATGAGAGC
CGTCGAGTACGGCGAGGCAGCGCTCGGCCACCGGCACCTGCCCACCTGGG
GAATGGCCGCCGCTCTGCCGCTGAGCAGCACCGTGGTTGCCGCGATCCGG
CTCGGCGACCTCGACAGGGCCGAGCGGTGGCTCGCCGAGCCGCTGCCGCA
GCAGACGCCGGAGAGCCTCTTCGGGCTGCACCTGCTCTGGGCCCGCGGGC
AGCACCACCTCGCGACCGGGCGGCACGGGGCGGCGTACACGGCGTTCAGG
GAATGCGGCGAGCGGATGCGGCGGTGGGCCGTCGACGTGCCGGGCCTGGC
CCTGTGGCGGGTCGACGCCGCCGAATCGCTGCTGCTGCTCGGCCGTGACC
GTGCCGAAGGACTGCGGCTCGTCTCCGAGCAGCTGTCCCGGCCGATGCGC
CCTCGCGCGCGCGTGCAGACGCTGCGGGTACAGGCGGCCTACAGTCCGCC
GCCCCAACGGATCGACCTGCTCGAAGAGGCCGCCGACCTGCTGGTCACCT
GCAACGACCAGTACGAACTGGCAAACGTACTCAGCGACTTGGCAGAGGCC
TCCAGCATGGTCCGGCAGCACAGCAGGGCGCGGGGTCTGCTCCGCCGGGC
ACGGCACCTCGCCACCCAGTGCGGCGCCGTGCCGCTCCTGCGGCGGCTCG
GCGCGGAACCCTCGGACATCGGCGGAGCCTGGGACGCGACGCTGGGACAG
CGGATCGCGTCACTGACGGAGTCGGAGCGGCGGGTGGCCGCGCTCGCCGC
GGTCGGGCGTACGAACAGGGAGATCGCCGAGCAGCTGTTCGTCACGGCCA
GCACGGTGGAACAGCACCTCACGAACGTGTTCCGCAAACTGGCGGTGAAG
GGCCGCCAGCAGCTTCCGAAGGAACTGGCCGACGTCGGCGAGCCGGCGGA
CCGCGACCGCCGGTGCGGGTAG

SEQ ID NO: 184

GTGATAGCGCGCTTATCTCCCCCAGACCTGATCGCCCGCGATGACGAGTT
CGGTTCCCTCCACCGGGCGCTCACCCGAGCGGGGGGCGGGCGGGGCGTCG
TCGCCGCCGTCACCGGGCCGATCGCCTGCGGCAAGACCGAACTCCTCGAC
GCCGCCGCGGCCAAGGCCGGCTTCGTCACCCTTCGCGCGGTGTGCTCCAT
GGAGGAGCGGGCCCTGCCGTACGGCATGCTCGGCCAGCTCCTCGACCAGC
CCGAGCTGGCCGCCCGGACACCGGAGCTGGTCCGGCTGACGGCATCGTGC
GAAAACCTGCCGGCCGACGTCGACAACCGCCTGGGGACCGAACTCACCCG
CACGGTGCTGACGCTCGCCGCGGAGCGGCCCGTACTGATCGGCATCGACG
ACGTGCACCACGCCGACGCGCCGTCGCTGCGCTGCCTGCTCCACCTCGCG
CGCCGCATCAGCCGGGCCCGTGTCGCCATCGTGCTGACCGAGCTGCTCCG
GCCGACGCCCGCCCACTCCCAATTCCGGGCGGCACTGCTGAGTCTGCGCC
ACTACCAGGAGATCGCGCTGCGCCCGCTCACCGAGGCGCAGACCACCGAA
CTCGTGCGCCGGCACCTCGGCCAGGACGCGCACGACGACGTGGTGGCCCA
GGCGTTCCGGGCGACCGGCGGCAACCTGCTCCTCGGCCACGGCCTGATCG
ACGACATCCGGGAGGCACGGACACGGACCTCAGGGTGCCTGGAAGTGGTC
GCGGGGCGGGCGTACCGGCTCGCCTACCTCGGGTCGCTCTATCGTTGCGG
CCCGGCCGCGCTGAGCGTCGCCCGAGCTTCCGCCGTGCTCGGCGAGAGTG
TCGAACTCACCCTCGTCCAGCGGATGACCGGCCTCGACACCGAGGCGGTC
GAGCAGGCCCACGAACAGCTGGTCGAGGGGCGGCTGCTGCGGGAAGGGCG
GTTCCCGCACCCCGCGGCCCGCTCCGTCGTACTCGACGACCTCTCCGCCG
CCGAGCGGCGTGGCCTGCACGAGCTGGCGCTGGAACTGCTGCGGGACCGC
GGCGTGGCCAGCAAGGTGCTCGCCCGCCACCAGATGGGTACCGGCCGGGT
GCACGGCGCCGAGGTCGCCGGGCTGTTCACCGACGCCGCGCGCGAGCACC
ACCTGCGCGGCGAGCTCGACGAGGCCGTCACCTACCTGGAGTTCGCCTAC
CGGGCCTCCGACGACCCCGCCGTCCACGCCGCACTGCGCGTCGACACCGC
CGCCATCGAGCGGCTCTGCGATCCCGCCAGATCCGGCCGGCATGTGCCCG
AGCTGCTCACCGCGTCGCGGGAACGGCTCCTCTCCAGCGAGCACGCCGTG
TCGCTCGCCTGCTGGCTGGCGATGGACGGGCGGCCGGGCGAGGCCGCCGA
GGTCCTGGCGGCCCAGCGCTCCGCCGCCCCGAGCGAGCAGGGCCGGGCGC
ACCTGCGCGTCGCGGACCTGTCCCTCGCGCTGATCTATCCCGGCGCGGCC
GATCCGCCGCGTCCGGCCGATCCGCCGGCCGAGGACGAGGTCGCCTCGTT
TTCCGGAGCCGTCCGGCACCGCGCCGTCGCCGACAAGGCCCTGAGCAACG
CGCTGCGCGGCTGGTCCGAACAGGCCGAGGCCAAAGCCGAGTACGTGCTC
CAGCACTCCCGGGTCACGACGGACCGGACCACGACCATGATGGCGTTGCT
GGCCCTGCTCTACGCCGAGGACACCGATGCCGTCCAGTCCTGGGTCGACA
AGCTGGCCGGTGACGACAACATGCGGACCCCGGCCGACGAGGCGGTCCAC
GCGGGGTTCCGCGCCGAGGCCGCGCTGCGCCGCGGCGACCTGACCGCCGC
CGTCGAATGCGGCGAGGCCGCGCTCGCCCCCCGGGTCGTGCCCTCCTGGG
GGATGGCCGCCGCATTGCCGCTGAGCAGCACCGTGGCCGCCGCGATCCGA
CTGGGCGACCTGGACCGGGCGGAGCGGTGGCTCGCCGAGCCGTTGCCGGA
GGAGACCTCCGACAGCCTCTTCGGACTGCACATGGTCTGGGCCCGTGGGC
AACACCATCTCGCGGCCGGGCGGTACCGGGCGGCGTACAACGCGTTCCGG
GACTGCGGGGAGCGGATGCGACGCTGGTCCGTCGACGTGCCGGGCCTGGC
CCTGTGGCGGGTCGACGCCGCCGAAGCGCTTCTGCTGCTCGGCCGCGGCC
GTGACGAGGGGCTGAGGCTCATCTCCGAGCAGCTGTCCCGGCCGATGGGG
TCCCGGGCGCGGGTGATGACGCTGCGGGTGCAGGCGGCCTACAGTCCGCC
GGCCAAGCGGATCGAACTGCTCGACGAGGCCGCCGATCTGCTCATCATGT
GCCGCGACCAGTACGAGCTGGCCCGCGTCCTCGCCGACATGGGCGAAGCG
TGCGGCATGCTCCGGCGGCACAGCCGTGCGCGGGGACTGTTCCGCCGCGC
ACGGCACCTCGCGACCCAGTGCGGAGCCGTGCCGCTCCTCCGGCGGCTCG
GTGGGGAGTCCTCGGACGCGGACGGCACCCAGGACGTGACGCCGGCGCAG
CGGATCACATCGCTGACCGAGGCGGAGCGGCGGGTGGCGTCGCACGCCGC
GGTCGGGCGCACCAACAAGGAGATCGCCAGCCAGCTGTTCGTCACCTCCA
GCACGGTGGAACAGCACCTCACCAACGTGTTCCGCAAGCTGGGGGTGAAG
GGCCGTCAGCAACTGCCCAAGGAACTGTCCGACGCCGGCTGA

SEQ ID NO: 185

ATGATAGCGCGCCTGTCTCCCCCAGACCTGATCGCCCGCGATGACGAGTT
CGGTTCCCTCCACCGGGCGCTCACCCGAGCGGGGGGCGGGCGGGGCGTCG
TCGCCGCCGTCACCGGGCCGATCGCCTGCGGCAAGACCGAACTCCTCGAC
GCCGCCGCGGCCAAGGCCGGCTTCGTCACCCTTCGCGCGGTGTGCTCCAT
GGAGGAGCGGGCCCTGCCGTACGGCATGCTCGGCCAGCTCCTCGACCAGC
CCGAGCTGGCCGCCCGGACACCGGAGCTGGTCCGGCTGACGGCATCGTGC
GAAAACCTGCCGGCCGACGTCGACAACCGCCTGGGGACCGAACTCACCCG
CACGGTGCTGACGCTCGCCGCGGAGCGGCCCGTACTGATCGGCATCGACG
ACGTGCACCACGCCGACGCGCCGTCGCTGCGCTGCCTGCTCCACCTCGCG
CGCCGCATCAGCCGGGCCCGTGTCGCCATCGTGCTGACCGAGCTGCTCCG
GCCGACGCCCGCCCACTCCCAATTCCGGGCGGCACTGCTGAGTCTGCGCC
ACTACCAGGAGATCGCGCTGCGCCCGCTCACCGAGGCGCAGACCACCGAA
CTCGTGCGCCGGCACCTCGGCCAGGACGCGCACGACGACGTGGTGGCCCA
GGCGTTCCGGGCGACCGGCGGCAACCTGCTCCTCGGCCACGGCCTGATCG
ACGACATCCGGGAGGCACGGACACGGACCTCAGGGTGCCTGGAAGTGGTC
GCGGGGCGGGCGTACCGGCTCGCCTACCTCGGGTCGCTCTATCGTTGCGG
CCCGGCCGCGCTGAGCGTCGCCCGAGCTTCCGCCGTGCTCGGCGAGAGTG
TCGAACTCACCCTCGTCCAGCGGATGACCGGCCTCGACACCGAGGCGGTC
GAGCAGGCCCACGAACAGCTGGTCGAGGGGCGGCTGCTGCGGGAAGGGCG
GTTCCCGCACCCCGCGGCCCGCTCCGTCGTACTCGACGACCTCTCCGCCG
CCGAGCGGCGTGGCCTGCACGAGCTGGCGCTGGAACTGCTGCGGGACCGC
GGCGTGGCCAGCAAGGTGCTCGCCCGCCACCAGATGGGTACCGGCCGGGT
GCACGGCGCCGAGGTCGCCGGGCTGTTCACCGACGCCGCGCGCGAGCACC
ACCTGCGCGGCGAGCTCGACGAGGCCGTCACCTACCTGGAGTTCGCCTAC
CGGGCCTCCGACGACCCCGCCGTCCACGCCGCACTGCGCGTCGACACCGC
CGCCATCGAGCGGCTCTGCGATCCCGCCAGATCCGGCCGGCATGTGCCCG
AGCTGCTCACCGCGTCGCGGGAACGGCTCCTCTCCAGCGAGCACGCCGTG
TCGCTCGCCTGCTGGCTGGCGATGGACGGGCGGCCGGGCGAGGCCGCCGA
GGTCCTGGCGGCCCAGCGCTCCGCCGCCCCGAGCGAGCAGGGCCGGGCGC
ACCTGCGCGTCGCGGACCTGTCCCTCGCGCTGATCTATCCCGGCGCGGCC
GATCCGCCGCGTCCGGCCGATCCGCCGGCCGAGGACGAGGTCGCCTCGTT
TTCCGGAGCCGTCCGGCACCGCGCCGTCGCCGACAAGGCCCTGAGCAACG
CGCTGCGCGGCTGGTCCGAACAGGCCGAGGCCAAAGCCGAGTACGTGCTC
CAGCACTCCCGGGTCACGACGGACCGGACCACGACCATGATGGCGTTGCT
GGCCCTGCTCTACGCCGAGGACACCGATGCCGTCCAGTCCTGGGTCGACA
AGCTGGCCGGTGACGACAACATGCGGACCCCGGCCGACGAGGCGGTCCAC
GCGGGGTTCCGCGCCGAGGCCGCGCTGCGCCGCGGCGACCTGACCGCCGC
CGTCGAATGCGGCGAGGCCGCGCTCGCCCCCCGGGTCGTGCCCTCCTGGG
GGATGGCCGCCGCATTGCCGCTGAGCAGCACCGTGGCCGCCGCGATCCGA
CTGGGCGACCTGGACCGGGCGGAGCGGTGGCTCGCCGAGCCGTTGCCGGA
GGAGACCTCCGACAGCCTCTTCGGACTGCACATGGTCTGGGCCCGTGGGC
AACACCATCTCGCGGCCGGGCGGTACCGGGCGGCGTACAACGCGTTCCGG
GACTGCGGGGAGCGGATGCGACGCTGGTCCGTCGACGTGCCGGGCCTGGC
CCTGTGGCGGGTCGACGCCGCCGAAGCGCTTCTGCTGCTCGGCCGCGGCC
GTGACGAGGGGCTGAGGCTCATCTCCGAGCAGCTGTCCCGGCCGATGGGG
TCCCGGGCGCGGGTGATGACGCTGCGGGTGCAGGCGGCCTACAGTCCGCC
GGCCAAGCGGATCGAACTGCTCGACGAGGCCGCCGATCTGCTCATCATGT
GCCGCGACCAGTACGAGCTGGCCCGCGTCCTCGCCGACATGGGCGAAGCG
TGCGGCATGCTCCGGCGGCACAGCCGTGCGCGGGGACTGTTCCGCCGCGC
ACGGCACCTCGCGACCCAGTGCGGAGCCGTGCCGCTCCTCCGGCGGCTCG
GTGGGGAGTCCTCGGACGCGGACGGCACCCAGGACGTGACGCCGGCGCAG
CGGATCACATCGCTGACCGAGGCGGAGCGGCGGGTGGCGTCGCACGCCGC
GGTCGGGCGCACCAACAAGGAGATCGCCAGCCAGCTGTTCGTCACCTCCA
GCACGGTGGAACAGCACCTCACCAACGTGTTCCGCAAGCTGGGGGTGAAG
GGCCGTCAGCAACTGCCCAAGGAACTGTCCGACGCCGGCTGA

SEQ ID NO: 186

GTGGAGTTTTACGACCTGGTCGCCCGCGATGACGAGCTCAGAAGGTTGGA
CCAGGCCCTCGGCCGCGCCGCCGGCGGACGGGGTGTCGTGGTCACCGTCA
CCGGACCGGTCGGCTGCGGCAAGACCGAACTGCTGGACGCGGCCGCGGCC
GAGGAGGAATTCATCACGTTGCGTGCGGTCTGCTCGGCCGAGGAGCGGGC
CCTGCCGTACGCCGTGATCGGCCAACTCCTCGACCATCCCGTACTCTCCG
CACGCGCGCCCGACCTGGCCTGCGTGACGGCTCCGGGCCGGACGCTGCCG
GCCGACACCGAGAACCGCCTGCGCCGCGACCTCACCCGGGCCCTGCTGGC
CCTGGCCTCCGAACGACCGGTTCTGATCTGCATCGACGACGTGCACCAGG
CCGACACCGCCTCGCTGAACTGCCTGCTGCACCTGGCCCGGCGGGTCGCC
TCGGCCCGGATCGCCATGATCCTCACCGAGTTGCGCCGGCTCACCCCGGC
TCACTCCCGGTTCGAGGCGGAACTGCTCAGCCTGCGGCACCGCCACGAGA
TCGCGCTGCGTCCCCTCGGCCCGGCCGACACCGCCGAACTGGCCCGCGCC
CGGCTCGGCGCCGGCGTCACCGCCGACGAGCTGGCCCAGGTCCACGAGGC
CACCAGCGGGAACCCCAACCTGGTCGGAGGCCTGGTCAACGACGTGCGAG
AGGCCTGGGCGGCCGGTGGCACGGGCATTGCGGCGGGGCGGGCGTACCGG
CTGGCGTACCTCAGCTCCGTGTACCGCTGTGGTCCGGTCCCGTTGCGGAT
CGCCCAGGCGGCGGCGGTGCTGGGTCCCAGCGCCACCGTCACGCTGGTGC
GCCGGATCAGCGGGCTCGACGCCGAGACGGTGGACGAGGCGACCGCGATC
CTCACCGAGGGCGGCCTGCTCCGGGACCACCGGTTCCCGCATCCGGCGGC
CCGCTCGGTCGTACTCGACGACATGTCCGCGCAGGAACGCCGCCGCCTGC
ACCGGTCCACGCTGGACGTGCTGGACGGCGTACCCGTCGACGTGCTCGCG
CACCACCAGGCCGGCGCCGGTCTGCTGCACGGCCCGCAGGCGGCCGAGAT
GTTCGCCCGGGCCAGCCAGGAGCTGCGGGTACGCGGCGAGCTGGACGCCG
CGACCGAGTACCTGCAACTGGCCTACCGGGCCTCCGACGACGCCGGCGCC
CGGGCCGCCCTGCAGGTGGAGACCGTGGCCGGCGAGCGCCGCCGCAACCC
GCTGGCCGCCAGCCGGCACCTGGACGAGCTGGCCGCCGCCGCCCGGGCCG
GCCTGCTGTCGGCCGAGCACGCCGCCCTGGTCGTGCACTGGCTGGCCGAC
GCCGGACGACCCGGCGAGGCCGCCGAGGTGCTGGCGCTGCAGCGGGCGCT
GGCCGTCACCGACCACGACCGGGCCCGCCTGCGGGCGGCCGAGGTGTCGC
TCGCGCTGTTCCACCCCGGCGTCCCCGGTTCGGACCCGCGGCCCCTCGCG
CCGGAGGAGCTCGCGAGCCTGTCCCTGTCGGCCCGGCACGGTGTGACCGC
CGACAACGCGGTGCTGGCGGCGCTGCGCGGCCGTCCCGAGTCGGCCGCCG
CCGAGGCGGAGAACGTGCTGCGCAACGCCGACGCCGCCGCGTCCGGCCCG
ACCGCCCTGGCCGCGCTGACGGCCCTGCTCTACGCCGAGAACACCGACGC
CGCCCAGCTCTGGGCGGACAAGCTGGCCGCGGGCATCGGGGCGGGGGAGG
GGGAGGCCGGCTACGCGGGGCCGCGGACCGTGGCCGCCCTGCGTCGCGGC
GACCTGACCACCGCGGTCCAGGCGGCCGGCGCGGTCCTGGACCGCGGCCG
GCCGTCGTCGCTCGGCATCACCGCCGTGTTGCCGTTGAGCGGCGCGGTCG
CCGCCGCGATCCGGCTGGGCGAGCTCGAGCGGGCCGAGAAGTGGCTGGCC
GAGCCGCTGCCCGAAGCCGTCCACGACAGCCTGTTCGGCCTGCACCTGCT
GATGGCGCGGGGCCGCTACAGCCTCGCGGTGGGCCGGCACGAGGCGGCGT
ACGCCGCGTTCCGGGACTGCGGTGAACGGATGCGCCGGTGGGACGTCGAC
GTGCCCGGGCTGGCCCTGTGGCGGGTGGACGCGGCCGAGGCGCTGCTGCC
CGGCGATGACCGGGCGGAGGGCCGGCGGCTGATCGACGAGCAGCTCACCC
GGCCGATGGGGCCCCGGTCACGAGCCCTGACCCTGCGGGTACGAGCGGCC
TACGCCCCGCCGGCGAAACGGATCGACCTGCTCGACGAAGCGGCCGACCT
GCTGCTCTCCAGCAACGACCAGTACGAGCGGGCACGGGTGCTGGCCGACC
TGAGCGAGGCGTTCAGCGCGCTCCGGCAGAACGGCCGGGCGCGCGGCATC
CTGCGGCAGGCCCGGCACCTGGCCGCCCAGTGCGGGGCGGTCCCCCTGCT
GCGCCGGCTGGGCGTCAAGGCCGGCCGGTCCGGTCGGCTCGGCCGGCCGC
CGCAGGGAATCCGCTCCCTGACCGAGGCCGAGCGCCGGGTGGCCACGCTG
GCCGCCGCCGGGCAGACCAACCGGGAGATCGCCGACCAGCTCTTCGTCAC
CGCCAGCACGGTCGAGCAGCACCTCACCAACGTGTTCCGCAAGCTCGGCG
TGAAGGGCCGCCAGCAATTGCCGGCCGAGCTGGCCGACCTGCGGCCGCCG
GGCTGA

SEQ ID NO: 187

ATGGAGTTTTACGACCTGGTCGCCCGCGATGACGAGCTCAGAAGGTTGGA
CCAGGCCCTCGGCCGCGCCGCCGGCGGACGGGGTGTCGTGGTCACCGTCA
CCGGACCGGTCGGCTGCGGCAAGACCGAACTGCTGGACGCGGCCGCGGCC
GAGGAGGAATTCATCACGTTGCGTGCGGTCTGCTCGGCCGAGGAGCGGGC
CCTGCCGTACGCCGTGATCGGCCAACTCCTCGACCATCCCGTACTCTCCG
CACGCGCGCCCGACCTGGCCTGCGTGACGGCTCCGGGCCGGACGCTGCCG
GCCGACACCGAGAACCGCCTGCGCCGCGACCTCACCCGGGCCCTGCTGGC
CCTGGCCTCCGAACGACCGGTTCTGATCTGCATCGACGACGTGCACCAGG
CCGACACCGCCTCGCTGAACTGCCTGCTGCACCTGGCCCGGCGGGTCGCC
TCGGCCCGGATCGCCATGATCCTCACCGAGTTGCGCCGGCTCACCCCGGC
TCACTCCCGGTTCGAGGCGGAACTGCTCAGCCTGCGGCACCGCCACGAGA
TCGCGCTGCGTCCCCTCGGCCCGGCCGACACCGCCGAACTGGCCCGCGCC
CGGCTCGGCGCCGGCGTCACCGCCGACGAGCTGGCCCAGGTCCACGAGGC
CACCAGCGGGAACCCCAACCTGGTCGGAGGCCTGGTCAACGACGTGCGAG
AGGCCTGGGCGGCCGGTGGCACGGGCATTGCGGCGGGGCGGGCGTACCGG
CTGGCGTACCTCAGCTCCGTGTACCGCTGTGGTCCGGTCCCGTTGCGGAT
CGCCCAGGCGGCGGCGGTGCTGGGTCCCAGCGCCACCGTCACGCTGGTGC
GCCGGATCAGCGGGCTCGACGCCGAGACGGTGGACGAGGCGACCGCGATC
CTCACCGAGGGCGGCCTGCTCCGGGACCACCGGTTCCCGCATCCGGCGGC
CCGCTCGGTCGTACTCGACGACATGTCCGCGCAGGAACGCCGCCGCCTGC
ACCGGTCCACGCTGGACGTGCTGGACGGCGTACCCGTCGACGTGCTCGCG
CACCACCAGGCCGGCGCCGGTCTGCTGCACGGCCCGCAGGCGGCCGAGAT
GTTCGCCCGGGCCAGCCAGGAGCTGCGGGTACGCGGCGAGCTGGACGCCG
CGACCGAGTACCTGCAACTGGCCTACCGGGCCTCCGACGACGCCGGCGCC
CGGGCCGCCCTGCAGGTGGAGACCGTGGCCGGCGAGCGCCGCCGCAACCC
GCTGGCCGCCAGCCGGCACCTGGACGAGCTGGCCGCCGCCGCCCGGGCCG
GCCTGCTGTCGGCCGAGCACGCCGCCCTGGTCGTGCACTGGCTGGCCGAC
GCCGGACGACCCGGCGAGGCCGCCGAGGTGCTGGCGCTGCAGCGGGCGCT
GGCCGTCACCGACCACGACCGGGCCCGCCTGCGGGCGGCCGAGGTGTCGC
TCGCGCTGTTCCACCCCGGCGTCCCCGGTTCGGACCCGCGGCCCCTCGCG
CCGGAGGAGCTCGCGAGCCTGTCCCTGTCGGCCCGGCACGGTGTGACCGC
CGACAACGCGGTGCTGGCGGCGCTGCGCGGCCGTCCCGAGTCGGCCGCCG
CCGAGGCGGAGAACGTGCTGCGCAACGCCGACGCCGCCGCGTCCGGCCCG
ACCGCCCTGGCCGCGCTGACGGCCCTGCTCTACGCCGAGAACACCGACGC
CGCCCAGCTCTGGGCGGACAAGCTGGCCGCGGGCATCGGGGCGGGGGAGG
GGGAGGCCGGCTACGCGGGGCCGCGGACCGTGGCCGCCCTGCGTCGCGGC
GACCTGACCACCGCGGTCCAGGCGGCCGGCGCGGTCCTGGACCGCGGCCG
GCCGTCGTCGCTCGGCATCACCGCCGTGTTGCCGTTGAGCGGCGCGGTCG
CCGCCGCGATCCGGCTGGGCGAGCTCGAGCGGGCCGAGAAGTGGCTGGCC
GAGCCGCTGCCCGAAGCCGTCCACGACAGCCTGTTCGGCCTGCACCTGCT
GATGGCGCGGGGCCGCTACAGCCTCGCGGTGGGCCGGCACGAGGCGGCGT
ACGCCGCGTTCCGGGACTGCGGTGAACGGATGCGCCGGTGGGACGTCGAC
GTGCCCGGGCTGGCCCTGTGGCGGGTGGACGCGGCCGAGGCGCTGCTGCC
CGGCGATGACCGGGCGGAGGGCCGGCGGCTGATCGACGAGCAGCTCACCC
GGCCGATGGGGCCCCGGTCACGAGCCCTGACCCTGCGGGTACGAGCGGCC
TACGCCCCGCCGGCGAAACGGATCGACCTGCTCGACGAAGCGGCCGACCT
GCTGCTCTCCAGCAACGACCAGTACGAGCGGGCACGGGTGCTGGCCGACC
TGAGCGAGGCGTTCAGCGCGCTCCGGCAGAACGGCCGGGCGCGCGGCATC
CTGCGGCAGGCCCGGCACCTGGCCGCCCAGTGCGGGGCGGTCCCCCTGCT
GCGCCGGCTGGGCGTCAAGGCCGGCCGGTCCGGTCGGCTCGGCCGGCCGC
CGCAGGGAATCCGCTCCCTGACCGAGGCCGAGCGCCGGGTGGCCACGCTG
GCCGCCGCCGGGCAGACCAACCGGGAGATCGCCGACCAGCTCTTCGTCAC
CGCCAGCACGGTCGAGCAGCACCTCACCAACGTGTTCCGCAAGCTCGGCG
TGAAGGGCCGCCAGCAATTGCCGGCCGAGCTGGCCGACCTGCGGCCGCCG
GGCTGA

SEQ ID NO: 188

GTGGTCACCGTCACCGGCCCAATCGCCTGCGGCAAGACAGAACTGCTTGA
CGCGGCTGCCGCGAAGGCTGAGGCCATCATTCTGCGCGCGGTCTGCGCGC
CAGAAGAGCGGGCTATGCCGTACGCCATGATCGGGCAGCTCATCGACGAC
CCGGCGCTCGCGCATCGGGCGCCGGGGCTGGCTGATCGGATAGCCCAGGG
CGGGCAGCTGTCGCTGAGGGCCGAGAACCGACTGCGCAGGGATCTCACCC
GTGCCCTGCTGGCGCTTGCCGTCGACCGGCCTGTGCTGATCGGCGTCGAC
GATGTGCATCACGCCGACACCGCCTCTTTGAACTGTCTGCTGCATTTGGC
GCGCCGGGTCCGTCCGGCCCGGATATCCATGATCTTCACCGAGTTGCGCA
GCCTCACCCCTACTCAGTCACGGTTCAAGGCGGAGCTGCTCAGCCTGCCG
TACCACCACGAGATCGCGCTGCGTCCGTTCGGACCGGAGCAATCGGCGGA
GCTGGCCCGCGCCGCCTTCGGCCCGGGCCTCGCCGAGGATGTGCTCGTGG
GGTTGTATAAAACGACCAGGGGCAATCTGAGTCTCAGCCGTGGACTGATC
AGCGATGTGCGGGAGGCCCTGGCCAACGGAGAGAGCGCCTTCGAGGCGGG
CCGCGCGTTCCGGCTGGCGTACCTCGGCTCGCTCTACCGCTGTGGCCCGG
TCGCGCTGCGGGTCGCCCGAGTGGCTGCCGTGCTGGGCCCGAGCGCCACC
ACCACGCTGGTGCGCCGTCTAAGCGGGCTCAGCGCGGAGACGATAGACCG
GGCAACCAAGATCCTCACCGAGGGCGGGCTGCTGCTCGACCAGCAGTTCC
CGCACCCGGCCGCCCGCTCGGTGGTGCTTGATGACATGTCCGCCCAGGAA
CGACGCGGCCTGCACACTCTCGCCCTGGAACTGCTGGACGAGGCGCCGGT
TGAAGTGCTCGCGCACCACCAGGTCGGCGCCGGTCTCATACACGGGCCCA
AGGCTGCGGAGATGTTCGCCAAGGCCGGCAAGGCTCTGGTCGTACGCAAC
GAGTTGGGCGACGCGGCAGAATACCTGCAACTGGCTCACCGGGCCTCCGA
CGATGTCTCCACCCGGGCCGCCTTACGGGTCGAGGCCGTGGCGATCGAGC
GCCGCCGCAATCCGCTGGCCTCCAGTCGGCACATGGACGAGCTGAGCGCC
GCCGGCCGCGCCGGTCTGCTTTCCCCCAAGCATGCGGCGCTGGCCGTCTT
CTGGCTGGCCGACGGCGGGCGATCCGGCGAGGCAGCCGAGGTGCTGGCGT
CGGAACGCCCGCTAGCGACCACCGATCAGAACCGGGCCCACTTGCGATTT
GTCGAGGTGACTCTCGCGCTGTTCTCTCCCGGCGCCTTCGGATCGGACCG
GCGCCCACCTCCGCTGACGCCGGACGAACTCGCCAGCCTGCCGAAGGCGG
CCTGGCAATGCGCGGTCGCCGACAACGCGGCCATGACCGCCTTGCACGGT
CATCCAGAACTTGCCACCGCTCAGGCGGAAACAGTTCTGCGGCAGGCTGA
TTCGGCAGCCGACGCGATCCCCGCCGCGCTGATCGCCCTGTTGTACGCGG
AGAACACCGAGTCCGCTCATATCTGGGCCGACAAGCTGGGCAGCACGAAT
GGCGGGGTATCGAACGAGGCGGAAGCGGGCTACGCCGGCCCGTGCGCCGA
GATCGCCCTGCGGCGCGGCGACCTGGCCACGGCGTTCGAGGCTGGTAGCA
CCGTCCTGGACGACCGGTCGCTGCCGTCGCTCGGCATCACCGCCGCATTG
CTGTTGAGCAGCAAGACGGCCGCCGCTGTCCGGCTGGGCGAACTCGAGCG
TGCGGAGAAGCTGCTCGCCGAGCCGCTTCCGAACGGCGTCCAGGACAGCC
TTTTCGGTCTGCACCTGCTCTCGGCATACGGCCAGTACAGCCTCGCGATG
GGCCGATATGAATCGGCTCTCCGGGCGTTTCACACCTGCGGAGAACGTAT
GCGCAGCTGGGATGTTGACGTGCCTGGTCTGGCCCTGTGGCGTGTCGACG
CCGCCGAGGCGCTGCTCAGCCTCGACCGGAACGAGGGCCAGCGGCTCATC
GACGAACAACTCACCCGTCCGATGGGGCCTCGTTCCCGCGCGTTAACGCT
GCGGATCAAGGCGGCATACCTCCCGCGGACGAAGCGGATCCCCCTGCTCC
ATGAGGCGGCCGAGCTGCTGCTCCCCTGCCCCGACCCGTACGAGCAAGCG
CGGGTGCTCGCCGATCTGGGCGACACGCTCAGCGCGCTCAGACGCTATAG
CCGGGCGCGGGGAGTTCTCCGGCAGGCTCGTCACCTGGCCGCCCAGTGCG
GTGCTGTCCCGCTGCTGCGCAGGCTCGGGGGCGAGCCCGGCCGGATCGAC
GACGCCGGCCTGCCGCAGCGGAGCACATCGTTGACCGATGCGGAGCGGCG
GGTGGCGGCGCTGGCCGCGGCCGGACAGACCAACCGGGAGATCGCCAAAC
AGCTGTTCGTCACGGCCAGCACAGTGGAACAGCACCTCACAAGCGTCTTC
CGCAAACTGGGGGTCAAGGGTCGCAAGCAGCTGCCGACCGCGCTGGCCGA
CGTGGAACAGACCTGA

SEQ ID NO: 189

ATGTATAGCGGTACCTGCCGTGAAGGATACGAACTCGTCGCACGCGAGGA
CGAACTCGGCATTCTACAGAGGTCTCTGGAACAAGCGAGCAGCGGCCAGG
GCGTCGTGGTCACCGTCACCGGCCCAATCGCCTGCGGCAAGACAGAACTG
CTTGACGCGGCTGCCGCGAAGGCTGAGGCCATCATTCTGCGCGCGGTCTG
CGCGCCAGAAGAGCGGGCTATGCCGTACGCCATGATCGGGCAGCTCATCG
ACGACCCGGCGCTCGCGCATCGGGCGCCGGGGCTGGCTGATCGGATAGCC
CAGGGCGGGCAGCTGTCGCTGAGGGCCGAGAACCGACTGCGCAGGGATCT
CACCCGTGCCCTGCTGGCGCTTGCCGTCGACCGGCCTGTGCTGATCGGCG
TCGACGATGTGCATCACGCCGACACCGCCTCTTTGAACTGTCTGCTGCAT
TTGGCGCGCCGGGTCCGTCCGGCCCGGATATCCATGATCTTCACCGAGTT
GCGCAGCCTCACCCCTACTCAGTCACGGTTCAAGGCGGAGCTGCTCAGCC
TGCCGTACCACCACGAGATCGCGCTGCGTCCGTTCGGACCGGAGCAATCG
GCGGAGCTGGCCCGCGCCGCCTTCGGCCCGGGCCTCGCCGAGGATGTGCT
CGTGGGGTTGTATAAAACGACCAGGGGCAATCTGAGTCTCAGCCGTGGAC
TGATCAGCGATGTGCGGGAGGCCCTGGCCAACGGAGAGAGCGCCTTCGAG
GCGGGCCGCGCGTTCCGGCTGGCGTACCTCGGCTCGCTCTACCGCTGTGG
CCCGGTCGCGCTGCGGGTCGCCCGAGTGGCTGCCGTGCTGGGCCCGAGCG
CCACCACCACGCTGGTGCGCCGTCTAAGCGGGCTCAGCGCGGAGACGATA
GACCGGGCAACCAAGATCCTCACCGAGGGCGGGCTGCTGCTCGACCAGCA
GTTCCCGCACCCGGCCGCCCGCTCGGTGGTGCTTGATGACATGTCCGCCC
AGGAACGACGCGGCCTGCACACTCTCGCCCTGGAACTGCTGGACGAGGCG
CCGGTTGAAGTGCTCGCGCACCACCAGGTCGGCGCCGGTCTCATACACGG
GCCCAAGGCTGCGGAGATGTTCGCCAAGGCCGGCAAGGCTCTGGTCGTAC
GCAACGAGTTGGGCGACGCGGCAGAATACCTGCAACTGGCTCACCGGGCC
TCCGACGATGTCTCCACCCGGGCCGCCCTGCGGGTCGAGGCCGTGGCGAT
CGAGCGCCGCCGCAATCCGCTGGCCTCCAGTCGGCACATGGACGAGCTGA
GCGCCGCCGGCCGCGCCGGTCTGCTTTCCCCCAAGCATGCGGCGCTGGCC
GTCTTCTGGCTGGCCGACGGCGGGCGATCCGGCGAGGCAGCCGAGGTGCT
GGCGTCGGAACGCCCGCTAGCGACCACCGATCAGAACCGGGCCCACTTGC
GATTTGTCGAGGTGACTCTCGCGCTGTTCTCTCCCGGCGCCTTCGGATCG
GACCGGCGCCCACCTCCGCTGACGCCGGACGAACTCGCCAGCCTGCCGAA
GGCGGCCTGGCAATGCGCGGTCGCCGACAACGCGGCCATGACCGCCTTGC
ACGGTCATCCAGAACTTGCCACCGCTCAGGCGGAAACAGTTCTGCGGCAG
GCTGATTCGGCAGCCGACGCGATCCCCGCCGCGCTGATCGCCCTGTTGTA
CGCGGAGAACACCGAGTCCGCTCATATCTGGGCCGACAAGCTGGGCAGCA
CGAATGGCGGGGTATCGAACGAGGCGGAAGCGGGCTACGCCGGCCCGTGC
GCCGAGATCGCCCTGCGGCGCGGCGACCTGGCCACGGCGTTCGAGGCTGG
TAGCACCGTCCTGGACGACCGGTCGCTGCCGTCGCTCGGCATCACCGCCG
CATTGCTGTTGAGCAGCAAGACGGCCGCCGCTGTCCGGCTGGGCGAACTC
GAGCGTGCGGAGAAGCTGCTCGCCGAGCCGCTTCCGAACGGCGTCCAGGA
CAGCCTTTTCGGTCTGCACCTGCTCTCGGCATACGGCCAGTACAGCCTCG
CGATGGGCCGATATGAATCGGCTCTCCGGGCGTTTCACACCTGCGGAGAA
CGTATGCGCAGCTGGGATGTTGACGTGCCTGGTCTGGCCCTGTGGCGTGT
CGACGCCGCCGAGGCGCTGCTCAGCCTCGACCGGAACGAGGGCCAGCGGC
TCATCGACGAACAACTCACCCGTCCGATGGGGCCTCGTTCCCGCGCGCTG
ACGCTGCGGATCAAGGCGGCATACCTCCCGCGGACGAAGCGGATCCCCCT
GCTCCATGAGGCGGCCGAGCTGCTGCTCCCCTGCCCCGACCCGTACGAGC
AAGCGCGGGTGCTCGCCGATCTGGGCGACACGCTCAGCGCGCTCAGACGC
TATAGCCGGGCGCGGGGAGTTCTCCGGCAGGCTCGTCACCTGGCCGCCCA
GTGCGGTGCTGTCCCGCTGCTGCGCAGGCTCGGGGGCGAGCCCGGCCGGA
TCGACGACGCCGGCCTGCCGCAGCGGAGCACATCGTTGACCGATGCGGAG
CGGCGGGTGGCGGCGCTGGCCGCGGCCGGACAGACCAACCGGGAGATCGC
CAAACAGCTGTTCGTCACGGCCAGCACAGTGGAACAGCACCTCACAAGCG
TCTTCCGCAAACTGGGGGTCAAGGGTCGCAAGCAGCTGCCGACCGCGCTG
GCCGACGTGGAACAGACCTGA

SEQ ID NO: 190

ATGCCTGCCGTGGAGAGCTATGAACTGGACGCCCGCGATGACGAGCTCAG
AAGACTGGAGGAGGCGGTAGGCCAGGCGGGCAACGGCCGGGGTGTGGTGG
TCACCATCACCGGGCCGATCGCCTGCGGCAAGACCGAACTGCTCGACGCG
GCCGCCGCGAAGAGCGACGCCATCACATTACGTGCGGTCTGCTCCGAGGA
GGAACGGGCCCTCCCGTACGCCCTGATCGGGCAGCTCATCGACAACCCGG
CGGTCGCCTCCCAGCTGCCGGATCCGGTCTCCATGGCCCTCCCGGGCGAG
CACCTGTCGCCGGAGGCCGAGAACCGGCTGCGCGGCGACCTCACCCGTAC
CCTGCTGGCGCTCGCCGCCGAACGGCCGGTGCTGATCGGCATCGACGACA
TGCACCACGCCGACACCGCCTCTTTGAACTGCCTGCTCCACCTGGCCCGG
AGGGTCGGCCCGGCCCGGATCGCCATGGTCCTCACCGAGCTGCGCCGGCT
CACCCCGGCCCACTCCCAGTTCCACGCCGAGCTGCTCAGCCTGGGGCACC
ACCGCGAGATCGCGCTGCGCCCGCTCGGCCCGAAGCACATCGCCGAGCTG
GCCCGCGCCGGCCTCGGTCCCGATGTCGACGAGGACGTGCTCACGGGGTT
GTACCGGGCGACCGGCGGCAACCTGAACCTCGGCCACGGACTGATCAAGG
ATGTGCGGGAGGCCTGGGCGACGGGCGGGACGGGCATCAACGCGGGCCGC
GCGTACCGGCTGGCGTACCTCGGTTCCCTCTACCGCTGCGGCCCGGTCCC
GTTGCGGGTCGCACGGGTGGCCGCCGTGCTGGGCCAGAGCGCCAACACCA
CCCTGGTGCGCTGGATCAGCGGGCTCAACGCGGACGCGGTGGGCGAGGCG
ACCGAGATCCTCACCGAGGGCGGCCTGCTGCACGACCTGCGGTTCCCGCA
TCCGGCGGCCCGTTCGGTCGTACTCAACGACCTGTCCGCCCGGGAACGCC
GCCGACTGCACCGGTCCGCTCTGGAAGTGCTGGATGACGTACCCGTTGAA
GTGGTCGCGCACCACCAGGCCGGTGCCGGTTTCATCCACGGTCCCAAGGC
CGCCGAGATCTTCGCCAAGGCCGGCCAGGAGCTGCATGTGCGCGGCGAGC
TGGACGCCGCGTCCGACTATCTGCAACTGGCCCACCACGCCTCCGACGAC
GCCGTCACCCGGGCCGCGCTGCGGGTCGAGGCCGTGGCGATCGAGCGCCG
CCGCAACCCGCTGGCCTCCAGCCGCCACCTCGACGAGCTGACCGTCGCCG
CCCGTGCCGGTCTGCTCTCCCTCGAGCACGCCGCGCTGATGATCCGCTGG
CTGGCTCTCGGCGGGCGGTCCGGCGAGGCGGCCGAGGTGCTGGCCGCGCA
GCGCCCGCGTGCGGTCACCGACCAGGACAGGGCCCACCTGCGGGCCGCCG
AGGTATCGCTGGCGCTGGTCAGCCCGGGCGCGTCCGGCGTCAGCCCGGGT
GCGTCCGGCCCGGATCGGCGGCCGCGTCCGCTCCCGCCGGATGAGCTCGC
GAACCTGCCGAAGGCGGCCCGGCTTTGTGCGATCGCCGACAACGCCGTCA
TATCGGCCCTGCACGGTCGTCCCGAGCTTGCCTCGGCCGAGGCGGAGAAC
GTCCTGAAGCAGGCTGACTCGGCGGCGGACGGCGCCACCGCCCTCTCCGC
GCTGACGGCCTTGCTGTACGCGGAGAACACCGACACCGCTCAGCTCTGGG
CCGACAAGCTCGTCTCCGAGACCGGGGCGTCGAACGAGGAGGAAGGCGCG
GGCTACGCGGGGCCGCGCGCCGAGACCGCGTTGCGCCGCGGCGACCTGGC
CGCGGCGGTCGAGGCGGGCAGCGCCATTCTGGACCACCGGCGGGGGTCGT
TGCTCGGCATCACCGCCGCGCTACCGCTGAGCAGCGCGGTAGCCGCCGCC
ATCCGGCTGGGCGAGACCGAGCGGGCGGAGAAGTGGCTCGCCGAGCCGCT
GCCGGAGGCCATTCGGGACAGCCTGTTCGGGCTGCACCTGCTCTCGGCGC
GCGGCCAGTACTGCCTCGCGACGGGCCGGCACGAGTCGGCGTACACGGCG
TTCCGCACCTGCGGGGAACGGATGCGGAACTGGGGCGTCGACGTGCCGGG
TCTGTCCCTGTGGCGCGTCGACGCCGCCGAGGCGCTGCTGCACGGCCGCG
ACCGGGACGAGGGCCGACGGCTCATCGACGAGCAGCTCACCCATGCGATG
GGACCCCGTTCCCGCGCTTTGACGCTGCGGGTGCAGGCGGCGTACAGCCC
GCAGGCGCAGCGGGTCGACCTGCTCGAAGAGGCGGCCGACCTGCTGCTCT
CCTGCAACGACCAGTACGAGCGGGCGCGGGTGCTCGCCGATCTGAGCGAG
GCGTTCAGCGCGCTCAGGCACCACAGCCGGGCGCGGGGACTGCTCCGGCA
GGCCCGGCACCTGGCCGCCCAGTGCGGCGCGACCCCGCTGCTGCGCCGGC
TCGGGGCCAAGCCCGGAGGCCCCGGCTGGCTGGAGGAATCCGGCCTGCCG
CAGCGGATCAAGTCGCTGACCGACGCGGAGCGGCGGGTGGCGTCGCTGGC
CGCCGGCGGCCAGACCAACCGCGTGATCGCCGACCAGCTCTTCGTCACGG
CCAGCACGGTGGAGCAGCACCTCACGAACGTCTTCCGCAAGCTGGGCGTC
AAGGGCCGCCAGCACCTGCCGGCCGAACTCGCCAACGCGGAATAG

SEQ ID NO: 191

ATGCCTGCCGTGGAGAGCTATGAACTGGACGCCCGCGATGACGAGCTCAG
AAGACTGGAGGAGGCGGTAGGCCAGGCGGGCAACGGCCGGGGTGTGGTGG
TCACCATCACCGGGCCGATCGCCTGCGGCAAGACCGAACTGCTCGACGCG
GCCGCCGCGAAGAGCGACGCCATCACACTGCGTGCGGTCTGCTCCGAGGA
GGAACGGGCCCTCCCGTACGCCCTGATCGGGCAGCTCATCGACAACCCGG
CGGTCGCCTCCCAGCTGCCGGATCCGGTCTCCATGGCCCTCCCGGGCGAG
CACCTGTCGCCGGAGGCCGAGAACCGGCTGCGCGGCGACCTCACCCGTAC
CCTGCTGGCGCTCGCCGCCGAACGGCCGGTGCTGATCGGCATCGACGACA
TGCACCACGCCGACACCGCCTCTTTGAACTGCCTGCTCCACCTGGCCCGG
AGGGTCGGCCCGGCCCGGATCGCCATGGTCCTCACCGAGCTGCGCCGGCT
CACCCCGGCCCACTCCCAGTTCCACGCCGAGCTGCTCAGCCTGGGGCACC
ACCGCGAGATCGCGCTGCGCCCGCTCGGCCCGAAGCACATCGCCGAGCTG
GCCCGCGCCGGCCTCGGTCCCGATGTCGACGAGGACGTGCTCACGGGGTT
GTACCGGGCGACCGGCGGCAACCTGAACCTCGGCCACGGACTGATCAAGG
ATGTGCGGGAGGCCTGGGCGACGGGCGGGACGGGCATCAACGCGGGCCGC
GCGTACCGGCTGGCGTACCTCGGTTCCCTCTACCGCTGCGGCCCGGTCCC
GTTGCGGGTCGCACGGGTGGCCGCCGTGCTGGGCCAGAGCGCCAACACCA
CCCTGGTGCGCTGGATCAGCGGGCTCAACGCGGACGCGGTGGGCGAGGCG
ACCGAGATCCTCACCGAGGGCGGCCTGCTGCACGACCTGCGGTTCCCGCA
TCCGGCGGCCCGTTCGGTCGTACTCAACGACCTGTCCGCCCGGGAACGCC
GCCGACTGCACCGGTCCGCTCTGGAAGTGCTGGATGACGTACCCGTTGAA
GTGGTCGCGCACCACCAGGCCGGTGCCGGTTTCATCCACGGTCCCAAGGC
CGCCGAGATCTTCGCCAAGGCCGGCCAGGAGCTGCATGTGCGCGGCGAGC
TGGACGCCGCGTCCGACTATCTGCAACTGGCCCACCACGCCTCCGACGAC
GCCGTCACCCGGGCCGCGCTGCGGGTCGAGGCCGTGGCGATCGAGCGCCG
CCGCAACCCGCTGGCCTCCAGCCGCCACCTCGACGAGCTGACCGTCGCCG
CCCGTGCCGGTCTGCTCTCCCTCGAGCACGCCGCGCTGATGATCCGCTGG
CTGGCTCTCGGCGGGCGGTCCGGCGAGGCGGCCGAGGTGCTGGCCGCGCA
GCGCCCGCGTGCGGTCACCGACCAGGACAGGGCCCACCTGCGGGCCGCCG
AGGTATCGCTGGCGCTGGTCAGCCCGGGCGCGTCCGGCGTCAGCCCGGGT
GCGTCCGGCCCGGATCGGCGGCCGCGTCCGCTCCCGCCGGATGAGCTCGC
GAACCTGCCGAAGGCGGCCCGGCTTTGTGCGATCGCCGACAACGCCGTCA
TATCGGCCCTGCACGGTCGTCCCGAGCTTGCCTCGGCCGAGGCGGAGAAC
GTCCTGAAGCAGGCTGACTCGGCGGCGGACGGCGCCACCGCCCTCTCCGC
GCTGACGGCCTTGCTGTACGCGGAGAACACCGACACCGCTCAGCTCTGGG
CCGACAAGCTCGTCTCCGAGACCGGGGCGTCGAACGAGGAGGAAGGCGCG
GGCTACGCGGGGCCGCGCGCCGAGACCGCGTTGCGCCGCGGCGACCTGGC
CGCGGCGGTCGAGGCGGGCAGCGCCATTCTGGACCACCGGCGGGGGTCGT
TGCTCGGCATCACCGCCGCGCTACCGCTGAGCAGCGCGGTAGCCGCCGCC
ATCCGGCTGGGCGAGACCGAGCGGGCGGAGAAGTGGCTCGCCGAGCCGCT
GCCGGAGGCCATTCGGGACAGCCTGTTCGGGCTGCACCTGCTCTCGGCGC
GCGGCCAGTACTGCCTCGCGACGGGCCGGCACGAGTCGGCGTACACGGCG
TTCCGCACCTGCGGGGAACGGATGCGGAACTGGGGCGTCGACGTGCCGGG
TCTGTCCCTGTGGCGCGTCGACGCCGCCGAGGCGCTGCTGCACGGCCGCG
ACCGGGACGAGGGCCGACGGCTCATCGACGAGCAGCTCACCCATGCGATG
GGACCCCGTTCCCGCGCTTTGACGCTGCGGGTGCAGGCGGCGTACAGCCC
GCAGGCGCAGCGGGTCGACCTGCTCGAAGAGGCGGCCGACCTGCTGCTCT
CCTGCAACGACCAGTACGAGCGGGCGCGGGTGCTCGCCGATCTGAGCGAG
GCGTTCAGCGCGCTCAGGCACCACAGCCGGGCGCGGGGACTGCTCCGGCA
GGCCCGGCACCTGGCCGCCCAGTGCGGCGCGACCCCGCTGCTGCGCCGGC
TCGGGGCCAAGCCCGGAGGCCCCGGCTGGCTGGAGGAATCCGGCCTGCCG
CAGCGGATCAAGTCGCTGACCGACGCGGAGCGGCGGGTGGCGTCGCTGGC
CGCCGGCGGCCAGACCAACCGCGTGATCGCCGACCAGCTCTTCGTCACGG
CCAGCACGGTGGAGCAGCACCTCACGAACGTCTTCCGCAAGCTGGGCGTC
AAGGGCCGCCAGCACCTGCCGGCCGAACTCGCCAACGCGGAATAG

SEQ ID NO: 192

GTGAAGCGCAACGATCTGGTTGCCCGCGATGGCGAGCTCAGGTGGATGCA
AGAGATTCTCAGTCAGGCGAGCGAGGGCCGGGGGGCCGTGGTCACCATCA
CGGGGGCGATCGCCTGTGGCAAGACGGTGCTGCTGGACGCCGCGGCAGCC
AGTCAAGACGTGATCCAACTGCGTGCGGTCTGCTCGGCGGAGGAGCAGGA
GCTGCCGTACGCGATGGTCGGACAACTACTCGACAATCCGGTGCTCGCCG
CGCGAGTGCCGGCCCTGGGCAACCTGGCTGCGGCGGGCGAGCGGCTGCTG
CCGGGCACCGAGAACAGGATCCGGCGGGAGCTCACCCGCACCCTGCTGGC
TCTCGCCGACGAACGACCGGTGCTGATCGGCGTCGACGACATGCACCATG
CGGACCCCGCCTCGCTGGACTGCCTGCTGCACCTGGCCCGGCGGGTCGGC
CCGGCCCGCATCGCGATCGTTCTGACCGAGTTGCGCCGGCTCACCCCGGC
TCACTCGCGCTTCCAGTCCGAGCTGCTCAGCCTGCGGTACCACCACGAGA
TCGGGTTGCAGCCGCTCACCGCGGAGCACACCGCCGACCTGGCCCGCGTC
GGCCTCGGTGCCGAGGTCGACGACGACGTGCTCACCGAGCTCTACGAGGC
GACCGGCGGCAACCCGAGTCTGTGCTGCGGCCTGATCAGGGACGTGCGGC
AGGACTGGGAGGCCGGGGTCACCGGTATCCACGTCGGCCGGGCGTACCGG
CTGGCCTATCTCAGTTCGCTCTACCGCTGCGGCCCGGCGGCGCTGCGGAC
CGCCCGCGCGGCCGCGGTGCTGGGCGACAGCGCCGACGCCTGCCTGATCC
GCCGGGTCAGCGGCCTCGGTACGGAGGCCGTGGGCCAGGCGATCCAGCAG
CTCACCGAGGGCGGCCTGCTGCGTGACCAGCAGTTCCCGCACCCGGCGGC
CCGCTCGGTCGTGCTCGACGACATGTCCGCGCAGGAACGCCACGCGATGT
ATCGCAGCGCCCGGGAGGCAGCCGCCGAAGGTCAGGCCGACCCCGGCACC
CCGGGCGAGCCGCGGGCGGCTACGGCGTACGCCGGGTGTGGTGAGCAAGC
CGGTGACTACCCGGAGCCGGCCGGCCGGGCCTGCGTGGACGGTGCCGGTC
CGGCCGAGTACTGCGGCGACCCGCACGGCGCCGACGACGACCCGGACGAG
CTGGTCGCCGCGCTGGGCGGGCTGCTGCCGAGCCGGCTCGTGGCGATGAA
GATCCGGCGCCTGGCGGTGGCCGGGCGCCCCGGGGCGGCTGCCGAGCTGC
TGACCTCGCAGCGGTTGCACGCGGTGACCAGCGAGGACCGGGCCAGCCTG
CGGGCCGCCGAGGTGGCGCTCGCCACGCTGTGGCCGGGTGCGACCGGCCC
GGACCGGCATCCGCTCACGGAGCAGGAGGCGGCGAGCCTGCCGGAGGGTC
CGCGCCTGCTCGCTGCCGCCGACGATGCCGTCGGGGCCGCCCTGCGCGGT
CGCGCCGAGTACGCCGCGGCCGAGGCGGAGAACGTCCTGCGGCACGCCGA
TCCGGCAGCCGGTGGTGACGCCTACGCCGCCATGATCGCCCTGCTGTACA
CGGAGCACCCCGAGAACGTGCTGTTCTGGGCCGACAAGCTCGACGCGGGC
CGCCCCGACGAGGAGACCAGTTATCCCGGGCTGCGGGCCGAGACCGCGGT
GCGGCTCGGTGACCTGGAAACGGCGATGGAGCTGGGCCGCACGGTGCTGG
ACCAGCGGCGGCTGCCGTCCCTGGGTGTCGCCGCGGGCCTGCTCCTGGGC
GGCGCGGTGACGGCCGCCATCCGGCTCGGCGACCTCGACCGGGCGGAGAA
GTGGCTCGCCGAGCCGATCCCCGACGCCATCCGTACCAGCCTCTACGGCC
TGCACGTGCTGGCCGCGCGGGGCCGGCTCGACCTGGCCGCGGGCCGCTAC
GAGGCGGCGTACACGGCGTTCCGGCTGTGTGGCGAGCGGATGGCAGGCTG
GGATGCCGATGTCTCCGGGCTGGCGCTGTGGCGCGTCGACGCCGCCGAGG
CCCTGCTGTCCGCGGGCATCCGCCCGGACGAGGGCCGCAAGCTCATCGAC
GACCAGCTCACCCGTGAGATGGGGGCCCGCTCCCGGGCGCTGACGCTGCG
GGCGCAAGCGGCGTACAGCCTGCCGGTGCACCGGGTGGGCCTGCTCGACG
AGGCGGCCGGCCTGCTGCTCGCCTGCCATGACGGGTACGAGCGGGCGCGG
GTGCTCGCGGACCTGGGGGAGACCCTGCGCACGCTGCGGCACACCGACGC
GGCCCAGCGGGTGCTCCGGCAGGCCGAGCAGGCGGCCGCGCGGTGCGGGT
CGGTCCCGCTGCTGCGGCGGCTCGGGGCCGAACCCGTACGCATCGGCACC
CGGCGTGGTGAACCCGGCCTGCCGCAGCGGATCAGGCTGCTGACCGATGC
CGAGCGGCGGGTTGCCGCGATGGCCGCCGCCGGGCAGACCAACCGGGAGA
TCGCCGGTCGGCTCTTCGTCACGGCCAGCACGGTGGAGCAGCACCTGACC
AGCGTCTTCCGCAAGCTGGGCGTCAAGGGCCGCCGGTTCCTGCCGACCGA
GCTCGCCCAAGCCGTCTGA

SEQ ID NO: 193

ATGCCTGCCGTGAAGCGCAACGATCTGGTTGCCCGCGATGGCGAGCTCAG
GTGGATGCAAGAGATTCTCAGTCAGGCGAGCGAGGGCCGGGGGGCCGTGG
TCACCATCACGGGGGCGATCGCCTGTGGCAAGACGGTGCTGCTGGACGCC
GCGGCAGCCAGTCAAGACGTGATCCAACTGCGTGCGGTCTGCTCGGCGGA
GGAGCAGGAGCTGCCGTACGCGATGGTCGGACAACTACTCGACAATCCGG
TGCTCGCCGCGCGAGTGCCGGCCCTGGGCAACCTGGCTGCGGCGGGCGAG
CGGCTGCTGCCGGGCACCGAGAACAGGATCCGGCGGGAGCTCACCCGCAC
CCTGCTGGCTCTCGCCGACGAACGACCGGTGCTGATCGGCGTCGACGACA
TGCACCATGCGGACCCCGCCTCGCTGGACTGCCTGCTGCACCTGGCCCGG
CGGGTCGGCCCGGCCCGCATCGCGATCGTTCTGACCGAGTTGCGCCGGCT
CACCCCGGCTCACTCGCGCTTCCAGTCCGAGCTGCTCAGCCTGCGGTACC
ACCACGAGATCGGGTTGCAGCCGCTCACCGCGGAGCACACCGCCGACCTG
GCCCGCGTCGGCCTCGGTGCCGAGGTCGACGACGACGTGCTCACCGAGCT
CTACGAGGCGACCGGCGGCAACCCGAGTCTGTGCTGCGGCCTGATCAGGG
ACGTGCGGCAGGACTGGGAGGCCGGGGTCACCGGTATCCACGTCGGCCGG
GCGTACCGGCTGGCCTATCTCAGTTCGCTCTACCGCTGCGGCCCGGCGGC
GCTGCGGACCGCCCGCGCGGCCGCGGTGCTGGGCGACAGCGCCGACGCCT
GCCTGATCCGCCGGGTCAGCGGCCTCGGTACGGAGGCCGTGGGCCAGGCG
ATCCAGCAGCTCACCGAGGGCGGCCTGCTGCGTGACCAGCAGTTCCCGCA
CCCGGCGGCCCGCTCGGTCGTGCTCGACGACATGTCCGCGCAGGAACGCC
ACGCGATGTATCGCAGCGCCCGGGAGGCAGCCGCCGAAGGTCAGGCCGAC
CCCGGCACCCCGGGCGAGCCGCGGGCGGCTACGGCGTACGCCGGGTGTGG
TGAGCAAGCCGGTGACTACCCGGAGCCGGCCGGCCGGGCCTGCGTGGACG
GTGCCGGTCCGGCCGAGTACTGCGGCGACCCGCACGGCGCCGACGACGAC
CCGGACGAGCTGGTCGCCGCGCTGGGCGGGCTGCTGCCGAGCCGGCTCGT
GGCGATGAAGATCCGGCGCCTGGCGGTGGCCGGGCGCCCCGGGGCGGCTG
CCGAGCTGCTGACCTCGCAGCGGTTGCACGCGGTGACCAGCGAGGACCGG
GCCAGCCTGCGGGCCGCCGAGGTGGCGCTCGCCACGCTGTGGCCGGGTGC
GACCGGCCCGGACCGGCATCCGCTCACGGAGCAGGAGGCGGCGAGCCTGC
CGGAGGGTCCGCGCCTGCTCGCTGCCGCCGACGATGCCGTCGGGGCCGCC
CTGCGCGGTCGCGCCGAGTACGCCGCGGCCGAGGCGGAGAACGTCCTGCG
GCACGCCGATCCGGCAGCCGGTGGTGACGCCTACGCCGCCATGATCGCCC
TGCTGTACACGGAGCACCCCGAGAACGTGCTGTTCTGGGCCGACAAGCTC
GACGCGGGCCGCCCCGACGAGGAGACCAGTTATCCCGGGCTGCGGGCCGA
GACCGCGGTGCGGCTCGGTGACCTGGAAACGGCGATGGAGCTGGGCCGCA
CGGTGCTGGACCAGCGGCGGCTGCCGTCCCTGGGTGTCGCCGCGGGCCTG
CTCCTGGGCGGCGCGGTGACGGCCGCCATCCGGCTCGGCGACCTCGACCG
GGCGGAGAAGTGGCTCGCCGAGCCGATCCCCGACGCCATCCGTACCAGCC
TCTACGGCCTGCACGTGCTGGCCGCGCGGGGCCGGCTCGACCTGGCCGCG
GGCCGCTACGAGGCGGCGTACACGGCGTTCCGGCTGTGTGGCGAGCGGAT
GGCAGGCTGGGATGCCGATGTCTCCGGGCTGGCGCTGTGGCGCGTCGACG
CCGCCGAGGCCCTGCTGTCCGCGGGCATCCGCCCGGACGAGGGCCGCAAG
CTCATCGACGACCAGCTCACCCGTGAGATGGGGGCCCGCTCCCGGGCGCT
GACGCTGCGGGCGCAAGCGGCGTACAGCCTGCCGGTGCACCGGGTGGGCC
TGCTCGACGAGGCGGCCGGCCTGCTGCTCGCCTGCCATGACGGGTACGAG
CGGGCGCGGGTGCTCGCGGACCTGGGGGAGACCCTGCGCACGCTGCGGCA
CACCGACGCGGCCCAGCGGGTGCTCCGGCAGGCCGAGCAGGCGGCCGCGC
GGTGCGGGTCGGTCCCGCTGCTGCGGCGGCTCGGGGCCGAACCCGTACGC
ATCGGCACCCGGCGTGGTGAACCCGGCCTGCCGCAGCGGATCAGGCTGCT
GACCGATGCCGAGCGGCGGGTTGCCGCGATGGCCGCCGCCGGGCAGACCA
ACCGGGAGATCGCCGGTCGGCTCTTCGTCACGGCCAGCACGGTGGAGCAG
CACCTGACCAGCGTCTTCCGCAAGCTGGGCGTCAAGGGCCGCCGGTTCCT
GCCGACCGAGCTCGCCCAAGCCGTCTGA

SEQ ID NO: 194

GTGGTCACCGTCACCGGCCCAATCGCCTGCGGCAAGACAGAACTGCTTGA
CGCGGCTGCCGCGAAGGCTGAGGCCATCATTCTGCGCGCGGTCTGCGCGC
CAGAAGAGCGGGCTATGCCGTACGCCATGATCGGGCAGCTCATCGACGAC
CCGGCGCTCGCGCATCGGGCGCCGGGGCTGGCTGATCGGATAGCCCAGGG
CGGGCAGCTGTCGCTGAGGGCCGAGAACCGACTGCGCAGGGATCTCACCC
GTGCCCTGCTGGCGCTTGCCGTGGACCGGCCTGTGCTGATCGGCGTCGAC
GATGTGCATCACGCCGACACCGCCTCTTTGAACTGTCTGCTGCATTTGGC
CCGCCGGGTCCGTCCGGCCCGGATATCCATGATCTTCACCGAGTTGCGCA
GCCTCACCCCTACTCAGTCACGGTTCAAGGCGGAGCTGCTCAGCCTGCCA
TACCACCACGAGATCGCGCTGCGTCCATTCGGACCGGAGCAATCGGCGGA
GCTGGCTCGCGCCGCCTTCGGCCCGGGCCTCGCCGAGGATGTGCTCGCGG
GGTTGTATAAAACGACCAGGGGCAATCTGAGTCTCAGCCGTGGACTGATC
AGCGATGTGCGGGAGGCCCTGGCCAACGGAGAGAGCGCTTTCGAGGCGGG
CCGCGCGTTCCGGCTGGCGTACCTCAGCTCGCTCTACCGCTGTGGCCCGG
TCGCGCTGCGGGTCGCCCGAGTGGCTGCCGTGCTGGGCCCAAGCGCCACC
ACCACGCTGGTGCGCCGGCTAAGCGGGCTCAGCGCGGAGACGATAGACCG
GGCAACCAAGATCCTCACTGAGGGCGGGCTGCTGCTCGACCAGCAGTTCC
CGCACCCGGCCGCCCGCTCGGTGGTGCTCGATGACATGTCCGCCCAGGAA
CGACGCAGCCTGCACACTCTCGCCCTGGAACTGCTGGACGAGGCGCCGGT
TGAAGTGCTCGCGCACCACCAGGTCGGCGCCGGTCTCATACACGGGCCCA
AGGCTGCGGAGATGTTCGCCAAGGCCGGCAAGGCTCTGGTCGTACGCAAC
GAGTTGGGCGACGCGGCCGAATACCTGCAACTGGCTCACCGGGCCTCCGA
CGATGTCTCCACCCGGGCCGCCTTACGGGTCGAGGCCGTGGCCATCGAGC
GCCGCCGCAATCCGCTGGCCTCCAGTCGGCACATGGACGAACTGAGCGCC
GCCGGCCGCGCCGGTCTGCTTTCCCCCAAGCATGCGGCGCTGGCCGTCTT
CTGGCTAGCCGACGGCGGGCGATCCGGCGAGGCAGCCGAAGTGCTGGCGT
CGGAACGCCCGCTCGCGACCACCGATCAGAACCGGGCCCACCTGCGATTT
GTCGAGGTGACTCTCGCGCTGTTCTCTCCCGGCGCCTTCGGATCGGACCG
GCGCCCACCTCCGCTGACGCCGGACGAACTCGCCAGCCTGCCGAAGGCGG
CCTGGCAATGCGCGGTCGCCGACAACGCGGCCATGACCGCCTTGCACGGC
CATCCAGAACTTGCCACCGCTCAGGCGGAAACAGTTCTGCGGCAGGCTGA
TTCGGCAGCCGACGCGATCCCCGCCGCGCTGATCGCCCTGTTGTACGCGG
AGAACACCGAGTCCGCTCATATCTGGGCCGACAAGCTGGGCAGCACGAAT
GCCGGGGTATCGAACGAGGCGGAAGCGGGCTACGCCGGCCCGTGCGCCGA
GATCGCCCTGCGGCGCGGCGACCTGGCCACGGCGTTCGAGGCTGGTAGCG
CCGTCCTGGACGACCGGTCGCTGCCGTCGCTCGGCATCACCGCCGCATTG
CTGTTGAGCAGCAAGACGGCCGCCGCTGTCCGGCTGGGCGAACTCGAGCG
TGCGGAGAAGCTGCTCGCCGAGCCGCTTCCGAACGGCGTCCAGGACAGCC
TTTTCGGTCTGCACCTGCTCTCGGCGTACGGCCAGTACAGCCTCGCGATG
GGCCGATATGAATCAGCTCACCGGGCGTTTCGCACCTGCGGAGAACGTAT
GCGCAGCTGGGATGTTGACGTGCCTGGTCTGGCCCTGTGGCGTGTCGACG
CCGCCGAGGCGCTGCTCAGCCTCGACCGGAACGAGGGCCAGCGGCTCATC
GACGAACAACTCACCCGTCCGATGGGGCCTCGTTCCCACGCGTTAACGCT
GCGGATCAAGGCGGCATACCTCCCGCGGACGAAGCGGATCCCCCTGCTCC
ATGAGGCGGCCGAGCTGCTGCTCCCCTGCCCCGACCCGTACGAGCAAGCG
CGGGTGCTCGCCGATCTGGGCGACACGCTCAGCGCGCTCAGACGCTATAG
CCGGGCGCGGGGAGTTCTCCGGCAGGCTCGTCACCTGGCCACCCAGTGCG
GTGCTGTCCCGCTGCTGCGCAGGCTCGGGGGCGAGCCCGGCCGGATCGAC
GACGCCGGCCTGCCGCAGCGGAGCACATCGTTGACCGATGCGGAGCGGCG
GGTGGCGGCGCTGGCCGCGGCCGGACAGACCAACCGGGAGATCGCCGAAC
AGCTGTTCGTCACGGCCAGCACAGTGGAACAGCACCTCACAAGCGTCTTC
CGCAAGCTGGGCGTCAAGGGCCGCAAGCAGCTGCCGACCGCGCTGGCCGA
CGTGGAACAGACCTGA

SEQ ID NO: 195

ATGTATAGCGGTACCTGCCGTGAAGGATACGAACTCGTCGCACGCGAGGA
CGAACTCGGTATTCTACAGAGGTCTCTGGAACAAGCGAGCAGCGGCCAGG
GCGTCGTGGTCACCGTCACCGGCCCAATCGCCTGCGGCAAGACAGAACTG
CTTGACGCGGCTGCCGCGAAGGCTGAGGCCATCATTCTGCGCGCGGTCTG
CGCGCCAGAAGAGCGGGCTATGCCGTACGCCATGATCGGGCAGCTCATCG
ACGACCCGGCGCTCGCGCATCGGGCGCCGGGGCTGGCTGATCGGATAGCC
CAGGGCGGGCAGCTGTCGCTGAGGGCCGAGAACCGACTGCGCAGGGATCT
CACCCGTGCCCTGCTGGCGCTTGCCGTGGACCGGCCTGTGCTGATCGGCG
TCGACGATGTGCATCACGCCGACACCGCCTCTTTGAACTGTCTGCTGCAT
TTGGCCCGCCGGGTCCGTCCGGCCCGGATATCCATGATCTTCACCGAGTT
GCGCAGCCTCACCCCTACTCAGTCACGGTTCAAGGCGGAGCTGCTCAGCC
TGCCATACCACCACGAGATCGCGCTGCGTCCATTCGGACCGGAGCAATCG
GCGGAGCTGGCTCGCGCCGCCTTCGGCCCGGGCCTCGCCGAGGATGTGCT
CGCGGGGTTGTATAAAACGACCAGGGGCAATCTGAGTCTCAGCCGTGGAC
TGATCAGCGATGTGCGGGAGGCCCTGGCCAACGGAGAGAGCGCTTTCGAG
GCGGGCCGCGCGTTCCGGCTGGCGTACCTCAGCTCGCTCTACCGCTGTGG
CCCGGTCGCGCTGCGGGTCGCCCGAGTGGCTGCCGTGCTGGGCCCAAGCG
CCACCACCACGCTGGTGCGCCGGCTAAGCGGGCTCAGCGCGGAGACGATA
GACCGGGCAACCAAGATCCTCACTGAGGGCGGGCTGCTGCTCGACCAGCA
GTTCCCGCACCCGGCCGCCCGCTCGGTGGTGCTCGATGACATGTCCGCCC
AGGAACGACGCAGCCTGCACACTCTCGCCCTGGAACTGCTGGACGAGGCG
CCGGTTGAAGTGCTCGCGCACCACCAGGTCGGCGCCGGTCTCATACACGG
GCCCAAGGCTGCGGAGATGTTCGCCAAGGCCGGCAAGGCTCTGGTCGTAC
GCAACGAGTTGGGCGACGCGGCCGAATACCTGCAACTGGCTCACCGGGCC
TCCGACGATGTCTCCACCCGGGCCGCCCTGCGGGTCGAGGCCGTGGCCAT
CGAGCGCCGCCGCAATCCGCTGGCCTCCAGTCGGCACATGGACGAACTGA
GCGCCGCCGGCCGCGCCGGTCTGCTTTCCCCCAAGCATGCGGCGCTGGCC
GTCTTCTGGCTAGCCGACGGCGGGCGATCCGGCGAGGCAGCCGAAGTGCT
GGCGTCGGAACGCCCGCTCGCGACCACCGATCAGAACCGGGCCCACCTGC
GATTTGTCGAGGTGACTCTCGCGCTGTTCTCTCCCGGCGCCTTCGGATCG
GACCGGCGCCCACCTCCGCTGACGCCGGACGAACTCGCCAGCCTGCCGAA
GGCGGCCTGGCAATGCGCGGTCGCCGACAACGCGGCCATGACCGCCTTGC
ACGGCCATCCAGAACTTGCCACCGCTCAGGCGGAAACAGTTCTGCGGCAG
GCTGATTCGGCAGCCGACGCGATCCCCGCCGCGCTGATCGCCCTGTTGTA
CGCGGAGAACACCGAGTCCGCTCATATCTGGGCCGACAAGCTGGGCAGCA
CGAATGCCGGGGTATCGAACGAGGCGGAAGCGGGCTACGCCGGCCCGTGC
GCCGAGATCGCCCTGCGGCGCGGCGACCTGGCCACGGCGTTCGAGGCTGG
TAGCGCCGTCCTGGACGACCGGTCGCTGCCGTCGCTCGGCATCACCGCCG
CATTGCTGTTGAGCAGCAAGACGGCCGCCGCTGTCCGGCTGGGCGAACTC
GAGCGTGCGGAGAAGCTGCTCGCCGAGCCGCTTCCGAACGGCGTCCAGGA
CAGCCTTTTCGGTCTGCACCTGCTCTCGGCGTACGGCCAGTACAGCCTCG
CGATGGGCCGATATGAATCAGCTCACCGGGCGTTTCGCACCTGCGGAGAA
CGTATGCGCAGCTGGGATGTTGACGTGCCTGGTCTGGCCCTGTGGCGTGT
CGACGCCGCCGAGGCGCTGCTCAGCCTCGACCGGAACGAGGGCCAGCGGC
TCATCGACGAACAACTCACCCGTCCGATGGGGCCTCGTTCCCACGCGCTG
ACGCTGCGGATCAAGGCGGCATACCTCCCGCGGACGAAGCGGATCCCCCT
GCTCCATGAGGCGGCCGAGCTGCTGCTCCCCTGCCCCGACCCGTACGAGC
AAGCGCGGGTGCTCGCCGATCTGGGCGACACGCTCAGCGCGCTCAGACGC
TATAGCCGGGCGCGGGGAGTTCTCCGGCAGGCTCGTCACCTGGCCACCCA
GTGCGGTGCTGTCCCGCTGCTGCGCAGGCTCGGGGGCGAGCCCGGCCGGA
TCGACGACGCCGGCCTGCCGCAGCGGAGCACATCGTTGACCGATGCGGAG
CGGCGGGTGGCGGCGCTGGCCGCGGCCGGACAGACCAACCGGGAGATCGC
CGAACAGCTGTTCGTCACGGCCAGCACAGTGGAACAGCACCTCACAAGCG
TCTTCCGCAAGCTGGGCGTCAAGGGCCGCAAGCAGCTGCCGACCGCGCTG
GCCGACGTGGAACAGACCTGA

SEQ ID NO: 196

GTGTATAGCGGTACCTGCCGTGAAGGATACGAACTCGTCGCCCGCGAGGA
CGAACTCGGCATTCTGCAGAGGTCTCTGGAAGAAGCAGGCAGCGGCCAGG
GCGCCGTGGTCACCGTCACCGGCCCGATCGCCTGCGGCAAGACAGAACTG
CTTGACGCGGCTGCCGCGAAGGCTGACGCCATCATTCTGCGCGCGGTCTG
CGCGCCCGAAGAGCGCGCTATGCCGTACGCCATGATCGGGCAGCTCATCG
ACGACCCGGCGCTCGCGCATCGGGCGCCGGAGCTGGCTGATCGGATAGCC
CAGGGCGGGCATCTGTCGCTGAGGGCCGAGAACCGACTGCGCAGGGATCT
CACCCGTGCCCTGCTGGCGCTTGCCGTCGACCGGCCTGTGCTGATCGGCG
TCGACGATGTGCATCACGCCGACACCGCCTCTTTGAACTGTCTGCTGCAT
TTAGCCCGCCGGGTCCGTCCGGCCCGGATATCCATGATCTTCACCGAGTT
GCGCAGCCTCACCCCTACTCAGTCACGATTCAAGGCGGAGCTGCTCAGCC
TGCCGTACCACCACGAGATCGCGCTGCGTCCACTCGGACCGGAGCAATCG
GCGGAGCTGGCCCACGCCGCCTTCGGCCCGGGCCTCGCCGAGGATGTGCT
CGCGGGGTTGTATGGGATGACCAGGGGCAACCTGAGTCTCAGCCGTGGAC
TGATCAGCGATGTGCGGGAGGCCCAGGCCAACGGAGAGAGCGCTTTCGAG
GTGGGCCGCGCGTTCCGGCTGGCGTACCTCAGCTCGCTCTACCGCTGTGG
CCCGATCGCGCTGCGGGTCGCCCGAGTGGCTGCCGTGCTGGGCCCAAGCG
CCACCACCACGCTGGTGCGCCGTCTAAGCGGGCTCAGCGCGGAGACGATA
GACCGGGCAACCAAGATCCTCACTGAGGGCGGGCTGCTGCTCGACCACCA
GTTCCCGCACCCGGCCGCCCGCTCGGTGGTGCTCGATGACATGTCCGCCC
AGGAACGACGCAGCCTGCACACTCTCGCCCTGGAACTGCTGGACGAGGCG
CCGGTTGAAGTGCTCGCGCACCACCAGGTCGGCGCCGGTCTCATACACGG
GCCCAAGGCTGCGGAGATATTCGCCAGGGCTGGCCAGGCTCTGGTTGTAC
GCAACGAGTTGGGCGACGCGGCCGAATACCTGCAACTGGCTCACCGAGCC
TCCGACGATGTCTCCACCCGGGCCGCCTTACGGGTCGAGGCCGTGGCAAT
CGAGCGCCGCCGCAATCCGCTGGCCTCCAGTCGTCACATGGACGAGCTGA
GCGCCGCCGGCCGCGCCGGTCTGCTTTCCCCCAAGCATGCAGCGCTGGCT
GTCTTCTGGCTGGCCGACGGCGGGCGATCCGGCGAGGCAGCCGAGGTGCT
GGCGTCGGAACACCCGCTCGCGACCACCGATCAGAACCGAGCACACCTGC
GATTTGCCGAGGTGACTCTCGCGCTGTTCTGTCCCGGCGCCTTCGGGTCG
GACCGGCGCCCACCTCCGCTGGCGCCGGACGAGCTCGCCAGCTTGCCGAA
GGCGGCCTGGCAATGCGCGGTCGCCGACAACGCGGTCATGACAGCGTTGC
ATGCTCATCCAGAACTTGCCACCGCTCAGGCGGAAACAGTTCTGCGGCAG
GCTGATTCGGCAGCCGACGCAATCCCCGCCGCACTGATCGCCCTGTTGTA
CGCAGAGAACACCGAGTCCGCTCAGATCTGGGCCGACAAGCTGGGCAGCA
CCAATGCCGGGGTATCGAACGAGGCGGAAGCGGGCTACGCCGGCCCGTGC
GCCGAGATCGCCCTGCGGCGCGGCGACCTGGCCACGGCGTTCGAGGCTGG
TGGCACCGTCCTGGACGACCGGCCGCTGCCGTCGCTCGGCATCACCGCCG
CATTGCTGTTGAGCAGCAAGACGGCAGCCGCTGTCCGCCTGGGCGAACTC
GAGCGTGCGGAGAAGCTGCTCGCTGAGCCGCTTCCGAACGGTGTCCAGGA
CAGCCTTTTCGGTCTGCACCTGCTCTCGGCGCACGGCCAGTACAGCCTCG
CGATGGGCCGATATGAATCGGCTCACCGGGCGTTTCACACCTGCGGAGAA
CGTATGCGCAGCTGGGGTGTTGACGTGCCTGGTCTAGCCCTGTGGCGTGT
CGACGCCGCCGAGGCACTGCTCAGCCTCGACCGGAACGAGGGCCAGCGGC
TCATCGACGAACAACTCGCCCGTCCGATGGGACCTCGTTCCCGCGCATTA
ACGCTGCGGATCAAGGCGGCATACCTCCCGCGGACGAAGCGGATCCCCCT
GCTCCATGAGGCAGCTGAGCTGCTGCTCTCCTGCCCCGACCCGTACGAGC
AAGCGCGGGTGCTCGCCGATCTGGGCGACACGCTCAGCGCGCTCAGACGC
TATAGCCGGGCGCGGGGAGTTCTCCGGCAGGCTCGTCACCTGGCCACCCA
GTGCGGTGCTGTCCCGCTGCTGCGCCGACTCGGGGGCGAGCCCGGCCGGA
TCGACGACGCCGGCCTGCCGCAGCGGAGCACATCGTTGACCGATGCGGAG
CGGCGGGTGTCGGCCCTGGCCGCGGCCGGACAGACCAACCGGGAGATCGC
CAAACAGCTATTCGTCACGGCCAGCACCGTGGAACAGCACCTCACAAGCG
TCTTCCGCAAGCTGGGCGTTAAGGGCCGCAGGCAGCTACCGACCGCGCTG
GCCGACGTGGAATAG

SEQ ID NO: 197

ATGTATAGCGGTACCTGCCGTGAAGGATACGAACTCGTCGCCCGCGAGGA
CGAACTCGGCATTCTGCAGAGGTCTCTGGAAGAAGCAGGCAGCGGCCAGG
GCGCCGTGGTCACCGTCACCGGCCCGATCGCCTGCGGCAAGACAGAACTG
CTTGACGCGGCTGCCGCGAAGGCTGACGCCATCATTCTGCGCGCGGTCTG
CGCGCCCGAAGAGCGCGCTATGCCGTACGCCATGATCGGGCAGCTCATCG
ACGACCCGGCGCTCGCGCATCGGGCGCCGGAGCTGGCTGATCGGATAGCC
CAGGGCGGGCATCTGTCGCTGAGGGCCGAGAACCGACTGCGCAGGGATCT
CACCCGTGCCCTGCTGGCGCTTGCCGTCGACCGGCCTGTGCTGATCGGCG
TCGACGATGTGCATCACGCCGACACCGCCTCTTTGAACTGTCTGCTGCAT
CTGGCCCGCCGGGTCCGTCCGGCCCGGATATCCATGATCTTCACCGAGTT
GCGCAGCCTCACCCCTACTCAGTCACGATTCAAGGCGGAGCTGCTCAGCC
TGCCGTACCACCACGAGATCGCGCTGCGTCCACTCGGACCGGAGCAATCG
GCGGAGCTGGCCCACGCCGCCTTCGGCCCGGGCCTCGCCGAGGATGTGCT
CGCGGGGTTGTATGGGATGACCAGGGGCAACCTGAGTCTCAGCCGTGGAC
TGATCAGCGATGTGCGGGAGGCCCAGGCCAACGGAGAGAGCGCTTTCGAG
GTGGGCCGCGCGTTCCGGCTGGCGTACCTCAGCTCGCTCTACCGCTGTGG
CCCGATCGCGCTGCGGGTCGCCCGAGTGGCTGCCGTGCTGGGCCCAAGCG
CCACCACCACGCTGGTGCGCCGTCTAAGCGGGCTCAGCGCGGAGACGATA
GACCGGGCAACCAAGATCCTCACTGAGGGCGGGCTGCTGCTCGACCACCA
GTTCCCGCACCCGGCCGCCCGCTCGGTGGTGCTCGATGACATGTCCGCCC
AGGAACGACGCAGCCTGCACACTCTCGCCCTGGAACTGCTGGACGAGGCG
CCGGTTGAAGTGCTCGCGCACCACCAGGTCGGCGCCGGTCTCATACACGG
GCCCAAGGCTGCGGAGATATTCGCCAGGGCTGGCCAGGCTCTGGTTGTAC
GCAACGAGTTGGGCGACGCGGCCGAATACCTGCAACTGGCTCACCGAGCC
TCCGACGATGTCTCCACCCGGGCCGCCCTGCGGGTCGAGGCCGTGGCAAT
CGAGCGCCGCCGCAATCCGCTGGCCTCCAGTCGTCACATGGACGAGCTGA
GCGCCGCCGGCCGCGCCGGTCTGCTTTCCCCCAAGCATGCAGCGCTGGCT
GTCTTCTGGCTGGCCGACGGCGGGCGATCCGGCGAGGCAGCCGAGGTGCT
GGCGTCGGAACACCCGCTCGCGACCACCGATCAGAACCGAGCACACCTGC
GATTTGCCGAGGTGACTCTCGCGCTGTTCTGTCCCGGCGCCTTCGGGTCG
GACCGGCGCCCACCTCCGCTGGCGCCGGACGAGCTCGCCAGCTTGCCGAA
GGCGGCCTGGCAATGCGCGGTCGCCGACAACGCGGTCATGACAGCGTTGC
ATGCTCATCCAGAACTTGCCACCGCTCAGGCGGAAACAGTTCTGCGGCAG
GCTGATTCGGCAGCCGACGCAATCCCCGCCGCACTGATCGCCCTGTTGTA
CGCAGAGAACACCGAGTCCGCTCAGATCTGGGCCGACAAGCTGGGCAGCA
CCAATGCCGGGGTATCGAACGAGGCGGAAGCGGGCTACGCCGGCCCGTGC
GCCGAGATCGCCCTGCGGCGCGGCGACCTGGCCACGGCGTTCGAGGCTGG
TGGCACCGTCCTGGACGACCGGCCGCTGCCGTCGCTCGGCATCACCGCCG
CATTGCTGTTGAGCAGCAAGACGGCAGCCGCTGTCCGCCTGGGCGAACTC
GAGCGTGCGGAGAAGCTGCTCGCTGAGCCGCTTCCGAACGGTGTCCAGGA
CAGCCTTTTCGGTCTGCACCTGCTCTCGGCGCACGGCCAGTACAGCCTCG
CGATGGGCCGATATGAATCGGCTCACCGGGCGTTTCACACCTGCGGAGAA
CGTATGCGCAGCTGGGGTGTTGACGTGCCTGGTCTAGCCCTGTGGCGTGT
CGACGCCGCCGAGGCACTGCTCAGCCTCGACCGGAACGAGGGCCAGCGGC
TCATCGACGAACAACTCGCCCGTCCGATGGGACCTCGTTCCCGCGCACTG
ACGCTGCGGATCAAGGCGGCATACCTCCCGCGGACGAAGCGGATCCCCCT
GCTCCATGAGGCAGCTGAGCTGCTGCTCTCCTGCCCCGACCCGTACGAGC
AAGCGCGGGTGCTCGCCGATCTGGGCGACACGCTCAGCGCGCTCAGACGC
TATAGCCGGGCGCGGGGAGTTCTCCGGCAGGCTCGTCACCTGGCCACCCA
GTGCGGTGCTGTCCCGCTGCTGCGCCGACTCGGGGGCGAGCCCGGCCGGA
TCGACGACGCCGGCCTGCCGCAGCGGAGCACATCGTTGACCGATGCGGAG
CGGCGGGTGTCGGCCCTGGCCGCGGCCGGACAGACCAACCGGGAGATCGC
CAAACAGCTATTCGTCACGGCCAGCACCGTGGAACAGCACCTCACAAGCG
TCTTCCGCAAGCTGGGCGTTAAGGGCCGCAGGCAGCTACCGACCGCGCTG
GCCGACGTGGAATAG

SEQ ID NO: 198

GTGTATAGCGGTACCTGCCGTGAAGGATACGAACTCGTCGCACGCGAGGA
CGAACTCGGCATTCTACAGAGGTCTCTGGAACAAGCGAGCAGCGGCCAGG
GCGTCGTGGTCACCGTCACCGGCCCAATCGCCTGCGGCAAGACAGAACTG
CTTGACGCGGCTGCCGCGAAGGCTGAGGCCATCATTCTGCGCGCGGTCTG
CGCGCCCGAAGAGCGGGCTATGCCGTACGCCATGATCGGGCAGCTCATCG
ACGACCCGGCGCTCGCGCATCGGGCGCCGGGGCTGGCTGATCGGATAGCC
CAGGGCGGGCAGCTGTCGCTGAGGGCCGAGAACCGACTGCGCAGGGATCT
CACCCGTGCCCTGCTGGCGCTTGCCGTGCACCGGCCTGTGCTGATCGGCG
TCGATGATGTGCATCACGCCGACACCGCCTCTTTGAACTGTCTGCTGCAT
TTGGCGCGCCGGGTCCGTCCGGCCCGGATATCCATGATCTTCACCGAGTT
GCGCAGCCTCACCCCTACTCAGTCACGATTCAAGGCGGAGCTGCTCAGCC
TGCCGTACCACCACGAGATCGCGCTGCGTCCATTCGGACCGGAGCAATCG
GCGGAGCTGGCTCGCGCCGCCTTCGGCCCGGGCCTCGCCGAGGATGTGCT
CGCGGGGTTGTATAAAACGACCAGGGGCAATCTGAGTCTCAGCCGTGGAC
TGATCAGCGATGTGCGGGAGGCCCTGGCCAACGGAGAGAGCGCTTTCGAG
GCGGGCCGCGCGTTCCGGCTGGCGTACCTCAGCTCGCTCTACCGCTGTGG
CCCGGTCGCGCTGCGGGTCGCCCGAGTGGCTGCCGTGCTGGGCCCAAGCG
CCACCACCACGCTGGTGCGCCGGCTAAGCGGGCTCAGCGCGGAGACGATA
GACCGGGCAACCAAGATCCTCACCGAGGGCGGGCTGCTGCTCGACCAGCA
GTTTCCGCACCCGGCCGCCCGCTCGGTGGTGCTCGATGACATGTCCGCCC
AGGAACGACGCGGCCTGCACACTCTCGCCCTGGAACTGCTGGACGAGGCG
CCGGTTGAAGTGCTCGCGCACCACCAGGTCGGCGCCGGTCTCATACACGG
GCCCAAGGCTGCGGAGATGTTCGCCAAGGCCGGCAAGGCTCTGGTCGTAC
GCAACGAGTTGGGCGACGCGGCCGAATACCTGCAACTGGCTCACCGGGCC
TCCGACGATGTCTCCACCCGGGCCGCCTTACGGGTCGAGGCCGTGGCGAT
CGAGCGCCGCCGCAATCCGCTGGCCTCCAGTCGGCACATGGACGAGCTGA
GCGCCGCCGGCCGCGCCGGTCTGCTTTCCCCCAAGCATGCGGCGCTGGCC
GTCTTCTGGCTGGCCGACGGCGGGCGATCCGGCGAGGCAGCCCAGGTGCT
GGCGTCGGAACGCCCGCTCGCGACCACCGATCAGAACCGGGCCCACCTGC
GATTTGTCGAGGTGACTCTCGCGCTGTTCTCTCCCGGCGCCTTCGGATCG
GACCGGCGCCCACCTCCGCTGACGCCGGACGAACTCGCCAGCCTGCCGAA
GGCGGCCTGGCAATGCGCGGTCGCCGACAACGCGGCCATGACCGCCTTGC
ACGGCCATCCAGAACTTGCCACCGCTCAGGCGGAAACAGTTCTGCGGCAG
GCTGATTCGGCAGCCGACGCGATCCCCGCCGCGCTGATCGCCCTGTTGTA
CGCGGAGAACACCGAGTCCGCTCATATCTGGGCCGACAAGCTGGGCAGCA
TGAATGCCGGGGTATCGAACGAGGCGGAAGCGGGCTACGCCGGCCCGTGC
GCCGAGATCGCCCTGCGGCGCGGCGACCTGGCCACGGCGTTCGAGGCTGG
TAGCACCGTCCTGGACGACCGGTCACTGCCGTCGCTCGGCATCACCGCCG
CATTGCTGTTGAGCAGCAAGACGGCCGCCGCTGTCCGGCTGGGCGAACTC
GAGCGTGCGGAGAAGCTGCTCGCCGAGCCGCTTCCGAACGGCGTCCAGGA
CAGCCTTTTCGGTCTGCACCTGCTCTCGGCGTACGGCCAGTACAGCCTCG
CGATGGGCCGATATGAATCGGCTCACCGGGCGTTTCGCACCTGCGGAGAA
CGTATGCGCAGCTGGGATGTTGACGTGCCTGGTCTGGCCCTGTGGCGTGT
CGACGCCGCCGAGGCGCTGCTCAGCCTCGACCGGAACGAGGGCCAGCGGC
TCATCGACGAACAACTCACCCGTCCGATGGGACCTCGTTCCCGCGCGTTA
ACGCTGCGGATCAAGGCGGCATACCTCCCGCGGACGAAGCGGATCCCCCT
GCTCCATGAGGCGGCCGAGCTGCTGCTCCCCTGCCCCGACCCGTACGAGC
AAGCGCGGGTGCTCGCCGATCTGGGCGACACGCTCAGCGCGCTCAGACGC
TATAGCCGGGCGCGGGGAGTTCTCCGGCAGGCTCGTCACCTGGCCACCCA
GTGCGGTGCTGTCCCGCTGCTGCGCCGACTCGGGGGCGAGCCCGGCCGGA
TCGACGACGCCGGCCTGCCGCAGCGGAGCACATCGTTGACCGATGCGGAG
CGGCGGGTGGCGGCGCTGGCCGCGGCCGGACAGACCAACCGGGAGATCGC
CGAACAGCTGTTCGTCACGGCCAGCACAGTGGAACAGCACCTCACAAGCG
TCTTCCGCAAGCTGGGCGTCAAGGGCCGCAAGCAGCTGCCGACCGCGCTG
GCCGACGTGGAACAGACCTGA

SEQ ID NO: 199

ATGTATAGCGGTACCTGCCGTGAAGGATACGAACTCGTCGCACGCGAGGA
CGAACTCGGCATTCTACAGAGGTCTCTGGAACAAGCGAGCAGCGGCCAGG
GCGTCGTGGTCACCGTCACCGGCCCAATCGCCTGCGGCAAGACAGAACTG
CTTGACGCGGCTGCCGCGAAGGCTGAGGCCATCATTCTGCGCGCGGTCTG
CGCGCCCGAAGAGCGGGCTATGCCGTACGCCATGATCGGGCAGCTCATCG
ACGACCCGGCGCTCGCGCATCGGGCGCCGGGGCTGGCTGATCGGATAGCC
CAGGGCGGGCAGCTGTCGCTGAGGGCCGAGAACCGACTGCGCAGGGATCT
CACCCGTGCCCTGCTGGCGCTTGCCGTGCACCGGCCTGTGCTGATCGGCG
TCGATGATGTGCATCACGCCGACACCGCCTCTTTGAACTGTCTGCTGCAT
TTGGCGCGCCGGGTCCGTCCGGCCCGGATATCCATGATCTTCACCGAGTT
GCGCAGCCTCACCCCTACTCAGTCACGATTCAAGGCGGAGCTGCTCAGCC
TGCCGTACCACCACGAGATCGCGCTGCGTCCATTCGGACCGGAGCAATCG
GCGGAGCTGGCTCGCGCCGCCTTCGGCCCGGGCCTCGCCGAGGATGTGCT
CGCGGGGTTGTATAAAACGACCAGGGGCAATCTGAGTCTCAGCCGTGGAC
TGATCAGCGATGTGCGGGAGGCCCTGGCCAACGGAGAGAGCGCTTTCGAG
GCGGGCCGCGCGTTCCGGCTGGCGTACCTCAGCTCGCTCTACCGCTGTGG
CCCGGTCGCGCTGCGGGTCGCCCGAGTGGCTGCCGTGCTGGGCCCAAGCG
CCACCACCACGCTGGTGCGCCGGCTAAGCGGGCTCAGCGCGGAGACGATA
GACCGGGCAACCAAGATCCTCACCGAGGGCGGGCTGCTGCTCGACCAGCA
GTTTCCGCACCCGGCCGCCCGCTCGGTGGTGCTCGATGACATGTCCGCCC
AGGAACGACGCGGCCTGCACACTCTCGCCCTGGAACTGCTGGACGAGGCG
CCGGTTGAAGTGCTCGCGCACCACCAGGTCGGCGCCGGTCTCATACACGG
GCCCAAGGCTGCGGAGATGTTCGCCAAGGCCGGCAAGGCTCTGGTCGTAC
GCAACGAGTTGGGCGACGCGGCCGAATACCTGCAACTGGCTCACCGGGCC
TCCGACGATGTCTCCACCCGGGCCGCCCTGCGGGTCGAGGCCGTGGCGAT
CGAGCGCCGCCGCAATCCGCTGGCCTCCAGTCGGCACATGGACGAGCTGA
GCGCCGCCGGCCGCGCCGGTCTGCTTTCCCCCAAGCATGCGGCGCTGGCC
GTCTTCTGGCTGGCCGACGGCGGGCGATCCGGCGAGGCAGCCCAGGTGCT
GGCGTCGGAACGCCCGCTCGCGACCACCGATCAGAACCGGGCCCACCTGC
GATTTGTCGAGGTGACTCTCGCGCTGTTCTCTCCCGGCGCCTTCGGATCG
GACCGGCGCCCACCTCCGCTGACGCCGGACGAACTCGCCAGCCTGCCGAA
GGCGGCCTGGCAATGCGCGGTCGCCGACAACGCGGCCATGACCGCCTTGC
ACGGCCATCCAGAACTTGCCACCGCTCAGGCGGAAACAGTTCTGCGGCAG
GCTGATTCGGCAGCCGACGCGATCCCCGCCGCGCTGATCGCCCTGTTGTA
CGCGGAGAACACCGAGTCCGCTCATATCTGGGCCGACAAGCTGGGCAGCA
TGAATGCCGGGGTATCGAACGAGGCGGAAGCGGGCTACGCCGGCCCGTGC
GCCGAGATCGCCCTGCGGCGCGGCGACCTGGCCACGGCGTTCGAGGCTGG
TAGCACCGTCCTGGACGACCGGTCACTGCCGTCGCTCGGCATCACCGCCG
CATTGCTGTTGAGCAGCAAGACGGCCGCCGCTGTCCGGCTGGGCGAACTC
GAGCGTGCGGAGAAGCTGCTCGCCGAGCCGCTTCCGAACGGCGTCCAGGA
CAGCCTTTTCGGTCTGCACCTGCTCTCGGCGTACGGCCAGTACAGCCTCG
CGATGGGCCGATATGAATCGGCTCACCGGGCGTTTCGCACCTGCGGAGAA
CGTATGCGCAGCTGGGATGTTGACGTGCCTGGTCTGGCCCTGTGGCGTGT
CGACGCCGCCGAGGCGCTGCTCAGCCTCGACCGGAACGAGGGCCAGCGGC
TCATCGACGAACAACTCACCCGTCCGATGGGACCTCGTTCCCGCGCGCTG
ACGCTGCGGATCAAGGCGGCATACCTCCCGCGGACGAAGCGGATCCCCCT
GCTCCATGAGGCGGCCGAGCTGCTGCTCCCCTGCCCCGACCCGTACGAGC
AAGCGCGGGTGCTCGCCGATCTGGGCGACACGCTCAGCGCGCTCAGACGC
TATAGCCGGGCGCGGGGAGTTCTCCGGCAGGCTCGTCACCTGGCCACCCA
GTGCGGTGCTGTCCCGCTGCTGCGCCGACTCGGGGGCGAGCCCGGCCGGA
TCGACGACGCCGGCCTGCCGCAGCGGAGCACATCGTTGACCGATGCGGAG
CGGCGGGTGGCGGCGCTGGCCGCGGCCGGACAGACCAACCGGGAGATCGC
CGAACAGCTGTTCGTCACGGCCAGCACAGTGGAACAGCACCTCACAAGCG
TCTTCCGCAAGCTGGGCGTCAAGGGCCGCAAGCAGCTGCCGACCGCGCTG
GCCGACGTGGAACAGACCTGA

SEQ ID NO: 200

GTGCGAGCTATTAATGCGTCCGACACCGGTCCTGAACTGGTCGCCCGCGA
AGACGAACTGGGACGTGTACGAAGTGCCCTGAACCGAGCGAACGGCGGCC
AAGGTGTCCTGATCTCCATTACCGGTCCGATCGCCTGCGGCAAGACCGAA
CTGCTTGAGGCTGCCGCCTCGGAAGTTGACGCCATCACTCTGCGCGCGGT
CTGTGCCGCCGAGGAACGGGCGATACCTTATGCCCTGATCGGGCAGCTTA
TCGACAACCCCGCGCTCGGCATTCCGGTTCCGGATCCGGCCGGCCTGACC
GCCCAGGGCGGACGACTGTCATCGAGCGCCGAGAACCGACTGCGTCGCGA
CCTCACCCGTGCCCTGCTGACGCTCGCCACCGACCGGCTGGTGCTGATCT
GTGTCGATGACGTGCAGCACGCCGACAACGCCTCGTTGAGCTGCCTTCTG
TATCTGGCCCGACGGCTTGTCCCGGCTCGAATCGCTCTGGTATTCACCGA
GTTGCGAGTCCTCACCTCGTCTCAGTTACGGTTCAACGCGGAGCTGCTCA
GCTTGCGGAACCACTGCGAGATCGCGCTGCGCCCACTCGGCCCGGGGCAT
GCGGCCGAGCTGGCCCGCGCCACCCTCGGCCCCGGCCTCTCCGACGAAAC
ACTCACGGAGCTGTACCGGGTGACCGGAGGCAACCTGAGTCTCAGCCGCG
GGCTGATCGACGATGTGCGGGACGCCTGGGCACGAGGGGAAACGGGCGTC
CAGGTGGGCCGGGCGTTCCGGCTGGCCTACCTCGGTTCCCTCCACCGCTG
TGGTCCGCTGGCGTTGCGGGTCGCCCGCGTAGCCGCCGTACTGGGCCCGA
GCGCCACCAGCGTCCTGGTGCGCCGGATCAGTGGGCTCAGCGCGGAGGCC
ATGGCCCAGGCGACCGATATCCTCGCTGACGGCGGCCTCCTGCGCGACCA
GCGGTTCACACATCCAGCGGCCCGCTCGGTGGTGCTCGACGACATGTCCG
CCGAGGAACGACGCAGCGTGCACAGCCTCGCCCTGGAACTGCTGGACGAG
GCACCGGCCGAGATGCTCGCGCACCACCGGGTCGGCGCCGGTCTCGTGCA
CGGGCCGAAGGCCGCGGAGACATTCACCGGGGCCGGCCGGGCACTGGCCG
TTCGCGGCATGCTGGGCGAGGCAGCCGACTACCTGCAACTGGCGTACCGG
GCCTCCGGCGACGCCGCTACCAAGGCCGCGATACGCGTCGAGTCCGTGGC
GGTCGAGCGCCGACGCAATCCGCTGGTCGTCAGTCGCCATTGGGACGAGC
TGAGCGTCGCGGCCCGCGCCGGTCTGCTCTCCTGCGAGCACGTGTCCAGG
ACGGCCCGCTGGCTGACCGTCGGTGGGCGGCCCGGCGAGGCGGCCAGGGT
GCTGGCGTCGCAACACCGACGGGTCGTCACCGATCAGGACCGGGCCCACC
TGCGGGTCGCCGAGTTCTCGCTCGCGCTGCTGTACCCCGGTACGTCCGGC
TCGGACCGGCGCCCGCACCCGCTCACGTCGGACGAACTCGCGGCCCTACC
GACTGCGACCAGACACTGCGCGATCGCCGATAACGCTGTCATGGCTGCCT
TGCGTGGTCATCCGGAGCTTGCCACCGCCGAGGCAGAAGCCGTTCTGCAG
CAAGCCGACGCGGCGGACGGCGCTGCTCTCACCGCGCTGATGGCCCTGCT
GTACGCGGAGAGCATCGAGGTCGCTGAAGTCTGGGCGGACAAGCTGGCGG
CAGAGGCCGGAGCATCGAACGGGCAGGACGCGGAGTACGCCGGTATACGC
GCCGAAATCGCCCTGCGGCGCGGCGATCTGACCGCGGCCGTCGAGACCGC
CGGCATGGTCCTGGACGGCCGGCCGCTGCCGTCGCTCGACATCACCGCCA
CGTTGCTGTTGGCCGGCAGGGCGTCCGTCGCCGTCCGGCTGGGCGAACTC
GACCACGCGGAGGAGCTGTTCGCCGCGCCGCCGGAGGACGCCTTCCAGGA
CAGCCTCTTCGGTCTGCATCTGCTCTCGGCGCACGGCCAGTACAGCCTCG
CGACAGGCCGGCCCGAGTCGGCATACCGGGCCTTTCGTGCCTGCGGCGAA
CGTATGCGCGATTGGGGCTTCGACGCGCCCGGTGTGGCCCTGTGGCGCGT
CGGCGCCGCCGAGGCGCTGCTCGGCCTCGACCGGAACGAGGGCCGACGGC
TCATCGACGAACAGCTGAGCCGGACGATGGCCCCCCGGTCCCACGCGTTG
ACGCTGCGGATAAAAGCGGCGTACATGCCGGAGCCGAAGCGGGTCGACCT
GCTCTACGAAGCGGCTGAGCTGCTGCTCTCCTGCCGGGACCAGTATGAGC
GAGCGCGGGTGCTCGCCGATCTGGGCGAGGCGCTCAGCGCGCTCGGGAAC
TACCGGCAGGCGCGAGGTGTGCTCCGGCAGGCTCGGCATCTGGCCATGCG
AACCGGCGCGGACCCGCTGCTGCGCCGGCTCGGAATCAGGCCCGGCCGGC
AGGACGACCCCGACCCGCAGCCGCGGAGCAGATCGCTGACCAACGCTGAG
CGGCGTGCGGCGTCGCTGGCCGCGACCGGACTGACCAACCGGGAGATCGC
CGACCGGCTCTTCGTCACCGCCAGCACCGTGGAGCAGCACCTCACCAACG
TCTTCCGCAAGCTGGGCGTCAAGGGCCGCAAGCAGCTGCCGGCCGAGTTG
GACGACATGGAATAG

SEQ ID NO: 201

ATGCGAGCTATTAATGCGTCCGACACCGGTCCTGAACTGGTCGCCCGCGA
AGACGAACTGGGACGTGTACGAAGTGCCCTGAACCGAGCGAACGGCGGCC
AAGGTGTCCTGATCTCCATTACCGGTCCGATCGCCTGCGGCAAGACCGAA
CTGCTTGAGGCTGCCGCCTCGGAAGTTGACGCCATCACTCTGCGCGCGGT
CTGTGCCGCCGAGGAACGGGCGATACCTTATGCCCTGATCGGGCAGCTTA
TCGACAACCCCGCGCTCGGCATTCCGGTTCCGGATCCGGCCGGCCTGACC
GCCCAGGGCGGACGACTGTCATCGAGCGCCGAGAACCGACTGCGTCGCGA
CCTCACCCGTGCCCTGCTGACGCTCGCCACCGACCGGCTGGTGCTGATCT
GTGTCGATGACGTGCAGCACGCCGACAACGCCTCGTTGAGCTGCCTTCTG
TATCTGGCCCGACGGCTTGTCCCGGCTCGAATCGCTCTGGTATTCACCGA
GTTGCGAGTCCTCACCTCGTCTCAGCTGCGGTTCAACGCGGAGCTGCTCA
GCTTGCGGAACCACTGCGAGATCGCGCTGCGCCCACTCGGCCCGGGGCAT
GCGGCCGAGCTGGCCCGCGCCACCCTCGGCCCCGGCCTCTCCGACGAAAC
ACTCACGGAGCTGTACCGGGTGACCGGAGGCAACCTGAGTCTCAGCCGCG
GGCTGATCGACGATGTGCGGGACGCCTGGGCACGAGGGGAAACGGGCGTC
CAGGTGGGCCGGGCGTTCCGGCTGGCCTACCTCGGTTCCCTCCACCGCTG
TGGTCCGCTGGCGTTGCGGGTCGCCCGCGTAGCCGCCGTACTGGGCCCGA
GCGCCACCAGCGTCCTGGTGCGCCGGATCAGTGGGCTCAGCGCGGAGGCC
ATGGCCCAGGCGACCGATATCCTCGCTGACGGCGGCCTCCTGCGCGACCA
GCGGTTCACACATCCAGCGGCCCGCTCGGTGGTGCTCGACGACATGTCCG
CCGAGGAACGACGCAGCGTGCACAGCCTCGCCCTGGAACTGCTGGACGAG
GCACCGGCCGAGATGCTCGCGCACCACCGGGTCGGCGCCGGTCTCGTGCA
CGGGCCGAAGGCCGCGGAGACATTCACCGGGGCCGGCCGGGCACTGGCCG
TTCGCGGCATGCTGGGCGAGGCAGCCGACTACCTGCAACTGGCGTACCGG
GCCTCCGGCGACGCCGCTACCAAGGCCGCGATACGCGTCGAGTCCGTGGC
GGTCGAGCGCCGACGCAATCCGCTGGTCGTCAGTCGCCATTGGGACGAGC
TGAGCGTCGCGGCCCGCGCCGGTCTGCTCTCCTGCGAGCACGTGTCCAGG
ACGGCCCGCTGGCTGACCGTCGGTGGGCGGCCCGGCGAGGCGGCCAGGGT
GCTGGCGTCGCAACACCGACGGGTCGTCACCGATCAGGACCGGGCCCACC
TGCGGGTCGCCGAGTTCTCGCTCGCGCTGCTGTACCCCGGTACGTCCGGC
TCGGACCGGCGCCCGCACCCGCTCACGTCGGACGAACTCGCGGCCCTACC
GACTGCGACCAGACACTGCGCGATCGCCGATAACGCTGTCATGGCTGCCT
TGCGTGGTCATCCGGAGCTTGCCACCGCCGAGGCAGAAGCCGTTCTGCAG
CAAGCCGACGCGGCGGACGGCGCTGCTCTCACCGCGCTGATGGCCCTGCT
GTACGCGGAGAGCATCGAGGTCGCTGAAGTCTGGGCGGACAAGCTGGCGG
CAGAGGCCGGAGCATCGAACGGGCAGGACGCGGAGTACGCCGGTATACGC
GCCGAAATCGCCCTGCGGCGCGGCGATCTGACCGCGGCCGTCGAGACCGC
CGGCATGGTCCTGGACGGCCGGCCGCTGCCGTCGCTCGACATCACCGCCA
CGTTGCTGTTGGCCGGCAGGGCGTCCGTCGCCGTCCGGCTGGGCGAACTC
GACCACGCGGAGGAGCTGTTCGCCGCGCCGCCGGAGGACGCCTTCCAGGA
CAGCCTCTTCGGTCTGCATCTGCTCTCGGCGCACGGCCAGTACAGCCTCG
CGACAGGCCGGCCCGAGTCGGCATACCGGGCCTTTCGTGCCTGCGGCGAA
CGTATGCGCGATTGGGGCTTCGACGCGCCCGGTGTGGCCCTGTGGCGCGT
CGGCGCCGCCGAGGCGCTGCTCGGCCTCGACCGGAACGAGGGCCGACGGC
TCATCGACGAACAGCTGAGCCGGACGATGGCCCCCCGGTCCCACGCGTTG
ACGCTGCGGATAAAAGCGGCGTACATGCCGGAGCCGAAGCGGGTCGACCT
GCTCTACGAAGCGGCTGAGCTGCTGCTCTCCTGCCGGGACCAGTATGAGC
GAGCGCGGGTGCTCGCCGATCTGGGCGAGGCGCTCAGCGCGCTCGGGAAC
TACCGGCAGGCGCGAGGTGTGCTCCGGCAGGCTCGGCATCTGGCCATGCG
AACCGGCGCGGACCCGCTGCTGCGCCGGCTCGGAATCAGGCCCGGCCGGC
AGGACGACCCCGACCCGCAGCCGCGGAGCAGATCGCTGACCAACGCTGAG
CGGCGTGCGGCGTCGCTGGCCGCGACCGGACTGACCAACCGGGAGATCGC
CGACCGGCTCTTCGTCACCGCCAGCACCGTGGAGCAGCACCTCACCAACG
TCTTCCGCAAGCTGGGCGTCAAGGGCCGCAAGCAGCTGCCGGCCGAGTTG
GACGACATGGAATAG

SEQ ID NO: 202

MPAVECYELDARDDELRKLEEVVTGRANGRGVVVTITGPIACGKTELLDA
AAAKADAITLRAVCSAEEQALPYALIGQLIDNPALASHALEPACPTLPGE
HLSPEAENRLRSDLTRTLLALAAERPVLIGIDESHANALCLLHLARRVGS
ARIAMVLTELRRLTPAHSQFQAELLSLGHHREIALRPLSPKHTAELVRAG
LGPDVDEDVLTGLYRATGGNLNLTRGLINDVREAWETGGTGISAGRAYRL
AYLGSLYRCGPVPLRVARVAAVLGQSANTTLVRWISGLNADAVGEATEIL
TEGGLLHDLRFPHPAARSVVLNDMSAQERRRLHRSALEVLDDVPVEVVAH
HQVGAGLLHGPKAAEIFAKAGQELHVRGELDTASDYLQLAHQASDDAVTG
MRAEAVAIERRRNPLASSRHLDELTVVARAGLLFPEHTALMIRWLGVGGR
SGEAAGLLASQRPRAVTDQDRAHMRAAEVSLALVSPGTSGPDRRPRPLTP
DELANLPKAARLCAIADNAVMSALRGRPELAAAEAENVLQHADSAAAGTT
ALAALTALLYAENTDTAQLWADKLVSETGASNEEEAGYAGPRAEAALRRG
DLAAAVEAGSTVLDHRRLSTLGITAALPLSSAVAAAIRLGETERAEKWLA
QPLPQAIQDGLFGLHLLSARGQYSLATGQHESAYTAFRTCGERMRNWGVD
VPGLSLWRVDAAEALLHGRDRDEGRRLVDEQLTRAMGPRSRALTLRVQAA
YSPPAKRVDLLDEAADLLLSCNDQYERARVLADLSETFSALRHHSRARGL
LRQARHLAAQRGAIPLLRRLGAKPGGPGWLEESGLPQRIKSLTDAERRVA
SLAAGGQTNRVIADQLFVTASTVEQHLTDVSTGSRPPAPAAELV

SEQ ID NO: 203

MVPEVRAAPDELIARDDELSRLQRALTRAGSGRGGVVAITGPIASGKTAL
LDAGAAKSGFVALRAVCSWEERTLPYGMLGQLFDHPELAAQAPDLAHFTA
SCESPQAGTDNRLRAEFTRTLLALAADWPVLIGIDDVHHADAESLRCLLH
LARRIGPARIAVVLTELRRPTPADSRFQAELLSLRSYQEIALRPLTEAQT
GELVRRHLGAETHEDVSADTFRATGGNLLLGHGLINDIREARTAGRPGVV
AGRAYRLAYLSSLYRCGPSALRVARASAVLGASAEAVLVQRMTGLNKDAV
EQVYEQLNEGRLLQGERFPHPAARSIVLDDLSALERRNLHESALELLRDH
GVAGNVLARHQIGAGRVHGEEAVELFTGAAREHHLRGELDDAAGYLELAH
RASDDPVTRAALRVGAAAIERLCNPVRAGRHLPELLTASRAGLLSSEHAV
SLADWLAMGGRPGEAAEVLATQRPAADSEQHRALLRSGELSLALVHPGAW
DPLRRTDRFAAGGLGSLPGPARHRAVADQAVIAALRGRLDRADANAESVL
QHTDATADRTTAIMALLALLYAENTDAVQFWVDKLAGDEGTRTPADEAVH
AGFNAEIALRRGDLMRAVEYGEAALGHRHLPTWGMAAALPLSSTVVAAIR
LGDLDRAERWLAEPLPQQTPESLFGLHLLWARGQHHLATGRHGAAYTAFR
ECGERMRRWAVDVPGLALWRVDAAESLLLLGRDRAEGLRLVSEQLSRPMR
PRARVQTLRVQAAYSPPPQRIDLLEEAADLLVTCNDQYELANVLSDLAEA
SSMVRQHSRARGLLRRARHLATQCGAVPLLRRLGAEPSDIGGAWDATLGQ
RIASLTESERRVAALAAVGRTNREIAEQLFVTASTVEQHLTNVFRKLAVK
GRQQLPKELADVGEPADRDRRCG

SEQ ID NO: 204

MIARLSPPDLIARDDEFGSLHRALTRAGGGRGVVAAVTGPIACGKTELLD
AAAAKAGFVTLRAVCSMEERALPYGMLGQLLDQPELAARTPELVRLTASC
ENLPADVDNRLGTELTRTVLTLAAERPVLIGIDDVHHADAPSLRCLLHLA
RRISRARVAIVLTELLRPTPAHSQFRAALLSLRHYQEIALRPLTEAQTTE
LVRRHLGQDAHDDVVAQAFRATGGNLLLGHGLIDDIREARTRTSGCLEVV
AGRAYRLAYLGSLYRCGPAALSVARASAVLGESVELTLVQRMTGLDTEAV
EQAHEQLVEGRLLREGRFPHPAARSVVLDDLSAAERRGLHELALELLRDR
GVASKVLARHQMGTGRVHGAEVAGLFTDAAREHHLRGELDEAVTYLEFAY
RASDDPAVHAALRVDTAAIERLCDPARSGRHVPELLTASRERLLSSEHAV
SLACWLAMDGRPGEAAEVLAAQRSAAPSEQGRAHLRVADLSLALIYPGAA
DPPRPADPPAEDEVASFSGAVRHRAVADKALSNALRGWSEQAEAKAEYVL
QHSRVTTDRTTTMMALLALLYAEDTDAVQSWVDKLAGDDNMRTPADEAVH
AGFRAEAALRRGDLTAAVECGEAALAPRVVPSWGMAAALPLSSTVAAAIR
LGDLDRAERWLAEPLPEETSDSLFGLHMVWARGQHHLAAGRYRAAYNAFR
DCGERMRRWSVDVPGLALWRVDAAEALLLLGRGRDEGLRLISEQLSRPMG
SRARVMTLRVQAAYSPPAKRIELLDEAADLLIMCRDQYELARVLADMGEA
CGMLRRHSRARGLFRRARHLATQCGAVPLLRRLGGESSDADGTQDVTPAQ
RITSLTEAERRVASHAAVGRTNKEIASQLFVTSSTVEQHLTNVFRKLGVK
GRQQLPKELSDAG

SEQ ID NO: 205

MEFYDLVARDDELRRLDQALGRAAGGRGVVVTVTGPVGCGKTELLDAAAA
EEEFITLRAVCSAEERALPYAVIGQLLDHPVLSARAPDLACVTAPGRTLP
ADTENRLRRDLTRALLALASERPVLICIDDVHQADTASLNCLLHLARRVA
SARIAMILTELRRLTPAHSRFEAELLSLRHRHEIALRPLGPADTAELARA
RLGAGVTADELAQVHEATSGNPNLVGGLVNDVREAWAAGGTGIAAGRAYR
LAYLSSVYRCGPVPLRIAQAAAVLGPSATVTLVRRISGLDAETVDEATAI
LTEGGLLRDHRFPHPAARSVVLDDMSAQERRRLHRSTLDVLDGVPVDVLA
HHQAGAGLLHGPQAAEMFARASQELRVRGELDAATEYLQLAYRASDDAGA
RAALQVETVAGERRRNPLAASRHLDELAAAARAGLLSAEHAALVVHWLAD
AGRPGEAAEVLALQRALAVTDHDRARLRAAEVSLALFHPGVPGSDPRPLA
PEELASLSLSARHGVTADNAVLAALRGRPESAAAEAENVLRNADAAASGP
TALAALTALLYAENTDAAQLWADKLAAGIGAGEGEAGYAGPRTVAALRRG
DLTTAVQAAGAVLDRGRPSSLGITAVLPLSGAVAAAIRLGELERAEKWLA
EPLPEAVHDSLFGLHLLMARGRYSLAVGRHEAAYAAFRDCGERMRRWDVD
VPGLALWRVDAAEALLPGDDRAEGRRLIDEQLTRPMGPRSRALTLRVRAA
YAPPAKRIDLLDEAADLLLSSNDQYERARVLADLSEAFSALRQNGRARGI
LRQARHLAAQCGAVPLLRRLGVKAGRSGRLGRPPQGIRSLTEAERRVATL
AAAGQTNREIADQLFVTASTVEQHLTNVFRKLGVKGRQQLPAELADLRPP
G

SEQ ID NO: 206

MYSGTCREGYELVAREDELGILQRSLEQASSGQGVVVTVTGPIACGKTEL
LDAAAAKAEAIILRAVCAPEERAMPYAMIGQLIDDPALAHRAPGLADRIA
QGGQLSLRAENRLRRDLTRALLALAVDRPVLIGVDDVHHADTASLNCLLH
LARRVRPARISMIFTELRSLTPTQSRFKAELLSLPYHHEIALRPFGPEQS
AELARAAFGPGLAEDVLVGLYKTTRGNLSLSRGLISDVREALANGESAFE
AGRAFRLAYLGSLYRCGPVALRVARVAAVLGPSATTTLVRRLSGLSAETI
DRATKILTEGGLLLDQQFPHPAARSVVLDDMSAQERRGLHTLALELLDEA
PVEVLAHHQVGAGLIHGPKAAEMFAKAGKALVVRNELGDAAEYLQLAHRA
SDDVSTRAALRVEAVAIERRRNPLASSRHMDELSAAGRAGLLSPKHAALA
VFWLADGGRSGEAAEVLASERPLATTDQNRAHLRFVEVTLALFSPGAFGS
DRRPPPLTPDELASLPKAAWQCAVADNAAMTALHGHPELATAQAETVLRQ
ADSAADAIPAALIALLYAENTESAHIWADKLGSTNGGVSNEAEAGYAGPC
AEIALRRGDLATAFEAGSTVLDDRSLPSLGITAALLLSSKTAAAVRLGEL
ERAEKLLAEPLPNGVQDSLFGLHLLSAYGQYSLAMGRYESALRAFHTCGE
RMRSWDVDVPGLALWRVDAAEALLSLDRNEGQRLIDEQLTRPMGPRSRAL
TLRIKAAYLPRTKRIPLLHEAAELLLPCPDPYEQARVLADLGDTLSALRR
YSRARGVLRQARHLAAQCGAVPLLRRLGGEPGRIDDAGLPQRSTSLTDAE
RRVAALAAAGQTNREIAKQLFVTASTVEQHLTSVFRKLGVKGRKQLPTAL
ADVEQT

SEQ ID NO: 207

MPAVESYELDARDDELRRLEEAVGQAGNGRGVVVTITGPIACGKTELLDA
AAAKSDAITLRAVCSEEERALPYALIGQLIDNPAVASQLPDPVSMALPGE
HLSPEAENRLRGDLTRTLLALAAERPVLIGIDDMHHADTASLNCLLHLAR
RVGPARIAMVLTELRRLTPAHSQFHAELLSLGHHREIALRPLGPKHIAEL
ARAGLGPDVDEDVLTGLYRATGGNLNLGHGLIKDVREAWATGGTGINAGR
AYRLAYLGSLYRCGPVPLRVARVAAVLGQSANTTLVRWISGLNADAVGEA
TEILTEGGLLHDLRFPHPAARSVVLNDLSARERRRLHRSALEVLDDVPVE
VVAHHQAGAGFIHGPKAAEIFAKAGQELHVRGELDAASDYLQLAHHASDD
AVTRAALRVEAVAIERRRNPLASSRHLDELTVAARAGLLSLEHAALMIRW
LALGGRSGEAAEVLAAQRPRAVTDQDRAHLRAAEVSLALVSPGASGVSPG
ASGPDRRPRPLPPDELANLPKAARLCAIADNAVISALHGRPELASAEAEN
VLKQADSAADGATALSALTALLYAENTDTAQLWADKLVSETGASNEEEGA
GYAGPRAETALRRGDLAAAVEAGSAILDHRRGSLLGITAALPLSSAVAAA
IRLGETERAEKWLAEPLPEAIRDSLFGLHLLSARGQYCLATGRHESAYTA
FRTCGERMRNWGVDVPGLSLWRVDAAEALLHGRDRDEGRRLIDEQLTHAM
GPRSRALTLRVQAAYSPQAQRVDLLEEAADLLLSCNDQYERARVLADLSE
AFSALRHHSRARGLLRQARHLAAQCGATPLLRRLGAKPGGPGWLEESGLP
QRIKSLTDAERRVASLAAGGQTNRVIADQLFVTASTVEQHLTNVFRKLGV
KGRQHLPAELANAE

SEQ ID NO: 208

MPAVKRNDLVARDGELRWMQEILSQASEGRGAVVTITGAIACGKTVLLDA
AAASQDVIQLRAVCSAEEQELPYAMVGQLLDNPVLAARVPALGNLAAAGE
RLLPGTENRIRRELTRTLLALADERPVLIGVDDMHHADPASLDCLLHLAR
RVGPARIAIVLTELRRLTPAHSRFQSELLSLRYHHEIGLQPLTAEHTADL
ARVGLGAEVDDDVLTELYEATGGNPSLCCGLIRDVRQDWEAGVTGIHVGR
AYRLAYLSSLYRCGPAALRTARAAAVLGDSADACLIRRVSGLGTEAVGQA
IQQLTEGGLLRDQQFPHPAARSVVLDDMSAQERHAMYRSAREAAAEGQAD
PGTPGEPRAATAYAGCGEQAGDYPEPAGRACVDGAGPAEYCGDPHGADDD
PDELVAALGGLLPSRLVAMKIRRLAVAGRPGAAAELLTSQRLHAVTSEDR
ASLRAAEVALATLWPGATGPDRHPLTEQEAASLPEGPRLLAAADDAVGAA
LRGRAEYAAAEAENVLRHADPAAGGDAYAAMIALLYTEHPENVLFWADKL
DAGRPDEETSYPGLRAETAVRLGDLETAMELGRTVLDQRRLPSLGVAAGL
LLGGAVTAAIRLGDLDRAEKWLAEPIPDAIRTSLYGLHVLAARGRLDLAA
GRYEAAYTAFRLCGERMAGWDADVSGLALWRVDAAEALLSAGIRPDEGRK
LIDDQLTREMGARSRALTLRAQAAYSLPVHRVGLLDEAAGLLLACHDGYE
RARVLADLGETLRTLRHTDAAQRVLRQAEQAAARCGSVPLLRRLGAEPVR
IGTRRGEPGLPQRIRLLTDAERRVAAMAAAGQTNREIAGRLFVTASTVEQ
HLTSVFRKLGVKGRRFLPTELAQAV

SEQ ID NO: 209

MYSGTCREGYELVAREDELGILQRSLEQASSGQGVVVTVTGPIACGKTEL
LDAAAAKAEAIILRAVCAPEERAMPYAMIGQLIDDPALAHRAPGLADRIA
QGGQLSLRAENRLRRDLTRALLALAVDRPVLIGVDDVHHADTASLNCLLH
LARRVRPARISMIFTELRSLTPTQSRFKAELLSLPYHHEIALRPFGPEQS
AELARAAFGPGLAEDVLAGLYKTTRGNLSLSRGLISDVREALANGESAFE
AGRAFRLAYLSSLYRCGPVALRVARVAAVLGPSATTTLVRRLSGLSAETI
DRATKILTEGGLLLDQQFPHPAARSVVLDDMSAQERRSLHTLALELLDEA
PVEVLAHHQVGAGLIHGPKAAEMFAKAGKALVVRNELGDAAEYLQLAHRA
SDDVSTRAALRVEAVAIERRRNPLASSRHMDELSAAGRAGLLSPKHAALA
VFWLADGGRSGEAAEVLASERPLATTDQNRAHLRFVEVTLALFSPGAFGS
DRRPPPLTPDELASLPKAAWQCAVADNAAMTALHGHPELATAQAETVLRQ
ADSAADAIPAALIALLYAENTESAHIWADKLGSTNAGVSNEAEAGYAGPC
AEIALRRGDLATAFEAGSAVLDDRSLPSLGITAALLLSSKTAAAVRLGEL
ERAEKLLAEPLPNGVQDSLFGLHLLSAYGQYSLAMGRYESAHRAFRTCGE
RMRSWDVDVPGLALWRVDAAEALLSLDRNEGQRLIDEQLTRPMGPRSHAL
TLRIKAAYLPRTKRIPLLHEAAELLLPCPDPYEQARVLADLGDTLSALRR
YSRARGVLRQARHLATQCGAVPLLRRLGGEPGRIDDAGLPQRSTSLTDAE
RRVAALAAAGQTNREIAEQLFVTASTVEQHLTSVFRKLGVKGRKQLPTAL
ADVEQT

SEQ ID NO: 210

MYSGTCREGYELVAREDELGILQRSLEEAGSGQGAVVTVTGPIACGKTEL
LDAAAAKADAIILRAVCAPEERAMPYAMIGQLIDDPALAHRAPELADRIA
QGGHLSLRAENRLRRDLTRALLALAVDRPVLIGVDDVHHADTASLNCLLH
LARRVRPARISMIFTELRSLTPTQSRFKAELLSLPYHHEIALRPLGPEQS
AELAHAAFGPGLAEDVLAGLYGMTRGNLSLSRGLISDVREAQANGESAFE
VGRAFRLAYLSSLYRCGPIALRVARVAAVLGPSATTTLVRRLSGLSAETI
DRATKILTEGGLLLDHQFPHPAARSVVLDDMSAQERRSLHTLALELLDEA
PVEVLAHHQVGAGLIHGPKAAEIFARAGQALVVRNELGDAAEYLQLAHRA
SDDVSTRAALRVEAVAIERRRNPLASSRHMDELSAAGRAGLLSPKHAALA
VFWLADGGRSGEAAEVLASEHPLATTDQNRAHLRFAEVTLALFCPGAFGS
DRRPPPLAPDELASLPKAAWQCAVADNAVMTALHAHPELATAQAETVLRQ
ADSAADAIPAALIALLYAENTESAQIWADKLGSTNAGVSNEAEAGYAGPC
AEIALRRGDLATAFEAGGTVLDDRPLPSLGITAALLLSSKTAAAVRLGEL
ERAEKLLAEPLPNGVQDSLFGLHLLSAHGQYSLAMGRYESAHRAFHTCGE
RMRSWGVDVPGLALWRVDAAEALLSLDRNEGQRLIDEQLARPMGPRSRAL
TLRIKAAYLPRTKRIPLLHEAAELLLSCPDPYEQARVLADLGDTLSALRR
YSRARGVLRQARHLATQCGAVPLLRRLGGEPGRIDDAGLPQRSTSLTDAE
RRVSALAAAGQTNREIAKQLFVTASTVEQHLTSVFRKLGVKGRRQLPTAL
ADVE

SEQ ID NO: 211

MYSGTCREGYELVAREDELGILQRSLEQASSGQGVVVTVTGPIACGKTEL
LDAAAAKAEAIILRAVCAPEERAMPYAMIGQLIDDPALAHRAPGLADRIA
QGGQLSLRAENRLRRDLTRALLALAVHRPVLIGVDDVHHADTASLNCLLH
LARRVRPARISMIFTELRSLTPTQSRFKAELLSLPYHHEIALRPFGPEQS
AELARAAFGPGLAEDVLAGLYKTTRGNLSLSRGLISDVREALANGESAFE
AGRAFRLAYLSSLYRCGPVALRVARVAAVLGPSATTTLVRRLSGLSAETI
DRATKILTEGGLLLDQQFPHPAARSVVLDDMSAQERRGLHTLALELLDEA
PVEVLAHHQVGAGLIHGPKAAEMFAKAGKALVVRNELGDAAEYLQLAHRA
SDDVSTRAALRVEAVAIERRRNPLASSRHMDELSAAGRAGLLSPKHAALA
VFWLADGGRSGEAAQVLASERPLATTDQNRAHLRFVEVTLALFSPGAFGS
DRRPPPLTPDELASLPKAAWQCAVADNAAMTALHGHPELATAQAETVLRQ
ADSAADAIPAALIALLYAENTESAHIWADKLGSMNAGVSNEAEAGYAGPC
AEIALRRGDLATAFEAGSTVLDDRSLPSLGITAALLLSSKTAAAVRLGEL
ERAEKLLAEPLPNGVQDSLFGLHLLSAYGQYSLAMGRYESAHRAFRTCGE
RMRSWDVDVPGLALWRVDAAEALLSLDRNEGQRLIDEQLTRPMGPRSRAL
TLRIKAAYLPRTKRIPLLHEAAELLLPCPDPYEQARVLADLGDTLSALRR
YSRARGVLRQARHLATQCGAVPLLRRLGGEPGRIDDAGLPQRSTSLTDAE
RRVAALAAAGQTNREIAEQLFVTASTVEQHLTSVFRKLGVKGRKQLPTAL
ADVEQT

SEQ ID NO: 212

MRAINASDTGPELVAREDELGRVRSALNRANGGQGVLISITGPIACGKTE
LLEAAASEVDAITLRAVCAAEERAIPYALIGQLIDNPALGIPVPDPAGLT
AQGGRLSSSAENRLRRDLTRALLTLATDRLVLICVDDVQHADNASLSCLL
YLARRLVPARIALVFTELRVLTSSQLRFNAELLSLRNHCEIALRPLGPGH
AAELARATLGPGLSDETLTELYRVTGGNLSLSRGLIDDVRDAWARGETGV
QVGRAFRLAYLGSLHRCGPLALRVARVAAVLGPSATSVLVRRISGLSAEA
MAQATDILADGGLLRDQRFTHPAARSVVLDDMSAEERRSVHSLALELLDE
APAEMLAHHRVGAGLVHGPKAAETFTGAGRALAVRGMLGEAADYLQLAYR
ASGDAATKAAIRVESVAVERRRNPLVVSRHWDELSVAARAGLLSCEHVSR
TARWLTVGGRPGEAARVLASQHRRVVTDQDRAHLRVAEFSLALLYPGTSG
SDRRPHPLTSDELAALPTATRHCAIADNAVMAALRGHPELATAEAEAVLQ
QADAADGAALTALMALLYAESIEVAEVWADKLAAEAGASNGQDAEYAGIR
AEIALRRGDLTAAVETAGMVLDGRPLPSLDITATLLLAGRASVAVRLGEL
DHAEELFAAPPEDAFQDSLFGLHLLSAHGQYSLATGRPESAYRAFRACGE
RMRDWGFDAPGVALWRVGAAEALLGLDRNEGRRLIDEQLSRTMAPRSHAL
TLRIKAAYMPEPKRVDLLYEAAELLLSCRDQYERARVLADLGEALSALGN
YRQARGVLRQARHLAMRTGADPLLRRLGIRPGRQDDPDPQPRSRSLTNAE
RRAASLAATGLTNREIADRLFVTASTVEQHLTNVFRKLGVKGRKQLPAEL
DDME

### LAL Binding Sites

In some embodiments, a gene cluster (e.g., a PKS gene cluster) includes one or more promoters that include one or more LAL binding sites. The LAL binding sites may include a polynucleotide consensus LAL binding site sequence (e.g., as described herein). In some instances, the LAL binding site includes a core AGGGGG (SEQ ID NO: 213) motif. In certain instances, the LAL binding site includes a sequence having at least 80% (e.g., 80%, 85%, 90%, 95%, 97%, 98%, 99%, or 100%) homology to SEQ ID NO: 213. The LAL binding site may include mutation sites that have been restored to match the sequence of a consensus or optimized LAL binding site. In some embodiments, the LAL binding site is a synthetic LAL binding site. In some embodiments, synthetic LAL binding sites may be identified by (a) providing a plurality of synthetic nucleic acids including at least eight nucleotides; (b) contacting one or more of the plurality of nucleotides including at least eight nucleotides with one or more LALs; (c) determining the binding affinity between a nucleic acid of step (a) and an LAL of step (b), wherein a synthetic nucleic acid is identified as a synthetic LAL binding site if the affinity between the synthetic nucleic acid and an LAL is greater than X. The identified synthetic LAL binding sites may then be introduced into a host cell in a compound-producing cluster (e.g., a PKS cluster).

In some embodiments, a pair of LAL binding site and a heterologous LAL or a heterologous LAL binding site and an LAL that have increased expression compared to a natural pair may be identified by (a) providing one or more LAL binding sites; (b) contacting one or more of the LAL binding sites with one or more LALs; (c) determining the binding affinity between a LAL binding site and an LAL, wherein a pair having increased expression is identified if the affinity between the LAL binding site and the LAL is greater than the affinity between the LAL binding site and its homologous LAL and/or the LAL at its homologous LAL binding site. In some embodiments, the binding affinity between the LAL binding site and the LAL is determined by determining the expression of a protein or compound by a cell which includes both the LAL and the LAL binding site.

### Constitutively Active LALs

In some embodiments, the recombinant LAL is a constitutively active LAL. For example, the amino acid sequence of the LAL has been modified in such a way that it does not require the presence of an inducer compound for the altered LAL to engage its cognate binding site and activate transcription of a compound producing protein (e.g., polyketide synthase). Introduction of a constitutively active LAL to a host cell would likely result in increased expression of the compound-producing protein (e.g., polyketide synthase) and, in turn, increased production of the corresponding compound (e.g., polyketide).

### Engineering Unidirectional LALs

FK gene clusters are arranged with a multicistronic architecture driven by multiple bidirectional promoter-operators that harbor conserved (in single or multiple, and inverted to each other and/or directly repeating) GGGGGT (SEQ ID NO: 179) motifs presumed to be LAL binding sites. Bidirectional LAL promoters may be converted to unidirectional ones (UniLALs) by strategically deleting one of the opposing promoters, but maintaining the tandem LAL binding sites (in case binding of LALs in the native promoter is cooperative, as was demonstrated for MalT). Functionally this is achieved by removal of all sequences 3′ of the conserved GGGGGT (SEQ ID NO: 179) motif present on the antisense strand (likely containing the −35 and −10 promoter sequences), but leaving intact the entire sequence on the sense strand. As a consequence of this deletion, transcription would be activated in one direction only. The advantages of this feed-forward circuit architecture would be to tune and/or maximize LAL expression during the complex life cycle of Streptomyces vegetative and fermentation growth conditions

### Host Cells

In some embodiments, the host cell is a bacteria such as an Actiobacterium. For example, in some embodiments, the host cell is a Streptomyces strain. In some embodiments, the host cell is Streptomyces anulatus, Streptomyces antibioticus, Streptomyces coelicolor, Streptomyces peucetius, Streptomyces sp. ATCC 700974, Streptomyces canus, Streptomyces nodosus, Streptomyces (multiple sp.), Streptoalloteicus hindustanus, Streptomyces hygroscopicus, Streptomyces avermitilis, Streptomyces viridochromogenes, Streptomyces verticillus, Streptomyces chartruensis, Streptomyces (multiple sp.), Saccharothrix mutabilis, Streptomyces halstedii, Streptomyces clavuligerus, Streptomyces venezuelae, Strteptomyces roseochromogenes, Amycolatopsis orientalis, Streptomyces clavuligerus, Streptomyces rishiriensis, Streptomyces lavendulae, Streptomyces roseosporus, Nonomuraea sp., Streptomyces peucetius, Saccharopolyspora erythraea, Streptomyces filipinensis, Streptomyces hygroscopicus, Micromonospora purpurea, Streptomyces hygroscopicus, Streptomyces narbonensis, Streptomyces kanamyceticus, Streptomyces coffinus, Streptomyces lasaliensis, Streptomyces lincolnensis, Dactosporangium aurantiacum, Streptomyces toxitricini, Streptomyces hygroscopicus, Streptomyces plicatus, Streptomyces lavendulae, Streptomyces ghanaensis, Streptomyces cinnamonensis, Streptomyces aureofaciens, Streptomyces natalensis, Streptomyces chattanoogensis L10, Streptomyces lydicus A02, Streptomyces fradiae, Streptomyces ambofaciens, Streptomyces tendae, Streptomyces noursei, Streptomyces avermitilis, Streptomyces rimosus, Streptomyces wedmorensis, Streptomyces cacaoi, Streptomyces pristinaespiralis, Streptomyces pristinaespiralis, Actinoplanes sp. ATCC 33076, Streptomyces hygroscopicus, Lechevalieria aerocolonegenes, Amycolatopsis mediterranei, Amycolatopsis lurida, Streptomyces albus, Streptomyces griseolus, Streptomyces spectabilis, Saccharopolyspora spinosa, Streptomyces ambofaciens, Streptomyces staurosporeus, Streptomyces griseus, Streptomyces (multiple species), Streptomyces acromogenes, Streptomyces tsukubaensis, Actinoplanes teichomyceticus, Streptomyces glaucescens, Streptomyces rimosus, Streptomyces cattleya, Streptomyces azureus, Streptoalloteicus hindustanus, Streptomyces chartreusis, Streptomyces fradiae, Streptomyces coelicolor, Streptomyces hygroscopicus, Streptomyces sp. 11861, Streptomyces virginiae, Amycolatopsis japonicum, Amycolatopsis balhimycini, Streptomyces albus J1074, Streptomyces coelicolor M1146, Streptomyces lividans, Streptomyces incarnates, Streptomyces violaceoruber, or Streptomyces griseofuscus. In some embodiments, the host cell is an Escherichia strain such as Escherichia coli. In some embodiments, the host cell is a Bacillus strain such as Bacillus subtilis. In some embodiments, the host cell is a Pseudomonas strain such as Pseudomonas putitda. In some embodiments, the host cell is a Myxococcus strain such as Myxococcus xanthus.

### EXAMPLES

### Example 1. Single Module Swapping to Produce an Engineered PKS

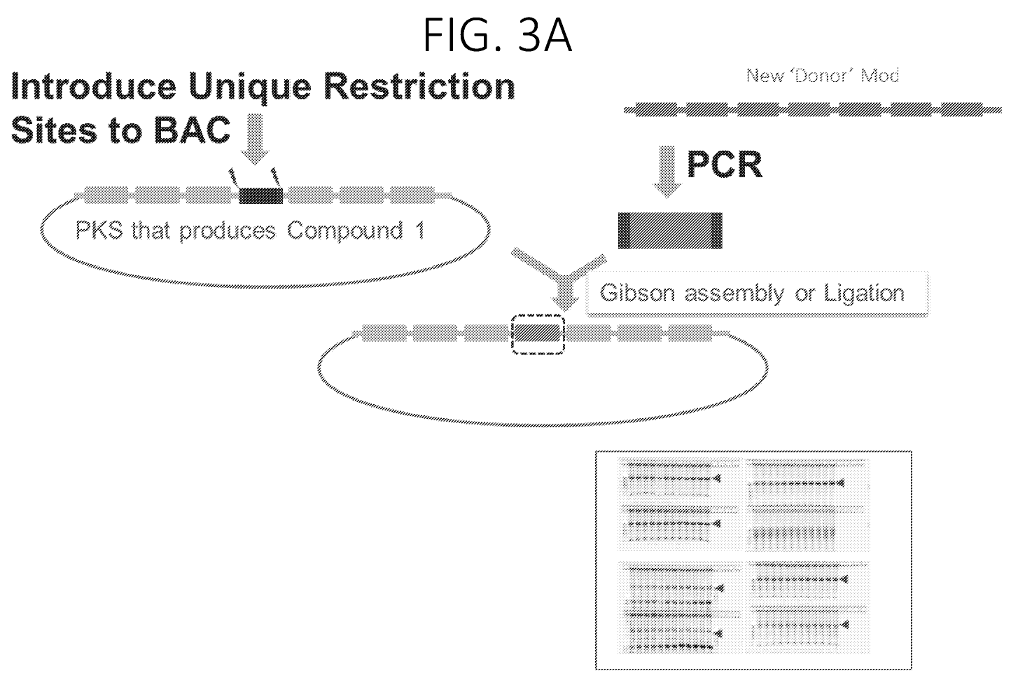

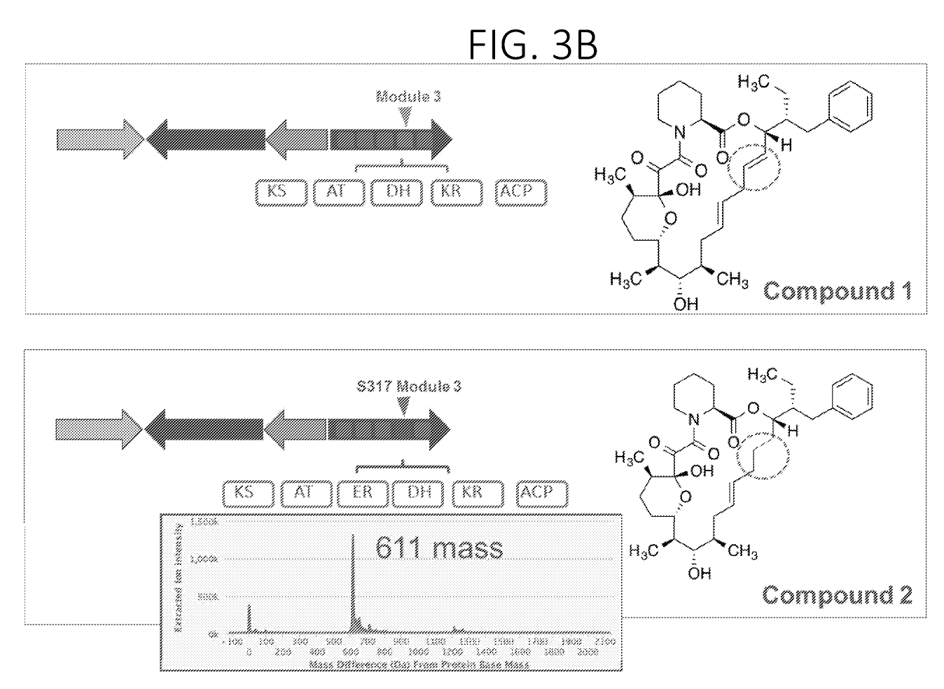

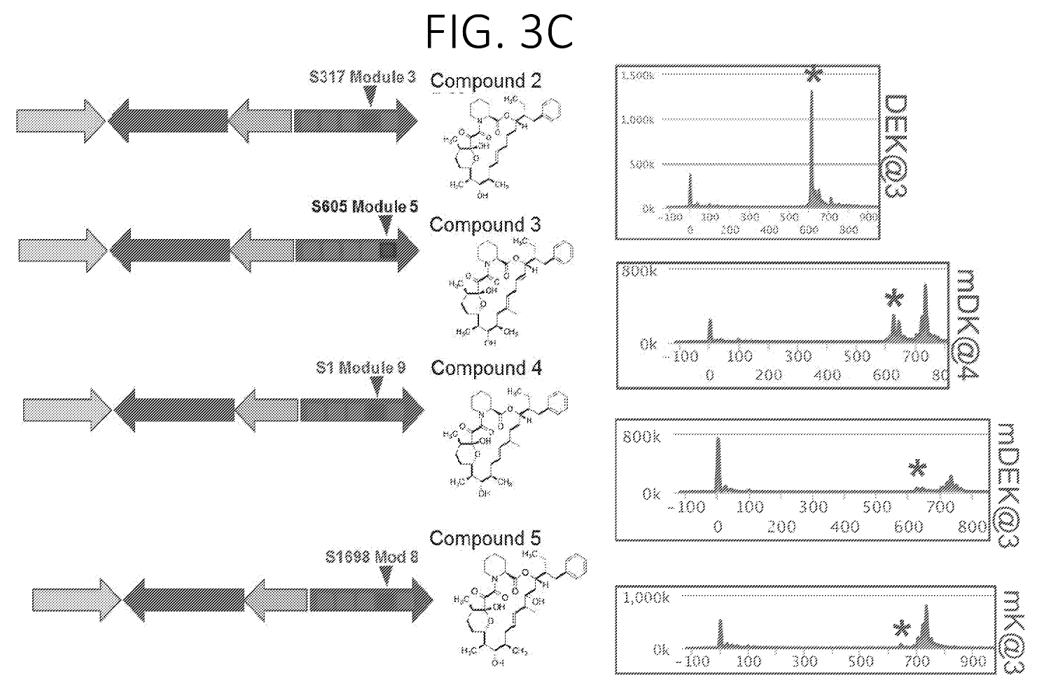

Inter-module residue covariation analysis and evolutionary trace analysis were used to predict 10 heterologous donor modules that would successfully replace module 3 of the PKS that produces Compound 1 (FIG. 3A). Seven of the 10 predicted donor modules, ranging in length from 4-6 kb, were selectively amplified in their entirety using a GC-rich long PCR method. In parallel, a bacterial artificial chromosome (BAC) that harbored the PKS that produces Compound 1 was converted to a module swap acceptor for heterologous donor modules by introducing the restriction sites AflII and SpeI to the flanking intermodule sequence of module 3. The modified acceptor BAC was linearized by digestion with AflII and SpeI, and the 7 donor modules were gel-purified and subcloned by Gibson cloning. The resulting constructs were subjected to Sanger sequencing of region of interest, PCR-based analysis to confirm cluster integrity, and Illumina NGS to sequence the entire BAC. The PCR-mediated error rate of the module amplification protocol was determined to be approximately 1 bp per 5000 bp, or approximately 1 mutation per module.

A single module was swapped to produce an engineered PKS by replacing module 3 of the PKS that produces Compound 1 with module 3 of Streptomyces strain S317. The donor S317 module 3 was PCR amplified and Gibson cloned into position 3 of the PKS that produces Compound 1 (FIG. 3B). The resulting clone was conjugated into a Streptomyces expression host and fermented. Production of compound was analyzed by LC-TOF mass spectrometry analysis by co-injecting purified native FKBP12, the protein to which both compounds are expected to bind, with either the product of the native PKS, Compound 1, or the compound produced by the engineered PKS cluster, Compound 2. Comparative LC-TOF analysis of indicated that Compound 2 had the expected mass of 611.38, corresponding to the conversion of the module 3 alkene to a fully reduced module at that position. Compound 2 was re-fermented at large scale, purified to homogeneity and the structure was confirmed by NMR spectroscopy.

To replace module 4 in the PKS that produces Compound 1, module swapping prediction algorithms based on inter-module covariation were used to generate a list of 16 modules encoding 4 chemistries. Gibson-based subcloning into module 4 was not as efficient as module 3. Gibson cloning, which involves a ssDNA intermediate, is difficult in high GC-rich regions, and direct ligation of donor modules to restriction sites with 4 bp overhangs may not be sensitive to local GC content. Therefore AM and SpeI sites were introduced at new positions in the inter-module flanking region to generate a direct ligation acceptor BAC. This direct ligation acceptor BAC was linearized by digestion with AflII and SpeI, and 12 donor modules were gel-purified, digested with AflII and XbaI and subcloned by ligation.

Single module swaps of either module 3 or module 4 in the PKS that produces Compound 1 generated novel Compounds 2-5 (FIG. 3C). Therefore, single module swapping was used to introduce a range of module encoded chemistries and generate novel compounds. LC-TOF mass spectrometry analysis indicated that of the module swaps at module 3 and module 4, the resulting hybrid clusters yielded a range of compound expression.

### Example 2. Library Construction by Combinatorial Dimodule Swapping

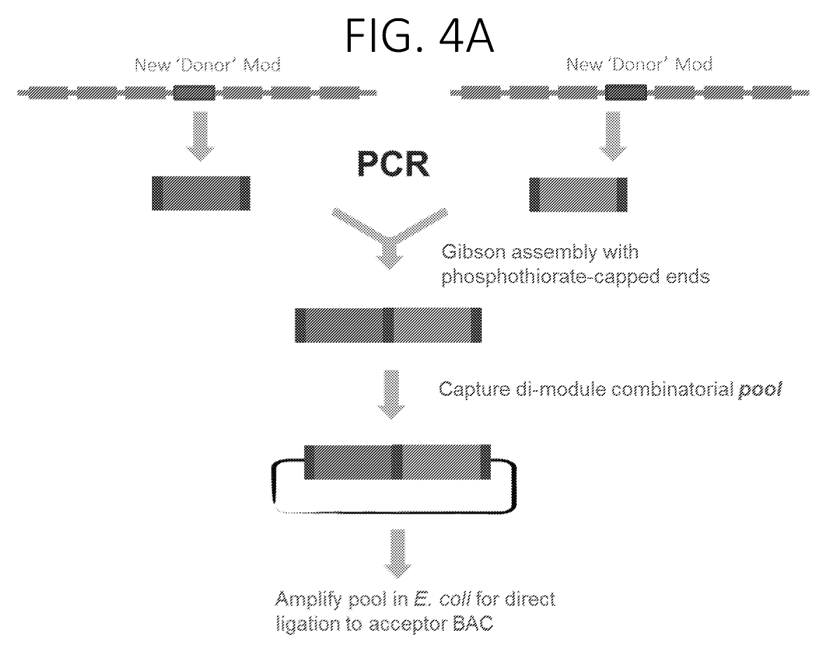

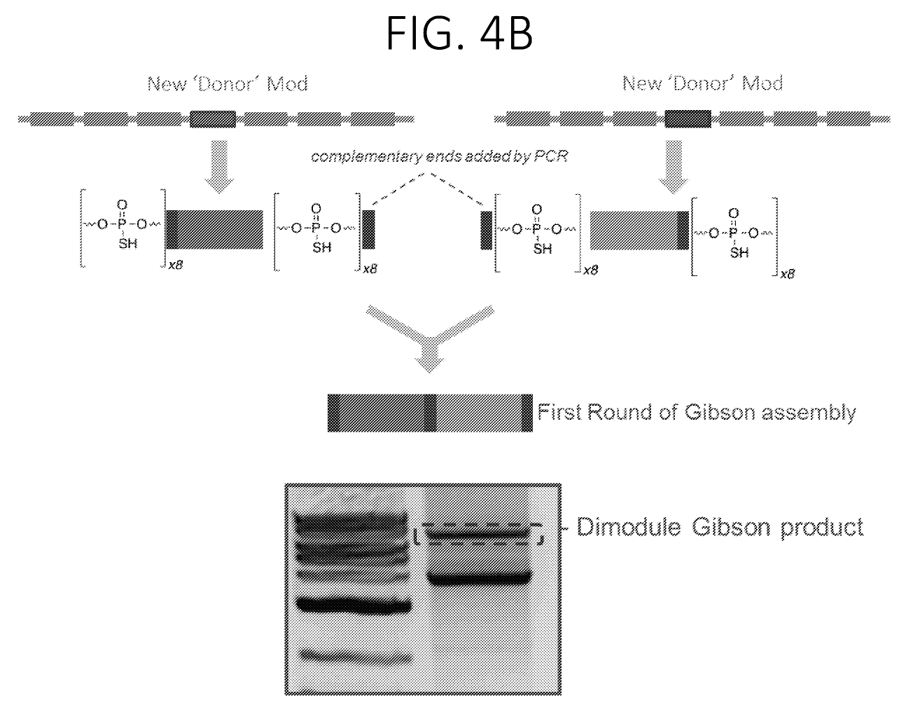

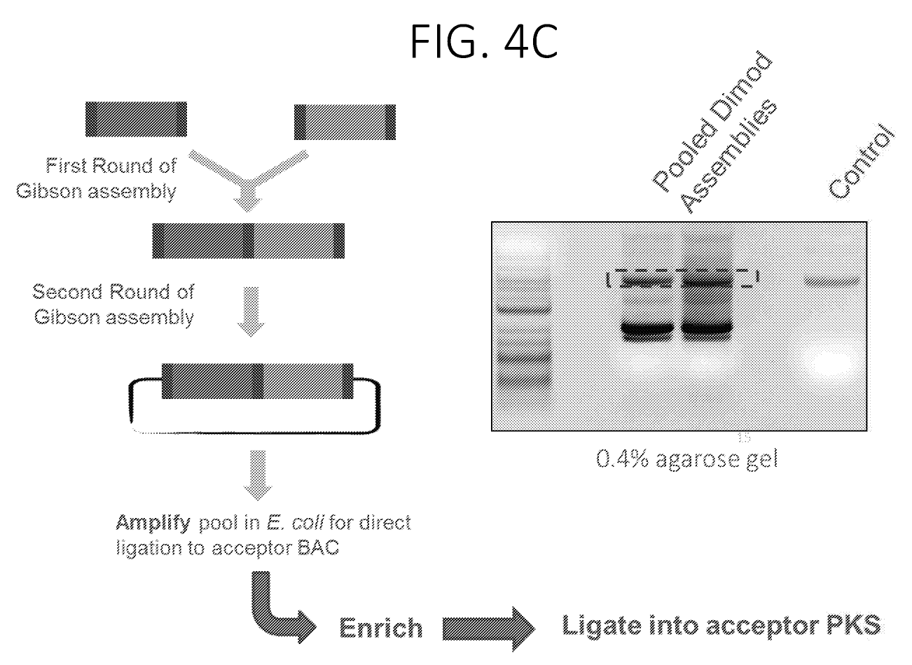

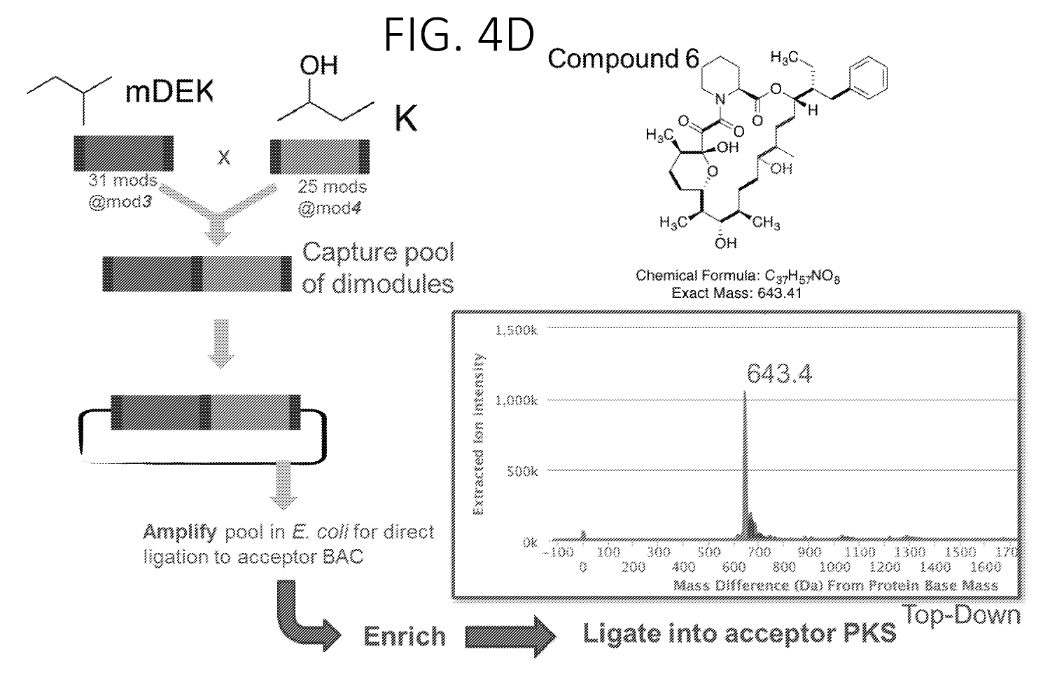

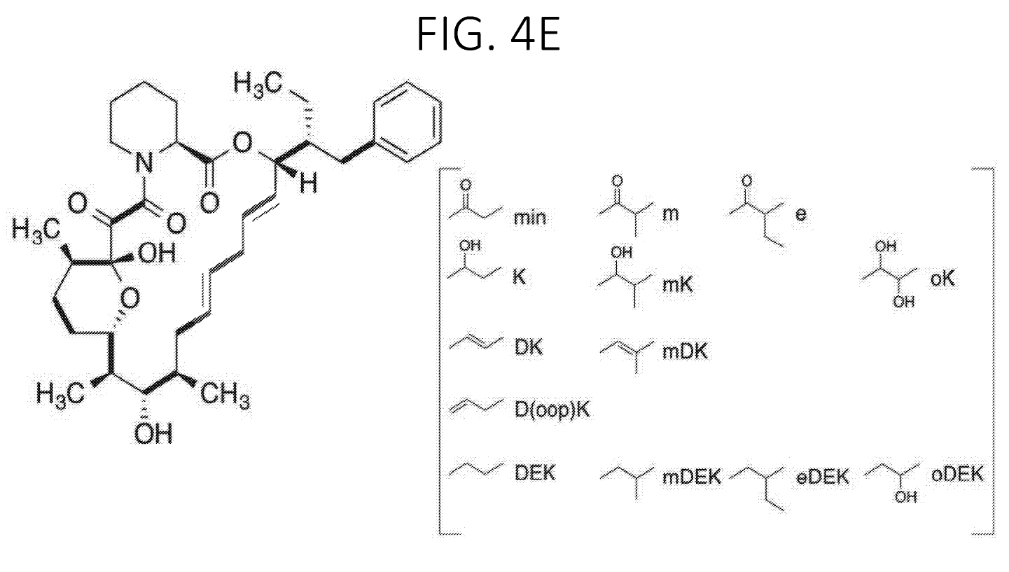

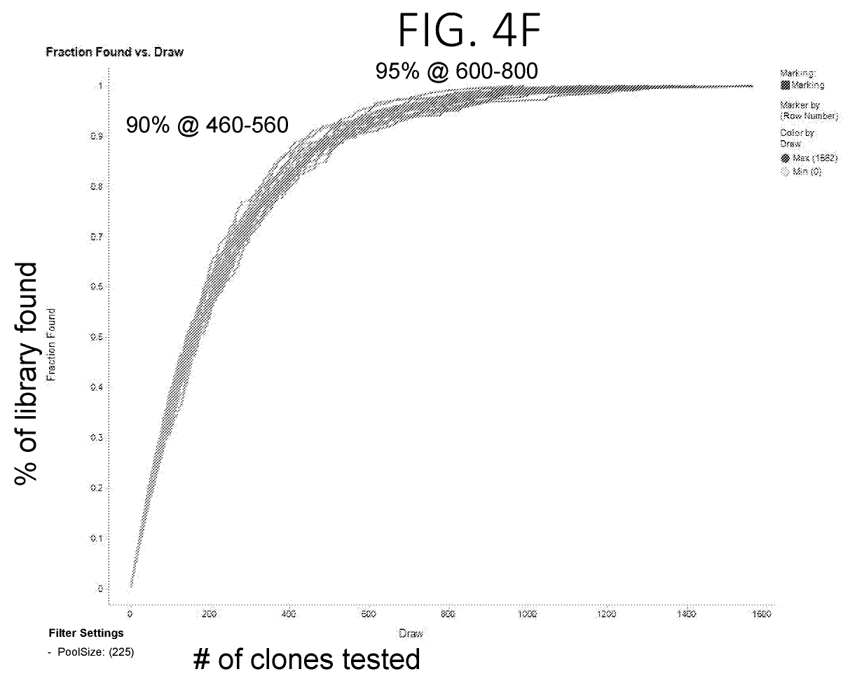

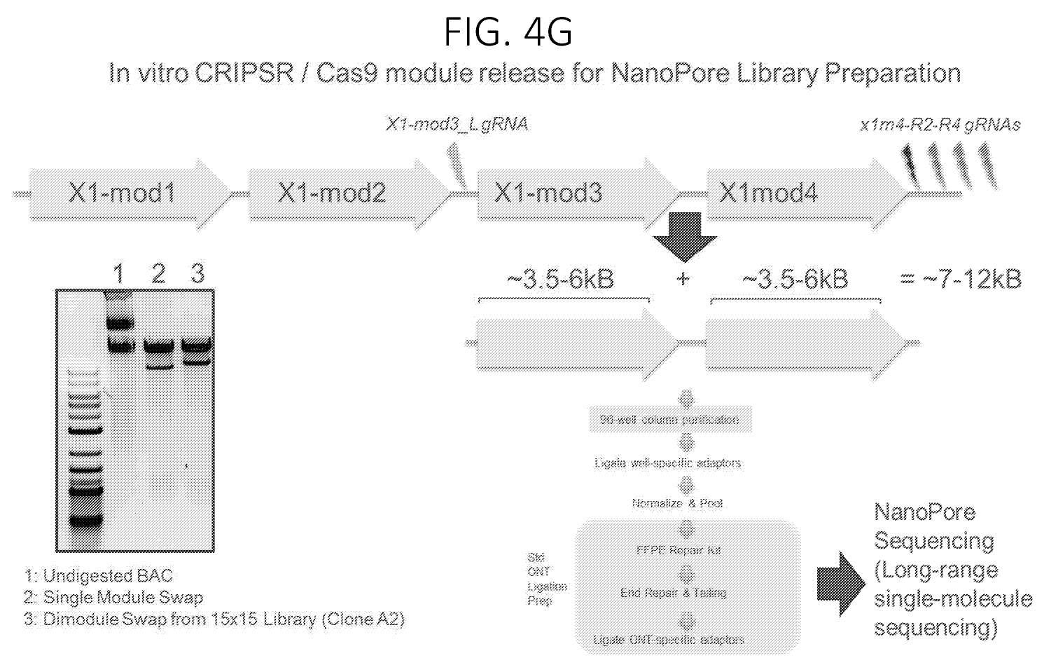

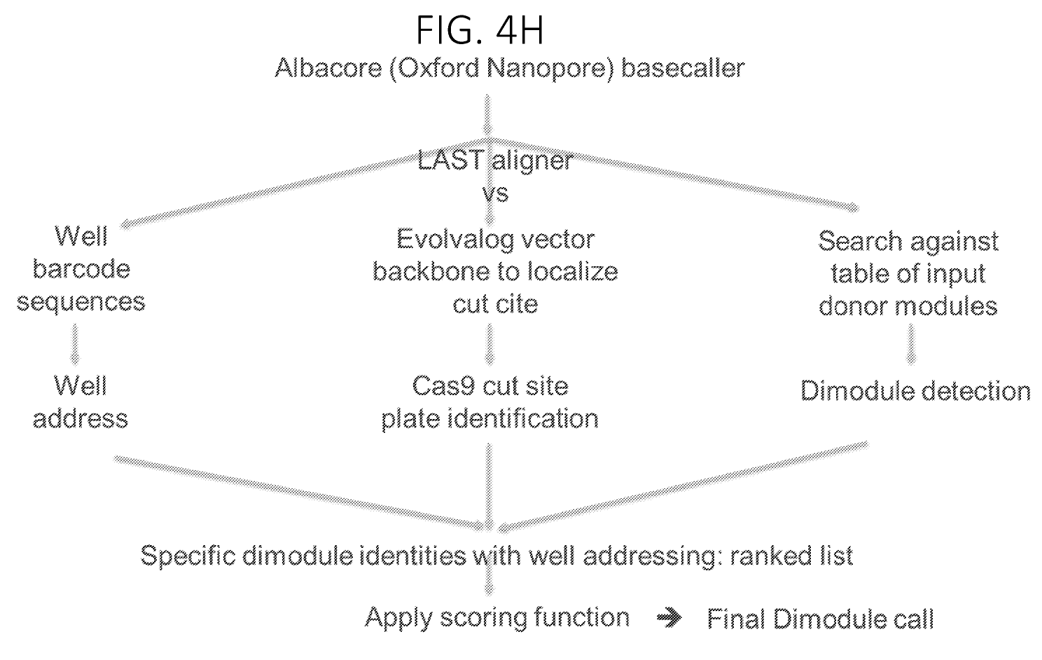

Pooled transfer of dimodule libraries was used to simultaneously replace modules 3 and 4 in the PKS that produces Compound 1 and generate a plurality of engineered PKS clusters (FIG. 4A). A total of 31 modules were amplified for transfer to the module 3 position and 25 modules for the module 4 position. To optimize Gibson dimodule assembly cloning, phosphothiorate-modified DNA oligos were synthesized for PCR amplification of the donor modules. Phosphothiorate-capped module ends function by constraining the exonuclease step of the Gibson cloning protocol, which resulted in a dramatic increase in Gibson capture of GC-rich DNA (FIG. 4B). An intermediate plasmid-based dimodule capture protocol was developed to assemble, capture, amplify, and enrich the dimodule units (FIG. 4C). Pooled module 3 and module 4 amplicons were mixed with a linear backbone amplicon based on pBR322 for a 3-part Gibson assembly reaction. Shuttle vectors containing dimodule assemblies could be resolved from empty vector by fractionating on a preparative 0.4% agarose gel. After dimodule capture, the assembled dimodule fragments were released from the shuttle vector by digestion with AflII and XbaI and subcloned by direct ligation to an expression vector containing the PKS that produces Compound 1, in which the PKS lacked the native module 3 and module 4.

Replicate BACs encoding single module and dimodule swaps were conjugated to optimized Streptomyces producer strain S2441 and solid-phase extracted samples were subjected to LC-TOF mass spectrometry with the expected protein binding partner, purified FKBP12 protein. Further analysis confirmed that dimodule library generation is capable of engineering PKS clusters that express novel compounds in high yield (FIG. 4D). As a representative example, Compound 6 was generated by dimodule swapping of a module encoding mDEK chemistry at module 3 and K chemistry at module 4 of the PKS tha produced Compound 1. The expected mass of Compound 6 was observed by LC-TOF analysis, confirming that the dimodule assembly protocol yields engineered derivatives Compound 1.

A 650-member combinatorial library of engineered derivatives of the PKS that produces Compound 1 was produced by dimodule swapping. A total of 31 modules were amplified for transfer the module 3 position and 25 modules for the module 4 position of the PKS that produces Compound 1 (FIG. 4E). Clusters were cloned onto BACs, and the cloned BACs were subsequently used as templates to PCR modules of diverse sources from multiple heterologous donors.

A subset of the library corresponding to 15 different donor modules at the module 3 position and 15 different donor modules at the module 4 position produced a potential combinatorial library of 225 novel PKS clusters and resulting novel compounds (the 15×15 dimodule library). Because the dimodule library was assembled as a pool, rarefaction analysis was performed to determine how many clones needed to be conjugated, fermented, and extracted to effectively sample >90% of the diversity of the library. Rarefaction analysis indicated that 650 clones corresponded to a statistical sampling >90% of the dimodule library (FIG. 4F). 650 clones were prosecuted and subjected to LC-TOF mass spectrometry analysis. 115 of the 650 sampled clones expressed compounds with novel masses.

### Example 3. Characterization of a Combinatorial Dimodule Library by Single-Molecule Long-Read Sequencing

A library corresponding to 15 different donor modules at the module 3 position and 15 different donor modules at the module 4 position (the 15×15 dimodule library), produced according to the methods of Example 2, was characterized by Nanopore sequencing (FIG. 4G). The dimodules present in the 15×15 dimodule library were excised from the PKS clusters using CRISPR/Cas9 (NEB). The resulting excised dimodules each had a length of approximately 7-12 kilobases. The dimodules were purified by 96-well column purification, and well-specific adaptors were ligated to the dimodules. The resulting dimodules were normalized and pooled and prepared for sequencing according to the standard ligation preparation protocol for Nanopore sequencing of oligonucleotides. Nine 96-well plates (864 dimodule clones total) were sequenced by Nanopore and the resulting sequencing data was analyzed according to the informatics workflow provided in FIG. 4H, with 73.1% of clones being called. The comparison of the resulting sequencing data against the table of input of the donor modules allows the deconvolution of the resulting combinatorial library by identification of the resulting dimodules. The results of Nanopore sequencing of the 15×15 dimodule library are provided in Table 1.

TABLE 1

| Library | | | Single | | Grand |
|---|---|---|---|---|---|
| Plate IDs | NoCall | Ambiguous | Read | Called | Total |
| 163846 | 45 | 4 | 11 | 36 | 96 |
| 163848 | 14 | 10 | 14 | 58 | 96 |
| 163851 | | 16 | | 80 | 96 |
| 163896 | 5 | 8 | 5 | 78 | 96 |
| 163897 | | 21 | 1 | 74 | 96 |
| 163898 | 3 | 10 | 11 | 72 | 96 |
| 163899 | 4 | 6 | 2 | 84 | 96 |
| 163900 | 1 | 26 | 3 | 66 | 96 |
| 50066321 | | 12 | | 84 | 96 |
| Grand Total | 72 | 113 | 47 | 632 | 864 |
| % | 8.3% | 13.1% | 5.4% | 73.1% | |

### Example 4. Library Construction by Combinatorial Trimodule Swapping

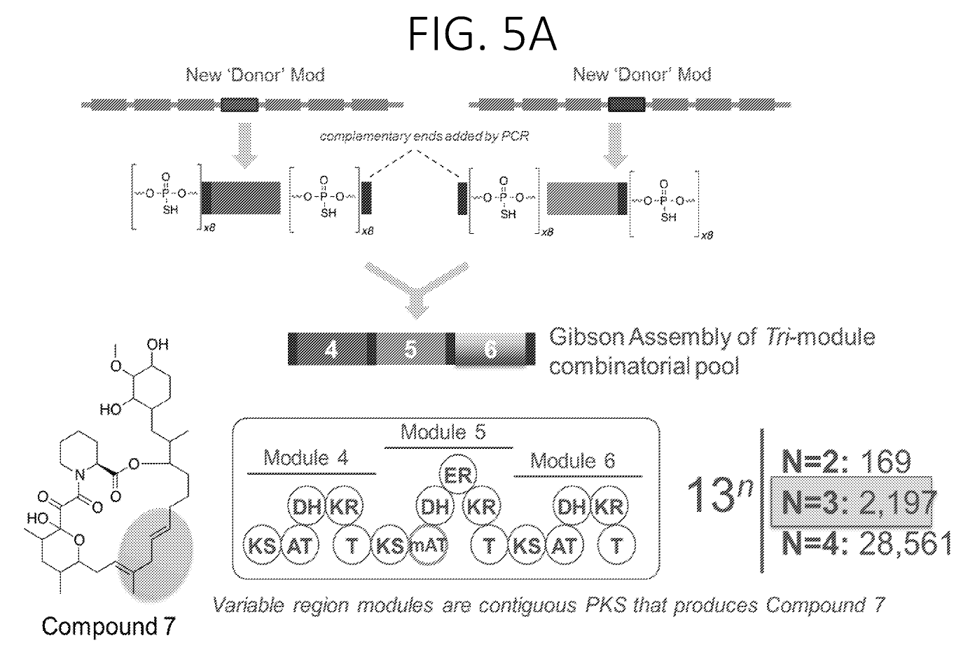

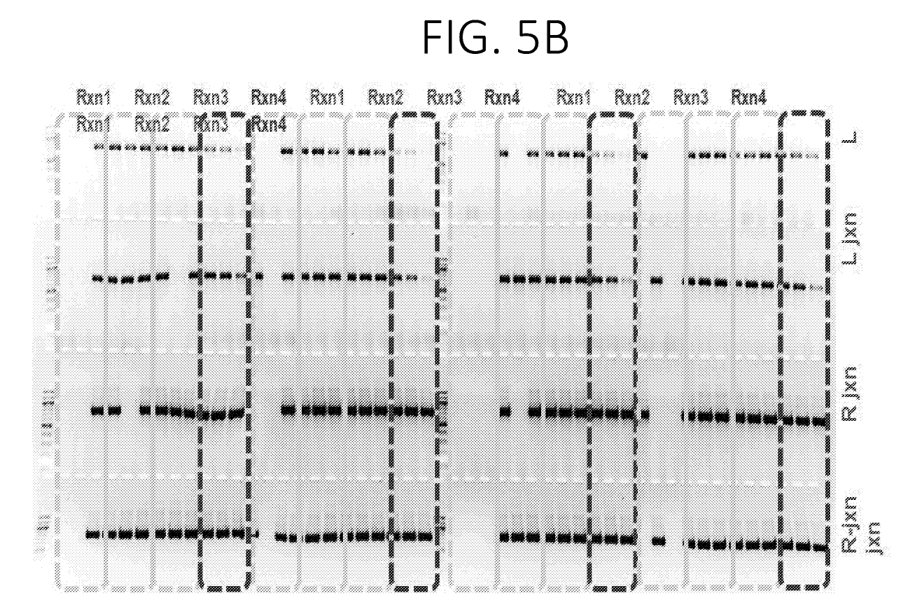

The combinatorial module swap protocols were modified to generate trimodule assemblies in the PKS that produces Compound 7 (FIG. 5A). Increasing the number of module swaps increases the size, and therefore diversity, of a PKS library. For example, given a collection of 13 different module-encoded chemistries, increasing size and diversity is based on the number of modules that are swapped such that the maximal library size of a single mod swap is 13; with a dimodule swap the maximal library size is 13²=169; and for a trimodule swap, the maximal library is 13³=2197.

Trimodule assembly leverages the technical advances of the dimodule protocol with an additional “proof-reading” Gibson cloning step to insert the captured trimodule assembly into the PKS that produces Compound 7 (FIG. 5B). As before, phosphorothioate chemistry was used to constrain the ssDNA intermediate for the first round of Gibson cloning into a shuttle vector. Shuttle vector clones harboring trimodule assemblies were enriched by preparative gel fractionation and isolation. Finally, Gibson-mediated “error correction” was used to trim restriction sites for scarless cloning in the expression vector. First, flanking PmeI restriction sites were introduced within the linker regions between Module 3 and Module 4, as well as between Module 6 and Module7. Sites with reduced GC content and secondary structure (as predicted by DNAfold; <8 kcal/ml) were selected for optimal Gibson homology arms. A Gibson Assembly Ultra Kit (SGI-DNA) was used to clone the trimodule assembly into the PKS that produces Compound 7 enabling the replacement of Modules 4, 5, and 6 and simultaneously removal of the additional extraneous PmeI sequence retained after digestion. This resulted in >95% correct assembly for the industrial scale production of compounds produced by trimodule swapped PKS clusters (>200 per week).

### Example 5. Ring Expansion by Swapping a Single Module Acceptor with a Dimodule Donor

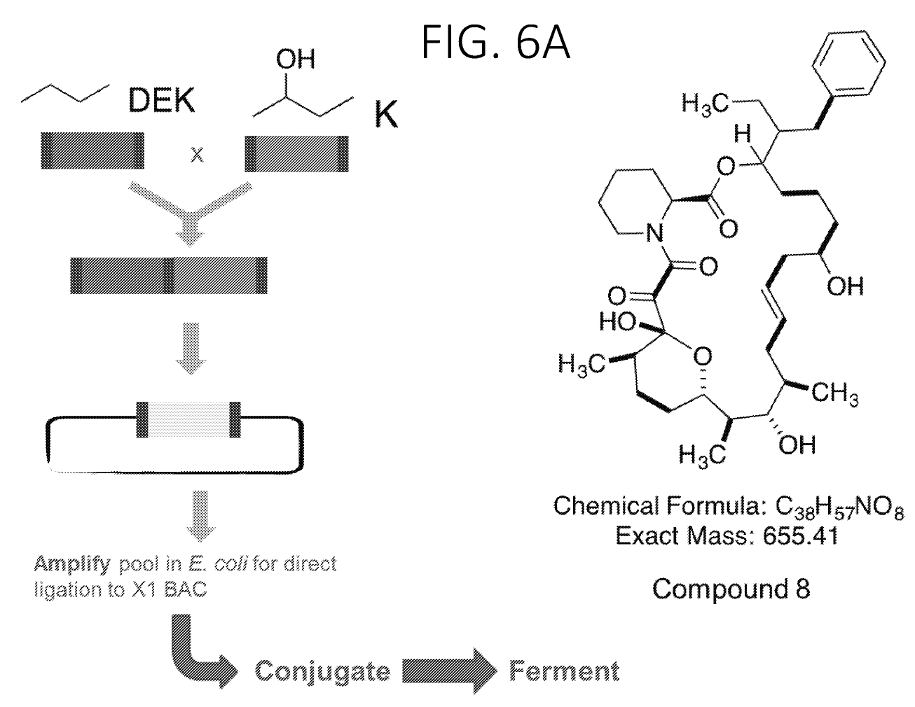

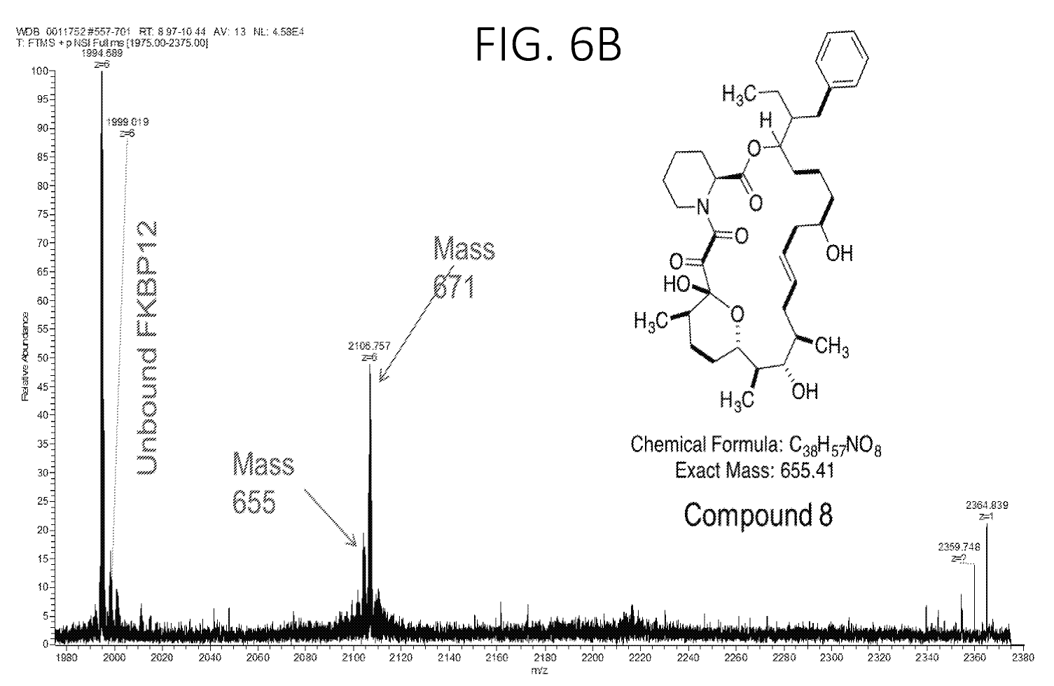

A heterologous dimodule donor assembly encoding mDEK chemistry and K chemistry was swapped into module 3, a single module acceptor, of the PKS that produces Compound 1 by the methods described above (FIG. 6A). The compound produced by engineered PKS, Compound 8, was observed in high yield and had a mass of 655.41, as determined by LC-TOF analysis (FIG. 6B). This corresponds to a ring-expanded compound product in which Compound 8 contains an additional 2-carbon extender unit. Thus reprogramming PKS biosynthesis via module swapping by insertion of a dimodule assembly to replace a single module may produce functional PKS expression.

### Example 6. Module Swapping of a PKS Loading Module

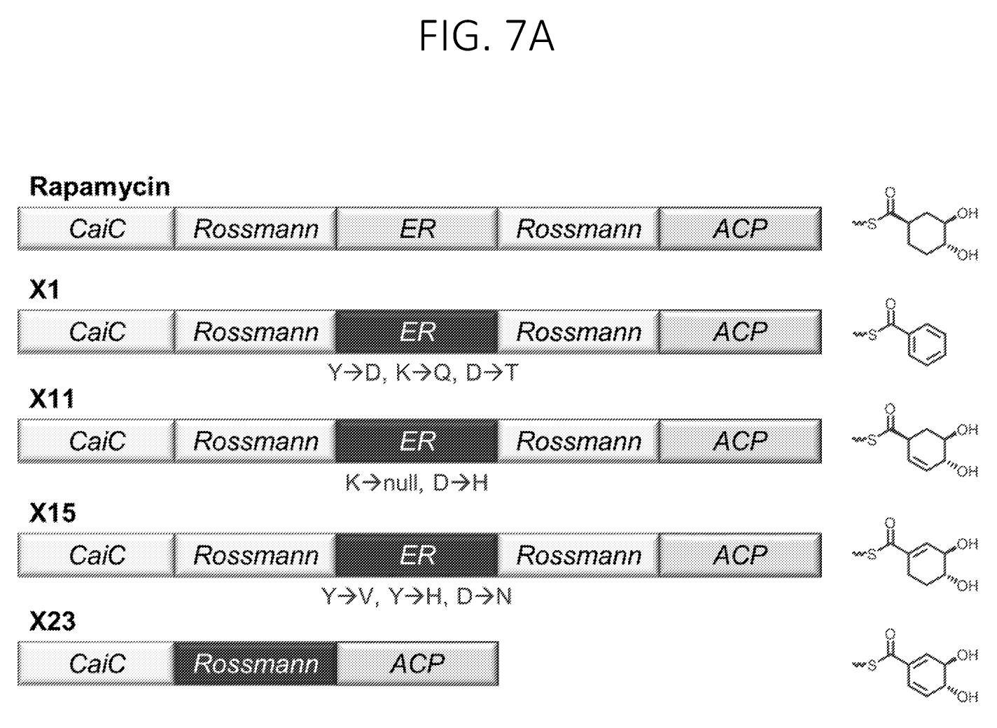

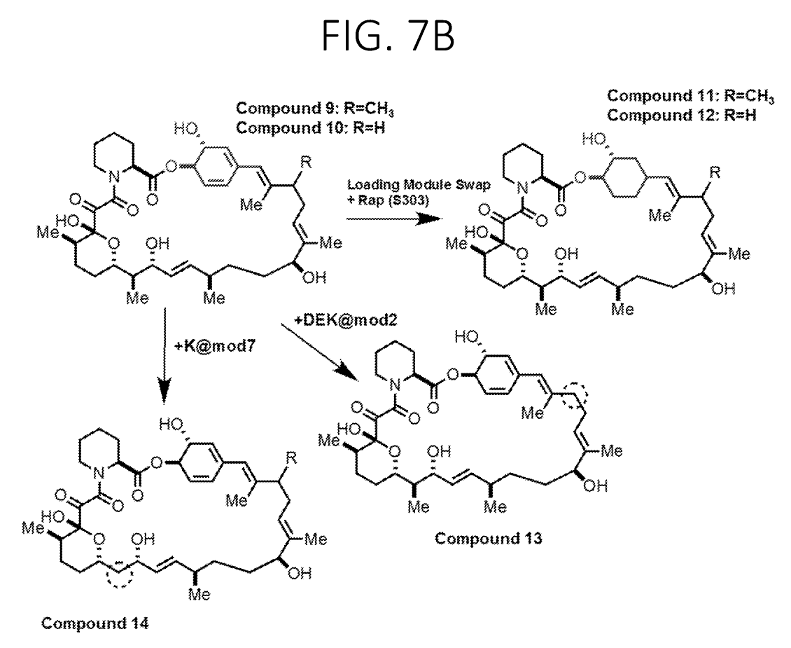

Rapamycin is a natural product synthesized by a mixed polyketide synthase (PKS)/nonribosomal peptide synthetase (NRPS) system. Rapamycin shares a common structural motif with related natural product FK506 which is responsible for binding to FK506-binding proteins (FKBPs). During biogenesis of Rapamycin, loading modules bind and load a 4,5-dihydroxycyclohexa-1,5-dienecarboxylic acid starter unit via a CaiC domain, which functions as a carboxylic acid ligase (CL) like domain (FIG. 7A). Loading modules may possess similar domain structure as conventional elongation PKS modules, including ketoreductase-like domains and an enoyl-reductase domain, which may or may not be catalytically active. The final chemistry of the starter unit depends on the presence and the sequence of the domains in the loading module, so the resulting “starter unit” can be engineered by swapping the loading module

The X23 PKS cluster produces Compound 9 and Compound 10 (FIG. 7B). The Rapamycin loading module from Streptomyces stain S303 was swapped into the X23 cluster by the methods described previously for a single module swap. The engineered PKS produced Compounds 11 and 12, in which the starter unit is replaced with the starter unit of Rapamycin. Additional single elongation module swaps of Module 2 and Module 7 of X23 produced Compounds 13 and 14, respectively.

### Other Embodiments

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any polynucleotide or protein encoded thereby; any method of production; any method of use) can be excluded from any one or more claims, for any reason, whether or not related to the existence of prior art.

What is claimed is:

1. An engineered polyketide synthase comprising one or more heterologous modules with altered enzymatic activity relative to a reference polyketide, wherein the engineered polyketide synthase is capable of producing a polyketide when expressed under conditions suitable to allow expression of a compound by the engineered polyketide synthase and wherein the one or more heterologous modules do not substantially inhibit polyketide translocation during polyketide biosynthesis.

2. An engineered polyketide synthase comprising one or more heterologous modules with altered enzymatic activity relative to a reference polyketide, wherein the engineered polyketide synthase is capable of producing a polyketide when expressed under conditions suitable to allow expression of a compound by the engineered polyketide synthase and wherein the one or more heterologous modules comprise linking sequences which are compatible to the linking sequences of the modules adjacent thereto.

3. An engineered polyketide synthase comprising one or more heterologous modules with altered enzymatic activity relative to a reference polyketide, wherein the engineered polyketide synthase is capable of producing a polyketide when expressed under conditions suitable to allow expression of a compound by the engineered polyketide synthase and wherein the polyketide expression level of the engineered polyketide synthase is at least 1% of the polyketide expression level of the reference polyketide synthase.

4. The engineered polyketide synthase of any one of claims 1 to 3, wherein the one or more heterologous modules comprise native linking sequences.

5. The engineered polyketide synthase of any one of claims 1 to 4, wherein the engineered polyketide synthase comprises two or more heterologous modules.

6. The engineered polyketide synthase of claim 5, wherein the two or more heterologous modules are adjacent.

7. The engineered polyketide synthase of any one of claims 1 to 6, wherein the engineered polyketide synthase comprises three or more heterologous modules.

8. The engineered polyketide synthase of claim 7, wherein the three or more heterologous modules are adjacent.

9. The engineered polyketide synthase of any one of claims 1 to 8, wherein the heterologous module is an elongation module which modifies a β-carbonyl unit in the variable region of the polyketide.

10. The engineered polyketide synthase of any one of claims 1 to 9, wherein at least one of the one or more heterologous modules comprises a portion having at least 90% identity to any one of SEQ ID NO: 1-174.

11. The engineered polyketide synthase of any one of claims 1 to 10, wherein at least one of the one or more heterologous modules comprises a portion having the sequence of any one of SEQ ID NO: 1-174.

12. A chimeric polyketide synthase, wherein at least one module of the chimeric polyketide synthase has been modified as compared to a polyketide synthase having the sequence of SEQ ID NO: 175-176.

13. The chimeric polyketide synthase of claim 12, wherein the at least one module comprises a portion having at least 90% identity to any one of SEQ ID NO: 1-174.

14. A nucleic acid encoding a polyketide synthase of any one of claims 1 to 13.

15. The nucleic acid of claim 15, wherein the nucleic acid further encodes an LAL, wherein the sequence encoding the LAL is operatively linked to the sequence encoding the polyketide synthase.

16. The nucleic acid of claim 15, wherein the LAL is a heterologous LAL.

17. The nucleic acid of claim 15 or 16, wherein LAL comprises a portion having at least 80% identity to SEQ ID NO: 177.

18. The nucleic acid of claim 17, wherein the LAL comprises a portion having the sequence of SEQ ID NO: 177.

19. The nucleic acid of claim 18, wherein the LAL has the sequence of SEQ ID NO: 177.

20. The nucleic acid of any one of claims 14 to 19, wherein the nucleic acid encoding the LAL lacks a TTA inhibitory codon in an open reading frame.

21. The nucleic acid of any one of claims 14 to 20, wherein the nucleic acid further comprises an LAL binding site, wherein the sequence encoding the LAL binding site is operatively linked to the sequence encoding the polyketide synthase.

22. The nucleic acid of claim 21, wherein the LAL binding site comprises a portion having at least 80% sequence identity to the sequence of SEQ ID NO: 178.

23. The nucleic acid of claim 22, wherein the LAL binding site comprises a portion having the sequence of SEQ ID NO: 178.

24. The nucleic acid of claim 23, wherein the LAL binding site has of the sequence of SEQ ID NO: 178.

25. The nucleic acid of claim 21, wherein the LAL binding site has the sequence GGGGGT (SEQ ID NO: 179).

26. The nucleic acid of any one of claims 21 to 25, wherein the binding of an LAL to the LAL binding site promotes expression of the polyketide synthase.

27. The nucleic acid of any one of claims 14 to 26, wherein the nucleic acid further encodes a nonribosomal peptide synthase.

28. The nucleic acid of any one of claims 14 to 27, wherein the nucleic acid further encodes a first P450 enzyme.

29. The nucleic acid of claim 28, wherein the nucleic acid further encodes a second P450 enzyme.

30. An expression vector comprising a nucleic acid of any one of claims 14 to 29.

31. The expression vector of claim 30, wherein the expression vector is an artificial chromosome.

32. The expression vector of claim 31, wherein the artificial chromosome is a bacterial artificial chromosome.

33. A host cell comprising an expression vector of any one of claims 30 to 32.

34. A host cell comprising a polyketide synthase of any one of claims 1 to 13, wherein the polyketide is heterologous to the host cell.

35. The host cell of claim 33 or 34, wherein the host cell naturally lacks an LAL.

36. The host cell of any one of claims 33 to 35, wherein the host cell naturally lacks an LAL binding site.

37. The host cell of any one of claims 33 to 36, wherein the host cell comprises an LAL capable of binding to an LAL binding site and regulating expression of a polyketide synthase.

38. The host cell of claim 37, wherein the LAL is heterologous.

39. The host cell of claim 37 or 38, wherein the LAL comprises a portion having at least 80% identity to the sequence of SEQ ID NO: 177.

40. The host cell of any one of claims 33 to 39, wherein the host cell is a bacterium.

41. The host cell of claim 40, wherein the bacterium is an actinobacterium.

42. The host cell of claim 41, wherein the actinobacterium is Streptomyces ambofaciens, Streptomyces hygroscopicus, or Streptomyces malayensis.

43. The host cell of claim 42, wherein the actinobaceterium is S1391, S1496, or S2441.

44. The host cell of any one of claims 33 to 43, wherein the host cell has been modified to enhance expression of a polyketide synthase.

45. The host cell of claim 44, wherein the host cell has been modified to enhance expression of a compound-producing protein by (i) deletion of an endogenous gene cluster which expresses a compound-producing protein; (ii) insertion of a heterologous gene cluster which expresses a compound-producing protein; (iii) exposure of the host cell to an antibiotic challenge; and/or (iv) introduction of a heterologous promoter that results in an at least 2-fold increase in expression of a compound compared to the homologous promoter.

46. A method of producing a polyketide, the method comprising culturing a host cell of any one of claims 33 to 45 under suitable conditions.

47. A method of producing a polyketide, the method comprising culturing a host cell engineered to express a polyketide synthase of any one of claims 1 to 13 under conditions suitable for polyketide synthase to produce a polyketide.

48. A method of producing a compound, the method comprising:
a) providing a parent polyketide synthase sequence capable of producing a compound;
(b) determining the compatibility of at least one module of a second polyketide synthase with at least two modules of the parent polyketide synthase;
(c) producing a nucleic acid encoding a modified polyketide synthase, wherein the modified polyketide synthase comprises at least one module of a second polyketide synthase which has been determined to be compatible with the at least two modules of the parent polyketide synthase.

49. A method of producing a compound, the method comprising:
(a) providing a parent nucleic acid encoding a parent polyketide synthase;
(b) modifying the parent nucleic acid to create a modified nucleic acid encoding a modified polyketide synthase capable of producing a compound, wherein the modification produces a modified polyketide synthase comprising at least one heterologous module.

50. A method of producing a compound, the method comprising:
(a) providing a parent polynucleotide sequence capable of producing a compound;
(b) identifying one or more heterologous modules suitable for replacement of one or more modules in the parent polynucleotide sequence;
(c) producing a nucleic acid encoding a modified polyketide synthase, wherein the modified polyketide synthase comprises at least one heterologous module identified in step (b).

51. A method of producing a plurality of polynucleotides, wherein each of the plurality of polynucleotides corresponds to an engineered polyketide synthase, and wherein each of the plurality of polynucleotides comprises one or more heterologous modules with altered enzymatic activity relative to a reference polyketide, wherein the method comprises:
(a) providing a parent polynucleotide sequence encoding a polyketide synthase;
(b) identifying one or more modules for replacement in the parent polynucleotide sequence;
(c) identifying two or more heterologous modules suitable for replacement for each of the modules identified in step (b);
(d) generating a plurality of polynucleotides, wherein each of the plurality of polynucleotides corresponds to an engineered polyketide synthase, and wherein each of the plurality of polynucleotides comprises a heterologous module selected from the two or more heterologous modules identified in step (c) in replacement of each of the one or more modules to be replaced identified in step (b).
